SEQUENCE LISTING (128 sequences)

SEQ ID NO: 1 - protein, 570 aa, Bacillus subtilis
MTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 2 - DNA, 1716 bp, Bacillus subtilis
atgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctag

SEQ ID NO: 3 - protein, 559 aa, Klebsiella pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAIEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYTFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLRAAMDVDGPAVVAIPVDYRDNPLLMGQLHLSQIL

SEQ ID NO: 4 - DNA, 2055 bp, Klebsiella pneumoniae
tcgaccacggggtgctgaccttcggcgaaattcacaagctgatgatcgacctgcccgccgacagcgcgttcctgcaggctaatctgcatcccgataatctcgatgccgccatccgttccgtagaaagttaagggggtcacatggacaaacagtatccggtacgccagtgggcgcacggcgccgatctcgtcgtcagtcagctggaagctcagggagtacgccaggtgttcggcatccccggcgccaaaatcgacaaggtctttgattcactgctggattcctccattcgcattattccggtacgccacgaagccaacgccgcatttatggccgccgccgtcggacgcattaccggcaaagcgggcgtggcgctggtcacctccggtccgggctgttccaacctgatcaccggcatggccaccgcgaacagcgaaggcgacccggtggtggccctgggcggcgcggtaaaacgcgccgataaagcgaagcaggtccaccagagtatggatacggtggcgatgttcagcccggtcaccaaatacgccatcgaggtgacggcgccggatgcgctggcggaagtggtctccaacgccttccgcgccgccgagcagggccggccgggcagcgcgttcgttagcctgccgcaggatgtggtcgatggcccggtcagcggcaaagtgctgccggccagcggggccccgcagatgggcgccgcgccggatgatgccatcgaccaggtggcgaagcttatcgcccaggcgaagaacccgatcttcctgctcggcctgatggccagccagccggaaaacagcaaggcgctgcgccgtttgctggagaccagccatattccagtcaccagcacctatcaggccgccggagcggtgaatcaggataacttctctcgcttcgccggccgggttgggctgtttaacaaccaggccggggaccgtctgctgcagctcgccgacctggtgatctgcatcggctacagcccggtggaatacgaaccggcgatgtggaacagcggcaacgcgacgctggtgcacatcgacgtgctgcccgcctatgaagagcgcaactacaccccggatgtcgagctggtgggcgatatcgccggcactctcaacaagctggcgcaaaatatcgatcatcggctggtgctctccccgcaggcggcggagatcctccgcgaccgccagcaccagcgcgagctgctggaccgccgcggcgcgcagctcaaccagtttgccctgcatcccctgcgcatcgttcgcgccatgcaggatatcgtcaacagcgacgtcacgttgaccgtggacatgggcagcttccatatctggattgcccgctacctgtacacgttccgcgcccgtcaggtgatgatctccaacggccagcagaccatgggcgtcgccctgccctgggctatcggcgcctggctggtcaatcctgagcgcaaagtggtctccgtctccggcgacggcggcttcctgcagtcgagcatggagctggagaccgccgtccgcctgaaagccaacgtgctgcatcttatctgggtcgataacggctacaacatggtcgctatccaggaagagaaaaaatatcagcgcctgtccggcgtcgagtttgggccgatggattttaaagcctatgccgaatccttcggcgcgaaagggtttgccgtggaaagcgccgaggcgctggagccgaccctgcgcgcggcgatggacgtcgacggcccggcggtagtggccatcccggtggattatcgcgataacccgctgctgatgggccagctgcatctgagtcagattctgtaagtcatcacaataaggaaagaaaaatgaaaaaagtcgcacttgttaccggcgccggccaggggattggtaaagctatcgcccttcgtctggtgaaggatggatttgccgtggccattgccgattataacgacgccaccgccaaagcggtcgcctccgaaatcaaccaggccggcggccgcgccatggcggtgaaagtggatgtttctgaccgcgaccaggtatttgccgccgtcga

SEQ ID NO: 5 - protein, 554 aa, Lactococcus lactis
MSEKQFGANLVVDSLINHKVKYVFGIPGAKIDRVFDLLENEEGPQMVVTRHEQGAAFMAQAVGRLTGEPGVVVVTSGPGVSNLATPLLTATSEGDAILAIGGQVKRSDRLKRAHQSMDNAGMMQSATKYSAEVLDPNTLSESIANAYRIAKSGHPGATFLSIPQDVTDAEVSIKAIQPLSDPKMGNASIDDINYLAQAIKNAVLPVILVGAGASDAKVASSLRNLLTHVNIPVVETFQGAGVISHDLEHTFYGRIGLFRNQPGDMLLKRSDLVIAVGYDPIEYEARNWNAEIDSRIIVIDNAIAEIDTYYQPERELIGDIAATLDNLLPAVRGYKIPKGTKDYLDGLHEVAEQHEFDTENTEEGRMHPLDLVSTFQEIVKDDETVTVDVGSLYIWMARHFKSYEPRHLLFSNGMQTLGVALPWAITAALLRPGKKVYSHSGDGGFLFTGQELETAVRLNLPIVQIIWNDGHYDMVKFQEEMKYGRSAAVDFGYVDYVKYAEAMRAKGYRAHSKEELAEILKSIPDTTGPVVIDVPLDYSDNIKLAEKLLPEEFY

SEQ ID NO: 6 - DNA, 3220 bp, Lactococcus lactis
tagatccggaaacaactgattacctgagttaacttagcagaaattgcagaagataacggtaatttggatgaagcattaaattacctttatcaaattccggtgaatgatgaaaattatattgctgctttaatcaaaattgctgacttatatcaatttgaagttgattttgaaacagcaatttctaagttagaagaagcaagagaattatcggattctcctctgattacttttgctttggctgagtcctactttgaacaaggtgattattcagctgccattaccgaatatgcaaaactttcagaacgaaaaattttacatgaaacaaaaatttctatttatcaaagaattggtgactcttatgcccaattaggtaattttgagaatgccatatcatttcttgaaaaatcacttgaatttgatgaaaaaccggaaaccttgtataaaattgctcttctttatggagaaactcataatgaaacaagagccattgctaatttcaaacggttagaaaaaatggatgttgaatttttgaactatgaattagcctatgcccaaaccctagaagctaatcaagaatttaaagctgcactagaaatggcaaagaaagggatgaaaaaaaatcctaatgccgttcctctcttacacttcgcttcaaaaatttgtttcaaacttaaggacaaagctgcagcagaacgttatctcgtggatgctttaaatttaccagaattacatgacgaaacagtctttttgcttgctaatttatacttcaacgaagaagattttgaagctgtcattaatcttgaagagcttttagaagatgaacatttattagctaaatggctttttgcaggagcacataaagctttggaaaatgattctgaagcggctgctttgtatgaagaactcattcaaaccaatctgtcagagaatccagagtttttagaagactatattgattttcttaaagaaattggtcaaatttctaaaacagaaccaattattgaacaatatttggaacttgttccagatgatgaaaatatgagaaatttactgacagacttaaaaaataattactgacaaagctgtcagtaattatttttattgtaagctagaaaattcaaaaacttgcgtcaaaataattgtaaaaggttctattatctgataaaatgattgtgaagtaatccaagagattatgaaatatgaattagaacaaatagaggtaaaataaaaaatgtctgagaaacaatttggggcgaacttggttgtcgatagtttgattaaccataaagtgaagtatgtatttgggattccaggagcaaaaattgaccgggtttttgatttattagaaaatgaagaaggccctcaaatggtcgtgactcgtcatgagcaaggagctgctttcatggctcaagctgtcggtcgtttaactggcgaacctggtgtagtagttgttacgagtgggcctggtgtatcaaaccttgcgactccgcttttgaccgcgacatcagaaggtgatgctattttggctatcggtggacaagttaaacgaagtgaccgtcttaaacgtgcgcaccaatcaatggataatgctggaatgatgcaatcagcaacaaaatattcagcagaagttcttgaccctaatacactttctgaatcaattgccaacgcttatcgtattgcaaaatcaggacatccaggtgcaactttcttatcaatcccccaagatgtaacggatgccgaagtatcaatcaaagccattcaaccactttcagaccctaaaatggggaatgcctctattgatgacattaattatttagcacaagcaattaaaaatgctgtattgccagtaattttggttggagctggtgcttcagatgctaaagtcgcttcatccttgcgtaatctattgactcatgttaatattcctgtcgttgaaacattccaaggtgcaggggttatttcacatgatttagaacatactttttatggacgtatcggtcttttccgcaatcaaccaggcgatatgcttctgaaacgttctgaccttgttattgctgttggttatgacccaattgaatatgaagctcgtaactggaatgcagaaattgatagtcgaattatcgttattgataatgccattgctgaaattgatacttactaccaaccagagcgtgaattaattggtgatatcgcagcaacattggataatcttttaccagctgttcgtggctacaaaattccaaaaggaacaaaagattatctcgatggccttcatgaagttgctgagcaacacgaatttgatactgaaaatactgaagaaggtagaatgcaccctcttgatttggtcagcactttccaagaaatcgtcaaggatgatgaaacagtaaccgttgacgtaggttcactctacatttggatggcacgtcatttcaaatcatacgaaccacgtcatctcctcttctcaaacggaatgcaaacactcggagttgcacttccttgggcaattacagccgcattgttgcgcccaggtaaaaaagtttattcacactctggtgatggaggcttccttttcacagggcaagaattggaaacagctgtacgtttgaatcttccaatcgttcaaattatctggaatgacggccattatgatatggttaaattccaagaagaaatgaaatatggtcgttcagcagccgttgattttggctatgttgattacgtaaaatatgctgaagcaatgagagcaaaaggttaccgtgcacacagcaaagaagaacttgctgaaattctcaaatcaatcccagatactactggaccggtggtaattgacgttcctttggactattctgataacattaaattagcagaaaaattattgcctgaagagttttattgattacaatcaagcaatttgtggcataacaaaataaaagaagaaggccttgaacacctaagcgttcagggcctttttttgtgaaataaattagatgaaatttacaatgagttttgtgaaactagcttctagtttgtgaaaaattgcctataattgccgaataaaaatacccatttaccactccaagaggatgcttcaaattagctaaatacccgttttagaggatgcgtaaaaacaacaaaagaggatgagtatagaacgataaaacttttttatgataggttgagagaattgaatataaaatataataagtagaaggcagcaatt

SEQ ID NO: 7 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 8 - DNA, 1476 bp, Escherichia coli
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 9 - protein, 395 aa, Saccharomyces cerevisiae
MLRTQAARLICNSRVITAKRTFALATRAAAYSRPAARFVKPMITTRGLKQINFGGTVETVYERADWPREKLLDYFKNDTFALIGYGSQGYGQGLNLRDNGLNVIIGVRKDGASWKAAIEDGWVPGKNLFTVEDAIKRGSYVMNLLSDAAQSETWPAIKPLLTKGKTLYFSHGFSPVFKDLTHVEPPKDLDVILVAPKGSGRTVRSLFKEGRGINSSYAVWNDVTGKAHEKAQALAVAIGSGYVYQTTFEREVNSDLYGERGCLMGGIHGMFLAQYDVLRENGHSPSEAFNETVEEATQSLYPLIGKYGMDYMYDACSTTARRGALDWYPIFKNALKPVFQDLYESTKNGTETKRSLEFNSQPDYREKLEKELDTIRNMEIWKVGKEVRKLRPENQ

SEQ ID NO: 10 - DNA, 1188 bp, Saccharomyces cerevisiae
atgttgagaactcaagccgccagattgatctgcaactcccgtgtcatcactgctaagagaacctttgctttggccacccgtgctgctgcttacagcagaccagctgcccgtttcgttaagccaatgatcactacccgtggtttgaagcaaatcaacttcggtggtactgttgaaaccgtctacgaaagagctgactggccaagagaaaagttgttggactacttcaagaacgacacttttgctttgatcggttacggttcccaaggttacggtcaaggtttgaacttgagagacaacggtttgaacgttatcattggtgtccgtaaagatggtgcttcttggaaggctgccatcgaagacggttgggttccaggcaagaacttgttcactgttgaagatgctatcaagagaggtagttacgttatgaacttgttgtccgatgccgctcaatcagaaacctggcctgctatcaagccattgttgaccaagggtaagactttgtacttctcccacggtttctccccagtcttcaaggacttgactcacgttgaaccaccaaaggacttagatgttatcttggttgctccaaagggttccggtagaactgtcagatctttgttcaaggaaggtcgtggtattaactcttcttacgccgtctggaacgatgtcaccggtaaggctcacgaaaaggcccaagctttggccgttgccattggttccggttacgtttaccaaaccactttcgaaagagaagtcaactctgacttgtacggtgaaagaggttgtttaatgggtggtatccacggtatgttcttggctcaatacgacgtcttgagagaaaacggtcactccccatctgaagctttcaacgaaaccgtcgaagaagctacccaatctctatacccattgatcggtaagtacggtatggattacatgtacgatgcttgttccaccaccgccagaagaggtgctttggactggtacccaatcttcaagaatgctttgaagcctgttttccaagacttgtacgaatctaccaagaacggtaccgaaaccaagagatctttggaattcaactctcaacctgactacagagaaaagctagaaaaggaattagacaccatcagaaacatggaaatctggaaggttggtaaggaagtcagaaagttgagaccagaaaaccaataa

SEQ ID NO: 11 - protein, 330 aa, Methanococcus maripaludis
MKVFYDSDFKLDALKEKTIAVIGYGSQGRAQSLNMKDSGLNVVVGLRKNGASWNNAKADGHNVMTIEEAAEKADIIHILIPDELQAEVYESQIKPYLKEGKTLSFSHGFNIHYGFIVPPKGVNVVLVAPKSPGKMVRRTYEEGFGVPGLICIEIDATNNAFDIVSAMAKGIGLSRAGVIQTTFKEETETDLFGEQAVLCGGVTELIKAGFETLVEAGYAPEMAYFETCHELKLIVDLIYQKGFKNMWNDVSNTAEYGGLTRRSRIVTADSKAAMKEILREIQDGRFTKEFLLEKQVSYAHLKSMRRLEGDLQIEEVGAKLRKMCGLEKEE

SEQ ID NO: 12 - DNA, 993 bp, Methanococcus maripaludis
atgaaggtattctatgactcagattttaaattagatgctttaaaagaaaaaacaattgcagtaatcggttatggaagtcaaggtagggcacagtccttaaacatgaaagacagcggattaaacgttgttgttggtttaagaaaaaacggtgcttcatggaacaacgctaaagcagacggtcacaatgtaatgaccattgaagaagctgctgaaaaagcggacatcatccacatcttaatacctgatgaattacaggcagaagtttatgaaagccagataaaaccatacctaaaagaaggaaaaacactaagcttttcacatggttttaacatccactatggattcattgttccaccaaaaggagttaacgtggttttagttgctccaaaatcacctggaaaaatggttagaagaacatacgaagaaggtttcggtgttccaggtttaatctgtattgaaattgatgcaacaaacaacgcatttgatattgtttcagcaatggcaaaaggaatcggtttatcaagagctggagttatccagacaactttcaaagaagaaacagaaactgaccttttcggtgaacaagctgttttatgcggtggagttaccgaattaatcaaggcaggatttgaaacactcgttgaagcaggatacgcaccagaaatggcatactttgaaacctgccacgaattgaaattaatcgttgacttaatctaccaaaaaggattcaaaaacatgtggaacgatgtaagtaacactgcagaatacggcggacttacaagaagaagcagaatcgttacagctgattcaaaagctgcaatgaaagaaatcttaagagaaatccaagatggaagattcacaaaagaattccttctcgaaaaacaggtaagctatgctcatttaaaatcaatgagaagactcgaaggagacttacaaatcgaagaagtcggcgcaaaattaagaaaaatgtgcggtcttgaaaaagaagaataa

SEQ ID NO: 13 - protein, 342 aa, Bacillus subtilis
MVKVYYNGDIKENVLAGKTVAVIGYGSQGHAHALNLKESGVDVIVGVRQGKSFTQAQEDGHKVFSVKEAAAQAEIIMVLLPDEQQQKVYEAEIKDELTAGKSLVFAHGFNVHFHQIVPPADVDVFLVAPKGPGHLVRRTYEQGAGVPALFAIYQDVTGEARDKALAYAKGIGGARAGVLETTFKEETETDLFGEQAVLCGGLSALVKAGFETLTEAGYQPELAYFECLHELKLIVDLMYEEGLAGMRYSISDTAQWGDFVSGPRVVDAKVKESMKEVLKDIQNGTFAKEWIVENQVNRPRFNAINASENEHQIEVVGRKLREMMPFVKQGKKKEAVVSVAQN

SEQ ID NO: 14 - DNA, 1476 bp, Bacillus subtilis; identical to SEQ ID NO: 8

SEQ ID NO: 15 - protein, 343 aa, Anaerostipes caccae
MEECKMAKIYYQEDCNLSLLDGKTIAVIGYGSQGHAHALNAKESGCNVIIGLYEGAKEWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 16 - protein, 343 aa, Anaerostipes caccae
MEECKMAKIYYQEDCNLSLLDGKTIAVIGYGSQGHAHALNAKESGCNVIIGLYEGAKDWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 17 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 18 - DNA, 1851 bp, Escherichia coli
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 19 - protein, 585 aa, Saccharomyces cerevisiae
MGLLTKVATSRQFSTTRCVAKKLNKYSYIITEPKGQGASQAMLYATGFKKEDFKKPQVGVGSCWWSGNPCNMHLLDLNNRCSQSIEKAGLKAMQFNTIGVSDGISMGTKGMRYSLQSREIIADSFETIMMAQHYDANIAIPSCDKNMPGVMMAMGRHNRPSIMVYGGTILPGHPTCGSSKISKNIDIVSAFQSYGEYISKQFTEEEREDVVEHACPGPGSCGGMYTANTMASAAEVLGLTIPNSSSFPAVSKEKLAECDNIGEYIKKTMELGILPRDILTKEAFENAITYVVATGGSTNAVLHLVAVAHSAGVKLSPDDFQRISDTTPLIGDFKPSGKYVMADLINVGGTQSVIKYLYENNMLHGNTMTVTGDTLAERAKKAPSLPEGQEIIKPLSHPIKANGHLQILYGSLAPGGAVGKITGKEGTYFKGRARVFEEEGAFIEALERGEIKKGEKTVVVIRYEGPRGAPGMPEMLKPSSALMGYGLGKDVALLTDGRFSGGSHGFLIGHIVPEAAEGGPIGLVRDGDEIIIDADNNKIDLLVSDKEMAQRKQSWVAPPPRYTRGTLSKYAKLVSNASNGCVLDA

SEQ ID NO: 20 - DNA, 1131 bp, Saccharomyces cerevisiae
atgaccttggcacccctagacgcctccaaagttaagataactaccacacaacatgcatctaagccaaaaccgaacagtgagttagtgtttggcaagagcttcacggaccacatgttaactgcggaatggacagctgaaaaagggtggggtaccccagagattaaaccttatcaaaatctgtctttagacccttccgcggtggttttccattatgcttttgagctattcgaagggatgaaggcttacagaacggtggacaacaaaattacaatgtttcgtccagatatgaatatgaagcgcatgaataagtctgctcagagaatctgtttgccaacgttcgacccagaagagttgattaccctaattgggaaactgatccagcaagataagtgcttagttcctgaaggaaaaggttactctttatatatcaggcctacattaatcggcactacggccggtttaggggtttccacgcctgatagagccttgctatatgtcatttgctgccctgtgggtccttattacaaaactggatttaaggcggtcagactggaagccactgattatgccacaagagcttggccaggaggctgtggtgacaagaaactaggtgcaaactacgccccctgcgtcctgccacaattgcaagctgcttcaaggggttaccaacaaaatttatggctatttggtccaaataacaacattactgaagtcggcaccatgaatgcttttttcgtgtttaaagatagtaaaacgggcaagaaggaactagttactgctccactagacggtaccattttggaaggtgttactagggattccattttaaatcttgctaaagaaagactcgaaccaagtgaatggaccattagtgaacgctacttcactataggcgaagttactgagagatccaagaacggtgaactacttgaagcctttggttctggtactgctgcgattgtttctcccattaaggaaatcggctggaaaggcgaacaaattaatattccgttgttgcccggcgaacaaaccggtccattggccaaagaagttgcacaatggattaatggaatccaatatggcgagactgagcatggcaattggtcaagggttgttactgatttgaactga

SEQ ID NO: 21 - protein, 550 aa, Methanococcus maripaludis
MISDNVKKGVIRTPNRALLKACGYTDEDMEKPFIGIVNSFTEVVPGHIHLRTLSEAAKHGVYANGGTPFEFNTIGICDGIAMGHEGMKYSLPSREIIADAVESMARAHGFDGLVLIPTCDKIVPGMIMGALRLNIPFIVVTGGPMLPGEFQGKKYELISLFEGVGEYQVGKITEEELKCIEDCACSGAGSCAGLYTANSMACLTEALGLSLPMCATTHAVDAQKVRLAKKSGSKIVDMVKEDLKPTDILTKEAFENAILVDLALGGSTNTTLHIPAIANEIENKFITLDDFDRLSDEVPHIASIKPGGEHYMIDLHNAGGIPAVLNVLKEKIRDTKTVDGRSILEIAESVKYINYDVIRKVEAPVHETAGLRVLKGNLAPNGCVVKIGAVHPKMYKHDGPAKVYNSEDEAISAILGGKIVEGDVIVIRYEGPSGGPGMREMLSPTSAICGMGLDDSVALITDGRFSGGSRGPCIGHVSPEAAAGGVIAAIENGDIIKIDMIEKEINVDLDESVIKERLSKLGEFEPKIKKGYLSRYSKLVSSADEGAVLK

SEQ ID NO: 22 - DNA, 1653 bp, Methanococcus maripaludis
atgataagtgataacgtcaaaaagggagttataagaactccaaaccgagctcttttaaaggcttgcggatatacagacgaagacatggaaaaaccatttattggaattgtaaacagctttacagaagttgttcccggccacattcacttaagaacattatcagaagcggctaaacatggtgtttatgcaaacggtggaacaccatttgaatttaataccattggaatttgcgacggtattgcaatgggccacgaaggtatgaaatactctttaccttcaagagaaattattgcagacgctgttgaatcaatggcaagagcacatggatttgatggtcttgttttaattcctacgtgtgataaaatcgttcctggaatgataatgggtgctttaagactaaacattccatttattgtagttactggaggaccaatgcttcccggagaattccaaggtaaaaaatacgaacttatcagcctttttgaaggtgtcggagaataccaagttggaaaaattactgaagaagagttaaagtgcattgaagactgtgcatgttcaggtgctggaagttgtgcagggctttacactgcaaacagtatggcctgccttacagaagctttgggactctctcttccaatgtgtgcaacaacgcatgcagttgatgcccaaaaagttaggcttgctaaaaaaagtggctcaaaaattgttgatatggtaaaagaagacctaaaaccaacagacatattaacaaaagaagcttttgaaaatgctattttagttgaccttgcacttggtggatcaacaaacacaacattacacattcctgcaattgcaaatgaaattgaaaataaattcataactctcgatgactttgacaggttaagcgatgaagttccacacattgcatcaatcaaaccaggtggagaacactacatgattgatttacacaatgctggaggtattcctgcggtattgaacgttttaaaagaaaaaattagagatacaaaaacagttgatggaagaagcattttggaaatcgcagaatctgttaaatacataaattacgacgttataagaaaagtggaagctccggttcacgaaactgctggtttaagggttttaaagggaaatcttgctccaaacggttgcgttgtaaaaatcggtgcagtacatccgaaaatgtacaaacacgatggacctgcaaaagtttacaattccgaagatgaagcaatttctgcgatacttggcggaaaaattgtagaaggggacgttatagtaatcagatacgaaggaccatcaggaggccctggaatgagagaaatgctctccccaacttcagcaatctgtggaatgggtcttgatgacagcgttgcattgattactgatggaagattcagtggtggaagtaggggcccatgtatcggacacgtttctccagaagctgcagctggcggagtaattgctgcaattgaaaacggggatatcatcaaaatcgacatgattgaaaaagaaataaatgttgatttagatgaatcagtcattaaagaaagactctcaaaactgggagaatttgagcctaaaatcaaaaaaggctatttatcaagatactcaaaacttgtctcatctgctgacgaaggggcagttttaaaataa

SEQ ID NO: 23 - protein, 558 aa, Bacillus subtilis
MAELRSNMITQGIDRAPHRSLLRAAGVKEEDFGKPFIAVCNSYIDIVPGHVHLQEFGKIVKEAIREAGGVPFEFNTIGVDDGIAMGHIGMRYSLPSREIIADSVETVVSAHWFDGMVCIPNCDKITPGMLMAAMRINIPTIFVSGGPMAAGRTSDGRKISLSSVFEGVGAYQAGKINENELQELEQFGCPTCGSCSGMFTANSMNCLSEALGLALPGNGTILATSPERKEFVRKSAAQLMETIRKDIKPRDIVTVKAIDNAFALDMALGGSTNTVLHTLALANEAGVEYSLERINEVAERVPHLAKLAPASDVFIEDLHEAGGVSAALNELSKKEGALHLDALTVTGKTLGETIAGHEVKDYDVIHPLDQPFTEKGGLAVLFGNLAPDGAIIKTGGVQNGITRHEGPAVVFDSQDEALDGIINRKVKEGDVVIIRYEGPKGGPGMPEMLAPTSQIVGMGLGPKVALITDGRFSGASRGLSIGHVSPEAAEGGPLAFVENGDHIIVDIEKRILDVQVPEEEWEKRKANWKGFEPKVKTGYLARYSKLVTSANTGGIMKI

SEQ ID NO: 24 - DNA, 1677 bp, Bacillus subtilis
atggcagaattacgcagtaatatgatcacacaaggaatcgatagagctccgcaccgcagtttgcttcgtgcagcaggggtaaaagaagaggatttcggcaagccgtttattgcggtgtgtaattcatacattgatatcgttcccggtcatgttcacttgcaggagtttgggaaaatcgtaaaagaagcaatcagagaagcagggggcgttccgtttgaatttaataccattggggtagatgatggcatcgcaatggggcatatcggtatgagatattcgctgccaagccgtgaaattatcgcagactctgtggaaacggttgtatccgcacactggtttgacggaatggtctgtattccgaactgcgacaaaatcacaccgggaatgcttatggcggcaatgcgcatcaacattccgacgatttttgtcagcggcggaccgatggcggcaggaagaacaagttacgggcgaaaaatctccctttcctcagtattcgaaggggtaggcgcctaccaagcagggaaaatcaacgaaaacgagcttcaagaactagagcagttcggatgcccaacgtgcgggtcttgctcaggcatgtttacggcgaactcaatgaactgtctgtcagaagcacttggtcttgctttgccgggtaatggaaccattctggcaacatctccggaacgcaaagagtttgtgagaaaatcggctgcgcaattaatggaaacgattcgcaaagatatcaaaccgcgtgatattgttacagtaaaagcgattgataacgcgtttgcactcgatatggcgctcggaggttctacaaataccgttcttcatacccttgcccttgcaaacgaagccggcgttgaatactctttagaacgcattaacgaagtcgctgagcgcgtgccgcacttggctaagctggcgcctgcatcggatgtgtttattgaagatcttcacgaagcgggcggcgtttcagcggctctgaatgagctttcgaagaaagaaggagcgcttcatttagatgcgctgactgttacaggaaaaactcttggagaaaccattgccggacatgaagtaaaggattatgacgtcattcacccgctggatcaaccattcactgaaaagggaggccttgctgttttattcggtaatctagctccggacggcgctatcattaaaacaggcggcgtacagaatgggattacaagacacgaagggccggctgtcgtattcgattctcaggacgaggcgcttgacggcattatcaaccgaaaagtaaaagaaggcgacgttgtcatcatcagatacgaagggccaaaaggcggacctggcatgccggaaatgctggcgccaacatcccaaatcgttggaatgggactcgggccaaaagtggcattgattacggacggacgtttttccggagcctcccgtggcctctcaatcggccacgtatcacctgaggccgctgagggcgggccgcttgcctttgttgaaaacggagaccatattatcgttgatattgaaaaacgcatcttggatgtacaagtgccagaagaagagtgggaaaaacgaaaagcgaactggaaaggttttgaaccgaaagtgaaaaccggctacctggcacgttattctaaacttgtgacaagtgccaacaccggcggtattatgaaaatctag

SEQ ID NO: 25 - protein, 547 aa, Lactococcus lactis
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 26 - DNA, 1828 bp, Lactococcus lactis
tttaaataagtcaatatcgttgacttatttagaagaaagagttattctttaaatgtcaagttagttgactaaattaaatataaaatatggaggaatgtgatgtatacagtaggagattacctgttagaccgattacacgagttgggaattgaagaaatttttggagttcctggtgactataacttacaatttttagatcaaattatttcacgcgaagatatgaaatggattggaaatgctaatgaattaaatgcttcttatatggctgatggttatgctcgtactaaaaaagctgccgcatttctcaccacatttggagtcggcgaattgagtgcgatcaatggactggcaggaagttatgccgaaaatttaccagtagtagaaattgttggttcaccaacttcaaaagtacaaaatgacggaaaatttgtccatcatacactagcagatggtgattttaaacactttatgaagatgcatgaacctgttacagcagcgcggactttactgacagcagaaaatgccacatatgaaattgaccgagtactttctcaattactaaaagaaagaaaaccagtctatattaacttaccagtcgatgttgctgcagcaaaagcagagaagcctgcattatctttagaaaaagaaagctctacaacaaatacaactgaacaagtgattttgagtaagattgaagaaagtttgaaaaatgcccaaaaaccagtagtgattgcaggacacgaagtaattagttttggtttagaaaaaacggtaactcagtttgtttcagaaacaaaactaccgattacgacactaaattttggtaaaagtgctgttgatgaatctttgccctcatttttaggaatatataacgggaaactttcagaaatcagtcttaaaaattttgtggagtccgcagactttatcctaatgcttggagtgaagcttacggactcctcaacaggtgcattcacacatcatttagatgaaaataaaatgatttcactaaacatagatgaaggaataattttcaataaagtggtagaagattttgattttagagcagtggtttcttctttatcagaattaaaaggaatagaatatgaaggacaatatattgataagcaatatgaagaatttattccatcaagtgctcccttatcacaagaccgtctatggcaggcagttgaaagtttgactcaaagcaatgaaacaatcgttgctgaacaaggaacctcattttttggagcttcaacaattttcttaaaatcaaatagtcgttttattggacaacctttatggggttctattggatatacttttccagcggctttaggaagccaaattgcggataaagagagcagacaccttttatttattggtgatggttcacttcaacttaccgtacaagaattaggactatcaatcagagaaaaactcaatccaatttgttttatcataaataatgatggttatacagttgaaagagaaatccacggacctactcaaagttataacgacattccaatgtggaattactcgaaattaccagaaacatttggagcaacagaagatcgtgtagtatcaaaaattgttagaacagagaatgaatttgtgtctgtcatgaaagaagcccaagcagatgtcaatagaatgtattggatagaactagttttggaaaaagaagatgcgccaaaattactgaaaaaaatgggtaaattatttgctgagcaaaataaatagatatcaacggatgatgaaaagtaaaatagacaaagtccaataattttataaaaagtaaaaacattaggattttcctaatgttttt

SEQ ID NO: 27 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 28 - DNA, 1954 bp, Lactococcus lactis
ctagagttttctttagtcataattcactccttttattagtctattatacttgataattcaaataagtcaatatcgttgacttatttaaagaaaagcgttattctataaatgtcaagttgattgaccaatatataataaaatatggaggaatgcgatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataatttataaatagtaaaaaacattaggaaatacctaatgtttttttgttgactaaatcaatccctctttatatagaaaaccttagtttctcaaagacaacttaattaagcctgccaaattggaactcgcaaaatgtaatctatcctctgctccta

SEQ ID NO: 29 - protein, 550 aa, Salmonella typhimurium
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVLQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 30 - DNA, 1653 bp, Salmonella typhimurium
ttatcccccgttgcgggcttccagcgcccgggtcacggtacgcagtaattccggcagatcggcttttggcaacatcacttcaataaatgacagacgttgtgggcgcgccaaccgttcgaggacctctgccagttggatagcctgcgtcacccgccagcactccgcctgttgcgccgcgtttagcgccggtggtatctgcgtccagttccagctcgcgatgtcgttataccgctgggccgcgccgtgaatggcgcgctctacggtatagccgtcattgttgagcagcaggatgaccggcgcctgcccgtcgcgtaacatcgagcccatctcctgaatcgtgagctgcgccgcgccatcgccgataatcagaatcacccgccgatcgggacaggcggtttgcgcgccaaacgcggcgggcaaggaatagccgatagacccccacagcggctgtaacacaacttccgcgccgtcaggaagcgacagcgcggcagcgccaaaagctgctgtcccctggtcgacaaggataatatctccgggtttgagatactgctgtaaggtttgccagaagctttcctgggtcagttctcctttatcaatccgcactggctgtccggcggaacgcgtcggcggcggcgcaaaagcgcattccaggcacagttcgcgcagcgtagacaccgcctgcgccatcgggaggttgaaccaggtttcgccgatgcgcgacgcgtaaggctgaatctccagcgtgcgttccgccggtaattgttgggtaaatccggccgtaagggtatcgacaaaacgggtgccgacgcagataaccctatcggcgtcctctatggcctgacgcacttctttgctgctggcgccagcgctataggtgccaacgaagttcgggtgctgttcatcaaaaagccccttccccatcagtagtgtcgcatgagcgatgggcgtttccgccatccagcgctgcaacagtggtcgtaaaccaaaacgcccggcaagaaagtcggccaatagcgcaatgcgccgactgttcatcaggcactgacgggcgtgataacgaaaggccgtctccacgccgctttgcgcttcatgcacgggcaacgccagcgcctgcgtaggtgggatggccgtttttttcgccacatcggcgggcaacatgatgtatcctggcctgcgtgcggcaagcatttcacccaacacgcggtcaatctcgaaacaggcgttctgttcatctaatattgcgctggcagcggatatcgcctgactcatgcgataaaaatgacgaaaatcgccgtcaccgagggtatggtgcatcaattcgccacgctgctgcgcagcgctacagggcgcgccgacgatatgcaagaccgggacatattccgcgtaactgcccgcgataccgttaatagcgctaagttctcccacgccaaaggtggtgagtagcgctccagcgcccgacatgcgcgcatagccgtccgcggcataagcggcgttcagctcattggcgcatcccacccaacgcagggtcgggtggtcaatcacatggtcaagaaactgcaagttataatcgcccggtacgccaaaaagatggccaatgccgcatcctgccagtctgtccagcaaatagtcggccacggtataggggttttgcat

SEQ ID NO: 31 - protein, 554 aa, Clostridium acetobutylicum
MKSEYTIGRYLLDRLSELGIRHIFGVPGDYNLSFLDYIMEYKGIDWVGNCNELNAGYAADGYARINGIGAILTTFGVGELSAINAIAGAYAEQVPVVKITGIPTAKVRDNGLYVHHTLGDGRFDHFFEMFREVTVAEALLSEENAAQEIDRVLISCWRQKRPVLINLPIDVYDKPINKPLKPLLDYTISSNKEAACEFVTEIVPIINRAKKPVILADYGVYRYQVQHVLKNLAEKTGFPVATLSMGKGVFNEAHPQFIGVYNGDVSSPYLRQRVDEADCIISVGVKLTDSTTGGFSHGFSKRNVIHIDPFSIKAKGKKYAPITMKDALTELTSKIEHRNFEDLDIKPYKSDNQKYFAKEKPITQKRFFERIAHFIKEKDVLLAEQGTCFFGASTIQLPKDATFIGQPLWGSIGYTLPALLGSQLADQKRRNILLIGDGAFQMTAQEISTMLRLQIKPIIFLINNDGYTIERAIHGREQVYNNIQMWRYHNVPKVLGPKECSLTFKVQSETELEKALLVADKDCEHLIFIEVVMDRYDKPEPLERLSKRFANQNN

SEQ ID NO: 32 - DNA, 1665 bp, Clostridium acetobutylicum
ttgaagagtgaatacacaattggaagatatttgttagaccgtttatcagagttgggtattcggcatatctttggtgtacctggagattacaatctatcctttttagactatataatggagtacaaagggatagattgggttggaaattgcaatgaattgaatgctgggtatgctgctgatggatatgcaagaataaatggaattggagccatacttacaacatttggtgttggagaattaagtgccattaacgcaattgctggggcatacgctgagcaagttccagttgttaaaattacaggtatccccacagcaaaagttagggacaatggattatatgtacaccacacattaggtgacggaaggtttgatcacttttttgaaatgtttagagaagtaacagttgctgaggcattactaagcgaagaaaatgcagcacaagaaattgatcgtgttcttatttcatgctggagacaaaaacgtcctgttcttataaatttaccgattgatgtatatgataaaccaattaacaaaccattaaagccattactcgattatactatttcaagtaacaaagaggctgcatgtgaatttgttacagaaatagtacctataataaatagggcaaaaaagcctgttattcttgcagattatggagtatatcgttaccaagttcaacatgtgcttaaaaacttggccgaaaaaaccggatttcctgtggctacactaagtatgggaaaaggtgttttcaatgaagcacaccctcaatttattggtgtttataatggtgatgtaagttctccttatttaaggcagcgagttgatgaagcagactgcattattagcgttggtgtaaaattgacggattcaaccacagggggattttctcatggattttctaaaaggaatgtaattcacattgatcctttttcaataaaggcaaaaggtaaaaaatatgcacctattacgatgaaagatgctttaacagaattaacaagtaaaattgagcatagaaactttgaggatttagatataaagccttacaaatcagataatcaaaagtattttgcaaaagagaagccaattacacaaaaacgtttttttgagcgtattgctcactttataaaagaaaaagatgtattattagcagaacagggtacatgcttttttggtgcgtcaaccatacaactacccaaagatgcaacttttattggtcaacctttatggggatctattggatacacacttcctgctttattaggttcacaattagctgatcaaaaaaggcgtaatattcttttaattggggatggtgcatttcaaatgacagcacaagaaatttcaacaatgcttcgtttacaaatcaaacctattatttttttaattaataacgatggttatacaattgaacgtgctattcatggtagagaacaagtatataacaatattcaaatgtggcgatatcataatgttccaaaggttttaggtcctaaagaatgcagcttaacctttaaagtacaaagtgaaactgaacttgaaaaggctcttttagtggcagataaggattgtgaacatttgatttttatagaagttgttatggatcgttatgataaacccgagcctttagaacgtctttcgaaacgttttgcaaatcaaaataattag

SEQ ID NO: 33 - DNA, 1641 bp, Clostridium acetobutylicum
atgaaacaacgtatcgggcaatacttgatcgatgccctacacgttaatggtgtcgataagatctttggagtcccaggtgatttcactttagcctttttggacgatatcataagacatgacaacgtggaatgggtgggaaatactaatgagttgaacgccgcttacgccgctgatggttacgctagagttaatggattagccgctgtatctaccacttttggggttggcgagttatctgctgtgaatggtattgctggaagttacgcagagcgtgttcctgtaatcaaaatctcaggcggtccttcatcagttgctcaacaagagggtagatatgtccaccattcattgggtgaaggaatctttgattcatattcaaagatgtacgctcacataaccgcaacaactacaatcttatccgttgacaacgcagtcgacgaaattgatagagttattcattgtgctttgaaggaaaagaggccagtgcatattcatttgcctattgacgtagccttaactgagattgaaatccctcatgcaccaaaagtttacacacacgaatcccagaacgtcgatgcttacattcaagctgttgagaaaaagttaatgtctgcaaaacaaccagtaatcatagcaggtcatgaaatcaattcattcaagttgcacgaacaactggaacagtttgtcaatcagacaaacatccctgttgcacaactttccttgggtaagtctgctttcaatgaagagaatgaacattaccttggtatctacgatggcaaaatcgcaaaggaaaatgtgagagagtacgtcgacaatgctgatgtcatattgaacataggtgccaaactgactgattctgctacagctggattttcctacaagttcgatacaaacaacataatctacattaaccataatgacttcaaagctgaagatgtgatttctgataatgtttcactgattgatcttgtgaatggcctgaattctattgactatagaaatgaaacacactacccatcttatcaaagatctgatatgaaatacgaattgaatgacgcaccacttacacaatctaactatttcaaaatgatgaacgcttttctagaaaaagatgacatcctactagctgaacaaggtacatcctttttcggcgcatatgacttatccctatacaagggaaatcagtttatcggtcagcctttatgggggtcaatagggtatacttttccatctttactaggaagtcaactagcagacatgcataggagaaacattttgcttataggcgatggtagtttacaacttactgttcaagccctaagtacaatgattagaaaggatatcaaaccaatcattttcgttatcaataacgacggttacaccgtcgaaagacttatccacggcatggaagagccatacaatgatatccaaatgtggaactacaagcaattgccagaagtatttggtggaaaagatactgtaaaagttcatgatgctaaaacctccaacgaactgaaaactgtaatggattctgttaaagcagacaaagatcacatgcatttcattgaagtgcatatggcagtagaggacgccccaaagaagttgattgatatagctaaagcctttagtgatgctaacaagtaa

SEQ ID NO: 34 - DNA, 1647 bp, Listeria grayi
atgtacaccgtcggccaatacttagtagaccgcttagaagagatcggcatcgataaggtttttggtgtcccgggtgactacaacctgacctttttggactacatccagaaccacgaaggtctgagctggcaaggtaatacgaatgaactgaatgccgcgtacgcagctgatggctatgctcgtgaacgcggtgttagcgctttggtcacgaccttcggcgttggtgagctgtccgcaatcaatggcaccgcaggtagcttcgcggagcaagttccggtgattcatatcgtgggcagcccgaccatgaatgttcagagcaacaagaaactggttcatcacagcctgggtatgggcaactttcacaacttcagcgagatggcgaaagaagtcaccgccgcaaccacgatgctgacggaagagaatgcggcgtcggagattgatcgtgttctggaaaccgccctgctggagaaacgcccagtgtacatcaatctgccgatcgacattgctcacaaggcgatcgtcaagccggcgaaagccctgcaaaccgagaagagctctggcgagcgtgaggcacaactggcggagatcattctgagccatctggagaaggctgcacagccgattgtgattgcgggtcacgagatcgcgcgcttccagatccgtgagcgtttcgagaattggattaatcaaacgaaactgccggtgaccaatctggcctacggcaagggtagcttcaacgaagaaaacgagcatttcattggtacctattatcctgcatttagcgataagaacgtgctggactacgtggataactccgactttgtcctgcactttggtggtaaaatcattgataacagcacctccagcttctcccaaggcttcaaaaccgagaacaccctgactgcggcgaacgatatcattatgctgccggacggtagcacgtattctggtattagcctgaatggcctgctggccgagctggaaaaactgaatttcacgtttgccgacaccgcagcaaagcaggcggagttggcggtgtttgagccgcaggctgaaaccccgttgaaacaggaccgttttcaccaggcggtgatgaattttctgcaagctgacgatgtcctggttacggaacagggcacctcttcttttggcttgatgctggcgcctctgaaaaagggtatgaacttgatctcgcaaacgctgtggggtagcattggttacacgttgccggcgatgattggtagccaaattgcggcaccggagcgtcgtcatatcctgagcattggtgatggtagctttcagctgactgcgcaggaaatgagcaccattttccgtgagaaactgaccccagtcatcttcatcattaacaatgatggctataccgttgagcgtgcgatccatggcgaagatgaaagctataacgacattccgacgtggaacttgcaactggtggcggaaaccttcggtggtgacgccgaaaccgtcgacactcacaatgtgttcacggagactgatttcgccaacaccctggcggcaattgacgcgacgccgcagaaagcacacgttgtggaagttcacatggaacaaatggatatgccggagagcctgcgccagatcggtctggcactgtccaagcagaatagctaa

SEQ ID NO: 35 - protein, 312 aa, Saccharomyces cerevisiae
MPATLKNSSATLKLNTGASIPVLGFGTWRSVDNNGYHSVIAALKAGYRHIDAAAIYLNEEEVGRAIKDSGVPREEIFITTKLWGTEQRDPEAALNKSLKRLGLDYVDLYLMHWPVPLKTDRVTDGNVLCIPTLEDGTVDIDTKEWNFIKTWELMQELPKTGKTKAVGVSNFSINNIKELLESPNNKVVPATNQIEIHPLLPQDELIAFCKEKGIVVEAYSPFGSANAPLLKEQAIIDMAKKHGVEPAQLIISWSIQRGYVVLAKSVNPERIVSNFKIFTLPEDDFKTISNLSKVHGTKRVVDMKWGSFPIFQ

SEQ ID NO: 36 - DNA, 939 bp, Saccharomyces cerevisiae
atgcctgctacgttaaagaattcttctgctacattaaaactaaatactggtgcctccattccagtgttgggtttcggcacttggcgttccgttgacaataacggttaccattctgtaattgcagctttgaaagctggatacagacacattgatgctgcggctatctatttgaatgaagaagaagttggcagggctattaaagattccggagtccctcgtgaggaaatttttattactactaagctttggggtacggaacaacgtgatccggaagctgctctaaacaagtctttgaaaagactaggcttggattatgttgacctatatctgatgcattggccagtgcctttgaaaaccgacagagttactgatggtaacgttctgtgcattccaacattagaagatggcactgttgacatcgatactaaggaatggaattttatcaagacgtgggagttgatgcaagagttgccaaagacgggcaaaactaaagccgttggtgtctctaatttttctattaacaacattaaagaattattagaatctccaaataacaaggtggtaccagctactaatcaaattgaaattcatccattgctaccacaagacgaattgattgccttttgtaaggaaaagggtattgttgttgaagcctactcaccatttgggagtgctaatgctcctttactaaaagagcaagcaattattgatatggctaaaaagcacggcgttgagccagcacagcttattatcagttggagtattcaaagaggctacgttgttctggccaaatcggttaatcctgaaagaattgtatccaattttaagattttcactctgcctgaggatgatttcaagactattagtaacctatccaaagtgcatggtacaaagagagtcgttgatatgaagtggggatccttcccaattttccaatga

SEQ ID NO: 37 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 38 - DNA, 1083 bp, Saccharomyces cerevisiae
ctagtctgaaaattctttgtcgtagccgactaaggtaaatctatatctaacgtcacccttttccatcctttcgaaggcttcatggacgccggcttcaccaacaggtaatgtttccacccaaattttgatatctttttcagagactaatttcaagagttggttcaattctttgatggaacctaaagcactgtaagaaatggagacagcctttaagccatatggctttagcgataacatttcgtgttgttctggtatagagattgagacaattctaccaccaaccttcatagcctttggcataatgttgaagtcaatgtcggtaagggaggaagcacagactacaatcaggtcgaaggtgtcaaagtacttttcaccccaatcaccttcttctaatgtagcaatgtagtgatcggcgcccatcttcattgcatcttctctttttctcgaagaacgagaaataacatacgtctctgcccccatggctttggaaatcaatgtacccatactgccgataccaccaagaccaactataccaacttttttacctggaccgcaaccgttacgaaccaatggagagtacacagtcaaaccaccacataatagtggagcagccaaatgtgatggaatattctctgggataggcaccacaaaatgttcatgaactctgacgtagtttgcatagccaccctgcgacacatagccgtcttcataaggctgactgtatgtggtaacaaacttggtgcagtatggttcattatcattcttacaacggtcacattccaagcatgaaaagacttgagcacctacaccaacacgttgaccgactttcaacccactgtttgacttgggccctagcttgacaactttaccaacgatttcatgaccaacgactagcggcatcttcatattgccccaatgaccagctgcacaatgaatatcactaccgcagacaccacatgcttcgatcttaatgtcaatgtcatgatcgtaaaatggttttgggtcatactttgtcttctttgggtttttccaatcttcgtgtgattgaatagcgataccttcaaatttctcaggataagacat

SEQ ID NO: 39 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 40 - protein, 387 aa, Escherichia coli; identical to SEQ ID NO: 39

SEQ ID NO: 41 - protein, 389 aa, Clostridium acetobutylicum
MLSFDYSIPTKVFFGKGKIDVIGEEIKKYGSRVLIVYGGGSIKRNGIYDRATAILKENNIAFYELSGVEPNPRITTVKKGIEICRENNVDLVLAIGGGSAIDCSKVIAAGVYYDGDTWDMVKDPSKITKVLPIASILTLSATGSEMDQIAVISNMETNEKLGVGHDDMRPKFSVLDPTYTFTVPKNQTAAGTADIMSHTFESYFSGVEGAYVQDGIAEAILRTCIKYGKIAMEKTDDYEARANLMWASSLAINGLLSLGKDRKWSCHPMEHELSAYYDITHGVGLAILTPNWMEYILNDDTLHKFVSYGINVWGIDKNKDNYEIAREAIKNTREYFNSLGIPSKLREVGIGKDKLELMAKQAVRNSGGTIGSLRPINAEDVLEIFKKSY

SEQ ID NO: 42 - DNA, 1170 bp, Clostridium acetobutylicum
ttaataagattttttaaatatctcaagaacatcctctgcatttattggtcttaaacttcctattgttcctccagaatttctaacagcttgctttgccattagttctagtttatcttttcctattccaacttctctaagctttgaaggaatacccaatgaattaaagtattctctcgtatttttaatagcctctcgtgctatttcatagttatctttgttcttgtctattccccaaacatttattccataagaaacaaatttatgaagtgtatcgtcatttagaatatattccatccaattaggtgttaaaattgcaagtcctacaccatgtgttatatcataatatgcacttaactcgtgttccataggatgacaactccattttctatccttaccaagtgataatagaccatttatagctaaacttgaagcccacatcaaattagctctagcctcgtaatcatcagtcttctccattgctatttttccatactttatacatgttcttaagattgcttctgctataccgtcctgcacataagcaccttcaacaccactaaagtaagattcaaaggtgtgactcataatgtcagctgttcccgctgctgtttgatttttaggtactgtaaaagtatatgtaggatctaacactgaaaatttaggtctcatatcatcatgtcctactccaagcttttcattagtctccatatttgaaattactgcaatttgatccatttcagaccctgttgctgaaagagtaagtatacttgcaattggaagaactttagttattttagatggatctttaaccatgtcccatgtatcgccatcataataaactccagctgcaattaccttagaacagtctattgcacttcctccccctattgctaatactaaatccacattattttctctacatatttctatgcctttttttactgttgttatcctaggatttggctctactcctgaaagttcatagaaagctatattgttttcttttaatatagctgttgctctatcatatataccgttcctttttatacttcctccgccataaactataagcactcttgagccatatttcttaatttcttctccaattacgtctatttttccttttccaaaaaaaactttagttggtattgaataatcaaaacttagcat

SEQ ID NO: 43 - protein, 390 aa, Clostridium acetobutylicum
MVDFEYSIPTRIFFGKDKINVLGRELKKYGSKVLIVYGGGSIKRNGIYDKAVSILEKNSIKFYELAGVEPNPRVTTVEKGVKICRENGVEVVLAIGGGSAIDCAKVIAAACEYDGNPWDIVLDGSKIKRVLPIASILTIAATGSEMDTWAVINNMDTNEKLIAAHPDMAPKFSILDPTYTYTVPTNQTAAGTADIMSHIFEVYFSNTKTAYLQDRMAEALLRTCIKYGGIALEKPDDYEARANLMWASSLAINGLLTYGKDTNWSVHLMEHELSAYYDITHGVGLAILTPNWMEYILNNDTVYKFVEYGVNVWGIDKEKNHYDIAHQAIQKTRDYFVNVLGLPSRLRDVGIEEEKLDIMAKESVKLTGGTIGNLRPVNASEVLQIFKKSV

SEQ ID NO: 44 - DNA, 1173 bp, Clostridium acetobutylicum
gtggttgatttcgaatattcaataccaactagaatttttttcggtaaagataagataaatgtacttggaagagagcttaaaaaatatggttctaaagtgcttatagtttatggtggaggaagtataaagagaaatggaatatatgataaagctgtaagtatacttgaaaaaaacagtattaaattttatgaacttgcaggagtagagccaaatccaagagtaactacagttgaaaaaggagttaaaatatgtagagaaaatggagttgaagtagtactagctataggtggaggaagtgcaatagattgcgcaaaggttatagcagcagcatgtgaatatgatggaaatccatgggatattgtgttagatggctcaaaaataaaaagggtgcttcctatagctagtatattaaccattgctgcaacaggatcagaaatggatacgtgggcagtaataaataatatggatacaaacgaaaaactaattgcggcacatccagatatggctcctaagttttctatattagatccaacgtatacgtataccgtacctaccaatcaaacagcagcaggaacagctgatattatgagtcatatatttgaggtgtattttagtaatacaaaaacagcatatttgcaggatagaatggcagaagcgttattaagaacttgtattaaatatggaggaatagctcttgagaagccggatgattatgaggcaagagccaatctaatgtgggcttcaagtcttgcgataaatggacttttaacatatggtaaagacactaattggagtgtacacttaatggaacatgaattaagtgcttattacgacataacacacggcgtagggcttgcaattttaacacctaattggatggagtatattttaaataatgatacagtgtacaagtttgttgaatatggtgtaaatgtttggggaatagacaaagaaaaaaatcactatgacatagcacatcaagcaatacaaaaaacaagagattactttgtaaatgtactaggtttaccatctagactgagagatgttggaattgaagaagaaaaattggacataatggcaaaggaatcagtaaagcttacaggaggaaccataggaaacctaagaccagtaaacgcctccgaagtcctacaaatattcaaaaaatctgtgtaa

SEQ ID NO: 45 - protein, 330 aa, Bacillus subtilis
MSTNRHQALGLTDQEAVDMYRTMLLARKIDERMWLLNRSGKIPFVISCQGQEAAQVGAAFALDREMDYVLPYYRDMGVVLAFGMTAKDLMMSGFAKAADPNSGGRQMPGHFGQKKNRIVTGSSPVTTQVPHAVGIALAGRMEKKDIAAFVTFGEGSSNQGDFHEGANFAAVHKLPVIFMCENNKYAISVPYDKQVACENISDRAIGYGMPGVTVNGNDPLEVYQAVKEARERARRGEGPTLIETISYRLTPHSSDDDDSSYRGREEVEEAKKSDPLLTYQAYLKETGLLSDEIEQTMLDEIMAIVNEATDEAENAPYAAPESALDYVYAK

SEQ ID NO: 46 - DNA, 993 bp, Bacillus subtilis
atgagtacaaaccgacatcaagcactagggctgactgatcaggaagccgttgatatgtatagaaccatgctgttagcaagaaaaatcgatgaaagaatgtggctgttaaaccgttctggcaaaattccatttgtaatctcttgtcaaggacaggaagcagcacaggtaggagcggctttcgcacttgaccgtgaaatggattatgtattgccgtactacagagacatgggtgtcgtgctcgcgtttggcatgacagcaaaggacttaatgatgtccgggtttgcaaaagcagcagatccgaactcaggaggccgccagatgccgggacatttcggacaaaagaaaaaccgcattgtgacgggatcatctccggttacaacgcaagtgccgcacgcagtcggtattgcgcttgcgggacgtatggagaaaaaggatatcgcagcctttgttacattcggggaagggtcttcaaaccaaggcgatttccatgaaggggcaaactttgccgctgtccataagctgccggttattttcatgtgtgaaaacaacaaatacgcaatctcagtgccttacgataagcaagtcgcatgtgagaacatttccgaccgtgccataggctatgggatgcctggcgtaactgtgaatggaaatgatccgctggaagtttatcaagcggttaaagaagcacgcgaaagggcacgcagaggagaaggcccgacattaattgaaacgatttcttaccgccttacaccacattccagtgatgacgatgacagcagctacagaggccgtgaagaagtagaggaagcgaaaaaaagtgatcccctgcttacttatcaagcttacttaaaggaaacaggcctgctgtccgatgagatagaacaaaccatgctggatgaaattatggcaatcgtaaatgaagcgacggatgaagcggagaacgccccatatgcagctcctgagtcagcgcttgattatgtttatgcgaagtag

SEQ ID NO: 47 - protein, 327 aa, Bacillus subtilis
MSVMSYIDAINLAMKEEMERDSRVFVLGEDVGRKGGVFKATAGLYEQFGEERVMDTPLAESAIAGVGIGAAMYGMRPIAEMQFADFIMPAVNQIISEAAKIRYRSNNDWSCPIVVRAPYGGGVHGALYHSQSVEAIFANQPGLKIVMPSTPYDAKGLLKAAVRDEDPVLFFEHKRAYRLIKGEVPADDYVLPIGKADVKREGDDITVITYGLCVHFALQAAERLEKDGISAHVVDLRTVYPLDKEAIIEAASKTGKVLLVTEDTKEGSIMSEVAAIISEHCLFDLDAPIKRLAGPDIPAMPYAPTMEKYFMVNPDKVEAAMRELAEF

SEQ ID NO: 48 - DNA, 984 bp, Bacillus subtilis
atgtcagtaatgtcatatattgatgcaatcaatttggcgatgaaagaagaaatggaacgagattctcgcgttttcgtccttggggaagatgtaggaagaaaaggcggtgtgtttaaagcgacagcgggactctatgaacaatttggggaagagcgcgttatggatacgccgcttgctgaatctgcaatcgcaggagtcggtatcggagcggcaatgtacggaatgagaccgattgctgaaatgcagtttgctgatttcattatgccggcagtcaaccaaattatttctgaagcggctaaaatccgctaccgcagcaacaatgactggagctgtccgattgtcgtcagagcgccatacggcggaggcgtgcacggagccctgtatcattctcaatcagtcgaagcaattttcgccaaccagcccggactgaaaattgtcatgccatcaacaccatatgacgcgaaagggctcttaaaagccgcagttcgtgacgaagaccccgtgctgttttttgagcacaagcgggcataccgtctgataaagggcgaggttccggctgatgattatgtcctgccaatcggcaaggcggacgtaaaaagggaaggcgacgacatcacagtgatcacatacggcctgtgtgtccacttcgccttacaagctgcagaacgtctcgaaaaagatggcatttcagcgcatgtggtggatttaagaacagtttacccgcttgataaagaagccatcatcgaagctgcgtccaaaactggaaaggttcttttggtcacagaagatacaaaagaaggcagcatcatgagcgaagtagccgcaattatatccgagcattgtctgttcgacttagacgcgccgatcaaacggcttgcaggtcctgatattccggctatgccttatgcgccgacaatggaaaaatactttatggtcaaccctgataaagtggaagcggcgatgagagaattagcggagttttaa

SEQ ID NO: 49 - protein, 424 aa, Bacillus subtilis
MAIEQMTMPQLGESVTEGTISKWLVAPGDKVNKYDPIAEVMTDKVNAEVPSSFTGTITELVGEEGQTLQVGEMICKIETEGANPAEQKQEQPAASEAAENPVAKSAGAADQPNKKRYSPAVLRLAGEHGIDLDQVTGTGAGGRITRKDIQRLIETGGVQEQNPEELKTAAPAPKSASKPEPKEETSYPASAAGDKEIPVTGVRKAIASNMKRSKTEIPHAWTMMEVDVTNMVAYRNSIKDSFKKTEGFNLTFFAFFVKAVAQALKEFPQMNSMWAGDKIIQKKDINISIAVATEDSLFVPVIKNADEKTIKGIAKDITGLAKKVRDGKLTADDMQGGTFTVNNTGSFGSVQSMGIINYPQAAILQVESIVKRPVVMDNGMIAVRDMVNLCLSLDHRVLDGLVCGRFLGRVKQILESIDEKTSVY

SEQ ID NO: 50 - DNA, 1275 bp, Bacillus subtilis
atggcaattgaacaaatgacgatgccgcagcttggagaaagcgtaacagaggggacgatcagcaaatggcttgtcgcccccggtgataaagtgaacaaatacgatccgatcgcggaagtcatgacagataaggtaaatgcagaggttccgtcttcttttactggtacgataacagagcttgtgggagaagaaggccaaaccctgcaagtcggagaaatgatttgcaaaattgaaacagaaggcgcgaatccggctgaacaaaaacaagaacagccagcagcatcagaagccgctgagaaccctgttgcaaaaagtgctggagcagccgatcagcccaataaaaagcgctactcgccagctgttctccgtttggccggagagcacggcattgacctcgatcaagtgacaggaactggtgccggcgggcgcatcacacgaaaagatattcagcgcttaattgaaacaggcggcgtgcaagaacagaatcctgaggagctgaaaacagcagctcctgcaccgaagtctgcatcaaaacctgagccaaaagaagagacgtcatatcctgcgtctgcagccggtgataaagaaatccctgtcacaggtgtaagaaaagcaattgcttccaatatgaagcgaagcaaaacagaaattccgcatgcttggacgatgatggaagtcgacgtcacaaatatggttgcatatcgcaacagtataaaagattcttttaagaagacagaaggctttaatttaacgttcttcgccttttttgtaaaagcggtcgctcaggcgttaaaagaattcccgcaaatgaatagcatgtgggcgggggacaaaattattcagaaaaaggatatcaatatttcaattgcagttgccacagaggattctttatttgttccggtgattaaaaacgctgatgaaaaaacaattaaaggcattgcgaaagacattaccggcctagctaaaaaagtaagagacggaaaactcactgcagatgacatgcagggaggcacgtttaccgtcaacaacacaggttcgttcgggtctgttcagtcgatgggcattatcaactaccctcaggctgcgattcttcaagtagaatccatcgtcaaacgcccggttgtcatggacaatggcatgattgctgtcagagacatggttaatctgtgcctgtcattagatcacagagtgcttgacggtctcgtgtgcggacgattcctcggacgagtgaaacaaattttagaatcgattgacgagaagacatctgtttactaa

SEQ ID NO: 51 - protein, 474 aa, Bacillus subtilis
MATEYDVVILGGGTGGYVAAIRAAQLGLKTAVVEKEKLGGTCLHKGCIPSKALLRSAEVYRTAREADQFGVETAGVSLNFEKVQQRKQAVVDKLAAGVNHLMKKGKIDVYTGYGRILGPSIFSPLPGTISVERGNGEENDMLIPKQVIIATGSRPRMLPGLEVDGKSVLTSDEALQMEELPQSIIIVGGGVIGIEWASMLHDFGVKVTVIEYADRILPTEDLEISKEMESLLKKKGIQFITGAKVLPDTMTKTSDDISIQAEKDGETVTYSAEKMLVSIGRQANIEGIGLENTDIVTENGMISVNESCQTKESHIYAIGDVIGGLQLAHVASHEGIIAVEHFAGLNPHPLDPTLVPKCIYSSPEAASVGLTEDEAKANGHNVKIGKFPFMAIGKALVYGESDGFVKIVADRDTDDILGVHMIGPHVTDMISEAGLAKVLDATPWEVGQTIHPHPTLSEAIGEAALAADGKAIHF

SEQ ID NO: 52 - DNA, 1425 bp, Bacillus subtilis
atggcaactgagtatgacgtagtcattctgggcggcggtaccggcggttatgttgcggccatcagagccgctcagctcggcttaaaaacagccgttgtggaaaaggaaaaactcgggggaacatgtctgcataaaggctgtatcccgagtaaagcgctgcttagaagcgcagaggtataccggacagctcgtgaagccgatcaattcggagtggaaacggctggcgtgtccctcaactttgaaaaagtgcagcagcgtaagcaagccgttgttgataagcttgcagcgggtgtaaatcatttaatgaaaaaaggaaaaattgacgtgtacaccggatatggacgtatccttggaccgtcaatcttctctccgctgccgggaacaatttctgttgagcggggaaatggcgaagaaaatgacatgctgatcccgaaacaagtgatcattgcaacaggatcaagaccgagaatgcttccgggtcttgaagtggacggtaagtctgtactgacttcagatgaggcgctccaaatggaggagctgccacagtcaatcatcattgtcggcggaggggttatcggtatcgaatgggcgtctatgcttcatgattttggcgttaaggtaacggttattgaatacgcggatcgcatattgccgactgaagatctagagatttcaaaagaaatggaaagtcttcttaagaaaaaaggcatccagttcataacaggggcaaaagtgctgcctgacacaatgacaaaaacatcagacgatatcagcatacaagcggaaaaagacggagaaaccgttacctattctgctgagaaaatgcttgtttccatcggcagacaggcaaatatcgaaggcatcggcctagagaacaccgatattgttactgaaaatggcatgatttcagtcaatgaaagctgccaaacgaaggaatctcatatttatgcaatcggagacgtaatcggtggcctgcagttagctcacgttgcttcacatgagggaattattgctgttgagcattttgcaggtctcaatccgcatccgcttgatccgacgcttgtgccgaagtgcatttactcaagccctgaagctgccagtgtcggcttaaccgaagacgaagcaaaggcgaacgggcataatgtcaaaatcggcaagttcccatttatggcgattggaaaagcgcttgtatacggtgaaagcgacggttttgtcaaaatcgtggctgaccgagatacagatgatattctcggcgttcatatgattggcccgcatgtcaccgacatgatttctgaagcgggtcttgccaaagtgctggacgcaacaccgtgggaggtcgggcaaacgattcacccgcatccaacgctttctgaagcaattggagaagctgcgcttgccgcagatggcaaagccattcatttttaa

SEQ ID NO: 53 - protein, 410 aa, Pseudomonas putida
MNEYAPLRLHVPEPTGRPGCQTDFSYLRLNDAGQARKPPVDVDAADTADLSYSLVRVLDEQGDAQGPWAEDIDPQILRQGMRAMLKTRIFDSRMVVAQRQKKMSFYMQSLGEEAIGSGQALALNRTDMCFPTYRQQSILMARDVSLVEMICQLLSNERDPLKGRQLPIMYSVREAGFFTISGNLATQFVQAVGWAMASAIKGDTKIASAWIGDGATAESDFHTALTFAHVYRAPVILNVVNNQWAISTFQAIAGGESTTFAGRGVGCGIASLRVDGNDFVAVYAASRWAAERARRGLGPSLIEWVTYRAGPHSTSDDPSKYRPADDWSHFPLGDPIARLKQHLIKIGHWSEEEHQATTAEFEAAVIAAQKEAEQYGTLANGHIPSAASMFEDVYKEMPDHLRRQRQELGV

SEQ ID NO: 54 - DNA, 6643 bp, Pseudomonas putida
gcatgcctgcaggccgccgatgaaatggtggaaggtatcggtaggctggccctgctcatcgctgaacacgttacgcccgctgccggtatcgaccaggctctggtgaatatgcatggaactgccaggcgtgcgcgccagcggtttggccatgcacaccacggtcagcccgtgcttgagtgccacttccttgagcaggtgtttgaacaggaaggtctggtcggccagcagcagcgggtcgccatgtagcaagttgatctcgaactggctgacgcccatttcgtgcatgaaggtgtcgcgcggcaggccgagcgcggccatgcactggtacacctcattgaagaacgggcgcaggccgttgttggaactgacactgaacgccgaatggcccagctcgcggcggccgtcggtgcccagcggtggctggaacggctgctgcgggtcactgttgggggcaaacacgaagaactcaagctcggtcgccactaccggtgccagacccaacgctgcgtagcgggcgatcacggccttcagctggccccgggtggacagtgccgagggccggccatccagttcattggcatcgcagatggccagggcgcgaccgtcatcgctccagggcaagcgatgaacctggctgggttccgctaccaacgccaggtcgccgtcgtcgcagccgtagaatttcgccggcgggtagccgcccatgatgcattgcagcagcaccccacgggccatctgcaggcggcggccttcgagaaagccttcggcggtcatcaccttgccgcgtgggacgccgttgaggtcgggggtgacgcattcgatttcatcgatgccctggagctgagcgatgctcatgacgcttgtccttgttgttgtaggctgacaacaacataggctgggggtgtttaaaatatcaagcagcctctcgaacgcctggggcctcttctattcgcgcaaggtcatgccattggccggcaacggcaaggctgtcttgtagcgcacctgtttcaaggcaaaactcgagcggatattcgccacacccggcaaccgggtcaggtaatcgagaaaccgctccagcgcctggatactcggcagcagtacccgcaacaggtagtccgggtcgcccgtcatcaggtagcactccatcacctcgggccgttcggcaatttcttcctcgaagcggtgcagcgactgctctacctgtttttccaggctgacatggatgaacacattcacatccagccccaacgcctcgggcgacaacaaggtcacctgctggcggatcacccccagttcttccatggcccgcacccggttgaaacagggcgtgggcgacaggttgaccgagcgtgccagctcggcgttggtgatgcgggcgttttcctgcaggctgttgagaatgccgatatcggtacgatcgagtttgcgcatgagacaaaatcaccggttttttgtgtttatgcggaatgtttatctgccccgctcggcaaaggcaatcaacttgagagaaaaattctcctgccggaccactaagatgtaggggacgctgacttaccagtcacaagccggtactcagcggcggccgcttcagagctcacaaaaacaaatacccgagcgagcgtaaaaagcatgaacgagtacgcccccctgcgtttgcatgtgcccgagcccaccggccggccaggctgccagaccgatttttcctacctgcgcctgaacgatgcaggtcaagcccgtaaaccccctgtcgatgtcgacgctgccgacaccgccgacctgtcctacagcctggtccgcgtgctcgacgagcaaggcgacgcccaaggcccgtgggctgaagacatcgacccgcagatcctgcgccaaggcatgcgcgccatgctcaagacgcggatcttcgacagccgcatggtggttgcccagcgccagaagaagatgtccttctacatgcagagcctgggcgaagaagccatcggcagcggccaggcgctggcgcttaaccgcaccgacatgtgcttccccacctaccgtcagcaaagcatcctgatggcccgcgacgtgtcgctggtggagatgatctgccagttgctgtccaacgaacgcgaccccctcaagggccgccagctgccgatcatgtactcggtacgcgaggccggcttcttcaccatcagcggcaacctggcgacccagttcgtgcaggcggtcggctgggccatggcctcggcgatcaagggcgataccaagattgcctcggcctggatcggcgacggcgccactgccgaatcggacttccacaccgccctcacctttgcccacgtttaccgcgccccggtgatcctcaacgtggtcaacaaccagtgggccatctcaaccttccaggccatcgccggtggcgagtcgaccaccttcgccggccgtggcgtgggctgcggcatcgcttcgctgcgggtggacggcaacgacttcgtcgccgtttacgccgcttcgcgctgggctgccgaacgtgcccgccgtggtttgggcccgagcctgatcgagtgggtcacctaccgtgccggcccgcactcgacctcggacgacccgtccaagtaccgccctgccgatgactggagccacttcccgctgggtgacccgatcgcccgcctgaagcagcacctgatcaagatcggccactggtccgaagaagaacaccaggccaccacggccgagttcgaagcggccgtgattgctgcgcaaaaagaagccgagcagtacggcaccctggccaacggtcacatcccgagcgccgcctcgatgttcgaggacgtgtacaaggagatgcccgaccacctgcgccgccaacgccaggaactgggggtttgagatgaacgaccacaacaacagcatcaacccggaaaccgccatggccaccactaccatgaccatgatccaggccctgcgctcggccatggatgtcatgcttgagcgcgacgacaatgtggtggtgtacggccaggacgtcggctacttcggcggcgtgttccgctgcaccgaaggcctgcagaccaagtacggcaagtcccgcgtgttcgacgcgcccatctctgaaagcggcatcgtcggcaccgccgtgggcatgggtgcctacggcctgcgcccggtggtggaaatccagttcgctgactacttctacccggcctccgaccagatcgtttctgaaatggcccgcctgcgctaccgttcggccggcgagttcatcgccccgctgaccctgcgtatgccctgcggtggcggtatctatggcggccagacacacagccagagcccggaagcgatgttcactcaggtgtgcggcctgcgcaccgtaatgccatccaacccgtacgacgccaaaggcctgctgattgcctcgatcgaatgcgacgacccggtgatcttcctggagcccaagcgcctgtacaacggcccgttcgacggccaccatgaccgcccggttacgccgtggtcgaaacacccgcacagcgccgtgcccgatggctactacaccgtgccactggacaaggccgccatcacccgccccggcaatgacgtgagcgtgctcacctatggcaccaccgtgtacgtggcccaggtggccgccgaagaaagtggcgtggatgccgaagtgatcgacctgcgcagcctgtggccgctagacctggacaccatcgtcgagtcggtgaaaaagaccggccgttgcgtggtagtacacgaggccacccgtacttgtggctttggcgcagaactggtgtcgctggtgcaggagcactgcttccaccacctggaggcgccgatcgagcgcgtcaccggttgggacaccccctaccctcacgcgcaggaatgggcttacttcccagggccttcgcgggtaggtgcggcattgaaaaaggtcatggaggtctgaatgggcacgcacgtcatcaagatgccggacattggcgaaggcatcgcgcaggtcgaattggtggaatggttcgtcaaggtgggcgacatcatcgccgaggaccaagtggtagccgacgtcatgaccgacaaggccaccgtggaaatcccgtcgccggtcagcggcaaggtgctggccctgggtggccagccaggtgaagtgatggcggtcggcagtgagctgatccgcatcgaagtggaaggcagcggcaaccatgtggatgtgccgcaagccaagccggccgaagtgcctgcggcaccggtagccgctaaacctgaaccacagaaagacgttaaaccggcggcgtaccaggcgtcagccagccacgaggcagcgcccatcgtgccgcgccagccgggcgacaagccgctggcctcgccggcggtgcgcaaacgcgccctcgatgccggcatcgaattgcgttatgtgcacggcagcggcccggccgggcgcatcctgcacgaagacctcgacgcgttcatgagcaaaccgcaaagcgctgccgggcaaacccccaatggctatgccaggcgcaccgacagcgagcaggtgccggtgatcggcctgcgccgcaagatcgcccagcgcatgcaggacgccaagcgccgggtcgcgcacttcagctatgtggaagaaatcgacgtcaccgccctggaagccctgcgccagcagctcaacagcaagcacggcgacagccgcggcaagctgacactgctgccgttcctggtgcgcgccctggtcgtggcactgcgtgacttcccgcagataaacgccacctacgatgacgaagcgcagatcatcacccgccatggcgcggtgcatgtgggcatcgccacccaaggtgacaacggcctgatggtacccgtgctgcgccacgccgaagcgggcagcctgtgggccaatgccggtgagatttcacgcctggccaacgctgcgcgcaacaacaaggccagccgcgaagagctgtccggttcgaccattaccctgaccagcctcggcgccctgggcggcatcgtcagcacgccggtggtcaacaccccggaagtggcgatcgtcggtgtcaaccgcatggttgagcggcccgtggtgatcgacggccagatcgtcgtgcgcaagatgatgaacctgtccagctcgttcgaccaccgcgtggtcgatggcatggacgccgccctgttcatccaggccgtgcgtggcctgctcgaacaacccgcctgcctgttcgtggagtgagcatgcaacagactatccagacaaccctgttgatcatcggcggcggccctggcggctatgtggcggccatccgcgccgggcaactgggcatccctaccgtgctggtggaaggccaggcgctgggcggtacctgcctgaacatcggctgcattccgtccaaggcgctgatccatgtggccgagcagttccaccaggcctcgcgctttaccgaaccctcgccgctgggcatcagcgtggcttcgccacgcctggacatcggccagagcgtggcctggaaagacggcatcgtcgatcgcctgaccactggtgtcgccgccctgctgaaaaagcacggggtgaaggtggtgcacggctgggccaaggtgcttgatggcaagcaggtcgaggtggatggccagcgcatccagtgcgagcacctgttgctggccacgggctccagcagtgtcgaactgccgatgctgccgttgggtgggccggtgatttcctcgaccgaggccctggcaccgaaagccctgccgcaacacctggtggtggtgggcggtggctacatcggcctggagctgggtatcgcctaccgcaagctcggcgcgcaggtcagcgtggtggaagcgcgcgagcgcatcctgccgacttacgacagcgaactgaccgccccggtggccgagtcgctgaaaaagctgggtatcgccctgcaccttggccacagcgtcgaaggttacgaaaatggctgcctgctggccaacgatggcaagggcggacaactgcgcctggaagccgaccgggtgctggtggccgtgggccgccgcccacgcaccaagggcttcaacctggaatgcctggacctgaagatgaatggtgccgcgattgccatcgacgagcgctgccagaccagcatgcacaacgtctgggccatcggcgacgtggccggcgaaccgatgctggcgcaccgggccatggcccagggcgagatggtggccgagatcatcgccggcaaggcacgccgcttcgaacccgctgcgatagccgccgtgtgcttcaccgacccggaagtggtcgtggtcggcaagacgccggaacaggccagtcagcaaggcctggactgcatcgtcgcgcagttcccgttcgccgccaacggccgggccatgagcctggagtcgaaaagcggtttcgtgcgcgtggtcgcgcggcgtgacaaccacctgatcctgggctggcaagcggttggcgtggcggtttccgagctgtccacggcgtttgcccagtcgctggagatgggtgcctgcctggaggatgtggccggtaccatccatgcccacccgaccctgggtgaagcggtacaggaagcggcactgcgtgccctgggccacgccctgcatatctgacactgaagcggccgaggccgatttggcccgccgcgccgagaggcgctgcgggtcttttttatacctg

SEQ ID NO: 55 - protein, 352 aa, Pseudomonas putida
MNDHNNSINPETAMATTTMTMIQALRSAMDVMLERDDNVVVYGQDVGYFGGVFRCTEGLQTKYGKSRVFDAPISESGIVGTAVGMGAYGLRPVVEIQFADYFYPASDQIVSEMARLRYRSAGEFIAPLTLRMPCGGGIYGGQTHSQSPEAMFTQVCGLRTVMPSNPYDAKGLLIASIECDDPVIFLEPKRLYNGPFDGHHDRPVTPWSKHPHSAVPDGYYTVPLDKAAITRPGNDVSVLTYGTTVYVAQVAAEESGVDAEVIDLRSLWPLDLDTIVESVKKTGRCVVVHEATRTCGFGAELVSLVQEHCFHHLEAPIERVTGWDTPYPHAQEWAYFPGPSRVGAALKKVMEV

SEQ ID NO: 56 - DNA, 6643 bp, Pseudomonas putida; identical to SEQ ID NO: 54

SEQ ID NO: 57 - protein, 423 aa, Pseudomonas putida
MGTHVIKMPDIGEGIAQVELVEWFVKVGDIIAEDQVVADVMTDKATVEIPSPVSGKVLALGGQPGEVMAVGSELIRIEVEGSGNHVDVPQAKPAEVPAAPVAAKPEPQKDVKPAAYQASASHEAAPIVPRQPGDKPLASPAVRKRALDAGIELRYVHGSGPAGRILHEDLDAFMSKPQSAAGQTPNGYARRTDSEQVPVIGLRRKIAQRMQDAKRRVAHFSYVEEIDVTALEALRQQLNSKHGDSRGKLTLLPFLVRALVVALRDFPQINATYDDEAQIITRHGAVHVGIATQGDNGLMVPVLRHAEAGSLWANAGEISRLANAARNNKASREELSGSTITLTSLGALGGIVSTPVVNTPEVAIVGVNRMVERPVVIDGQIVVRKMMNLSSSFDHRVVDGMDAALFIQAVRGLLEQPACLFVE

SEQ ID NO: 58 - DNA, 6643 bp, Pseudomonas putida; identical to SEQ ID NO: 54

SEQ ID NO: 59 - protein, 459 aa, Pseudomonas putida
MQQTIQTTLLIIGGGPGGYVAAIRAGQLGIPTVLVEGQALGGTCLNIGCIPSKALIHVAEQFHQASRFTEPSPLGISVASPRLDIGQSVAWKDGIVDRLTTGVAALLKKHGVKVVHGWAKVLDGKQVEVDGQRIQCEHLLLATGSSSVELPMLPLGGPVISSTEALAPKALPQHLVVVGGGYIGLELGIAYRKLGAQVSVVEARERILPTYDSELTAPVAESLKKLGIALHLGHSVEGYENGCLLANDGKGGQLRLEADRVLVAVGRRPRTKGFNLECLDLKMNGAAIAIDERCQTSMHNVWAIGDVAGEPMLAHRAMAQGEMVAEIIAGKARRFEPAAIAAVCFTDPEVVVVGKTPEQASQQGLDCIVAQFPFAANGRAMSLESKSGFVRVVARRDNHLILGWQAVGVAVSELSTAFAQSLEMGACLEDVAGTIHAHPTLGEAVQEAALRALGHALHI

SEQ ID NO: 60 - DNA, 6643 bp, Pseudomonas putida; identical to SEQ ID NO: 54

SEQ ID NO: 61 - protein, 468 aa, Clostridium beijerinckii
MNKDTLIPTTKDLKLKTNVENINLKNYKDNSSCFGVFENVENAINSAVHAQKILSLHYTKEQREKIITEIRKAALENKEVLATMILEETHMGRYEDKILKHELVAKYTPGTEDLTTTAWSGDNGLTVVEMSPYGVIGAITPSTNPTETVICNSIGMIAAGNAVVFNGHPGAKKCVAFAIEMINKAIISCGGPENLVTTIKNPTMESLDAIIKHPLIKLLCGTGGPGMVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYFINKKWVGKDAKLFSDEIDVESPSNIKCIVCEVNANHPFVMTELMMPILPIVRVKDIDEAVKYTKIAEQNRKHSAYIYSKNIDNLNRFEREIDTTIFVKNAKSFAGVGYEAEGFTTFTIAGSTGEGITSARNFTRQRRCVLAG

SEQ ID NO: 62 - DNA, 6558 bp, Clostridium beijerinckii
aagcttaaaatatccataggctattgttaataagactatagcgcttaatactctaagcgcaccatctaaaaaattatacataggaattggataaactccaatcttctccataatacttttcataggtgaaattgatattataattaaggctgcataaagcaaactcatatctccaataaatgtcactatcataggtaatttccttttcatgttcacatactcccccgtattctataataatttacaataaacttccatcacaaataaattataacatatattgtaaatatagttttattattcgcatatttatagataaacaatatataacttagacttatgcaataacctaccgaaagtaaaaacattgttatttcacaggactatgaaaatttcgctgaaagtactaaattgcaggttgcaccactaatgcttgctcccaatttcattgtgacaagcattagttgaacaacctacaattaagagcctttaacagctcatttccaatgcctgctacataaaaatgtttctactttctaatatggtatttacttcaaagtgtaaatcaaattcttaagttgtttatctatatagggttccatattgataacaatacatatctactgtttcaatttcatgaataccagcgatattgaaattttgtaagtttgaatatcattgtgaaaccctatatatcaaatacaatctcaaaattatacaaaaagatcccaacttcacaatatgaatttgagatcttctatcttaacttcttcaatatttttaagattatttatactgcgtgcaaatcttctataagtatcaattatcagctatgtacattgatagtggacttgtaatctgaacagcttatatatttacacttttaagttctcttccactaacccctgctttgaaacttctcataaacaaatcgaaagtacctttaattagctgtgcattctcaaactcagaatttaacttaagtacttcaattgccgcttcgatagtatagagctctccttctgaagcaccttttcttaaagtatattctgatttattaattggatttaatgaaattcttggaagcttctttaagtagtcgctctttcttagtatcttctctgcttctttccatgtgccatctaagataataaatgctggaattttttctgaatctttacatttagactttctttctagaggttcatcatcatccataggaaataatacacgtatttcataatcatcgctattaatatattcaattaatttttcaggagtctttactctctcccaaagaattaactcagttgattctggattcaccaatttcaataatctagcggtatttgaaggcctactaaattctctttctgttgataatatcaatatctttgcttttgtctctattttaggcacaatatcgcagatacaatttattattggcaacccacatttattgcagctctcatataacttagtaatttgcttaactttaaattcagactccattttacctccattattagttggttagtgtgtcatatcttcttgctattactaactgattataacatatgtattcaatatatcactcctagttttcaaagcactggcaatacgaattacaaattaatttctggatttatgtcagtatttcattaataaaaggtcggacttttaagatacttgttttagctattgatcatatttattaaagactatgcatttaatgtataattataatgaatattatcaataatatttattttatattacaatcttacagtctttattctaaatttcactcaaataccaaacgagctttattcataaacaatatataacaataattccaaaataatacgatattttatctgtaacagccatataaaaaaaatatcatatagtcttgtcatttgataacgttttgtcttccttatatttactttttcggtttaataggttgattctgtaaattttagtgataacatatatttgatgacattaaaaatttaatatttcatataaatttttaatgtctattaatttttaaatcacaaggaggaatagttcatgaataaagacacactaatacctacaactaaagatttaaaattaaaaacaaatgttgaaaacattaatttaaagaactacaaggataattcttcatgtttcggagtattcgaaaatgttgaaaatgctataaacagcgctgtacacgcgcaaaagatattatcccttcattatacaaaagaacaaagagaaaaaatcataactgagataagaaaggccgcattagaaaataaagaggttttagctaccatgattctggaagaaacacatatgggaaggtatgaagataaaatattaaagcatgaattagtagctaaatatactcctggtacagaagatttaactactactgcttggtcaggtgataatggtcttacagttgtagaaatgtctccatatggcgttataggtgcaataactccttctacgaatccaactgaaactgtaatatgtaatagcatcggcatgatagctgctggaaatgctgtagtatttaacggacacccaggcgctaaaaaatgtgttgcttttgctattgaaatgataaataaagcaattatttcatgtggcggtcctgagaatttagtaacaactataaaaaatccaactatggaatccctagatgcaattattaagcatcctttaataaaacttctttgcggaactggaggtccaggaatggtaaaaaccctcttaaattctggcaagaaagctataggtgctggtgctggaaatccaccagttattgtagatgataccgctgatatagaaaaggctggtaagagtatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattctcagatgaaatagatgttgagtctccttcaaatattaaatgcatagtctgcgaagtaaatgcaaatcatccatttgtcatgacagaactcatgatgccaatattaccaattgtaagagttaaagatatagatgaagctgttaaatatacaaagatagcagaacaaaatagaaaacatagtgcctatatttattctaaaaatatagacaacctaaatagatttgaaagagaaattgatactactatttttgtaaagaatgctaaatcttttgctggtgttggttatgaagctgaaggatttacaactttcactattgctggatctactggtgaaggcataacctctgcaagaaattttacaagacaaagaagatgtgtacttgccggctaacttcttgctaaatttatacatttattcacataactttaatatgcaatgttcccacaaaatattaaaaactatttagaagggagatattaaatgaataaattagtaaaattaacagatttaaagcgcattttcaaagatggtatgacaattatggttgggggttttttagattgtggaactcctgaaaatattatagatatgctagttgatttaaatataaaaaatctgactattataagcaatgatacagcttttcctaataaaggaataggaaaacttattgtaaatggtcaagtttctaaagtaattgcttcacatattggaactaatcctgaaactgggaaaaaaatgagctctggtgaacttaaagttgagctttctccacaaggaacactgatcgaaagaattcgtgcagctggatctggactcggaggtgtattaactccaaccggacttgggactatcgttgaagaaggtaagaaaaaagttactatcggtggcaaagaatatctattagaacttcctttatccgctgatgtttcattaataaaaggtagcattgtagatgaatttggaaataccttctatagagctgctactaaaaatttcaatccatatatggcaatggctgcaaaaacagttatagttgaagcagaaaatttagttaaatgtgaagatttaaaaagagatgccataatgactcctggcgtattagtagattatatcgttaaggaggcggcttaattgattgtagataaagttttagcaaaagagataattgccaaaagagttgcaaaagaactaaaaaaaggccaactcgtaaaccttggaataggacttccaactttagtagctaattatgtgccaaaagaaatgaacattactttcgaatcagaaaatggcatggttggcatggcacaaatggcctcatcaggtgaaaatgacccagatataataaatgctggtggggaatatgtaacattattacctcaaggtgcattttttgatagttcaacgtcttttgcactaataagaggaggacatgttgatgttgctgttcttggtgctctagaagttgatgaagaaggtaatttagctaactggattgttccaaataaaattgtcccaggtatgggaggcgccatggatttggcaataggcgcaaaaaaaataatagtggcaatgcaacatacaggaaaaggtaaacctaaaatcgtaaaaaaatgtactctcccacttactgctaaggctcaggtagatttaattgttacagaactttgtgtaattgatgtaacaaatgatggtttacttttcagagaaattcataaagatacaactattgatgaaataaaatttttaacagatgcagatttaattattcccgacaacttaaaaattatggatatctaaatcattctattttaaatatataactttaaaaatcttatgtattaaaaactaagaaaagaggttgattattttatgttagaaagtgaagtatctaaacaaattacaactccacttgctgctccagcgtttcctagaggaccatatagatttcacaatagagaatatctaaacattatttatcgaactgatttagatgctcttcgaaaaatagtaccagagccacttgaattagatggagcatatgttaggtttgagatgatggctatgcctgatacaaccggactaggctcatatactgagtgtggtcaagccattccagtaaaatataatgaggttaaaggtgactacttgcatatgatgtacctagataatgaacctgctattgctgttggaagagaaagcagtgcttatcccaaaaagttcggctatccaaagctatttgttgattcagacgccctagttggcgcccttaagtatggtgcattaccggtagttactgcgacgatgggatataagcatgagcccctagatcttaaagaagcctatactcaaattgcaagacccaatttcatgctaaaaatcattcaaggttatgatggtaagccaagaatttgtgaactcatctgtgcagaaaatactgatataactatccacggtgcttggactggaagtgcacgcctacaattatttagccatgcactagctcctcttgctgatttacctgtattagagatcgtatcagcatctcatatcctaacagatttaactcttggaacacctaaggttgtacatgattatctttcagtaaaataaaagcaatatagaataaccactacaaaagtagtggttattctatattttaaatcaaactgtaaaacttaagttttatagtacctaataatattttactaccagcattagattagttaaaatacaaagtttgtggtaaaagtattttagattgcataatagccttctatacttttaacaatataaccaattgctcaccatctgcttagaatatgcttctttaagctctaaaatacatataaaaaagtaggaatttcttattaaaattcctacttatattatatataaatttaatcgttaggttttattcgcattgttcctctttaatttatctcttataacattttattataattgttcatataattaattcaatatactattatatattttcaagcattaataattattcagcatctgtcattacatatgcttccatactttgacttcttattaaatcatagctaatccatccatagccattgattccccagtctttaccccatgaatttattatttttacagcttttttactatcatcataaccaactacgcaaactgcatgaccacctctattttctccatcaatctggtcataaattggattatcagaatttaaattatcaaaatctggatatactgatattccaataactactggatttccagctgctatttgtgcctttattgcattatagtcaccatctggaagttgactccaactttttgctttatatttggctgcattagccttttgttcatctgtaggtgtaacctcccaactatattcactaccatcataaggcatatcagataatgtagtacaaccttgttcttctaataatttaaatgcat

SEQ ID NO: 63 - protein, 862 aa, Clostridium acetobutylicum
MKVTTVKELDEKLKVIKEAQKKFSCYSQEMVDEIFRNAAMAAIDARIELAKAAVLETGMGLVEDKVIKNHFAGEYIYNKYKDEKTCGIIERNEPYGITKIAEPIGVVAAIIPVTNPTSTTIFKSLISLKTRNGIFFSPHPRAKKSTILAAKTILDAAVKSGAPENIIGWIDEPSIELTQYLMQKADITLATGGPSLVKSAYSSGKPAIGVGPGNTPVIIDESAHIKMAVSSIILSKTYDNGVICASEQSVIVLKSIYNKVKDEFQERGAYIIKKNELDKVREVIFKDGSVNPKIVGQSAYTIAAMAGIKVPKTTRILIGEVTSLGEEEPFAHEKLSPVLAMYEADNFDDALKKAVTLINLGGLGHTSGIYADEIKARDKIDRFSSAMKTVRTFVNIPTSQGASGDLYNFRIPPSFTLGCGFWGGNSVSENVGPKHLLNIKTVAERRENMLWFRVPHKVYFKFGCLQFALKDLKDLKKKRAFIVTDSDPYNLNYVDSIIKILEHLDIDFKVFNKVGREADLKTIKKATEEMSSFMPDTIIALGGTPEMSSAKLMWVLYEHPEVKFEDLAIKFMDIRKRIYTFPKLGKKAMLVAITTSAGSGSEVTPFALVTDNNTGNKYMLADYEMTPNMAIVDAELMMKMPKGLTAYSGIDALVNSIEAYTSVYASEYTNGLALEAIRLIFKYLPEAYKNGRTNEKAREKMAHASTMAGMASANAFLGLCHSMAIKLSSEHNIPSGIANALLIEEVIKFNAVDNPVKQAPCPQYKYPNTIFRYARIADYIKLGGNTDEEKVDLLINKIHELKKALNIPTSIKDAGVLEENFYSSLDRISELALDDQCTGANPRFPLTSEIKEMYINCFKKQP

SEQ ID NO: 64 - DNA, 1665 bp, Clostridium acetobutylicum; identical to SEQ ID NO: 32

SEQ ID NO: 65 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 66 - DNA, 2589 bp, Clostridium acetobutylicum
atgaaagtcacaacagtaaaggaattagatgaaaaactcaaggtaattaaagaagctcaaaaaaaattctcttgttactcgcaagaaatggttgatgaaatctttagaaatgcagcaatggcagcaatcgacgcaaggatagagctagcaaaagcagctgttttggaaaccggtatgggcttagttgaagacaaggttataaaaaatcattttgcaggcgaatacatctataacaaatataaggatgaaaaaacctgcggtataattgaacgaaatgaaccctacggaattacaaaaatagcagaacctataggagttgtagctgctataatccctgtaacaaaccccacatcaacaacaatatttaaatccttaatatcccttaaaactagaaatggaattttcttttcgcctcacccaagggcaaaaaaatccacaatactagcagctaaaacaatacttgatgcagccgttaagagtggtgccccggaaaatataataggttggatagatgaaccttcaattgaactaactcaatatttaatgcaaaaagcagatataacccttgcaactggtggtccctcactagttaaatctgcttattcttccggaaaaccagcaataggtgttggtccgggtaacaccccagtaataattgatgaatctgctcatataaaaatggcagtaagttcaattatattatccaaaacctatgataatggtgttatatgtgcttctgaacaatctgtaatagtcttaaaatccatatataacaaggtaaaagatgagttccaagaaagaggagcttatataataaagaaaaacgaattggataaagtccgtgaagtgatttttaaagatggatccgtaaaccctaaaatagtcggacagtcagcttatactatagcagctatggctggcataaaagtacctaaaaccacaagaatattaataggagaagttacctccttaggtgaagaagaaccttttgcccacgaaaaactatctcctgttttggctatgtatgaggctgacaattttgatgatgctttaaaaaaagcagtaactctaataaacttaggaggcctcggccatacctcaggaatatatgcagatgaaataaaagcacgagataaaatagatagatttagtagtgccatgaaaaccgtaagaacctttgtaaatatcccaacctcacaaggtgcaagtggagatctatataattttagaataccaccttctttcacgcttggctgcggattttggggaggaaattctgtttccgagaatgttggtccaaaacatcttttgaatattaaaaccgtagctgaaaggagagaaaacatgctttggtttagagttccacataaagtatattttaagttcggttgtcttcaatttgctttaaaagatttaaaagatctaaagaaaaaaagagcctttatagttactgatagtgacccctataatttaaactatgttgattcaataataaaaatacttgagcacctagatattgattttaaagtatttaataaggttggaagagaagctgatcttaaaaccataaaaaaagcaactgaagaaatgtcctcctttatgccagacactataatagctttaggtggtacccctgaaatgagctctgcaaagctaatgtgggtactatatgaacatccagaagtaaaatttgaagatcttgcaataaaatttatggacataagaaagagaatatatactttcccaaaactcggtaaaaaggctatgttagttgcaattacaacttctgctggttccggttctgaggttactccttttgctttagtaactgacaataacactggaaataagtacatgttagcagattatgaaatgacaccaaatatggcaattgtagatgcagaacttatgatgaaaatgccaaagggattaaccgcttattcaggtatagatgcactagtaaatagtatagaagcatacacatccgtatatgcttcagaatacacaaacggactagcactagaggcaatacgattaatatttaaatatttgcctgaggcttacaaaaacggaagaaccaatgaaaaagcaagagagaaaatggctcacgcttcaactatggcaggtatggcatccgctaatgcatttctaggtctatgtcattccatggcaataaaattaagttcagaacacaatattcctagtggcattgccaatgcattactaatagaagaagtaataaaatttaacgcagttgataatcctgtaaaacaagccccttgcccacaatataagtatccaaacaccatatttagatatgctcgaattgcagattatataaagcttggaggaaatactgatgaggaaaaggtagatctcttaattaacaaaatacatgaactaaaaaaagctttaaatataccaacttcaataaaggatgcaggtgttttggaggaaaacttctattcctcccttgatagaatatctgaacttgcactagatgatcaatgcacaggcgctaatcctagatttcctcttacaagtgagataaaagaaatgtatataaattgttttaaaaaacaaccttaa

SEQ ID NO: 67 - protein, 307 aa, Pseudomonas putida
MSKKLKAAIIGPGNIGTDLVMKMLRSEWIEPVWMVGIDPNSDGLKRARDFGMKTTAEGVDGLLPHVLDDDIRIAFDATSAYVHAENSRKLNALGVLMVDLTPAAIGPYCVPPVNLKQHVGRLEMNVNMVTCGGQATIPMVAAVSRVQPVAYAEIVATVSSRSVGPGTRKNIDEFTRTTAGAIEQVGGAREGKAIIVINPAEPPLMMRDTIHCLTDSEPDQAAITASVHAMIAEVQKYVPGYRLKNGPVFDGNRVSIFMEVEGLGDYLPKYAGNLDIMTAAALRTGEMFAEEIAAGTIQLPRRDIALA

SEQ ID NO: 68 - DNA, 2180 bp, Pseudomonas putida
ggtacccctggagccggtcaaggccggcgacttcatgcgcgtcgagatcggcggcatcggcagcgcctccgtgcgcttcacctgatcgaacagaggacaaacccatgagcaagaaactcaaggcggccatcataggccccggcaatatcggtaccgatctggtgatgaagatgctccgttccgagtggattgagccggtgtggatggtcggcatcgaccccaactccgacggcctcaaacgcgcccgcgatttcggcatgaagaccacagccgaaggcgtcgacggcctgctcccgcacgtgctggacgacgacatccgcatcgccttcgacgccacctcggcctatgtgcatgccgagaatagccgcaagctcaacgcgcttggcgtgctgatggtcgacctgaccccggcggccatcggcccctactgcgtgccgccggtcaacctcaagcagcatgtcggccgcctggaaatgaacgtcaacatggtcacctgcggcggccaggccaccatccccatggtcgccgcggtgtcccgcgtgcagccggtggcctacgccgagatcgtcgccaccgtctcctcgcgctcggtcggcccgggcacgcgcaagaacatcgacgagttcacccgcaccaccgccggcgccatcgagcaggtcggcggcgccagggaaggcaaggcgatcatcgtcatcaacccggccgagccgccgctgatgatgcgcgacaccatccactgcctgaccgacagcgagccggaccaggctgcgatcaccgcttcggttcacgcgatgatcgccgaggtgcagaaatacgtgcccggctaccgcctgaagaacggcccggtgttcgacggcaaccgcgtgtcgatcttcatggaagtcgaaggcctgggcgactacctgcccaagtacgccggcaacctcgacatcatgaccgccgccgcgctgcgtaccggcgagatgttcgccgaggaaatcgccgccggcaccattcaactgccgcgtcgcgacatcgcgctggcttgaggagtagcaccatgaatttgcacggcaagagcgtcatcctgcacgacatgagcctgcgcgacggcatgcacgccaagcgccaccagatcagcctggagcagatggtcgcggtcgccaccggcctcgatcaagccggtatgccgctgatcgagatcacccacggcgacggcctcggcggtcgttcgatcaactacggcttcccggcccacagtgacgaggagtacctgcgcgcggtgatcccgcagctcaagcaggccaaagtctcggcgctgctgctgcccggcatcggcaccgtcgaccacctgaagatggccctggactgcggcgtctcgactattcgcgtggccacccactgtaccgaggcggatgtctccgagcagcacatcggcatggcgcgcaagctgggggtcgacaccgtcggcttcctgatgatggcgcacatgatcagcgccgagaaagtcctggagcaggccaagctgatggaaagctatggtgccaactgcatctactgcaccgactcggccggctacatgctgcctgatgaagtcagcgagaaaatcggcctcctgcgcgccgagctgaacccggccaccgaagtcggcttccacggccaccacaacatgggcatggctatcgccaactcgctggccgccatcgaagccggtgccgcgcgcatcgacggctcggtcgccggcctcggcgccggtgccggcaacaccccgctggaagtgttcgtcgcagtgtgcaaacgcatgggcgtggagaccggcatcgacctgtacaagatcatggacgtggccgaggacctggtggtgccgatgatggatcagccgatccgcgtcgaccgcgacgccctgaccctgggctacgccggggtgtacagctcgttcctgctgttcgcccagcgcgccgagaagaaatatggcgtgtcggcccgcgacatcctggtcgaactgggccggcgcggcaccgtcggtggccaggaagacatgatcgaagacctcgccctggacatggcccgggcccgtcagcagcagaaggtgagcgcatgaaccgtaccctgacccgcgaacaggtgctggccctggccgagcacatcgaaaacgccgagctgaatgtccacgacatcggcaaggtgaccaacgattttcc

SEQ ID NO: 69 - protein, 307 aa, Thermus thermophilus
MSERVKVAILGSGNIGTDLMYKLLKNPGHMELVAVVGIDPKSEGLARARALGLEASHEGIAYILERPEIKIVFDATSAKAHVRHAKLLREAGKIAIDLTPAARGPYVVPPVNLKEHLDKDNVNLITCGGQATIPLVYAVHRVAPVLYAEMVSTVASRSAGPGTRQNIDEFTFTTARGLEAIGGAKKGKAIIILNPAEPPILMTNTVRCIPEDEGFDREAVVASVRAMEREVQAYVPGYRLKADPVFERLPTPWGERTVVSMLLEVEGAGDYLPKYAGNLDIMTASARRVGEVFAQHLLGKPVEEVVA

SEQ ID NO: 70 - DNA, 924 bp, Thermus thermophilus
atgtccgaaagggttaaggtagccatcctgggctccggcaacatcgggacggacctgatgtacaagctcctgaagaacccgggccacatggagcttgtggcggtggtggggatagaccccaagtccgagggcctggcccgggcgcgggccttagggttagaggcgagccacgaagggatcgcctacatcctggagaggccggagatcaagatcgtctttgacgccaccagcgccaaggcccacgtgcgccacgccaagctcctgagggaggcggggaagatcgccatagacctcacgccggcggcccggggcccttacgtggtgcccccggtgaacctgaaggaacacctggacaaggacaacgtgaacctcatcacctgcggggggcaggccaccatccccctggtctacgcggtgcaccgggtggcccccgtgctctacgcggagatggtctccacggtggcctcccgctccgcgggccccggcacccggcagaacatcgacgagttcaccttcaccaccgcccggggcctggaggccatcgggggggccaagaaggggaaggccatcatcatcctgaacccggcggaaccccccatcctcatgaccaacaccgtgcgctgcatccccgaggacgagggctttgaccgggaggccgtggtggcgagcgtccgggccatggagcgggaggtccaggcctacgtgcccggctaccgcctgaaggcggacccggtgtttgagaggcttcccaccccctggggggagcgcaccgtggtctccatgctcctggaggtggagggggcgggggactatttgcccaaatacgccggcaacctggacatcatgacggcttctgcccggagggtgggggaggtcttcgcccagcacctcctggggaagcccgtggaggaggtggtggcgtga

SEQ ID NO: 71 - protein, 417 aa, Escherichia coli
MTFSLFGDKFTRHSGITLLMEDLNDGLRTPGAIMLGGGNPAQIPEMQDYFQTLLTDMLESGKATDALCNYDGPQGKTELLTLLAGMLREKLGWDIEPQNIALTNGSQSAFFYLFNLFAGRRADGRVKKVLFPLAPEYIGYADAGLEEDLFVSARPNIELLPEGQFKYHVDFEHLHIGEETGMICVSRPTNPTGNVITDEELLKLDALANQHGIPLVIDNAYGVPFPGIIFSEARPLWNPNIVLCMSLSKLGLPGSRCGIIIANEKIITAITNMNGIISLAPGGIGPAMMCEMIKRNDLLRLSETVIKPFYYQRVQETIAIIRRYLPENRCLIHKPEGAIFLWLWFKDLPITTKQLYQRLKARGVLMVPGHNFFPGLDKPWPHTHQCMRMNYVPEPEKIEAGVKILAEEIERAWAESH

SEQ ID NO: 72 - protein, 417 aa, Escherichia coli; identical to SEQ ID NO: 71

SEQ ID NO: 73 - protein, 425 aa, Bacillus licheniformis
MKPPLSKIGEKMIEKTGVRAVMSDIQEVLAGGERSYINLSAGNPMILPGVSAMWKSALADLLDDDRFSSVIGQYGSSYGTDELIASVVRFFSERYSAGIRKENVLITAGSQQLFFLAINSFCGMGSGSVMKKALIPMLPDYSGYSGAALEREMIEGIPPLISKLDDHTFRYELDRKGFLERMRIGAVLLSRPNNPCGNILPKEDVAFISDACREANVPLFIDSAYAPPFPAIHFIDMEPIFNEQIIHCMSLSKAGLPGERIGIAIGPSRYIQAMEAFQSNAAIHSSRLGQYMAASVLNDGRLADVSLNEVRPYYRNKFMLLKETLLCKMPEDIKWYLHQGEGSLFGWLWFEDLPVTDAALYEYMKADGVIIVPGSSFFHRQSRRLAHSHQCIRISLTAADEDIIRGIDVLAKIAKGVYEKQVEYL

SEQ ID NO: 74 - DNA, 1278 bp, Bacillus licheniformis
ttataagtattcaacctgtttctcatatacacccttcgcaattttagctaaaacatcgattccccttataatatcttcatccgccgcggttaggctgattcgtatacactggtgtgaatgcgccaggcgccgggattgacggtgaaagaaagatgatccgggaacgataatgactccatccgctttcatatactcatacagcgctgcatcggtcaccggcaggtcttcaaaccacagccatccgaaaagcgatccttccccttgatgcagataccatttgatgtcttcaggcatcttgcataaaagcgtttccttgagcagcatgaatttattgcggtaatatggcctgacttcattcagcgacacgtcggcgaggcgcccgtcattcaatactgatgcagccatatactgccccagccttgaagaatggatcgccgcattcgactgaaaagcttccattgcctgaatataccgggacggcccgatggcgattccgatcctttcgccaggcaggccggcttttgaaaggctcatacagtgaatgatctgctcgttgaaaatcggttccatgtcgataaagtgaatcgccggaaaaggcggagcatatgcggaatcaatgaacagcggaacattcgcttctcggcatgcgtctgaaatgaatgctacatcttctttaggcaagatgtttccgcaaggattgttcgggcgcgatagcaagacagcaccgatgcgcatcctctctaaaaaccccttacggtcgagctcatatcgaaacgtatgatcatccaatttcgatatgagcggagggatcccctcaatcatctcccgctccagtgccgccccgctgtatcccgaatagtcaggcagcatcgggatcaaggcttttttcatcacagatccgcttcccattccgcaaaacgaattgatcgccagaaaaaacagctgctggcttccggctgtaatcaacacgttctcttttcgaatgccggcgctataccgctctgaaaagaagcggacaacacttgcaatcagttcatcggttccatagctcgatccgtattggccgatcaccgaagaaaacctgtcatcgtcaaggagatcggcaagagccgacttccacatggctgacacgccgggcaaaatcatcggattgcccgcacttaaattaatgtatgaccgttcaccgccggccaggacttcctgaatatcgctcatcacagccctgacccctgttttctcaatcattttctctccgattttgcttaatggcggcttcac

SEQ ID NO: 75 - protein, 309 aa, Escherichia coli
MTTKKADYIWFNGEMVRWEDAKVHVMSHALHYGTSVFEGIRCYDSHKGPVVFRHREHMQRLHDSAKIYRFPVSQSIDELMEACRDVIRKNNLTSAYIRPLIFVGDVGMGVNPPAGYSTDVIIAAFPWGAYLGAEALEQGIDAMVSSWNRAAPNTIPTAAKAGGNYLSSLLVGSEARRHGYQEGIALDVNGYISEGAGENLFEVKDGVLFTPPFTSSALPGITRDAIIKLAKELGIEVREQVLSRESLYLADEVFMSGTAAEITPVRSVDGIQVGEGRCGPVTKRIQQAFFGLFTGETEDKWGWLDQVNQ

SEQ ID NO: 76 - DNA, 1476 bp, Escherichia coli; identical to SEQ ID NO: 8

SEQ ID NO: 77 - protein, 376 aa, Saccharomyces cerevisiae
MTLAPLDASKVKITTTQHASKPKPNSELVFGKSFTDHMLTAEWTAEKGWGTPEIKPYQNLSLDPSAVVFHYAFELFEGMKAYRTVDNKITMFRPDMNMKRMNKSAQRICLPTFDPEELITLIGKLIQQDKCLVPEGKGYSLYIRPTLIGTTAGLGVSTPDRALLYVICCPVGPYYKTGFKAVRLEATDYATRAWPGGCGDKKLGANYAPCVLPQLQAASRGYQQNLWLFGPNNNITEVGTMNAFFVFKDSKTGKKELVTAPLDGTILEGVTRDSILNLAKERLEPSEWTISERYFTIGEVTERSKNGELLEAFGSGTAAIVSPIKEIGWKGEQINIPLLPGEQTGPLAKEVAQWINGIQYGETEHGNWSRVVTDLN

SEQ ID NO: 78 - protein, 376 aa, Saccharomyces cerevisiae; identical to SEQ ID NO: 77

SEQ ID NO: 79 - protein, 330 aa, Methanobacterium thermoautotrophicum
MRLWRALYRPPTITYPSKSPEVIIMSCEASGKIWLNGEMVEWEEATVHVLSHVVHYGSSVFEGIRCYRNSKGSAIFRLREHVKRLFDSAKIYRMDIPYTQEQICDAIVETVRENGLEECYIRPVVFRGYGEMGVHPVNCPVDVAVAAWEWGAYLGAEALEVGVDAGVSTWRRMAPNTMPNMAKAGGNYLNSQLAKMEAVRHGYDEAIMLDYHGYISEGSGENIFLVSEGEIYTPPVSSSLLRGITRDSVIKIARTEGVTVHEEPITREMLYIADEAFFTGTAAEITPIRSVDGIEIGAGRRGPVTKLLQDEFFRIIRAETEDSFGWLTYI

SEQ ID NO: 80 - DNA, 993 bp, Methanobacterium thermoautotrophicum
tcagatgtaggtgagccatccgaagctgtcctctgtctctgccctgattatcctgaagaactcatcctgcagcagctttgtaacgggaccccttcgcccggcacctatctctataccatcaactgatctgatgggtgttatctctgcggctgtacctgtgaagaaggcctcatctgcgatgtagagcatctccctggttatgggttcctcatgcacggtaacaccctcggtcctggctatctttattacggagtcccttgttatccccctcagaagggatgatgaaacagggggggtgtaaatttcaccctcactgacgaggaatatgttctccccgctaccctcacttatgtagccatggtagtccagcattatggcctcatcatagccgtgtctcacagcctccatcttggcaagctgtgagttgaggtagttaccgccggcctttgccatgttgggcattgtgtttggtgccatcctccgccaggttgaaacaccagcatcgacaccaacctcaagggcctctgcacccagataggccccccattcccaggcagccacagcgacgtccactgggcagttcaccgggtgaacacccatctcaccgtatcccctgaataccacgggtcttatatagcactcctcaagtccgttctccctgacggtctcaactatggcatcacatatctgctcctgggtgtagggtatgtccatccggtatatctttgcagaatcaaaaaggcgtttaacatgctcccgcaaacggaagatggctgaccccttactgttcctgtagcaccttattccctcaaagacagatgatccataatgcacaacatgtgagagtacgtggacggtggcttcttcccattcaaccatttcaccgtttaaccatatctttccactggcttcgcatgacatgataataacctcaggtgatttactaggataggttatggttggaggcctatataatgctctccataaccgcaa

SEQ ID NO: 81 - protein, 364 aa, Streptomyces coelicolor
MTDVNGAPADVLHTLFHSDQGGHEQVVLCQDRASGLKAVIALHSTALGPALGGTRFYPYASEAEAVADALNLARGMSYKNAMAGLDHGGGKAVIIGDPEQIKSEELLLAYGRFVASLGGRYVTACDVGTYVADMDVVARECRWTTGRSPENGGAGDSSVLTSFGVYQGMRAAAQHLWGDPTLRDRTVGIAGVGKVGHHLVEHLLAEGAHVVVTDVRKDVVRGITERHPSVVAVADTDALIRVENLDIYAPCALGGALNDDTVPVLTAKVVCGAANNQLAHPGVEKDLADRGILYAPDYVVNAGGVIQVADELHGFDFDRCKAKASKIYDTTLAIFARAKEDGIPPAAAADRIAEQRMAEARPRP

SEQ ID NO: 82 - DNA, 1095 bp, Streptomyces coelicolor
tcacggccggggacgggcctccgccatccgctgctcggcgatccggtcggccgccgcggccggcggaataccgtcctccttcgcacgtgcgaatatggccagcgtggtgtcgtagatcttcgaggccttcgccttgcaccggtcgaagtcgaacccgtgcagctcgtcggcgacctggatgacaccgccggcgttcaccacatagtccggcgcgtagaggatcccgcggtcggcgaggtccttctcgacgcccgggtgggcgagctggttgttggccgcgccgcacaccaccttggcggtcagcaccggcacggtgtcgtcgttcagcgcgccgccgagcgcgcagggcgcgtagatgtccaggttctccacccggatcagcgcgtcggtgtcggcgacggcgaccaccgacgggtgccgctccgtgatcccgcgcaccacgtccttgcgcacgtccgtgacgacgacgtgggcgccctcggcgagcaggtgctcgaccaggtggtggccgaccttgccgacgcccgcgatgccgacggtgcggtcgcgcagcgtcgggtcgccccacaggtgctgggcggcggcccgcatgccctggtagacgccgaaggaggtgagcacggaggagtcgcccgcgccgccgttctccggggaacgcccggtcgtccagcggcactcgcgggccacgacgtccatgtcggcgacgtaggtgccgacgtcgcacgcggtgacgtagcggccgcccagcgaggcgacgaaccggccgtaggcgaggagcagctcctcgctcttgatctgctccggatcgccgatgatcacggccttgccgccaccgtggtccagaccggccatggcgttcttgtacgacatcccgcgggcgaggttcagcgcgtcggcgacggcctccgcctcgctcgcgtacgggtagaagcgggtaccgccgagcgccgggcccagggcggtggagtggagggcgatcacggccttgaggccgctggcacggtcctggcagagcacgacttgctcatgtcccccctgatccgagtggaacagggtgtgcagtacatcagcaggtgcgccgtttacgtcggtcac

SEQ ID NO: 83 - protein, 364 aa, Bacillus subtilis
MELFKYMEKYDYEQLVFCQDEQSGLKAIIAIHDTTLGPALGGTRMWTYENEEAAIEDALRLARGMTYKNAAAGLNLGGGKTVIIGDPRKDKNEEMFRAFGRYIQGLNGRYITAEDVGTTVEDMDIIHDETDYVTGISPAFGSSGNPSPVTAYGVYRGMKAAAKAAFGTDSLEGKTIAVQGVGNVAYNLCRHLHEEGANLIVTDINKQSVQRAVEDFGARAVDPDDIYSQDCDIYAPCALGATINDDTIKQLKAKVIAGAANNQLKETRHGDQIHEMGIVYAPDYVINAGGVINVADELYGYNAERALKKVEGIYGNIERVLEISQRDGIPAYLAADRLAEERIERMRRSRSQFLQNGHSVLSRR

SEQ ID NO: 84 - protein, 364 aa, Bacillus subtilis; identical to SEQ ID NO: 83

SEQ ID NO: 85 - protein, 594 aa, Streptomyces viridifaciens
MSTSSASSGPDLPFGPEDTPWQKAFSRLRAVDGVPRVTAPSSDPREVYMDIPEIPFSKVQIPPDGMDEQQYAEAESLFRRYVDAQTRNFAGYQVTSDLDYQHLSHYLNRHLNNVGDPYESSSYTLNSKVLERAVLDYFASLWNAKWPHDASDPETYWGYVLTMGSSEGNLYGLWNARDYLSGKLLRRQHREAGGDKASVVYTQALRHEGQSPHAYEPVAFFSQDTHYSLTKAVRVLGIDTFHSIGSSRYPDENPLGPGTPWPTEVPSVDGAIDVDKLASLVRFFASKGYPILVSLNYGSTFKGAYDDVPAVAQAVRDICTEYGLDRRRVYHDRSKDSDFDERSGFWIHIDAALGAGYAPYLQMARDAGMVEEAPPVFDFRLPEVHSLTMSGHKWMGTPWACGVYMTRTGLQMTPPKSSEYIGAADTTFAGSRNGFSSLLLWDYLSRHSYDDLVRLAADCDRLAGYAHDRLLTLQDKLGMDLWVARSPQSLTVRFRQPCADIVRKYSLSCETVYEDNEQRTYVHLYAVPHLTRELVDELVRDLRQPGAFTNAGALEGEAWAGVIDALGRPDPDGTYAGALSAPASGPRSEDGGGS

SEQ ID NO: 86 - DNA, 1785 bp, Streptomyces viridifaciens
gtgtcaacttcctccgcttcttccgggccggacctccccttcgggcccgaggacacgccatggcagaaggccttcagcaggctgcgggcggtggatggcgtgccgcgcgtcaccgcgccgtccagtgatccgcgtgaggtctacatggacatcccggagatccccttctccaaggtccagatccccccggacggaatggacgagcagcagtacgcagaggccgagagcctcttccgccgctacgtagacgcccagacccgcaacttcgcgggataccaggtcaccagcgacctcgactaccagcacctcagtcactatctcaaccggcatctgaacaacgtcggcgatccctatgagtccagctcctacacgctgaactccaaggtccttgagcgagccgttctcgactacttcgcctccctgtggaacgccaagtggccccatgacgcaagcgatccggaaacgtactggggttacgtgctgaccatgggctccagcgaaggcaacctgtacgggttgtggaacgcacgggactatctgtcgggcaagctgctgcggcgccagcaccgggaggccggcggcgacaaggcctcggtcgtctacacgcaagcgctgcgacacgaagggcagagtccgcatgcctacgagccggtggcgttcttctcgcaggacacgcactactcgctcacgaaggccgtgcgggttctgggcatcgacaccttccacagcatcggcagcagtcggtatccggacgagaacccgctgggccccggcactccgtggccgaccgaagtgccctcggttgacggtgccatcgatgtcgacaaactcgcctcgttggtccgcttcttcgccagcaagggctacccgatactggtcagcctcaactacgggtcaacgttcaagggcgcctacgacgacgtcccggccgtggcacaggccgtgcgggacatctgcacggaatacggtctggatcggcggcgggtataccacgaccgcagtaaggacagtgacttcgacgagcgcagcggcttctggatccacatcgatgccgccctgggggcgggctacgctccctacctgcagatggcccgggatgccggcatggtcgaggaggcgccgcccgttttcgacttccggctcccggaggtgcactcgctgaccatgagcggccacaagtggatgggaacaccgtgggcatgcggtgtctacatgacacggaccgggctgcagatgaccccgccgaagtcgtccgagtacatcggggcggccgacaccaccttcgcgggctcccgcaacggcttctcgtcactgctgctgtgggactacctgtcccggcattcgtatgacgatctggtgcgcctggccgccgactgcgaccggctggccggctacgcccacgaccggttgctgaccttgcaggacaaactcggcatggatctgtgggtcgcccgcagcccgcagtccctcacggtgcgcttccgtcagccatgtgcagacatcgtccgcaagtactcgctgtcgtgtgagacggtctacgaagacaacgagcaacggacctacgtacatctctacgccgttccccacctcactcgggaactcgtggatgagctcgtgcgcgatctgcgccagcccggagccttcaccaacgctggtgcactggagggggaggcctgggccggggtgatcgatgccctcggccgcccggaccccgacggaacctatgccggcgccttgagcgctccggcttccggcccccgctccgaggacggcggcgggagctga

SEQ ID NO: 87 - protein, 440 aa, Alcaligenes denitrificans
MSAAKLPDLSHLWMPFTANRQFKANPRLLASAKGMYYTSFDGRQILDGTAGLWCVNAGHCREEIVSAIASQAGVMDYAPGFQLGHPLAFEAATAVAGLMPQGLDRVFFTNSGSESVDTALKIALAYHRARGEAQRTRLIGRERGYHGVGFGGISVGGISPNRKTFSGALLPAVDHLPHTHSLEHNAFTRGQPEWGAHLADELERIIALHDASTIAAVIVEPMAGSTGVLVPPKGYLEKLREITARHGILLIFDEVITAYGRLGEATAAAYFGVTPDLITMAKGVSNAAVPAGAVAVRREVHDAIVNGPQGGIEFFHGYTYSAHPLAAAAVLATLDIYRREDLFARARKLSAAFEEAAHSLKGAPHVIDVRNIGLVAGIELSPREGAPGARAAEAFQKCFDTGLMVRYTGDILAVSPPLIVDENQIGQIFEGIGKVLKEVA

SEQ ID NO: 88 - DNA, 1947 bp, Alcaligenes denitrificans
ttcgatggcgcgctgcacggcggccaccagctgctccaccaggggtgggcgcctgcccgcgcgcgcggtcgggctggaaatcgatcatggatgaatctatacagttgtcatgattgcaactatacagttagcccgttttgcggcaattgtatattttcattcgctcgtggacgtccgagaatcggtttgatcgcgccgcccgcccctttccgcgcagcggcgtttcttttcctccggagtctccccatgagcgctgccaaactgcccgacctgtcccacctctggatgccctttaccgccaaccggcagttcaaggcgaacccccgcctgctggcctcggccaagggcatgtactacacgtctttcgacggccgccagatcctggacggcacggccggcctgtggtgcgtgaacgccggccactgccgcgaagaaatcgtctccgccatcgccagccaggccggcgtcatggactacgcgccggggttccagctcggccacccgctggccttcgaggccgccaccgccgtggccggcctgatgccgcagggcctggaccgcgtgttcttcaccaattcgggctccgaatcggtggacaccgcgctgaagatcgccctggcctaccaccgcgcgcgcggcgaggcgcagcgcacccgcctcatcgggcgcgagcgcggctaccacggcgtgggcttcggcggcatttccgtgggcggcatctcgcccaaccgcaagaccttctccggcgcgctgctgccggccgtggaccacctgccgcacacccacagcctggaacacaacgccttcacgcgcggccagcccgagtggggcgcgcacctggccgacgagttggaacgcatcatcgccctgcacgacgcctccaccatcgcggccgtgatcgtcgagcccatggccggctccaccggcgtgctcgtcccgcccaagggctatctcgaaaaactgcgcgaaatcaccgcccgccacggcattctgctgatcttcgacgaagtcatcaccgcgtacggccgcctgggcgaggccaccgccgcggcctatttcggcgtaacgcccgacctcatcaccatggccaagggcgtgagcaacgccgccgttccggccggcgccgtcgcggtgcgccgcgaagtgcatgacgccatcgtcaacggaccgcaaggcggcatcgagttcttccacggctacacctactcggcccacccgctggccgccgccgccgtgctcgccacgctggacatctaccgccgcgaagacctgttcgcccgcgcccgcaagctgtcggccgcgttcgaggaagccgcccacagcctcaagggcgcgccgcacgtcatcgacgtgcgcaacatcggcctggtggccggcatcgagctgtcgccgcgcgaaggcgccccgggcgcgcgcgccgccgaagccttccagaaatgcttcgacaccggcctcatggtgcgctacacgggcgacatcctcgcggtgtcgcctccgctcatcgtcgacgaaaaccagatcggccagatcttcgagggcatcggcaaggtgctcaaggaagtggcttagggtgaacacgccctgagccggccccggcaggaaacgcgccgccgcgcggcggcgcgtccatcgaactcccgcatcgagcttttgcattcatgaagaaaatcacgcatttcatcaacggccagccccacgaaggccgcagcaaccgctacaccgagggcttcaacccggccacgggcgagtcgtctcctcgatctgcctgggcggggccgaagaagtggacctggccgtggcggccgcccgcgcggcctttcccgcctggtccgaaacgccggcgctcaagcgcgcgcgcgtgctgttcaacttcaaggcgctgctggacaagcaccaggacgagctggccgcgctcatcacgcgcgagcacggcaaggtgttttccga

SEQ ID NO: 89 - protein, 443 aa, Ralstonia eutropha
MDAAKTVIPDLDALWMPFTANRQYKAAPRLLASASGMYYTTHDGRQILDGCAGLWCVAAGHCRKEIAEAVARQAATLDYAPPFQMGHPLSFEAATKVAAIMPQGLDRIFFTNSGSESVDTALKIALAYHRARGEGQRTRFIGRERGYHGVGFGGMAVGGIGPNRKAFSANLMPGTDHLPATLNIAEAAFSKGQPTWGAHLADELERIVALHDPSTIAAVIVEPLAGSAGVLVPPVGYLDKLREITTKHGILLIFDEVITAFGRLGTATAAERFKVTPDLITMAKAINNAAVPMGAVAVRREVHDTVVNSAAPGAIELAHGYTYSGHPLAAAAAIATLDLYQRENLFGRAAELSPVFEAAVHSVRSAPHVKDIRNLGMVAGIELEPRPGQPGARAYEAFLKCLERGVLVRYTGDILAFSPPLIISEAQIAELFDTVKQALQEVQ

SEQ ID NO: 90 - DNA, 1341 bp, Ralstonia eutropha
atggccgactcacccaacaacctcgctcacgaacatccttcacttgaacactattggatgccttttaccgccaatcgccaattcaaagcgagccctcgtttactcgcccaagctgaaggtatgtattacacagatatcaatggcaacaaggtattagactctacagcgggcttatggtgttgtaatgctggccatggtcgccgtgagatcagtgaagccgtcagcaaacaaattcggcagatggattacgctccctccttccaaatgggccatcccatcgcttttgaactggccgaacgtttaaccgaactcagcccagaaggactcaacaaagtattctttaccaactcaggctctgagtcggttgataccgcgctaaaaatggctctttgctaccatagagccaatggccaagcgtcacgcacccgctttattggccgtgaaatgggttaccatggcgtaggatttggtgggatctcggtgggtggtttaagcaataaccgtaaagccttcagcggccagctattgcaaggcgtggatcacctgccccacaccttagacattcaacatgccgcctttagtcgtggcttaccgagcctcggtgctgaaaaagctgaggtattagaacaattagtcacactccatggcgccgaaaatattgccgccgttattgttgaacccatgtcaggttctgcaggggtaattttaccacctcaaggctacttaaaacgcttacgtgaaatcactaaaaaacacggcatcttattgattttcgatgaagtcattaccgcatttggccgtgtaggtgcagcattcgccagccaacgttggggcgttattccagacataatcaccacggctaaagccattaataatggcgccatccccatgggcgcagtgtttgtacaggattatatccacgatacttgcatgcaagggccaaccgaactgattgaatttttccacggttatacctattcgggccacccagtcgccgcagcagcagcactcgccacgctctccatctaccaaaacgagcaactgtttgagcgcagttttgagcttgagcggtatttcgaagaagccgttcatagcctcaaagggttaccgaatgtgattgatattcgcaacaccggattagtcgcgggtttccagctagcaccgaatagccaaggtgttggtaaacgcggatacagcgtgttcgagcattgtttccatcaaggcacactcgtgcgggcaacgggcgatattatcgccatgtccccaccactcattgttgagaaacatcagattgaccaaatggtaaatagccttagcgatgcaattcacgccgttggatga

SEQ ID NO: 91 - protein, 446 aa, Shewanella oneidensis
MADSPNNLAHEHPSLEHYWMPFTANRQFKASPRLLAQAEGMYYTDINGNKVLDSTAGLWCCNAGHGRREISEAVSKQIRQMDYAPSFQMGHPIAFELAERLTELSPEGLNKVFFTNSGSESVDTALKMALCYHRANGQASRTRFIGREMGYHGVGFGGISVGGLSNNRKAFSGQLLQGVDHLPHTLDIQHAAFSRGLPSLGAEKAEVLEQLVTLHGAENIAAVIVEPMSGSAGVILPPQGYLKRLREITKKHGILLIFDEVITAFGRVGAAFASQRWGVIPDIITTAKAINNGAIPMGAVFVQDYIHDTCMQGPTELIEFFHGYTYSGHPVAAAAALATLSIYQNEQLFERSFELERYFEEAVHSLKGLPNVIDIRNTGLVAGFQLAPNSQGVGKRGYSVFEHCFHQGTLVRATGDIIAMSPPLIVEKHQIDQMVNSLSDAIHAVG

SEQ ID NO: 92 - DNA, 1341 bp, Shewanella oneidensis; identical to SEQ ID NO: 90

SEQ ID NO: 93 - protein, 448 aa, Pseudomonas putida
MNMPETGPAGIASQLKLDAHWMPYTANRNFQRDPRLIVAAEGNYLVDDHGRKIFDALSGLWTCGAGHTRKEIADAVTRQLSTLDYSPAFQFGHPLSFQLAEKIAELVPGNLNHVFYTNSGSECADTALKMVRAYWRLKGQATKTKIIGRARGYHGVNIAGTSLGGVNGNRKMFGQLLDVDHLPHTVLPVNAFSKGLPEEGGIALADEMLKLIELHDASNIAAVIVEPLAGSAGVLPPPKGYLKRLREICTQHNILLIFDEVITGFGRMGAMTGSEAFGVTPDLMCIAKQVTNGAIPMGAVIASSEIYQTFMNQPTPEYAVEFPHGYTYSAHPVACAAGLAALDLLQKENLVQSAAELAPHFEKLLHGVKGTKNIVDIRNYGLAGAIQIAARDGDAIVRPYEAAMKLWKAGFYVRFGGDTLQFGPTFNTKPQELDRLFDAVGETLNLID

SEQ ID NO: 94 - DNA, 930 bp, Pseudomonas putida
atgaccacgaagaaagctgattacatttggttcaatggggagatggttcgctgggaagacgcgaaggtgcatgtgatgtcgcacgcgctgcactatggcacttcggtttttgaaggcatccgttgctacgactcgcacaaaggaccggttgtattccgccatcgtgagcatatgcagcgtctgcatgactccgccaaaatctatcgcttcccggtttcgcagagcattgatgagctgatggaagcttgtcgtgacgtgatccgcaaaaacaatctcaccagcgcctatatccgtccgctgatcttcgtcggtgatgttggcatgggagtaaacccgccagcgggatactcaaccgacgtgattatcgctgctttcccgtggggagcgtatctgggcgcagaagcgctggagcaggggatcgatgcgatggtttcctcctggaaccgcgcagcaccaaacaccatcccgacggcggcaaaagccggtggtaactacctctcttccctgctggtgggtagcgaagcgcgccgccacggttatcaggaaggtatcgcgctggatgtgaacggttatatctctgaaggcgcaggcgaaaacctgtttgaagtgaaagatggtgtgctgttcaccccaccgttcacctcctccgcgctgccgggtattacccgtgatgccatcatcaaactggcgaaagagctgggaattgaagtacgtgagcaggtgctgtcgcgcgaatccctgtacctggcggatgaagtgtttatgtccggtacggcggcagaaatcacgccagtgcgcagcgtagacggtattcaggttggcgaaggccgttgtggcccggttaccaaacgcattcagcaagccttcttcggcctcttcactggcgaaaccgaagataaatggggctggttagatcaagttaatcaataa

SEQ ID NO: 95 - protein, 566 aa, Streptomyces cinnamonensis
MDADAIEEGRRRWQARYDKARKRDADFTTLSGDPVDPVYGPRPGDTYDGFERIGWPGEYPFTRGLYATGYRGRTWTIRQFAGFGNAEQTNERYKMILANGGGGLSVAFDMPTLMGRDSDDPRSLGEVGHCGVAIDSAADMEVLFKDIPLGDVTTSMTISGPAVPVFCMYLVAAERQGVDPAVLNGTLQTDIFKEYIAQKEWLFQPEPHLRLIGDLMEHCARDIPAYKPLSVSGYHIREAGATAAQELAYTLADGFGYVELGLSRGLDVDVFAPGLSFFFDAHVDFFEEIAKFRAARRIWARWLRDEYGAKTEKAQWLRFHTQTAGVSLTAQQPYNNVVRTAVEALAAVLGGTNSLHTNALDETLALPSEQAAEIALRTQQVLMEETGVANVADPLGGSWYIEQLTDRIEADAEKIFEQIRERGRRACPDGQHPIGPITSGILRGIEDGWFTGEIAESAFQYQRSLEKGDKRVVGVNCLEGSVTGDLEILRVSHEVEREQVRELAGRKGRRDDARVRASLDAMLAAARDGSNMIAPMLEAVRAEATLGEICGVLRDEWGVYVEPPGF

SEQ ID NO: 96 - DNA, 4362 bp, Streptomyces cinnamonensis
tgaggcgctggatcgcctcggagagcagctggtaacggtccgcgtggtactcggccggggtgcagccgtccacgatgtgcgggatcgcgtcgggctcgaggatcaccagggcgggggcgtcgccgatcgcgtcggcgaacgtgtccacccagctccggtaggcctccgcactggccgcgccgcccgcggagtgctgaccgcagtcgcggtgcgggatgttgtacgcgacgagtacggcggtgcggtcctccttgaccgcgccccgcgtcgccttcgcgacgtcgggcgccggatcgtccccggccggccacacggccatggcccgttcggagatgcgcctgagcgtctcggcgtcctcggcgcggccctgttcctcccactgcctgacctggcgcgcggcggggctgtcggggtcgacccagaaggtgccggcggggggcccggcgctcgcggtggcgggcttgcgcacggccgcctcctccttcgtgccgtcggaccccgggtctgaggaggagcagcctgccgggagcccgagggcggcgagggccgcgagtgccgtgaacgtgcggagcagccggtgcatccagcccccttgggcgatggtgacagtgacggtcagtcagcccggcaatcgttacataaaggactattcaagctcttgtgccacaccgcctccggtgccgagcgcgaacccggcgggcaccagagccccgccgcggccgcggagccgtacgtacgaccgaattgcgagacggggctgaccaccatatgaccggcgggtaaggtcgatgccgtgccgaagccgctcagcctccccttcgatcccatcgcccgcgccgacgagctctggaagcagcgctggggatcggtcccggccatgggcgcgatcacctcgatcatgcgggcgcaccagatcctgctcgccgaggtcgacgcggtcgtcaagccgtacggactgaccttcgcgcgctacgaggcgctggtgctcctcaccttctcgcaggccggcgagttgccgatgtcgaagatcggcgagcggctcatggtgcacccgacctcggtcacgaacaccgtggaccgcctggtgaagtccggcctggtcgacaagcgcccgaaccccaacgacggccgcggcacgctcgcctccatcacggagaagggccgcgaggtcgtcgaggcggccacccgcgagctgatggcgatggacttcgggctcggggtgtacgacgcggaggagtgcggggagatcttcgcgatgctgcggcccctgcgggtggcggcgcgcgatttcgaggagcagtagggcccgcccggtgagaagtgggatcgggtcgtcccggtacgggcgggggcggcgaagatcgcgtgaaaagggcggttacgctcgtagccatgaaacgcagcgtgctgacccgctaccgggtgatggcctacgtcaccgccgtcatgctcctcatcctgtgcgcctgcatggtggccaagtacggcttcgacaagggcgagggtctgaccctcgtcgtgtcgcaggtgcacggcgtgctctacatcatctacctgatcttcgccttcgacctgggctccaaggcgaagtggccgttcggcaagctgctctgggtgctggtctcgggcacgatcccgaccgccgccttcttcgtcgagcgcaaggtcgcccgtgacgtcgagccgctgatcgccgacggctccccggtcaccgcgaaggcgtaacccgcaccgccacggacaggtccgtggcggttggccatcgacttttactaggacgtcctagtaaattcgatggtatggacgctgacgcgatcgaggaaggccgccgacgctggcaggcccgttacgacaaggcccgcaagcgcgacgcggacttcaccacgctctccggggaccccgtcgaccccgtctacggcccccggcccggggacacgtacgacgggttcgagcggatcggctggccgggggagtaccccttcacccgcgggctctacgccaccgggtaccgcggccgcacctggaccatccgccagttcgccggcttcggcaacgccgagcagacgaacgagcgctacaagatgatcctggccaacggcggcggcggcctctccgtcgccttcgacatgccgaccctcatgggccgcgactccgacgacccgcgctcgctcggcgaggtcggccactgcggtgtcgccatcgactccgccgccgacatggaggtcctcttcaaggacatcccgctcggcgacgtcacgacgtccatgaccatcagcgggcccgccgtgcccgtcttctgcatgtacctcgtcgcggccgagcgccagggcgtcgacccggccgtcctcaacggcacgctgcagaccgacatcttcaaggagtacatcgcccagaaggagtggctcttccagcccgagccgcacctgcgcctcatcggcgacctgatggagcactgcgcgcgcgacatccccgcgtacaagccgctctcggtctccggctaccacatccgcgaggccggggcgacggccgcgcaggagctcgcgtacaccctcgcggacggcttcgggtacgtggaactgggcctctcgcgcggcctggacgtggacgtcttcgcgcccggcctctccttcttcttcgacgcgcacgtcgacttcttcgaggagatcgcgaagttccgcgccgcacgccgcatctgggcgcgctggctccgggacgagtacggagcgaagaccgagaaggcacagtggctgcgcttccacacgcagaccgcgggggtctcgctcacggcccagcagccgtacaacaacgtggtgcggacggcggtggaggccctcgccgcggtgctcggcggcacgaactccctgcacaccaacgctctcgacgagacccttgccctccccagcgagcaggccgcggagatcgcgctgcgcacccagcaggtgctgatggaggagaccggcgtcgccaacgtcgcggacccgctgggcggctcctggtacatcgagcagctcaccgaccgcatcgaggccgacgccgagaagatcttcgagcagatcagggagcgggggcggcgggcctgccccgacgggcagcacccgatcgggccgatcacctccggcatcctgcgcggcatcgaggacggctggttcaccggcgagatcgccgagtccgccttccagtaccagcggtccctggagaagggcgacaagcgggtcgtcggcgtcaactgcctcgaaggctccgtcaccggcgacctggagatcctgcgcgtcagccacgaggtcgagcgcgagcaggtgcgggagcttgcggggcgcaaggggcggcgtgacgatgcgcgggtgcgggcctcgctcgacgcgatgctcgccgctgcgcgggacgggtcgaacatgattgcccccatgctggaggcggtgcgggccgaggcgaccctcggggagatctgcggggtgcttcgcgatgagtggggggtctacgtggagccgcccgggttctgagggcgcgctccctttgcctgcgggtctgctgtggctggtcgcgcagttccccgcacccctgaaagaccccggcgctttcccttcctggctcgcctcgtcgctgtctgcggggccgtgggggctggtcgcgcagttccccgcgcccctgcccgcacctgcgccccgccgcctgcatgccgcccccaccctgacgggggcgttcggggcccaccctgacgggtgcggtcggggcgtgccggggtcttttaggggcgcggggaactgcgcgagcaacccccacccacccgcaggtgcacgcggagcggcggacgccccgcagacgggggcaaaacgggcggagtgcccccgcccgccgggcggcgcgaattcgtaggtttaaggggcaggggtcagggcaggcgccgagccggtcaaccgcccccgtcccaggagaccccgtgacctcgaccggccacgcccgcaccgccgccatcgccatcggagccgccaccgccaccgtcctcggcgcgctgctggtcggcggctccggcgaggtgagtgcgagcccgccgcccgagcccaaggtccaggacgacttcgactccctcggccccgaggtgcgcgccgcgaagctctccgacgggcggacggcccactactcggacacgggcgacaaggacggcaagccggccctgttcatcggcggcaccggcacgagcgcccgcgcctcccacatgaccgacttcttccgctcgacgcgcgaggacctgggcctgcgcctcatctccgtggagcgcaacggcttcggcgacaccgcgttcgacgagaagctgggcaccgccgacttcgcgaaggacgccctcgaagtcctcgaccggctcgg

SEQ ID NO: 97 - protein, 136 aa, Streptomyces cinnamonensis
MGVAAGPIRVVVAKPGLDGHDRGAKVIARALRDAGMEVIYTGLHQTPEQVVDTAIQEDADAIGLSILSGAHNTLFARVLELLKERDAEDIKVFGGGIIPEADIAPLKEKGVAEIFTPGATTTSIVEWVRGNVRQAV

SEQ ID NO: 98 - DNA, 1643 bp, Streptomyces cinnamonensis
gtcgacctcccgtttggcgcacggaagggaggctctgtcccccgtgtgccctagggggagtcgtggtcgaggagtcggctgtgcgatggcgatcccggccaccgccctgcggtgactccgtgcccccgttgcatcgccgatgcgcggtgtcaccacgccgtgcggctgccggcgcggtggcccggcgtctcgttgcggctcccctcgcgcctggtccggatgcggagcgtgaacccctgggttacggacgggcgcgcagcgaacgtgtcccacgtgtgatttccccctcgctctccaccgcgaaactgccgcttgcgcgatgctggggataacgttcgttcacttccccggccggtgcggtgcggggtatctgtgccgggacagactttgtcggtacggatatcggtacatggaggcagtgatgggtgtggcagccgggccgatccgcgtggtggtcgccaagccggggctcgacgggcacgatcgcggggccaaggtgatcgcgcgggcgttgcgtgacgcgggtatggaggtcatctacaccgggctgcaccagacgcccgagcaggtggtggacaccgcgatccaggaggacgccgacgcgatcggcctctccatcctctccggagcgcacaacacgctgttcgcgcgcgtgttggagctcttgaaggagcgggacgcggaggacatcaaggtgtttggtggcggcatcatcccggaggcggacatcgcgccgctgaaggagaagggcgtcgcggagatcttcacgcccggggccaccaccacgtcgatcgtggagtgggttcgggggaacgtgcgacaggccgtctgaggcattccccgtcgcccgtctgccgtggtcggcgtcatatcggcggacatcgtctcggtggacgtcatggcggcggggggagttcgtcgcgtatcgccgcgcggaggcgcagggtggtgaccaggcgctggaacgcttccgaccagtagctgcccgcgccgggtgacgcgtcctccgcttcgtcggggaccgcggtgagcgcttccaggcggaccgcctcggccgggtccagacagcgttccgccaggcccatcactccgctgaagctccatgggtaactgcccgcgtcgcgcgcgatgttcagggcgtccaccacggcccggccgagagggccggcccagggcaccgcgcagacgccgagcagttggaacgcctccgacaggccgtgtgccgctatgaaccccgccacccagtccgcgcgctcggcggcaggcatggaggcgagcagtttggcccgctcggcgagggacacggcgccaggccccgccgcgtcgggtgaggcgggggcgccgagcagcgctctggaccaggcgacgtcacgctggcgtacggccgcgcggcaccatgcggcgtgcagttcgccccgccagtcgtcggccaccgggagcgccacgatctccgccggggtgcggttgccgagccggggcggccaggtggcgagcggggccgattccacgagctggccgagccaccaggagcgctcgccccggccggtggggggcttcgggacgacgccgtcccgctccatgcccgcgtcgcactcgtgcggcgcctcgacggtgagggtcggcgtgctcgatgtgtggtcgac

SEQ ID NO: 99 - protein, 566 aa, Streptomyces coelicolor
MDAHAIEEGRLRWQARYDAARKRDADFTTLSGDPVEPVYGPRPGDEYEGFERIGWPGEYPFTRGLYPTGYRGRTWTIRQFAGFGNAEQTNERYKMILRNGGGGLSVAFDMPTLMGRDSDDPRSLGEVGHCGVAIDSAADMEVLFKDIPLGDVTTSMTISGPAVPVFCMYLVAAERQGVDASVLNGTLQTDIFKEYIAQKEWLFQPEPHLRLIGDLMEYCAAGIPAYKPLSVSGYHIREAGATAAQELAYTLADGFGYVELGLSRGLDVDVFAPGLSFFFDAHLDFFEEIAKFRAARRIWARWMRDVYGARTDKAQWLRFHTQTAGVSLTAQQPYNNVVRTAVEALAAVLGGTNSLHTNALDETLALPSEQAAEIALRTQQVLMEETGVANVADPLGGSWFIEQLTDRIEADAEKIFEQIKERGLRAHPDGQHPVGPITSGLLRGIEDGWFTGEIAESAFRYQQSLEKDDKKVVGVNVHTGSVTGDLEILRVSHEVEREQVRVLGERKDARDDAAVRGALDAMLAAARSGGNMIGPMLDAVRAEATLGEICGVLRDEWGVYTEPAGF

SEQ ID NO: 100 - DNA, 1701 bp, Streptomyces coelicolor
atggacgctcatgccatagaggagggccgccttcgctggcaggcccggtacgacgcggcgcgcaagcgcgacgcggacttcaccacgctctccggagaccccgtggagccggtgtacgggccccgccccggggacgagtacgagggcttcgagcggatcggctggccgggcgagtaccccttcacccgcggcctgtatccgaccgggtaccgggggcgtacgtggaccatccggcagttcgccgggttcggcaacgccgagcagaccaacgagcgctacaagatgatcctccgcaacggcggcggcgggctctcggtcgccttcgacatgccgaccctgatgggccgcgactccgacgacccgcgctcgctgggcgaggtcgggcactgcggggtggccatcgactcggccgccgacatggaagtgctgttcaaggacatcccgctcggggacgtgacgacctccatgacgatcagcgggcccgccgtgcccgtgttctgcatgtacctcgtcgccgccgagcgccagggcgtcgacgcatccgtgctcaacggcacgctgcagaccgacatcttcaaggagtacatcgcccagaaggagtggctcttccagcccgagccccacctccggctcatcggcgacctcatggagtactgcgcggccggcatccccgcctacaagccgctctccgtctccggctaccacatccgcgaggcgggcgcgacggccgcgcaggagctggcgtacacgctcgccgacggcttcggatacgtggagctgggcctcagccgcgggctcgacgtggacgtcttcgcgcccggcctctccttcttcttcgacgcgcacctcgacttcttcgaggagatcgccaagttccgcgcggcccgcaggatctgggcccgctggatgcgcgacgtgtacggcgcgcggaccgacaaggcccagtggctgcggttccacacccagaccgccggagtctcgctcaccgcgcagcagccgtacaacaacgtcgtacgcaccgcggtggaggcgctggcggccgtgctcggcggcaccaactccctgcacaccaacgcgctcgacgagaccctcgccctgcccagcgagcaggccgccgagatcgccctgcgcacccagcaggtgctgatggaggagaccggcgtcgccaacgtcgccgacccgctgggcggttcctggttcatcgagcagctgaccgaccgcatcgaggccgacgccgagaagatcttcgagcagatcaaggagcgggggctgcgcgcccaccccgacgggcagcaccccgtcggaccgatcacctccggcctgctgcgcggcatcgaggacggctggttcaccggcgagatcgccgagtccgccttccgctaccagcagtccttggagaaggacgacaagaaggtggtcggcgtcaacgtccacaccggctccgtcaccggcgacctggagatcctgcgggtcagccacgaggtcgagcgcgagcaggtgcgggtcctgggcgagcgcaaggacgcccgggacgacgccgccgtgcgcggcgccctggacgccatgctggccgcggcccgctccggcggcaacatgatcgggccgatgctggacgcggtgcgcgcggaggcgacgctgggcgagatctgcggtgtgctgcgcgacgagtggggggtgtacacggaaccggcggggttctga

SEQ ID NO: 101 - protein, 138 aa, Streptomyces coelicolor
MGVAAGPIRVVVAKPGLDGHDRGAKVIARALRDAGMEVIYTGLHQTPEQIVDTAIQEDADAIGLSILSGAHNTLFAAVIELLRERDAADILVFGGGIIPEADIAPLKEKGVAEIFTPGATTASIVDWVRANVREPAGA

SEQ ID NO: 102 - DNA, 417 bp, Streptomyces coelicolor
atgggtgtggcagccggtccgatccgcgtggtggtggccaagccggggctcgacggccacgatcgcggggccaaggtgatcgcgagggccctgcgtgacgccggtatggaggtgatctacaccgggctccaccagacgcccgagcagatcgtcgacaccgcgatccaggaggacgccgacgcgatcgggctgtccatcctctccggtgcgcacaacacgctcttcgccgccgtgatcgagctgctccgggagcgggacgccgcggacatcctggtcttcggcggcgggatcatccccgaggcggacatcgccccgctgaaggagaagggcgtcgcggagatcttcacgcccggcgccaccacggcgtccatcgtggactgggtccgggcgaacgtgcgggagcccgcgggagcatag

SEQ ID NO: 103 - protein, 566 aa, Streptomyces avermitilis
MDADAIEEGRRRWQARYDASRKREADFTTLSGDPVEPAYGPRPGDAYEGFERIGWPGEYPFTRGLYPTGYRGRTWTIRQFAGFGNAEQTNERYKKILANGGGGLSVAFDMPTLMGRDSDDRRALGEVGHCGVAIDSAADMEVLFKDIPLGDVTTSMTISGPAVPVFCMYLVAAERQGVDPSVLNGTLQTDIFKEYIAQKEWLFQPEPHLRLIGDLMEHCASKIPAYKPLSVSGYHIREAGATAAQELAYTLADGFGYVELGLSRGLDVDVFAPGLSFFFDAHVDFFEEIAKFRAARRIWARWLRDVYGAKSEKAQWLRFHTQTAGVSLTAQQPYNNVVRTAVEALAAVLGGTNSLHTNALDETLALPSEQAAEIALRTQQVLMEETGVANVADPLGGSWYVEQLTDRIEADAEKIFEQIRERGLRAHPDGRHPIGPITSGILRGIEDGWFTGEIAESAFQYQQALEKGDKRVVGVNVHHGSVTGDLEILRVSHEVEREQVRVLGERKSGRDDTAVTAALDAMLAAARDGSNMIAPMLDAVRAEATLGEICDVLREEWGVYTEPAGF

SEQ ID NO: 104 - DNA, 1701 bp, Streptomyces avermitilis
tcagaaaccggcgggctccgtgtagaccccccactcctcccggaggacatcgcagatctcgcccagcgtggcctccgcgcggaccgcgtccagcatcggggcgatcatgttcgacccgtcgcgcgcggcggcgagcatcgcgtccagggccgcggttacggccgtgtcgtcgcgccccgacttccgctcgcccagcacccgcacctgctcgcgctccacctcgtggctgacgcgcaggatctccaggtcgcccgtcacggacccgtggtggacgttgacgccgacgacccgcttgtcgcccttctccagcgcctgctggtactggaaggccgactcggcgatctccccggtgaaccagccgtcctcgatgccgcgcaggatgccggaggtgatgggcccgatcgggtgccgcccgtccgggtgggcccgcagcccgcgctccctgatctgttcgaagatcttctcggcgtcggcctcgatccggtcggtcagctgctccacgtaccaggaaccgcccagcggatcggccacgttggcgacgcccgtctcctccatcagcacctgctgggtgcgcagggcgatctcggccgcctgctcggacggcagggcgagggtctcgtcgagggcgttggtgtgcagcgagttcgtcccgccgagcaccgcggcgagggcctccacggccgtccgtacgacgttgttgtacggctgctgcgcggtgagcgagacgcccgcggtctgggtgtggaagcgcagccactgcgccttctccgacttcgccccgtacacgtcccgcagccagcgcgcccagatgcgccgcgccgcacggaacttggcgatctcctcgaagaagtcgacgtgcgcgtcgaagaagaaggagagcccgggcgcgaacacgtccacgtccaggccgcggctcagccccagctccacgtatccgaaaccgtcggcgagggtgtacgccagctcctgggcggccgtggcaccggcctcccggatgtggtacccggagacggacagcggcttgtacgcggggatcttcgaggcgcagtgctccatcaggtcgccgatgagccgcagatggggctcgggctggaagagccactccttctgcgcgatgtactccttgaagatgtcggtctggagggtgccgttgaggacggaggggtcgacgccctgccgctcggccgcgaccaggtacatgcagaagacgggcacggcgggcccgctgatcgtcatcgacgtcgtcacgtcacccagcgggatgtccttgaacaggacctccatgtcggccgccgagtcgatcgcgaccccgcagtgcccgacctcgccgagcgcgcggcggtcgtcggagtcgcgccccatgagcgtcggcatgtcgaaggccacggacagcccaccgccgccgttggcgaggatcttcttgtagcgctcgttggtctgctcggcgttgccgaacccggcgaactgccggatggtccaggtccggccccggtagccggtcggatacagaccgcgcgtgaaggggtactcacccggccagccgatccgctcgaaaccctcgtacgcgtccccgggccggggcccgtacgccggctccacgggatcgccggagagcgtggtgaaatcggcctcgcgcttgcgtgaggcgtcgtagcgggcctgccagcgtcggcggccttcctcgatggcgtcagcgtccat

SEQ ID NO: 105 - protein, 138 aa, Streptomyces avermitilis
MGVAAGPIRVVVAKPGLDGHDRGAKVIARALRDAGMEVIYTGLHQTPEQIVGTAIQEDADAIGLSILSGAHNTLFAAVIDLLKERDAEDIKVFGGGIIPEADIAPLKEKGVAEIFTPGATTASIVEWVRANVRQPAGA

SEQ ID NO: 106 - DNA, 1701 bp, Streptomyces avermitilis; identical to SEQ ID NO: 104

SEQ ID NO: 107 - protein, 139 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMI

SEQ ID NO: 108 - DNA, 1689 bp, Saccharomyces cerevisiae
atgtctgaaattactttgggtaaatatttgttcgaaagattaaagcaagtcaacgttaacaccgttttcggtttgccaggtgacttcaacttgtccttgttggacaagatctacgaagttgaaggtatgagatgggctggtaacgccaacgaattgaacgctgcttacgccgctgatggttacgctcgtatcaagggtatgtcttgtatcatcaccaccttcggtgtcggtgaattgtctgctttgaacggtattgccggttcttacgctgaacacgtcggtgttttgcacgttgttggtgtcccatccatctctgctcaagctaagcaattgttgttgcaccacaccttgggtaacggtgacttcactgttttccacagaatgtctgccaacatttctgaaaccactgctatgatcactgacattgctaccgccccagctgaaattgacagatgtatcagaaccacttacgtcacccaaagaccagtctacttaggtttgccagctaacttggtcgacttgaacgtcccagctaagttgttgcaaactccaattgacatgtctttgaagccaaacgatgctgaatccgaaaaggaagtcattgacaccatcttggctttggtcaaggatgctaagaacccagttatcttggctgatgcttgttgttccagacacgacgtcaaggctgaaactaagaagttgattgacttgactcaattcccagctttcgtcaccccaatgggtaagggttccattgacgaacaacacccaagatacggtggtgtttacgtcggtaccttgtccaagccagaagttaaggaagccgttgaatctgctgacttgattttgtctgtcggtgctttgttgtctgatttcaacaccggttctttctcttactcttacaagaccaagaacattgtcgaattccactccgaccacatgaagatcagaaacgccactttcccaggtgtccaaatgaaattcgttttgcaaaagttgttgaccactattgctgacgccgctaagggttacaagccagttgctgtcccagctagaactccagctaacgctgctgtcccagcttctaccccattgaagcaagaatggatgtggaaccaattgggtaacttcttgcaagaaggtgatgttgtcattgctgaaaccggtacctccgctttcggtatcaaccaaaccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactatgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaa

SEQ ID NO: 109 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 110 - DNA, 1689 bp, Saccharomyces cerevisiae
atgtctgaaataaccttaggtaaatatttatttgaaagattgagccaagtcaactgtaacaccgtcttcggtttgccaggtgactttaacttgtctcttttggataagctttatgaagtcaaaggtatgagatgggctggtaacgctaacgaattgaacgctgcctatgctgctgatggttacgctcgtatcaagggtatgtcctgtattattaccaccttcggtgttggtgaattgtctgctttgaatggtattgccggttcttacgctgaacatgtcggtgttttgcacgttgttggtgttccatccatctcttctcaagctaagcaattgttgttgcatcataccttgggtaacggtgacttcactgttttccacagaatgtctgccaacatttctgaaaccactgccatgatcactgatattgctaacgctccagctgaaattgacagatgtatcagaaccacctacactacccaaagaccagtctacttgggtttgccagctaacttggttgacttgaacgtcccagccaagttattggaaactccaattgacttgtctttgaagccaaacgacgctgaagctgaagctgaagttgttagaactgttgttgaattgatcaaggatgctaagaacccagttatcttggctgatgcttgtgcttctagacatgatgtcaaggctgaaactaagaagttgatggacttgactcaattcccagtttacgtcaccccaatgggtaagggtgctattgacgaacaacacccaagatacggtggtgtttacgttggtaccttgtctagaccagaagttaagaaggctgtagaatctgctgatttgatattgtctatcggtgctttgttgtctgatttcaataccggttctttctcttactcctacaagaccaaaaatatcgttgaattccactctgaccacatcaagatcagaaacgccaccttcccaggtgttcaaatgaaatttgccttgcaaaaattgttggatgctattccagaagtcgtcaaggactacaaacctgttgctgtcccagctagagttccaattaccaagtctactccagctaacactccaatgaagcaagaatggatgtggaaccatttgggtaacttcttgagagaaggtgatattgttattgctgaaaccggtacttccgccttcggtattaaccaaactactttcccaacagatgtatacgctatcgtccaagtcttgtggggttccattggtttcacagtcggcgctctattgggtgctactatggccgctgaagaacttgatccaaagaagagagttattttattcattggtgacggttctctacaattgactgttcaagaaatctctaccatgattagatggggtttgaagccatacatttttgtcttgaataacaacggttacaccattgaaaaattgattcacggtcctcatgccgaatataatgaaattcaaggttgggaccacttggccttattgccaacttttggtgctagaaactacgaaacccacagagttgctaccactggtgaatgggaaaagttgactcaagacaaggacttccaagacaactctaagattagaatgattgaagttatgttgccagtctttgatgctccacaaaacttggttaaacaagctcaattgactgccgctactaacgctaaacaa

SEQ ID NO: 111 - protein, 533 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVI

SEQ ID NO: 112 - DNA, 1599 bp, Saccharomyces cerevisiae
atgtctgaaattactcttggaaaatacttatttgaaagattgaagcaagttaatgttaacaccatttttgggctaccaggcgacttcaacttgtccctattggacaagatttacgaggtagatggattgagatgggctggtaatgcaaatgagctgaacgccgcctatgccgccgatggttacgcacgcatcaagggtttatctgtgctggtaactacttttggcgtaggtgaattatccgccttgaatggtattgcaggatcgtatgcagaacacgtcggtgtactgcatgttgttggtgtcccctctatctccgctcaggctaagcaattgttgttgcatcataccttgggtaacggtgattttaccgtttttcacagaatgtccgccaatatctcagaaactacatcaatgattacagacattgctacagccccttcagaaatcgataggttgatcaggacaacatttataacacaaaggcctagctacttggggttgccagcgaatttggtagatctaaaggttcctggttctcttttggaaaaaccgattgatctatcattaaaacctaacgatcccgaagctgaaaaggaagttattgataccgtactagaattgatccagaattcgaaaaaccctgttatactatcggatgcctgtgcttctaggcacaacgttaaaaaagaaacccagaagttaattgatttgacgcaattcccagcttttgtgacacctctaggtaaagggtcaatagatgaacagcatcccagatatggcggtgtttatgtgggaacgctgtccaaacaagacgtgaaacaggccgttgagtcggctgatttgatcctttcggtcggtgctttgctctctgattttaacacaggttcgttttcctactcctacaagactaaaaatgtagtggagtttcattccgattacgtaaaggtgaagaacgctacgttcctcggtgtacaaatgaaatttgcactacaaaacttactgaaggttattcccgatgttgttaagggctacaagagcgttcccgtaccaaccaaaactcccgcaaacaaaggtgtacctgctagcacgcccttgaaacaagagtggttgtggaacgaattgtccaaattcttgcaagaaggtgatgttatcatttccgagaccggcacgtctgccttcggtatcaatcaaactatctttcctaaggacgcctacggtatctcgcaggtgttgtgggggtccatcggttttacaacaggagcaactttaggtgctgcctttgccgctgaggagattgaccccaacaagagagtcatcttattcataggtgacgggtctttgcagttaaccgtccaagaaatctccaccatgatcagatgggggttaaagccgtatctttttgtccttaacaacgacggctacactatcgaaaagctgattcatgggcctcacgcagagtacaacgaaatccagacctgggatcacctcgccctgttgcccgcatttggtgcgaaaaagtacgaaaatcacaagatcgccactacgggtgagtgggatgccttaaccactgattcagagttccagaaaaactcggtgatc

SEQ ID NO: 113 - protein, 564 aa, Candida glabrata
MSEITLGRYLFERLNQVDVKTIFGLPGDFNLSLLDKIYEVEGMRWAGNANELNAAYAADGYARIKGMSCIITTFGVGELSALNGIAGSYAEHVGVLHVVGVPSISSQAKQLLLHHTLGNGDFTVFHRMSANISETTAMVTDIATAPAEIDRCIRTTYITQRPVYLGLPANLVDLKVPAKLLETPIDLSLKPNDPEAETEVVDTVLELIKAAKNPVILADACASRHDVKAETKKLIDATQFPSFVTPMGKGSIDEQHPRFGGVYVGTLSRPEVKEAVESADLILSVGALLSDFNTGSFSYSYKTKNIVEFHSDYIKIRNATFPGVQMKFALQKLLNAVPEAIKGYKPVPVPARVPENKSCDPATPLKQEWMWNQVSKFLQEGDVVITETGTSAFGINQTPFPNNAYGISQVLWGSIGFTTGACLGAAFAAEEIDPKKRVILFIGDGSLQLTVQEISTMIRWGLKPYLFVLNNDGYTIERLIHGEKAGYNDIQNWDHLALLPTFGAKDYENHRVATTGEWDKLTQDKEFNKNSKIRMIEVMLPVMDAPTSLIEQAKLTASINAKQE

SEQ ID NO: 114 - DNA, 1692 bp, Candida glabrata
atgtctgagattactttgggtagatacttgttcgagagattgaaccaagtcgacgttaagaccatcttcggtttgccaggtgacttcaacttgtccctattggacaagatctacgaagttgaaggtatgagatgggctggtaacgctaacgaattgaacgctgcttacgctgctgacggttacgctagaatcaagggtatgtcctgtatcatcaccaccttcggtgtcggtgaattgtctgccttgaacggtattgccggttcttacgctgaacacgtcggtgtcttgcacgtcgtcggtgtcccatccatctcctctcaagctaagcaattgttgttgcaccacaccttgggtaacggtgacttcactgtcttccacagaatgtccgctaacatctctgagaccaccgctatggtcactgacatcgctaccgctccagctgagatcgacagatgtatcagaaccacctacatcacccaaagaccagtctacttgggtctaccagctaacttggtcgacctaaaggtcccagccaagcttttggaaaccccaattgacttgtccttgaagccaaacgacccagaagccgaaactgaagtcgttgacaccgtcttggaattgatcaaggctgctaagaacccagttatcttggctgatgcttgtgcttccagacacgacgtcaaggctgaaaccaagaagttgattgacgccactcaattcccatccttcgttaccccaatgggtaagggttccatcgacgaacaacacccaagattcggtggtgtctacgtcggtaccttgtccagaccagaagttaaggaagctgttgaatccgctgacttgatcttgtctgtcggtgctttgttgtccgatttcaacactggttctttctcttactcttacaagaccaagaacatcgtcgaattccactctgactacatcaagatcagaaacgctaccttcccaggtgtccaaatgaagttcgctttgcaaaagttgttgaacgccgtcccagaagctatcaagggttacaagccagtccctgtcccagctagagtcccagaaaacaagtcctgtgacccagctaccccattgaagcaagaatggatgtggaaccaagtttccaagttcttgcaagaaggtgatgttgttatcactgaaaccggtacctccgcttttggtatcaaccaaaccccattcccaaacaacgcttacggtatctcccaagttctatggggttccatcggtttcaccaccggtgcttgtttgggtgccgctttcgctgctgaagaaatcgacccaaagaagagagttatcttgttcattggtgacggttctttgcaattgactgtccaagaaatctccaccatgatcagatggggcttgaagccatacttgttcgtcttgaacaacgacggttacaccatcgaaagattgattcacggtgaaaaggctggttacaacgacatccaaaactgggaccacttggctctattgccaaccttcggtgctaaggactacgaaaaccacagagtcgccaccaccggtgaatgggacaagttgacccaagacaaggaattcaacaagaactccaagatcagaatgatcgaagttatgttgccagttatggacgctccaacttccttgattgaacaagctaagttgaccgcttccatcaacgctaagcaagaa

SEQ ID NO: 115 - protein, 596 aa, Pichia stipitis
MAEVSLGRYLFERLYQLQVQTIFGVPGDFNLSLLDKIYEVEDAHGKNSFRWAGNANELNASYAADGYSRVKRLGCLVTTFGVGELSALNGIAGSYAEHVGLLHVVGVPSISSQAKQLLLHHTLGNGDFTVFHRMSNNISQTTAFISDINSAPAEIDRCIREAYVKQRPVYIGLPANLVDLNVPASLLESPINLSLEKNDPEAQDEVIDSVLDLIKKSSNPIILVDACASRHDCKAEVTQLIEQTQFPVFVTPMGKGTVDEGGVDGELLEDDPHLIAKVAARLSAGKNAASRFGGVYVGTLSKPEVKDAVESADLILSVGALLSDFNTGSFSYSYRTKNIVEFHSDYTKIRQATFPGVQMKEALQELNKKVSSAASHYEVKPVPKIKLANTPATREVKLTQEWLWTRVSSWFREGDIIITETGTSSFGIVQSRFPNNTIGISQVLWGSIGFSVGATLGAAMAAQELDPNKRTILFVGDGSLQLTVQEISTIIRWGTTPYLFVLNNDGYTIERLIHGVNASYNDIQPWQNLEILPTFSAKNYDAVRISNIGEAEDILKDKEFGKNSKIRLIEVMLPRLDAPSNLAKQAAITAATNAEA

SEQ ID NO: 116 - DNA, 1788 bp, Pichia stipitis
atggctgaagtctcattaggaagatatctcttcgagagattgtaccaattgcaagtgcagaccatcttcggtgtccctggtgatttcaacttgtcgcttttggacaagatctacgaagtggaagatgcccatggcaagaattcgtttagatgggctggtaatgccaacgaattgaatgcatcgtacgctgctgacggttactcgagagtcaagcgtttagggtgtttggtcactacctttggtgtcggtgaattgtctgctttgaatggtattgccggttcttatgccgaacatgttggtttgcttcatgtcgtaggtgttccatcgatttcctcgcaagctaagcaattgttacttcaccacactttgggtaatggtgatttcactgttttccatagaatgtccaacaacatttctcagaccacagcctttatctccgatatcaactcggctccagctgaaattgatagatgtatcagagaggcctacgtcaaacaaagaccagtttatatcgggttaccagctaacttagttgatttgaatgttccggcctctttgcttgagtctccaatcaacttgtcgttggaaaagaacgacccagaggctcaagatgaagtcattgactctgtcttagacttgatcaaaaagtcgctgaacccaatcatcttggtcgatgcctgtgcctcgagacatgactgtaaggctgaagttactcagttgattgaacaaacccaattcccagtatttgtcactccaatgggtaaaggtaccgttgatgagggtggtgtagacggagaattgttagaagatgatcctcatttgattgccaaggtcgctgctaggttgtctgctggcaagaacgctgcctctagattcggaggtgtttatgtcggaaccttgtcgaagcccgaagtcaaggacgctgtagagagtgcagatttgattttgtctgtcggtgcccttttgtctgatttcaacactggttcattttcctactcctacagaaccaagaacatcgtcgaattccattctgattacactaagattagacaagccactttcccaggtgtgcagatgaaggaagccttgcaagaattgaacaagaaagtttcatctgctgctagtcactatgaagtcaagcctgtgcccaagatcaagttggccaatacaccagccaccagagaagtcaagttaactcaggaatggttgtggaccagagtgtcttcgtggttcagagaaggtgatattattatcaccgaaaccggtacatcctccttcggtatagttcaatccagattcccaaacaacaccatcggtatctcccaagtattgtggggttctattggtttctctgttggtgccactttgggtgctgccatggctgcccaagaactcgaccctaacaagagaaccatcttgtttgttggagatggttctttgcaattgaccgttcaggaaatctccaccataatcagatggggtaccacaccttaccttttcgtgttgaacaatgacggttacaccatcgagcgtttgatccacggtgtaaatgcctcatataatgacatccaaccatggcaaaacttggaaatcttgcctactttctcggccaagaactacgacgctgtgagaatctccaacatcggagaagcagaagatatcttgaaagacaaggaattcggaaagaactccaagattagattgatagaagtcatgttaccaagattggatgcaccatctaaccttgccaaacaagctgccattacagctgccaccaacgccgaagct

SEQ ID NO: 117 - protein, 569 aa, Pichia stipitis
MVSTYPESEVTLGRYLFERLHQLKVDTIFGLPGDFNLSLLDKVYEVPDMRWAGNANELNAAYAADGYSRIKGLSCLVTTFGVGELSALNGVGGAYAEHVGLLHVVGVPSISSQAKQLLLHHTLGNGDFTVFHRMSNSISQTTAFLSDISIAPGQIDRCIREAYVHQRPVYVGLPANMVDLKVPSSLLETPIDLKLKQNDPEAQEVVETVLKLVSQATNPIILVDACALRHNCKEEVKQLVDATNFQVFTTPMGKSGISESHPRLGGVYVGTMSSPQVKKAVENADLILSVGSLLSDFNTGSFSYSYKTKNVVEFHSDYMKIRQATFPGVQMKEALQQLIKRVSSYINPSYIPTRVPKRKQPLKAPSEAPLTQEYLWSKVSGWFREGDIIVTETGTSAFGIIQSHFPSNTIGISQVLWGSIGFTVGATVGAAMAAQEIDPSRRVILFVGDGSLQLTVQEISTLCKWDCNNTYLYVLNNDGYTIERLIHGKSASYNDIQPWNHLSLLRLFNAKKYQNVRVSTAGELDSLFSDKKFASPDRIRMIEVMLSRLDAPANLVAQAKLSERVNLEN

SEQ ID NO: 118 - DNA, 1707 bp, Pichia stipitis
atggtatcaacctacccagaatcagaggttactctaggaaggtacctctttgagcgactccaccaattgaaagtggacaccattttcggcttgccgggtgacttcaacctttccttattggacaaagtgtatgaagttccggatatgaggtgggctggaaatgccaacgaattgaatgctgcctatgctgccgatggttactccagaataaagggattgtcttgcttggtcacaacttttggtgttggtgaattgtctgctttaaacggagttggtggtgcctatgctgaacacgtaggacttctacatgtcgttggagttccatccatatcgtcacaggctaaacagttgttgctccaccataccttgggtaatggtgacttcactgtttttcacagaatgtccaatagcatttctcaaactacagcatttctctcagatatctctattgcaccaggtcaaatagatagatgcatcagagaagcatatgttcatcagagaccagtttatgttggtttaccggcaaatatggttgatctcaaggttccttctagtctcttagaaactccaattgatttgaaattgaaacaaaatgatcctgaagctcaagaagttgttgaaacagtcctgaagttggtgtcccaagctacaaaccccattatcttggtagacgcttgtgccctcagacacaattgcaaagaggaagtcaaacaattggttgatgccactaattttcaagtctttacaactccaatgggtaaatctggtatctccgaatctcatccaagattgggcggtgtctatgtcgggacaatgtcgagtcctcaagtcaaaaaagccgttgaaaatgccgatcttatactatctgttggttcgttgttatcggacttcaatacaggttcattttcatactcctacaagacgaagaatgttgttgaattccactctgactatatgaaaatcagacaggccaccttcccaggagttcaaatgaaagaagccttgcaacagttgataaaaagggtctcttcttacatcaatccaagctacattcctactcgagttcctaaaaggaaacagccattgaaagctccatcagaagctcctttgacccaagaatatttgtggtctaaagtatccggctggtttagagagggtgatattatcgtaaccgaaactggtacatctgctttcggaattattcaatcccattttcccagcaacactatcggtatatcccaagtcttgtggggctcaattggtttcacagtaggtgcaacagttggtgctgccatggcagcccaggaaatcgaccctagcaggagagtaattttgttcgtcggtgatggttcattgcagttgacggttcaggaaatctctacgttgtgtaaatgggattgtaacaatacttatctttacgtgttgaacaatgatggttacactatagaaaggttgatccacggcaaaagtgccagctacaacgatatacagccttggaaccatttatccttgcttcgcttattcaatgctaagaaataccaaaatgtcagagtatcgactgctggagaattggactctttgttctctgataagaaatttgcttctccagataggataagaatgattgaggtgatgttatcgagattggatgcaccagcaaatcttgttgctcaagcaaagttgtctgaacgggtaaaccttgaaaat

SEQ ID NO: 119 - protein, 563 aa, Kluyveromyces lactis
MSEITLGRYLFERLKQVEVQTIFGLPGDFNLSLLDNIYEVPGMRWAGNANELNAAYAADGYARLKGMSCIITTFGVGELSALNGIAGSYAEHVGVLHVVGVPSVSSQAKQLLLHHTLGNGDFTVFHRMSSNISETTAMITDINTAPAEIDRCIRTTYVSQRPVYLGLPANLVDLTVPASLLDTPIDLSLKPNDPEAEEEVIENVLQLIKEAKNPVILADACCSRHDAKAETKKLIDLTQFPAFVTPMGKGSIDEKHPRFGGVYVGTLSSPAVKEAVESADLVLSVGALLSDFNTGSFSYSYKTKNIVEFHSDYTKIRSATFPGVQMKFALQKLLTKVADAAKGYKPVPVPSEPEHNEAVADSTPLKQEWVWTQVGEFLREGDVVITETGTSAFGINQTHFPNNTYGISQVLWGSIGFTTGATLGAAFAAEEIDPKKRVILFIGDGSLQLTVQEISTMIRWGLKPYLFVLNNDGYTIERLIHGETAQYNCIQNWQHLELLPTFGAKDYEAVRVSTTGEWNKLTTDEKFQDNTRIRLIEVMLPTMDAPSNLVKQAQLTAATNAKN

SEQ ID NO: 120 - DNA, 1689 bp, Kluyveromyces lactis
atgtctgaaattacattaggtcgttacttgttcgaaagattaaagcaagtcgaagttcaaaccatctttggtctaccaggtgatttcaacttgtccctattggacaatatctacgaagtcccaggtatgagatgggctggtaatgccaacgaattgaacgctgcttacgctgctgatggttacgccagattaaagggtatgtcctgtatcatcaccaccttcggtgtcggtgaattgtctgctttgaacggtattgccggttcttacgctgaacacgttggtgtcttgcacgttgtcggtgttccatccgtctcttctcaagctaagcaattgttgttgcaccacaccttgggtaacggtgacttcactgttttccacagaatgtcctccaacatttctgaaaccactgctatgatcaccgatatcaacactgccccagctgaaatcgacagatgtatcagaaccacttacgtttcccaaagaccagtctacttgggtttgccagctaacttggtcgacttgactgtcccagcttctttgttggacactccaattgatttgagcttgaagccaaatgacccagaagccgaagaagaagtcatcgaaaacgtcttgcaactgatcaaggaagctaagaacccagttatcttggctgatgcttgttgttccagacacgatgccaaggctgagaccaagaagttgatcgacttgactcaattcccagccttcgttaccccaatgggtaagggttccattgacgaaaagcacccaagattcggtggtgtctacgtcggtaccctatcttctccagctgtcaaggaagccgttgaatctgctgacttggttctatcggtcggtgctctattgtccgatttcaacactggttctttctcttactcttacaagaccaagaacattgtcgaattccactctgactacaccaagatcagaagcgctaccttcccaggtgtccaaatgaagttcgctttacaaaaattgttgactaaggttgccgatgctgctaagggttacaagccagttccagttccatctgaaccagaacacaacgaagctgtcgctgactccactccattgaagcaagaatgggtctggactcaagtcggtgaattcttgagagaaggtgatgttgttatcactgaaaccggtacctctgccttcggtatcaaccaaactcatttcccaaacaacacatacggtatctctcaagttttatggggttccattggtttcaccactggtgctaccttgggtgctgccttcgctgccgaagaaattgatccaaagaagagagttatcttattcattggtgacggttctttgcaattgactgttcaagaaatctccaccatgatcagatggggcttgaagccatacttgttcgtattgaacaacgacggttacaccattgaaagattgattcacggtgaaaccgctcaatacaactgtatccaaaactggcaacacttggaattattgccaactttcggtgccaaggactacgaagctgtcagagtttccaccactggtgaatggaacaagttgaccactgacgaaaagttccaagacaacaccagaatcagattgatcgaagttatgttgccaactatggatgctccatctaacttggttaagcaagctcaattgactgctgctaccaacgctaagaac

SEQ ID NO: 121 - protein, 571 aa, Yarrowia lipolytica
MSDSEPQMVDLGDYLFARFKQLGVDSVFGVPGDFNLTLLDHVYNVDMRWVGNTNELNAGYSADGYSRVKRLACLVTTFGVGELSAVAAVAGSYAEHVGVVHVVGVPSTSAENKHLLLHHTLGNGDFRVFAQMSKLISEYTHHIEDPSEAADVIDTAIRIAYTHQRPVYIAVPSNFSEVDIADQARLDTPLDLSLQPNDPESQYEVIEEICSRIKAAKKPVILVDACASRYRCVDETKELAKITNFAYFVTPMGKGSVDEDTDRYGGTYVGSLTAPATAEVVETADLIISVGALLSDFNTGSFSYSYSTKNVVELHSDHVKIKSATYNNVGMKMLFPPLLEAVKKLVAETPDFASKALAVPDTTPKIPEVPDDHITTQAWLWQRLSYFLRPTDIVVTETGTSSFGIIQTKFPHNVRGISQVLWGSIGYSVGAACGASIAAQEIDPQQRVILFVGDGSLQLTVTEISCMIRNNVKPYIFVLNNDGYTIERLIHGENASYNDVHMWKYSKILDTFNAKAHESIVVNTKGEMDALFDNEEFAKPDKIRLIEVMCDKMDAPASLIKQAELSAKTNV

SEQ ID NO: 122 - DNA, 1713 bp, Yarrowia lipolytica
atgagcgactccgaaccccaaatggtcgacctgggcgactatctctttgcccgattcaagcagctaggcgtggactccgtctttggagtgcccggcgacttcaacctcaccctgttggaccacgtgtacaatgtcgacatgcggtgggttgggaacacaaacgagctgaatgccggctactcggccgacggctactcccgggtcaagcggctggcatgtcttgtcaccacctttggcgtgggagagctgtctgccgtggctgctgtggcaggctcgtacgccgagcatgtgggcgtggtgcatgttgtgggcgttcccagcacctctgctgagaacaagcatctgctgctgcaccacacactcggtaacggcgacttccgggtctttgcccagatgtccaaactcatctccgagtacacccaccatattgaggaccccagcgaggctgccgacgtaatcgacaccgccatccgaatcgcctacacccaccagcggcccgtttacattgctgtgccctccaacttctccgaggtcgatattgccgaccaggctagactggatacccccctggacctttcgctgcagcccaacgaccccgagagccagtacgaggtgattgaggagatttgctcgcgtatcaaggccgccaagaagcccgtgattctcgtcgacgcctgcgcttcgcgatacagatgtgtggacgagaccaaggagctggccaagatcaccaactttgcctactttgtcactcccatgggtaagggttctgtggacgaggatactgaccggtacggaggaacatacgtcggatcgctgactgctcctgctactgccgaggtggttgagacagctgatctcatcatctccgtaggagctcttctgtcggacttcaacaccggttccttctcgtactcctactccaccaaaaacgtggtggaattgcattcggaccacgtcaaaatcaagtccgccacctacaacaacgtcggcatgaaaatgctgttcccgcccctgctcgaagccgtcaagaaactggttgccgagacccctgactttgcatccaaggctctggctgttcccgacaccactcccaagatccccgaggtacccgatgatcacattacgacccaggcatggctgtggcagcgtctcagttactttctgaggcccaccgacatcgtggtcaccgagaccggaacctcgtcctttggaatcatccagaccaagttcccccacaacgtccgaggtatctcgcaggtgctgtggggctctattggatactcggtgggagcagcctgtggagcctccattgctgcacaggagattgacccccagcagcgagtgattctgtttgtgggcgacggctctcttcagctgacggtgaccgagatctcgtgcatgatccgcaacaacgtcaagccgtacatttttgtgctcaacaacgacggctacaccatcgagaggctcattcacggcgaaaacgcctcgtacaacgatgtgcacatgtggaagtactccaagattctcgacacgttcaacgccaaggcccacgagtcgattgtggtcaacaccaagggcgagatggacgctctgttcgacaacgaagagtttgccaagcccgacaagatccggctcattgaggtcatgtgcgacaagatggacgcgcctgcctcgttgatcaagcaggctgagctctctgccaagaccaacgtt

SEQ ID NO: 123 - protein, 571 aa, Schizosaccharomyces pombe
MSGDILVGEYLFKRLEQLGVKSILGVPGDFNLALLDLIEKVGDEKFRWVGNTNELNGAYAADGYARVNGLSAIVTTFGVGELSAINGVAGSYAEHVPVVHIVGMPSTKVQDTGALLHHTLGDGDFRTFMDMFKKVSAYSIMIDNGNDAAEKIDEALSICYKKARPVYIGIPSDAGYFKASSSNLGKRLKLEEDTNDPAVEQEVINHISEMVVNAKKPVILIDACAVRHRVVPEVHELIKLTHFPTYVTPMGKSAIDETSQFFDGVYVGSISDPEVKDRIESTDLLLSIGALKSDFNTGSFSYHLSQKNAVEFHSDHMRIRYALYPNVAMKYILRKLLKVLDASMCHSKAAPTIGYNIKPKHAEGYSSNEITHCWFWPKFSEFLKPRDVLITETGTANFGVLDCRFPKDVTAISQVLWGSIGYSVGAMFGAVLAVHDSKEPDRRTILVVGDGSLQLTITEISTCIRHNLKPIIFIINNDGYTIERLIHGLHASYNEINTKWGYQQIPKFFGAAENHFRTYCVKTPTDVEKLFSDKEFANADVIQVVELVMPMLDAPRVLVEQAKLTSKINKQ

SEQ ID NO: 124 - DNA, 1713 bp, Schizosaccharomyces pombe
atgagtggggatattttagtcggtgaatatctattcaaaaggcttgaacaattaggggtcaagtccattcttggtgttccaggagatttcaatttagctctacttgacttaattgagaaagttggagatgagaaatttcgttgggttggcaataccaatgagttgaatggtgcttatgccgctgatggttatgctcgtgttaatggtctttcagccattgttacaacgttcggcgtgggagagctttccgctattaatggagtggcaggttcttatgcggagcatgtcccagtagttcatattgttggaatgccttccacaaaggtgcaagatactggagctttgcttcatcatactttaggagatggagactttcgcactttcatggatatgtttaagaaagtttctgcctacagtataatgatcgataacggaaacgatgcagctgaaaagatcgatgaagccttgtcgatttgttataaaaaggctaggcctgtttacattggtattccttctgatgctggctacttcaaagcatcttcatcaaatcttgggaaaagactaaagctcgaggaggatactaacgatccagcagttgagcaagaagtcatcaatcatatctcggaaatggttgtcaatgcaaagaaaccagtgattttaattgacgcttgtgctgtaagacatcgtgtcgttccagaagtacatgagctgattaaattgacccatttccctacatatgtaactcccatgggtaaatctgcaattgacgaaacttcgcaattttttgacggcgtttatgttggttcaatttcagatcctgaagttaaagacagaattgaatccactgatctgttgctatccatcggtgctctcaaatcagactttaacacgggttccttctcttaccacctcagccaaaagaatgccgttgagtttcattcagaccacatgcgcattcgatatgctctttatccaaatgtagccatgaagtatattcttcgcaaactgttgaaagtacttgatgcttctatgtgtcattccaaggctgctcctaccattggctacaacatcaagcctaagcatgcggaaggatattcttccaacgagattactcattgctggttttggcctaaatttagtgaatttttgaagccccgagatgttttgatcaccgagactggaactgcaaactttggtgtccttgattgcaggtttccaaaggatgtaacagccatttcccaggtattatggggatctattggatactccgttggtgcaatgtttggtgctgttttggccgtccacgattctaaagagcccgatcgtcgtaccattcttgtagtaggtgatggatccttacaactgacgattacagagatttcaacctgcattcgccataacctcaaaccaattattttcataattaacaacgacggttacaccattgagcgtttaattcatggtttgcatgctagctataacgaaattaacactaaatggggctaccaacagattcccaagtttttcggagctgctgaaaaccacttccgcacttactgtgttaaaactcctactgacgttgaaaagttgtttagcgacaaggagtttgcaaatgcagatgtcattcaagtagttgagcttgtaatgcctatgttggatgcacctcgtgtcctagttgagcaagccaagttgacgtctaagatcaataagcaa

SEQ ID NO: 125 - protein, 563 aa, Zygosaccharomyces rouxii
MSEITLGRYLFERLKQVDTNTIFGVPGDFNLSLLDKVYEVQGLRWAGNANELNAAYAADGYARVKGLAALITTFGVGELSALNGIAGSYAEHVGVLHIVGVPSVSSQAKQLLLHHTLGNGDFTVFHRMSANISETTAMLTDITAAPAEIDRCIRVAYVNQRPVYLGLPANLVDQKVPASLLNTPIDLSLKENDPEAETEVVDTVLELIKEAKNPVILADACCSRHDVKAETKKLIDLTQFPSFVTPMGKGSIDEQNPRFGGVYVGTLSSPEVKEAVESADLVLSVGALLSDFNTGSFSYSYKTKNVVEFHSDHIKIRNATFPGVQMKFVLKKLLQAVPEAVKNYKPGPVPAPPSPNAEVADSTTLKQEWLWRQVGSFLREGDVVITETGTSAFGINQTHFPNQTYGISQVLWGSIGYTTGSTLGAAFAAEEIDPKKRVILFIGDGSLQLTVQEISTMIRWGLKPYLFVLNNDGYTIERLIHGETAEYNCIQPWKHLELLNTFGAKDYENHRVSTVGEWNKLTQDPKFNENSRIRMIEVMLEVMDAPSSLVAQAQLTAATNAKQ

SEQ ID NO: 126 - DNA, 1689 bp, Zygosaccharomyces rouxii
atgtctgaaattactctaggtcgttacttgttcgaaagattaaagcaagttgacactaacaccatcttcggtgttccaggtgacttcaacttgtccttgttggacaaggtctacgaagtgcaaggtctaagatgggctggtaacgctaacgaattgaacgctgcctacgctgctgacggttacgccagagttaagggtttggctgctttgatcaccaccttcggtgtcggtgaattgtctgctttgaacggtattgcaggttcttacgctgaacacgttggtgttttgcacattgttggtgttccatctgtctcttctcaagctaagcaattgttgttgcaccacaccttgggtaacggtgacttcactgttttccacagaatgtccgccaacatctctgaaaccaccgctatgttgaccgacatcactgctgctccagctgaaattgaccgttgcatcagagttgcttacgtcaaccaaagaccagtctacttgggtctaccagctaacttggttgaccaaaaggtcccagcttctttgttgaacactccaattgatctatctctaaaggagaacgacccagaagctgaaaccgaagttgttgacaccgttttggaattgatcaaggaagctaagaacccagttatcttggctgatgcttgctgctccagacacgacgtcaaggctgaaaccaagaagttgatcgacttgactcaattcccatctttcgttactcctatgggtaagggttccatcgacgaacaaaacccaagattcggtggtgtctacgtcggtactctatccagcccagaagttaaggaagctgttgaatctgctgacttggttctatctgtcggtgctctattgtccgatttcaacactggttctttctcttactcttacaagaccaagaacgttgttgaattccactctgaccacatcaagatcagaaacgctaccttcccaggtgttcaaatgaaattcgttttgaagaaactattgcaagctgtcccagaagctgtcaagaactacaagccaggtccagtcccagctccgccatctccaaacgctgaagttgctgactctaccaccttgaagcaagaatggttatggagacaagtcggtagcttcttgagagaaggtgatgttgttattaccgaaactggtacctctgctttcggtatcaaccaaactcacttccctaaccaaacttacggtatctctcaagtcttgtggggttctattggttacaccactggttccactttgggtgctgccttcgctgctgaagaaattgaccctaagaagagagttatcttgttcattggtgacggttctctacaattgaccgttcaagaaatctccaccatgatcagatggggtctaaagccatacttgttcgttttgaacaacgatggttacaccattgaaagattgattcacggtgaaaccgctgaatacaactgtatccaaccatggaagcacttggaattgttgaacaccttcggtgccaaggactacgaaaaccacagagtctccactgtcggtgaatggaacaagttgactcaagatccaaaattcaacgaaaactctagaattagaatgatcgaagttatgcttgaagtcatggacgctccatcttctttggtcgctcaagctcaattgaccgctgctactaacgctaagcaa

SEQ ID NO: 127 - protein, 267 aa, Saccharomyces cerevisiae
MSQGRKAAERLAKKTVLITGASAGIGKATALEYLEASNGDMKLILAARRLEKLEELKKTIDQEFPNAKVHVAQLDITQAEKIKPFIENLPQEFKDIDILVNNAGKALGSDRVGQIATEDIQDVFDTNVTALINITQAVLPIFQAKNSGDIVNLGSIAGRDAYPTGSIYCASKFAVGAFTDSLRKELINTKIRVILIAPGLVETEFSLVRYRGNEEQAKNVYKDTTPLMADDVADLIVYATSRKQNTVIADTLIFPTNQASPHHIFRG

SEQ ID NO: 128 - DNA, 804 bp, Saccharomyces cerevisiae
atgtcccaaggtagaaaagctgcagaaagattggctaagaagactgtcctcattacaggtgcatctgctggtattggtaaggcgaccgcattagagtacttggaggcatccaatggtgatatgaaactgatcttggctgctagaagattagaaaagctcgaggaattgaagaagaccattgatcaagagtttccaaacgcaaaagttcatgtggcccagctggatatcactcaagcagaaaaaatcaagcccttcattgaaaacttgccacaagagttcaaggatattgacattctggtgaacaatgccggaaaggctcttggcagtgaccgtgtgggccagatcgcaacggaggatatccaggacgtgtttgacaccaacgtcacggctttaatcaatatcacacaagctgtactgcccatattccaagccaagaattcaggagatattgtaaatttgggttcaatcgctggcagagacgcatacccaacaggttctatctattgtgcctctaagtttgccgtgggggcgttcactgatagtttgagaaaggagctcatcaacactaaaattagagtcattctaattgcaccagggctagtcgagactgaattttcactagttagatacagaggtaacgaggaacaagccaagaatgtttacaaggatactaccccattgatggctgatgacgtggctgatctgatcgtctatgcaacttccagaaaacaaaatactgtaattgcagacactttaatctttccaacaaaccaagcgtcacctcatcatatcttccgtggataa

(19) United States
(12) Patent Application Publication
(10) Pub. No.: US 2014/0106419 A1
(43) Pub. Date: Apr. 17, 2014

(54) PROCESSES AND SYSTEMS FOR THE PRODUCTION OF FERMENTATION PRODUCTS

(71) Applicant: BUTAMAX ADVANCED BIOFUELS LLC, Wilmington, DE (US)

(72) Inventors: Stephane Francois Bazzana, Wilmington, DE (US); Adam Bernfeld, Newark, DE (US); Keith H. Burlew, Middleton, DE (US); Duncan Coffey, Hockessin, DE (US); James Timothy Cronin, Townsend, DE (US); Benjamin Fuchs, Wilmington, DE (US); John W. Hallam, Wilmington, DE (US); Adam Jones, Wilmington, DE (US); David J. Lowe, Wilmington, DE (US); Brian Michael Roesch, Middletown, DE (US); Mathias E. Stolarski, Swarthmore, PA (US); James Gregory Wood, Newark, DE (US); Joseph J. Zaher, Newark, DE (US)

(73) Assignee: BUTAMAX ADVANCED BIOFUELS LLC, Wilmington, DE (US)

(21) Appl. No.: 14/052,184

(22) Filed: Oct. 11, 2013

Related U.S. Application Data
(60) Provisional application No. 61/712,385, filed on Oct. 11, 2012.

(51) Int. Cl. C12P 7/16; C12P 7/64

(52) U.S. Cl. CPC C12P 7/16; C12P 7/649
USPC 435/134; 435/155; 435/160; 435/163; 435/157; 252/182.12

(57) ABSTRACT

The present invention relates to processes and systems for the production of fermentation products such as alcohols. The present invention also provides methods for separating feed stream components for improved biomass processing and productivity.

This application claims the benefit of U.S. Provisional Application No. 61/712,385, filed Oct. 11, 2012; the entire contents are herein incorporated by reference.

The Sequence Listing associated with this application is filed in electronic form via EFS-Web and hereby incorporated by reference into the specification in its entirety.

### FIELD OF THE INVENTION

The present invention relates to processes and systems for the production of fermentation products such as alcohols. The present invention also provides processes for separating feed stream components for improved biomass processing productivity.

### BACKGROUND OF THE INVENTION

Alcohols have a variety of industrial and scientific applications such as fuels, reagents, and solvents. For example, butanol is an important industrial chemical with a variety of applications including use as a fuel additive, as a feedstock chemical in the plastics industry, and as a food-grade extractant in the food and flavor industry. Accordingly, there is a high demand for alcohols such as butanol as well as for efficient and environmentally friendly production methods including, for example, the use of biomass as feedstock for these methods.

Production of alcohols by fermentation is one such environmentally friendly production method. Some microorganisms that produce alcohols in high yields also have low toxicity thresholds, such that the alcohol needs to be removed from the fermentor as it is being produced. One method, in situ product removal (ISPR), may be used to remove alcohol from the fermentor as it is produced, thereby allowing the microorganism to produce alcohol at high yields. An example of ISPR that has been described in the art is liquid-liquid extraction (see, e.g., U.S. Patent Application Publication No. 2009/0305370). In order to be technically and economically viable, liquid-liquid extraction requires contact between the extractant and the fermentation broth for efficient mass transfer; phase separation of the extractant from the fermentation broth; efficient recovery and recycle of the extractant; and minimal degradation and/or contamination of the extractant over a long-term operation.

When the aqueous stream entering the fermentor contains undissolved solids from feedstock, the undissolved solids may interfere with liquid-liquid extraction and the extraction method may not be technically and economically viable, for example, leading to increases in capital and operating costs. The presence of undissolved solids during extractive fermentation may lower the mass transfer coefficient, impede phase separation, result in the accumulation of oil from the undissolved solids in the extractant leading to reduced extraction efficiency over time, increase the loss of extractant because it becomes trapped in solids and ultimately removed as Dried Distillers Grains with Solubles (DDGS), slow the disengagement of extractant droplets from the fermentation broth, and/or result in a lower fermentor volume efficiency. Thus, solids removal provides an efficient means to produce and recover an alcohol from a fermentation process.

In addition to solids removal, removal of oil from feedstock may also provide beneficial effects on the production of alcohols as well as commercial benefits. For example, some oils such as corn oil and soybean oil may be used as feedstock for biodiesel and thus, provide an additional revenue stream for alcohol producers. In addition, removing oil can result in energy savings for the production plant due to more efficient fermentation, less fouling due to the removal of the oil, increased fermentor volume efficiency, and decreased energy requirements, for example, the energy needed to dry distillers grains.

There is a continuing need to develop more efficient processes and systems for producing alcohols such as ethanol and butanol. The present invention satisfies this need and provides processes and systems for producing alcohols including processes and systems for separating feed stream components prior to the fermentation and controlling the amount of undissolved solids and/or oil in the fermentation process.

### SUMMARY OF THE INVENTION

The present invention relates to processes and systems for separating feed stream components and controlling the amount of undissolved solids and/or oil in a fermentor feed stream in the production of fermentation products. The separated components provide a mechanism for increasing biomass processing productivity, including improving alcohol fermentation co-product profiles. By separating the feed streams into certain components including (1) an aqueous stream comprising fermentable carbon sources, (2) a feed stream comprising oil, and (3) a feed stream comprising undissolved solids, these components may be recombined in a controlled manner, or removed from the system for other uses. This provides a mechanism to develop co-product compositions to meet the needs of different markets, such as animal feed markets requiring higher protein and/or higher fat feeds.

The present invention also relates to processes and systems for removing oil from a fermentor feed stream in the production of fermentation products. In some embodiments, undissolved solids and oil may be removed from a fermentor feed stream.

The present invention is directed to a method for producing fermentation products such as product alcohols comprising: providing a feedstock slurry comprising fermentable carbon source, undissolved solids, and oil; separating a portion of the undissolved solids and oil from the feedstock slurry whereby an aqueous solution comprising fermentable carbon source, a wet cake comprising solids, and an oil stream are formed; and adding the aqueous solution to a fermentation broth comprising microorganisms whereby a fermentation product is produced. In some embodiments, the method may further comprise the step of recovering the oil stream. In some embodiments, the method may further comprise the step of washing the wet cake wherein a second aqueous solution comprising carbohydrate is generated. In some embodiments, the method may further comprise the step of adding the second aqueous solution to the fermentation broth. In some embodiments, the second aqueous solution may be added to feedstock slurry. In some embodiments, the second aqueous solution may be added to liquefaction and/or feedstock preparation. In some embodiments, the aqueous solution may contain no more than about 5% by weight of undissolved solids.

In some embodiments, the oil may be corn oil and may comprise one or more of triglycerides, fatty acids, diglycerides, monoglycerides, and phospholipids. In some embodiments, the method may further comprise the step of combining a portion of the wet cake and a portion of oil to produce a wet cake comprising triglycerides, fatty acids, diglycerides, monoglycerides, and phospholipids.

In some embodiments, the method may further comprise the step of combining the aqueous solution with a portion of the wet cake to produce a mixture of the aqueous solution and wet cake and adding the mixture to the fermentation broth. In some embodiments, separating the feedstock slurry may be a single step process. In some embodiments, the undissolved solids and oil may be separated from feedstock slurry by decanter bowl centrifugation, three-phase centrifugation, disk stack centrifugation, filtering centrifugation, decanter centrifugation, filtration, microfiltration, vacuum filtration, beltfilter, pressure filtration, membrane filtration, crossflow filtration, drum filter, filtration using a screen, screen separation, rotary screen, grating, porous grating, flotation, hydrocyclone, filter press, screwpress, gravity settler, vortex separator, or combination thereof. In some embodiments, the undissolved solids and oil may be separated from feedstock slurry using two or more separation means such as decanter bowl centrifugation, three-phase centrifugation, disk stack centrifugation, filtering centrifugation, decanter centrifugation, filtration, microfiltration, vacuum filtration, beltfilter, pressure filtration, membrane filtration, crossflow filtration, drum filter, filtration using a screen, screen separation, rotary screen, grating, porous grating, flotation, hydrocyclone, filter press, screwpress, gravity settler, vortex separator, or combination thereof. In some embodiments, the undissolved solids and oil may be separated from feedstock slurry using two or three separation means.

In some embodiments, the two separation means may be three-phase centrifuges. In some embodiments, the two separation means may be a three-phase centrifuge and a disk stack centrifuge. In some embodiments, the two separation means may be a three-phase centrifuge and a decanter centrifuge. In some embodiments, the two separation means may be centrifugation and filtration. In some embodiments, the two separation means may be a three-phase centrifuge and crossflow filtration. In some embodiments, the two separation means may be a three-phase centrifuge and a drum filter. In some embodiments, the two separation means may be a three-phase centrifuge and a rotary screen.

In some embodiments, the three separation means may be three-phase centrifuges. In some embodiments, the three separation means may be a three-phase centrifuge, and two disk stack centrifuges. In some embodiments, the three separation means may be a three-phase centrifuge, and two decanter centrifuges. In some embodiments, the three separation means may be a combination of centrifugation and filtration. In some embodiments, the three separation means may be a three-phase centrifuge, a rotary screen, and a disk stack centrifuge. In some embodiments, the three separation means may be a three-phase centrifuge, crossflow filtration, and a decanter centrifuge. In some embodiments, the three separation means may be a three-phase centrifuge, a rotary screen, and a disk stack centrifuge. In some embodiments, the three separation means may be a three-phase centrifuge, crossflow filtration, and a decanter centrifuge.

In some embodiments, one or more control parameters of the separation means or devices may be adjusted to improve separation of the feedstock slurry. In some embodiments, the one or more control parameters may be differential speed, bowl speed, flow rate, impeller position, weir position, scroll pitch, residence time, discharge volume, or combinations thereof. In some embodiments, real-time measurements may be used to monitor separation of the feedstock slurry. In some embodiments, separation may be monitored by Fourier transform infrared spectroscopy, near-infrared spectroscopy, Raman spectroscopy, high pressure liquid chromatography, viscometers, densitometers, tensiometers, droplet size analyzers, particle analyzers, or combinations thereof.

In some embodiments, the fermentation product may be a product alcohol. In some embodiments, the product alcohol may be ethanol, propanol, butanol, pentanol, hexanol, and/or fusel alcohols. In some embodiments, the microorganism may comprise a butanol biosynthetic pathway. In some embodiments, the butanol biosynthetic pathway may be a 1-butanol biosynthetic pathway, a 2-butanol biosynthetic pathway, or an isobutanol biosynthetic pathway.

The present invention is also directed to a method comprising providing a feedstock slurry comprising fermentable carbon source and undissolved solids; separating at least a portion of the undissolved solids from the feedstock slurry whereby an aqueous solution comprising fermentable carbon source and a wet cake comprising solids are generated; contacting the wet cake with a liquid to form a wet cake mixture; and separating at least a portion of undissolved solids from the wet cake mixture whereby a second aqueous solution comprising fermentable carbon source and a second wet cake comprising solids are generated. In some embodiments, the liquid may be fresh water, backset, cook water, process water, lutter water, evaporation water, or combinations thereof. In some embodiments, the steps contacting and separating steps may be repeated. In some embodiments, the steps contacting and separating steps may be repeated two times. In some embodiments, the steps contacting and separating steps may be repeated three times. In some embodiments, the steps contacting and separating steps may be repeated four or more times. In some embodiments, the second aqueous solution may be added to feedstock slurry. In some embodiments, the second aqueous solution may be added to liquefaction and/or feedstock preparation.

The present invention is directed to a method comprising: providing a feedstock slurry comprising fermentable carbon source, undissolved solids, and oil; separating at least a portion of the oil and undissolved solids from the feedstock slurry whereby an aqueous solution comprising fermentable carbon source, an oil stream; and a wet cake comprising solids are generated; and contacting the wet cake with a liquid to form a wet cake mixture; and separating at least a portion of undissolved solids and oil from the wet cake mixture whereby a second aqueous solution comprising fermentable carbon source, a second oil stream; and a second wet cake comprising solids are generated. In some embodiments, separating the feedstock slurry may be a single step process. In some embodiments, the undissolved solids and oil may be separated from feedstock slurry by decanter bowl centrifugation, three-phase centrifugation, disk stack centrifugation, filtering centrifugation, decanter centrifugation, filtration, microfiltration vacuum filtration, beltfilter, pressure filtration, membrane filtration, crossflow filtration, drum filter, filtration using a screen, screen separation, rotary screen, grating, porous grating, flotation, hydrocyclone, filter press, screwpress, gravity settler, vortex separator, or combinations thereof. In some embodiments, one or more control parameters of the separation device may be adjusted to improve separation of the feedstock slurry. In some embodiments, the one or more control parameters may be differential speed, bowl speed, flow rate, impeller position, weir position, scroll pitch, residence time, discharge volume, or combinations thereof. In some embodiments, real-time measurements may be used to monitor separation of the feedstock slurry. In some embodiments, separation may be monitored by Fourier transform infrared spectroscopy, near-infrared spectroscopy, Raman spectroscopy, high pressure liquid chromatography, viscometers, densitometers, tensiometers, droplet size analyzers, particle analyzers, or combinations thereof.

The present invention is directed to a method comprising providing a feedstock slurry comprising fermentable carbon source, undissolved solids, and oil; separating the feedstock slurry whereby (i) a first aqueous solution comprising a fermentable carbon source, (ii) a first wet cake comprising solids, and (iii) a stream comprising oil, solids, and an aqueous stream comprising a fermentable carbon source are formed; and adding the first aqueous solution to a fermentation broth comprising microorganisms whereby a fermentation product is produced. In some embodiments, the method may further comprise separating the stream comprising oil, solids, and aqueous stream comprising a fermentable carbon source whereby (i) a second aqueous solution comprising a fermentable carbon source, (ii) a second wet cake comprising solids, and (iii) an oil stream are formed. In some embodiments, the first aqueous solution may be added to fermentation broth. In some embodiments, the second aqueous solution may be added to fermentation broth. In some embodiments, the second aqueous solution may be added to feedstock slurry. In some embodiments, the second aqueous solution may be added to liquefaction and/or feedstock preparation. In some embodiments, the first and second aqueous solutions may be combined prior to the addition to the fermentation broth. In some embodiments, the second aqueous solution may further comprise oil. In some embodiments, the oil of the second aqueous solution or portion thereof may be treated to generate an extractant. In some embodiments, the oil may be treated chemically or enzymatically. In some embodiments, separation may be a single step process. In some embodiments, separation may be performed by decanter bowl centrifugation, three-phase centrifugation, disk stack centrifugation, filtering centrifugation, decanter centrifugation, filtration, microfiltration, membrane filtration, vacuum filtration, beltfilter, pressure filtration, crossflow filtration, drum filter, filtration using a screen, screen separation, rotary screen, grating, porous grating, flotation, hydrocyclone, filter press, screwpress, gravity settler, vortex separator, or combination thereof.

The present invention is directed to a method comprising providing a feedstock slurry comprising a fermentable carbon source, undissolved solids, and oil; separating the feedstock slurry whereby (i) a first aqueous solution comprising a fermentable carbon source and solids, (ii) a first wet cake comprising solids, and (iii) a first oil stream are formed; and adding oil to the first aqueous solution whereby an oil layer comprising solids and a second aqueous solution comprising a fermentable carbon source are formed. In some embodiments, the oil layer comprising solids may be separated forming (i) a second oil stream, (ii) a second wet cake comprising solids, and (iii) a third aqueous solution comprising a fermentable carbon source. In some embodiments, the second aqueous solution and the third aqueous solution may be added to a fermentation broth comprising microorganisms whereby a fermentation product is produced. In some embodiments, the second aqueous solution and the third aqueous solution may further comprise oil. In some embodiments, the second aqueous solution and the third aqueous solution may be combined and the oil of the combined second aqueous solution and the third aqueous solution or portions thereof may be treated to generate an extractant. In some embodiments, the oil may be treated chemically or enzymatically. In some embodiments, separation may be a single step process. In some embodiments, separation may be performed by decanter bowl centrifugation, three-phase centrifugation, disk stack centrifugation, filtering centrifugation, decanter centrifugation, filtration, microfiltration, membrane filtration, vacuum filtration, beltfilter, pressure filtration, crossflow filtration, drum filter, filtration using a screen, screen separation, rotary screen, grating, porous grating, flotation, hydrocyclone, filter press, screwpress, gravity settler, vortex separator, or combination thereof.

The present invention is also directed to a system comprising one or more liquefaction units configured to liquefy a feedstock to create a feedstock slurry, the liquefaction unit comprising: an inlet for receiving the feedstock; and an outlet for discharging a feedstock slurry, wherein the feedstock slurry comprises fermentable carbon source, oil, and undissolved solids; and one or more separation units configured to remove the oil and undissolved solids from the feedstock slurry to create an aqueous solution comprising the fermentable carbon source, an oil stream, and a wet cake comprising the portion of the undissolved solids, the one or more separation units comprising: an inlet for receiving the feedstock slurry; a first outlet for discharging the aqueous solution; a second outlet for discharging the wet cake; and a third outlet for discharging the oil. In some embodiments, the system may further comprise one or more wash systems configured to recover the fermentable carbon source from the wet cake comprising: one or more mixing units; and one or more separation units. In some embodiments, the one or more separation units may be decanter bowl centrifugation, three-phase centrifugation, disk stack centrifugation, filtering centrifugation, decanter centrifugation, filtration, microfiltration, vacuum filtration, beltfilter, pressure filtration, membrane filtration, crossflow filtration, drum filter, filtration using a screen, screen separation, rotary screen, grating, porous grating, flotation, hydrocyclone, filter press, screwpress, gravity settler, vortex separator, or combinations thereof. In some embodiments, the system may further comprise one or more fermentors configured to ferment the aqueous solution to produce one or fermentation products, the fermentors comprising: an inlet for receiving the aqueous solution and/or wet cake; and an outlet for discharging fermentation broth comprising one or fermentation products. In some embodiments, the system may further comprise on-line measurement devices. In some embodiments, the on-line measurement devices may be particle size analyzers, Fourier transform infrared spectroscopes, near-infrared spectroscopes, Raman spectroscopes, high pressure liquid chromatography, viscometers, densitometers, tensiometers, droplet size analyzers, pH meters, dissolved oxygen probes, or combinations thereof.

### DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 schematically illustrates an exemplary process and system of the present invention, in which undissolved solids are removed by separation after liquefaction and before fermentation.

FIG. 2 schematically illustrates an exemplary alternative process and system of the present invention, in which feedstock is milled.

FIG. 3 schematically illustrates another exemplary alternative process and system of the present invention, in which undissolved solids and oil are removed by separation.

FIGS. 4A and 4B schematically illustrate another exemplary alternative process and system of the present invention, in which the wet cake is subjected to one or more wash cycles.

FIGS. 5A and 5B schematically illustrate another exemplary alternative process and system of the present invention, in which undissolved solids and oil are removed by separation and wet cake is subjected to one or more wash cycles.

FIG. 6 schematically illustrates another exemplary alternative process and system of the present invention, in which the aqueous solution and wet cake are combined and conducted to fermentation.

FIG. 7 schematically illustrates another exemplary alternative process and system of the present invention, in which the aqueous solution is saccharified prior to fermentation.

FIG. 8 schematically illustrates another exemplary alternative process and system of the present invention, in which the feedstock slurry is saccharified prior to separation.

FIGS. 9A to 9F schematically illustrate exemplary alternative processes and systems of the present invention, in which additional separation units are utilized to remove undissolved solids and oil.

FIG. 10 schematically illustrates an exemplary fermentation process utilizing on-line, in-line, at-line, and/or real-time measurements for monitoring fermentation processes.

FIG. 11 schematically illustrates an exemplary fermentation process of the present invention including downstream processing.

FIG. 12 illustrates the effect of the presence of undissolved corn mash solids on the overall volumetric mass transfer coefficient, k_{L}a, for the transfer of i-BuOH from an aqueous solution of liquefied corn starch to a dispersion of oleyl alcohol droplets flowing up through a bubble column when a nozzle with an inner diameter of 2.03 mm is used to disperse the oleyl alcohol.

FIG. 13 illustrates the effect of the presence of undissolved corn mash solids on the overall volumetric mass transfer coefficient, k_{L}a, for the transfer of i-BuOH from an aqueous solution of liquefied corn starch to a dispersion of oleyl alcohol droplets flowing up through a bubble column when a nozzle with an inner diameter of 0.76 mm is used to disperse the oleyl alcohol.

FIG. 14 illustrates the position of the liquid-liquid interface in the fermentation sample tubes as a function of (gravity) settling time. Phase separation data shown for run times: 5.3, 29.3, 53.3, and 70.3 hr run time. Sample data from extractive-fermentation where solids were removed from the mash feed, and oleyl alcohol was the solvent.

FIG. 15 illustrates the position of the liquid-liquid interface of the final fermentation broth as a function of (gravity) settling time. Data from extractive-fermentation where solids were removed from the mash feed, and oleyl alcohol was the solvent.

FIG. 16 illustrates the concentration of glucose in the aqueous phase of the slurries as a function of time for Batch 1 and Batch 2.

FIG. 17 illustrates concentration of glucose in the aqueous phase of the slurries as a function of time for Batch 3 and Batch 4.

FIG. 18 illustrates the effect of enzyme loading and +/−a high temperature stage was applied at some time during the liquefaction on starch conversion.

FIGS. 19A to 19E illustrate the effect of three-phase centrifuge conditions on separation of feedstock slurry.

### DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents, and other references mentioned herein are incorporated by reference in their entireties for all purposes.

In order to further define this invention, the following terms and definitions are herein provided.

As used herein, the terms “comprises,” “comprising,” “includes,” “including,” “has,” “having,” “contains,” or “containing,” or any other variation thereof, will be understood to imply the inclusion of a stated integer or group of integers but not the exclusion of any other integer or group of integers. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the application.

As used herein, the term “about” modifying the quantity of an ingredient or reactant of the invention employed refers to variation in the numerical quantity that can occur, for example, through typical measuring and liquid handling procedures used for making concentrates or solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about,” the claims include equivalents to the quantities. In one embodiment, the term “about” means within 10% of the reported numerical value, alternatively within 5% of the reported numerical value.

“Biomass” as used herein refers to a natural product containing hydrolyzable polysaccharides that provide fermentable sugars including any sugars and starch derived from natural resources such as corn, sugar cane (or cane), wheat, cellulosic or lignocellulosic material, and materials comprising cellulose, hemicellulose, lignin, starch, oligosaccharides, disaccharides, and/or monosaccharides, and mixtures thereof. Biomass may also comprise additional components such as protein and/or lipids. Biomass may be derived from a single source or biomass may comprise a mixture derived from more than one source. For example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, and wood and forestry waste (e.g., forest thinnings). Examples of biomass include, but are not limited to, corn grain, corn cobs, crop residues such as corn husks, corn stover, grasses, wheat, rye, wheat straw, spelt, triticale, barley, barley straw, oats, hay, rice, rice straw, switchgrass, potato, sweet potato, cassava, Jerusalem artichoke, sugar cane bagasse, sorghum, sugar cane, sugar beet, fodder beet, soy, palm, coconut, rapeseed, safflower, sunflower, millet, eucalyptus, miscanthus, waste paper, components obtained from milling of grains, trees, branches, roots, leaves, wood chips, sawdust, shrubs and bushes, vegetables, fruits, flowers, animal manure, and mixtures thereof. Mash, juice, molasses, or hydrolysate may be formed from biomass by any method known in the art for processing biomass for purposes of fermentation such as milling, treating (e.g., enzymatic, chemical), and/or liquefying. Treated biomass may comprise fermentable sugar and/or water. Cellulosic and/or lignocellulosic biomass may be processed to obtain a hydrolysate containing fermentable sugars by any method known to one skilled in the art. For example, a low ammonia pretreatment is disclosed in U.S. Patent Application Publication No. 2007/0031918A1, the entire contents of which are herein incorporated by reference. Enzymatic saccharification of cellulosic and/or lignocellulosic biomass typically makes use of enzyme mixtures for hydrolysis of cellulose and hemicellulose to produce a hydrolysate containing sugars including glucose, xylose, and arabinose. Saccharification enzymes suitable for cellulosic and/or lignocellulosic biomass are reviewed in Lynd, et al. (Microbiol. Mol. Biol. Rev. 66:506-577, 2002).

“Fermentable carbon source” or “fermentable carbon substrate” as used herein refers to a carbon source capable of being metabolized by microorganisms. Suitable fermentable carbon sources include, but are not limited to, monosaccharides such as glucose or fructose; disaccharides such as lactose or sucrose; oligosaccharides; polysaccharides such as starch or cellulose; one carbon substrates; and mixtures thereof.

“Fermentable sugar” as used herein refers to one or more sugars capable of being metabolized by microorganisms for the production of fermentation products such as alcohols.

“Feedstock” as used herein refers to a feed in a fermentation process. The feed may comprise a fermentable carbon source and may comprise undissolved solids and/or oil. Where applicable, the feed may comprise a fermentable carbon source before or after the fermentable carbon source has been liberated from starch or obtained from the hydrolysis of complex sugars by further processing such as by liquefaction, saccharification, or other process. Feedstock includes or may be derived from biomass. Suitable feedstocks include, but are not limited to, rye, wheat, corn, corn mash, sugar cane, cane mash, barley, cellulosic material, lignocellulosic material, or mixtures thereof. Where reference is made to “feedstock oil,” it will be appreciated that the term encompasses the oil produced from a given feedstock.

“Fermentation broth” as used herein refers to a mixture of water, fermentable carbon sources (e.g., sugars, starch), dissolved solids, optionally microorganisms producing fermentation products, optionally fermentation products (e.g., product alcohols), optionally undissolved solids, and other constituents of the material held in the fermentor in which fermentation product is being made by the metabolism of fermentable carbon sources by the microorganisms to form fermentation products, water, and carbon dioxide (CO₂). From time to time as used herein, the term “fermentation medium” and “fermented mixture” may be used synonymously with “fermentation broth.”

“Fermentor” or “fermentation vessel” as used herein refers to a vessel, unit, or tank in which the fermentation reaction is carried out whereby fermentation product (e.g., product alcohols such as ethanol or butanol) is made from fermentable carbon sources. Fermentor may also refer to a vessel, unit, or tank in which growth of microorganism occurs. In some instances, both microbial growth and fermentation may occur in a fermentor. The term “fermentor” may be used synonymously herein with “fermentation vessel.”

“Saccharification vessel” as used herein refers to a vessel, unit, or tank in which saccharification (i.e., the hydrolysis of oligosaccharides to monosaccharides) is carried out. Where fermentation and saccharification occur simultaneously, the saccharification vessel and the fermentor may be the same vessel.

“Saccharification enzyme” as used herein refers to one or more enzymes that are capable of hydrolyzing polysaccharides and/or oligosaccharides, for example, alpha-1,4-glucosidic bonds of glycogen, or starch. Saccharification enzymes may include enzymes capable of hydrolyzing cellulosic or lignocellulosic materials as well.

“Liquefaction vessel” as used herein refers to a vessel, unit, or tank in which liquefaction is carried out. Liquefaction is a process in which starch is hydrolyzed, for example, by an enzymatic process to obtain oligosaccharides. In embodiments where the feedstock is corn, oligosaccharides are hydrolyzed from the corn starch content during liquefaction.

“Sugar” as used herein refers to oligosaccharides, disaccharides, monosaccharides, and/or mixtures thereof. The term “saccharide” may also include carbohydrates such as starches, dextrans, glycogens, cellulose, pentosans, as well as sugars.

“Undissolved solids” as used herein refers to non-fermentable portions of feedstock which are not dissolved in the liquid or aqueous phase, for example, germ, fiber, and gluten. The non-fermentable portions of feedstock include the portion of feedstock that remains as solids and can absorb liquid from the fermentation broth. From time to time as used herein, the term “undissolved solids” may be used synonymously with “solids” or “suspended solids.”

“Extractant” as used herein refers to a solvent used to extract a fermentation product. From time to time as used herein, the term “extractant” may be used synonymously with “solvent.”

“In Situ Product Removal” (ISPR) as used herein refers to the selective removal of a product from a biological process such as fermentation to control the product concentration in the biological process as the product is produced.

“Product alcohol” as used herein refers to any alcohol that may be produced by a microorganism in a fermentation process that utilizes biomass as a fermentable carbon source. Product alcohols include, but are not limited to, C₁ to C₈ alkyl alcohols. In some embodiments, the product alcohols are C₂ to C₈ alkyl alcohols. In other embodiments, the product alcohols are C₂ to C₅ alkyl alcohols. It will be appreciated that C₁ to C₈ alkyl alcohols include, but are not limited to, methanol, ethanol, propanol, butanol, pentanol, hexanol, and isomers thereof. Likewise, C₂ to C₈ alkyl alcohols include, but are not limited to, ethanol, propanol, butanol, pentanol, hexanol, and isomers thereof. The term “alcohol” may also be used herein with reference to a product alcohol.

“Butanol” as used herein refers to butanol isomers: 1-butanol (1-BuOH), 2-butanol (2-BuOH), tertiary-butanol (tert-BuOH), and/or isobutanol (iBuOH, i-BuOH, or I-BUOH), either individually or as mixtures thereof.

“Propanol” as used herein refers to the propanol isomers: isopropanol or 1-propanol, either individually or as mixtures thereof.

“Pentanol” as used herein refers to the pentanol isomers: 1-pentanol, 3-methyl-1-butanol, 2-methyl-1-butanol, 2,2-dimethyl-1-propanol, 3-pentanol, 2-pentanol, 3-methyl-2-butanol, or 2-methyl-2-butanol, either individually or as mixtures thereof.

“Effective titer” as used herein refers to the total amount of a particular fermentation product (e.g., product alcohol) produced by fermentation. In some embodiments wherein the fermentation product is a product alcohol, effective titer may refer to the alcohol equivalent of an alcohol ester produced by alcohol esterification per liter of fermentation medium.

“Water-immiscible” or “insoluble” as used herein refers to a chemical component such as an extractant or solvent, which is incapable of mixing with an aqueous solution such as a fermentation broth, in such a manner as to form one liquid phase.

“Aqueous phase” as used herein refers to the aqueous phase of at least a biphasic mixture obtained by contacting a fermentation broth with an extractant, for example, a water-immiscible organic extractant. In an embodiment of a process described herein that includes fermentative extraction, the term “fermentation broth” then may refer to the aqueous phase in biphasic fermentative extraction.

“Aqueous phase titer” as used herein refers to the concentration of a fermentation product (e.g., product alcohol) in the aqueous phase.

“Organic phase” as used herein refers to the non-aqueous phase of at least a biphasic mixture obtained by contacting a fermentation broth with an extractant, for example, a water-immiscible organic extractant.

“Portion” as used herein with reference to a process stream refers to any fractional part of the stream which retains the composition of the stream, including the entire stream, as well as any component or components of the stream, including all components of the stream.

“By-product” or “co-product” as used herein refers to a product produced during the production of another product. In some instances, the term “co-product” may be used synonymously with the term “by-product.” Co-products include for example, oil recovered from the feedstock slurry, wet cake, and DDGS. Co-products may also include modification of the oil, wet cake, and DDGS for the purposes of improving value and/or for the manufacture of other products, such as biodiesel from the oil.

“Distillers co-products” as used herein refers to by-products from a product alcohol production process that can be isolated before or during fermentation. Distillers co-products include non-fermentable products remaining after product alcohol is removed from a fermented mash and solids isolated from a mash. As used herein, distillers co-products may be used in a variety of animal feed and non-animal feed applications. Examples of distillers co-products include, but are not limited to, fatty acids from oil hydrolysis, lipids from evaporation of thin stillage, syrup, distillers grains, distillers grains and solubles, solids from mash before fermentation, solids from whole stillage after fermentation, biodiesel, and acyl glycerides.

“Distillers co-products for animal feed” as used herein refers to distillers co-products that are suitable for use in or as animal feed. Examples of distillers co-products for animal feed include, but are not limited to, fatty acids from oil hydrolysis, lipids from evaporation of thin stillage, syrup, distillers grains, distillers grains and solubles, solids from mash before fermentation, and solids from whole stillage after fermentation.

“Distillers grains” or “DG” as used herein refer to the non-fermentable products remaining after product alcohol is removed from a fermented mash. Distillers grains that are dried are known as “distillers dried grains” or “DDG.” Distillers grains that are not dried are known as “wet distillers grains” or “WDG.”

“Distillers grains and solubles” or “DGS” as used herein refer to the non-fermentable products remaining after product alcohol is removed from a fermented mash, that have been blended with solubles. Distillers grains and solubles that are dried are known as “distillers dried grains and solubles” or “DDGS.” Distillers grains and solubles that are not dried are known as “wet distillers grains and solubles” or “WDGS.”

“Dried Distillers Grains with Solubles” (DDGS) as used herein refer to a co-product or by-product from a fermentation of a feedstock or biomass (e.g., fermentation of grain or grain mixture that produces a product alcohol). In some embodiments, DDGS may also refer to an animal feed produced from a process of making a product alcohol.

“Lipid” as used herein refers to any of a heterogeneous group of fats and fat-like substances including fatty acids, neutral fats, waxes, and steroids, which are water-insoluble and soluble in nonpolar solvents. Examples of lipids include monoglycerides, diglycerides, triglycerides, and phospholipids.

“Lipids from evaporation” as used herein in reference to a process stream refer to a lipid by-product produced by evaporation and centrifugation of thin stillage following fermentation in a product alcohol production process.

“Syrup” or “condensed distillers solubles” (CDS) as used herein in reference to a process stream refers to a by-product produced by evaporation of thin stillage following fermentation in a product alcohol production process.

“Process stream” as used herein refers to any by-product or co-product formed by a fermentation product production process. Examples of process streams include, but are not limited to, COFA, lipids from evaporation, syrup, DG, DDG, WDG, DGS, DDGS, and WDGS. Another example of a process stream is solids removed (e.g., by centrifugation) from a mash before fermentation in a fermentation product production process (e.g., the solids removed from a corn mash before fermentation). These solids may be referred to as “wet cake” when they have not been dried, and may be referred to as “dry cake” when they have been dried. Another example of a process stream is solids removed (e.g., by centrifugation) from whole stillage following fermentation in a fermentation product production process. These solids may be referred to as “WS wet cake” when they have not been dried, and may be referred to as “WS dry cake” when they have been dried. Process stream may also refer to any stream of the fermentation process such as feedstock slurry, aqueous solutions and streams, extractant, product stream, and the like. From time to time as used herein, the term “process stream” may be used synonymously with “stream.”

The present invention provides processes and methods for producing fermentation products such as product alcohols using fermentation. Other fermentation products that may be produced using the processes and methods described herein include propanediol, butanediol, acetone; acids such as lactic acid, acetic acid, butyric acid, and propionic acid; gases such as hydrogen methane, and carbon dioxide; amino acids; vitamins such as biotin, vitamin B2 (riboflavin), vitamin B12 (e.g., cobalamin), ascorbic acid (e.g., vitamin C), vitamin E (e.g., α-tocopherol), and vitamin K (e.g., menaquinone); antibiotics such as erythromycin, penicillin, streptomycin, and tetracycline; and other products such as citric acid, invertase, sorbitol, pectinase, and xylitol.

As an example of the processes and methods provided herein, a feedstock may be liquefied to create a feedstock slurry which comprises a fermentable carbon source (e.g., soluble sugar) and undissolved solids. In some instances, the terms “feedstock slurry” and “mash” may be used interchangeably. In some embodiments, the feedstock slurry comprises soluble sugar, undissolved solids, and oil. If the feedstock slurry is fed directly to a fermentor, the undissolved solids and/or oil may interfere with efficient removal and recovery of the fermentation product such as a product alcohol. For example, if liquid-liquid extraction is utilized to extract product alcohol from fermentation broth, the presence of undissolved solids may cause system inefficiencies including, but not limited to, decreasing the mass transfer rate of the product alcohol to the extractant by interfering with the contact between the extractant and the fermentation broth; creating an emulsion in the fermentor and thereby interfering with phase separation of the extractant and the fermentation broth; slowing disengagement of the extractant from the fermentation broth; reducing the efficiency of recovering and recycling the extractant because at least a portion of the extractant and product alcohol becomes “trapped” in the solids; shortening the life cycle of the extractant by contamination with oil; and lowering fermentor volume efficiency because there are solids taking up volume in the fermentor. These effects can result in higher capital and operating costs. In addition, the extractant “trapped” in undissolved solids used to generate Distillers Dried Grains with Solubles (DDGS), may detract from DDGS value and qualification for sale as animal feed. Therefore, in order to avoid and/or minimize these problems, at least a portion of the undissolved solids may be removed from the feedstock slurry prior to the addition of the feedstock slurry to the fermentor. Extraction activity and the efficiency of product alcohol production can be increased when extraction is performed on fermentation broth containing an aqueous solution where undissolved solids have been removed relative to extraction performed on fermentation broth containing an aqueous solution where undissolved solids have not been removed.

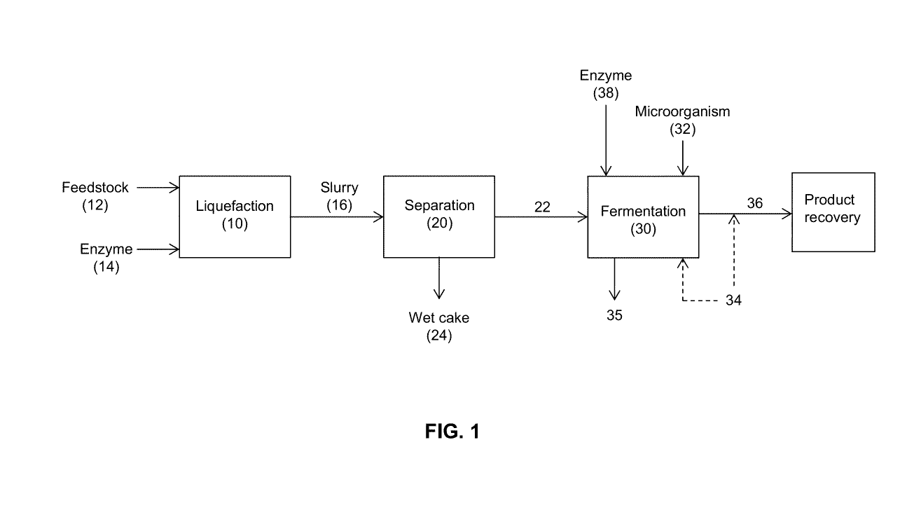

The processes and systems of the present invention will be described with reference to the Figures. In some embodiments, as shown, for example, in FIG. 1, the system includes liquefaction 10 configured to liquefy a feedstock to create a feedstock slurry.

For example, feedstock 12 may be introduced to an inlet in liquefaction 10. Feedstock 12 may be any suitable biomass material that contains a fermentable carbon source such as starch including, but not limited to, barley, oat, rye, sorghum, wheat, triticale, spelt, millet, cane, corn, or combinations thereof. Water may also be introduced to liquefaction 10.

The process of liquefying feedstock 12 involves hydrolysis of feedstock 12 generating water-soluble sugars. Any known liquefying processes utilized by the industry may be used including, but not limited to, an acid process, an enzyme process, or an acid-enzyme process. Such processes may be used alone or in combination. In some embodiments, the enzyme process may be utilized and an appropriate enzyme 14, for example, alpha-amylase, is introduced to an inlet in liquefaction 10. Examples of alpha-amylases that may be used in the processes and systems of the present invention are described in U.S. Pat. No. 7,541,026; U.S. Patent Application Publication No. 2009/0209026; U.S. Patent Application Publication No. 2009/0238923; U.S. Patent Application Publication No. 2009/0252828; U.S. Patent Application Publication No. 2009/0314286; U.S. Patent Application Publication No. 2010/02278970; U.S. Patent Application Publication No. 2010/0048446; and U.S. Patent Application Publication No. 2010/0021587, the entire contents of each are herein incorporated by reference.

In some embodiments, enzymes for liquefaction and/or saccharification may be produced by the microorganism (e.g., microorganism 32). Examples of microorganisms producing such enzymes are described in U.S. Pat. No. 7,498,159; U.S. Patent Application Publication No. 2012/0003701; U.S. Patent Application Publication No. 2012/0129229; PCT International Publication No. WO 2010/096562; and PCT International Publication No. WO 2011/153516, the entire contents of each are herein incorporated by reference.

The process of liquefying feedstock 12 produces feedstock slurry 16 that comprises a fermentable carbon source and undissolved solids. In some embodiments, feedstock slurry 16 may comprise a fermentable carbon source, oil, and undissolved solids. The undissolved solids are non-fermentable portions of feedstock 12. In some embodiments, feedstock 12 may be corn, such as dry milled, unfractionated corn kernels, and the undissolved solids may comprise germ, fiber, and gluten. In some embodiments, feedstock 12 is corn or corn kernels and feedstock slurry 16 is corn mash slurry. Feedstock slurry 16 may be discharged from an outlet of liquefaction 10 and conducted to separation 20.

In some embodiments, nutrients such as amino acids, nitrogen, minerals, trace elements, and/or vitamins may be added to feedstock slurry 16 or fermentation 30. For example, one or more of the following: biotin, pantothenate, folic acid, niacin, aminobenzoic acid, pyridoxine, riboflavin, thiamine, vitamin A, vitamin C, vitamin D, vitamin E, vitamin K, inositol, potassium (e.g., potassium phosphate), boric acid, calcium (e.g., calcium chloride), chromium, copper (e.g., copper sulfate), iodide (e.g., potassium iodide), iron (e.g., ferric chloride), lithium, magnesium (e.g., magnesium sulfate, magnesium chloride), manganese (e.g., manganese sulfate), molybdenum, phosphorus, potassium, sodium chloride, vanadium, zinc (e.g., zinc sulfate), yeast extract, soy peptone, and the like may be added to feedstock slurry 16 or fermentation 30. Examples of amino acids include essential amino acids such as histidine, isoleucine, leucine, lysine, methionine, phenylalanine, threonine, tryptophan, and valine as well as other amine acids such as alanine, arginine, aspartic acid, asparagine, cysteine, glutamic acid, glutamine, glycine, hydroxylysine, hydroxyproline, ornithine, proline, serine, and tyrosine.

Separation 20 via an inlet may be configured to remove undissolved solids from feedstock slurry 16. Separation 20 may also be configured to remove oil, or to remove both oil and undissolved solids. Separation 20 may be any device capable of separating solids and liquids. For example, separation 20 may be any conventional centrifuge utilized in the industry, including, for example, a decanter bowl centrifuge, three-phase centrifuge, disk stack centrifuge, filtering centrifuge, or decanter centrifuge. In some embodiments, separation may be accomplished by filtration, microfiltration, vacuum filtration, beltfilter, membrane filtration, crossflow filtration, drum filter, pressure filtration, filtration using a screen, screen separation, rotary screen, grates or grating, porous grating, flotation, hydrocyclone, filter press, screwpress, gravity settler, vortex separator, or any method or separation device that may be used to separate solids and liquids.

Feedstock slurry 16, conducted to separation 20, may be separated to form a liquid phase or aqueous solution 22 (also known as thin mash) and a solid phase, solid stream, or wet cake 24. Aqueous solution 22 may comprise sugar, for example, in the form of oligosaccharides, and water. In some embodiments, aqueous solution 22 may comprise at least about 10% by weight oligosaccharides, at least about 20% by weight of oligosaccharides, or at least about 30% by weight of oligosaccharides. In some embodiments, aqueous solution 22 may be discharged from an outlet located near the top of separation 20. In some embodiments, aqueous solution 22 may have a viscosity of less than about 20 centipoise (cP). In some embodiments, aqueous solution 22 may comprise less than about 20 g/L of monomeric glucose, less than about 10 g/L of monomeric glucose, or less than about 5 g/L of monomeric glucose. Suitable methodology to determine the amount of monomeric glucose is well known in the art such as high performance liquid chromatography (HPLC).

Wet cake 24 may be discharged from separation 20. In some embodiments, wet cake 24 may be discharged from an outlet located near the bottom of separation 20. Wet cake 24 may comprise undissolved solids. In some embodiments, wet cake 24 may also comprise a portion of sugar and water. Wet cake 24 may be washed with additional water using separation 20 once aqueous solution 22 has been discharged from separation 20. In some embodiments, wet cake 24 may be washed with additional water using additional separation devices. Washing wet cake 24 will recover the sugar or sugar source (e.g., oligosaccharides) present in the wet cake, and the recovered sugar and water may be recycled to liquefaction 10. After washing, wet cake 24 may be processed to form DDGS using any suitable known process. The formation of DDGS from wet cake 24 has several benefits. For example, since the undissolved solids are not added to the fermentor, the undissolved solids are not subjected to the conditions of the fermentor and therefore, the undissolved solids do not contact the microorganisms present in the fermentor, and fermentation product such as product alcohol or other components such as extractant are not trapped in the undissolved solids. These effects provide benefits to subsequent processing and use of DDGS, for example, as animal feed because the DDGS would not contain microorganism or other components (e.g., product alcohol, extractant) of the fermentation broth.

In some embodiments, undissolved solids may be separated from feedstock slurry to form two product streams, for example, an aqueous solution of oligosaccharides which contains a lower concentration of solids as compared to the feedstock slurry, and a wet cake which contains a higher concentration of solids as compared to the feedstock slurry. In addition, a third stream containing oil may be generated. As such, a number of product streams may be generated by using different separation techniques or a combination thereof. As an example, feedstock slurry 16 may be separated using a three-phase centrifuge. A three-phase centrifuge allows for three-phase separation yielding two liquid phases (e.g., aqueous stream and oil stream) and a solid stream (e.g., solids or wet cake) (see, e.g., Flottweg Tricanter®, Flottweg AG, Vilsibiburg, Germany). The two liquid phases may be separated and decanted, for example, from a bowl via two discharge systems to prevent cross-contamination and the solids stream may be removed via a separate discharge system.

In some embodiments using corn as feedstock 12, a three-phase centrifuge may be used to remove solids and corn oil simultaneously from feedstock slurry 16 (e.g., liquefied corn mash). The solids are the undissolved solids remaining after the starch is hydrolyzed to soluble oligosaccharides during liquefaction, and the corn oil is free oil that is released from the germ during grinding and/or liquefaction. In some embodiments, the three-phase centrifuge may have one feed stream and three outlet streams. The feed stream may consist of liquefied corn mash produced during liquefaction. The mash may consist of an aqueous solution of liquefied starch (e.g., oligosaccharides); undissolved solids which comprise insoluble, non-starch components from the corn; and corn oil which comprise glycerides and free fatty acids. The three outlet streams from the three-phase centrifuge may be a wet cake (e.g., wet cake 24) which contains the undissolved solids from the feedstock slurry; a heavy centrate stream which contains the liquefied starch from the feedstock slurry; and a light centrate stream which contains the corn oil from the feedstock slurry. In some embodiments, the light centrate stream (e.g., oil 26) may be conducted to a storage tank or any vessel that is suitable for oil storage. In some embodiments, the oil may be sold as a co-product, converted to another co-product, or used in processing such as the case in converting corn oil to corn oil fatty acids. In some embodiments, the heavy centrate stream (e.g., aqueous solution 22) may be used for fermentation. In some embodiments, the wet cake may be washed with process recycle water, such as evaporator condensate and/or backset as described herein, to recover the soluble starch in the liquid phase of the cake.

In some embodiments, wet cake 24 is a composition formed from feedstock slurry 16, and may comprise at least about 50% by weight of the undissolved solids present in the feedstock slurry, at least about 55% by weight of the undissolved solids present in the feedstock slurry, at least about 60% by weight of the undissolved solids present in the feedstock slurry, at least about 65% by weight of the undissolved solids present in the feedstock slurry, at least about 70% by weight of the undissolved solids present in the feedstock slurry, at least about 75% by weight of the undissolved solids present in the feedstock slurry, at least about 80% by weight of the undissolved solids present in the feedstock slurry, at least about 85% by weight of the undissolved solids present in the feedstock slurry, at least about 90% by weight of the undissolved solids present in the feedstock slurry, at least about 95% by weight of the undissolved solids present in the feedstock slurry, or at least about 99% by weight of the undissolved solids present in the feedstock slurry.

In some embodiments, aqueous solution 22, formed from feedstock slurry 16, may comprise no more than about 50% by weight of the undissolved solids present in the feedstock slurry, no more than about 45% by weight of the undissolved solids present in the feedstock slurry, no more than about 40% by weight of the undissolved solids present in the feedstock slurry, no more than about 35% by weight of the undissolved solids present in the feedstock slurry, no more than about 30% by weight of the undissolved solids present in the feedstock slurry, no more than about 25% by weight of the undissolved solids present in the feedstock slurry, no more than about 20% by weight of the undissolved solids present in the feedstock slurry, no more than about 15% by weight of the undissolved solids present in the feedstock slurry, no more than about 10% by weight of the undissolved solids present in the feedstock slurry, no more than about 5% by weight of the undissolved solids present in the feedstock slurry, or about 1% by weight of the undissolved solids present in the feedstock slurry.

Fermentation 30 configured to ferment aqueous solution 22 to produce a fermentation product such as a product alcohol has an inlet for receiving aqueous solution 22. Fermentation 30 may include fermentation broth. In some embodiments, microorganism 32 may be bacteria, cyanobacteria, filamentous fungi, or yeasts, and may be introduced to fermentation 30 to be included in the fermentation broth. In some embodiments, microorganism 32 may be bacteria such as Escherichia coli. In some embodiments, microorganism 32 may be Saccharomyces cerevisiae. In some embodiments, microorganism 32 consumes the sugar in aqueous solution 22 and produces a product alcohol such as ethanol or butanol. In some embodiments, microorganism 32 may be a recombinant microorganism.

In some embodiments, microorganism 32 may be engineered to contain a biosynthetic pathway. In some embodiments, the biosynthetic pathway may be a butanol biosynthetic pathway. In some embodiments, the butanol biosynthetic pathway may be a 1-butanol biosynthetic pathway, a 2-butanol biosynthetic pathway, or an isobutanol biosynthetic pathway. In some embodiments, the biosynthetic pathway converts pyruvate to a fermentation product. In some embodiments, the biosynthetic pathway converts pyruvate as well as amino acids to a fermentation product. In some embodiments, the biosynthetic pathway comprises at least one heterologous polynucleotide encoding a polypeptide which catalyzes a substrate to product conversion of the biosynthetic pathway. In some embodiments, each substrate to product conversion of the biosynthetic pathway is catalyzed by a polypeptide encoded by a heterologous polynucleotide. Examples of the production of a product alcohol by a microorganism comprising a biosynthetic pathway are disclosed, for example, in U.S. Pat. No. 7,851,188, and U.S. Patent Application Publication Nos. 2007/0092957; 2007/0259410; 2007/0292927; 2008/0182308; 2008/0274525; 2009/0155870; 2009/0305363; and 2009/0305370, the entire contents of each are herein incorporated by reference.

In some embodiments, microorganism 32 may also be immobilized, such as by adsorption, covalent bonding, crosslinking, entrapment, and encapsulation. Methods for encapsulating cells are known in the art such as methods described in U.S. Patent Application Publication No. 2011/0306116, the entire contents of which are herein incorporated by reference.

In some embodiments of the processes and systems described herein, in situ product removal (ISPR) may be utilized to remove a fermentation product such as a product alcohol from fermentation broth. In some embodiments, ISPR may be conducted in fermentation 30 as the fermentation product is produced by the microorganism, or external to fermentation 30, using, for example, liquid-liquid extraction. Methods for producing and recovering product alcohols from a fermentation broth using extractive fermentation are described in U.S. Patent Application Publication No. 2009/0305370; U.S. Patent Application Publication No. 2010/0221802; U.S. Patent Application Publication No. 2011/0097773; U.S. Patent Application Publication No. 2011/0312044; U.S. Patent Application Publication No. 2011/0312043; and U.S. Patent Application Publication No. 2012/0156738; the entire contents of each are herein incorporated by reference.

In some embodiments, fermentation 30 may have an inlet for receiving extractant 34. In some embodiments, extractant 34 may be added to the fermentation broth external to fermentation 30. In some embodiments, extractant 34 may be added to an external extractor or external extraction loop. Alternative means of additions of extraction 34 to fermentation 30 or external to fermentation 30 are represented by the dotted lines. In some embodiments, extractant 34 may be immiscible organic solvents. In some embodiments, extractant 34 may be water-immiscible organic solvents. In some embodiments, extractant 34 may be an organic extractant selected from the group consisting of saturated, monounsaturated, polyunsaturated compounds, and mixtures thereof. In some embodiments, extractant 34 may be selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides, and mixtures thereof. In some embodiments, extractant 34 may also be selected from the group consisting of C₄ to C₂₂ fatty alcohols, C₄ to C₂₈ fatty acids, esters of C₄ to C₂₈ fatty acids, C₄ to C₂₂ fatty aldehydes, and mixtures thereof. In some embodiments, extractant 34 may include a first extractant selected from C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides, and mixtures thereof; and a second extractant selected from C₇ to C₁₁ fatty alcohols, C₇ to C₁₁ fatty acids, esters of C₇ to C₁₁ fatty acids, C₇ to C₁₁ fatty aldehydes, and mixtures thereof. In some embodiments, extractant 34 may be carboxylic acids. In some embodiments, extractant 34 may be corn oil fatty acids (COFA) or soybean oil fatty acids (SOFA). In some embodiments, extractant 34 may be an organic extractant such as oleyl alcohol, behenyl alcohol, cetyl alcohol, lauryl alcohol, myristyl alcohol, stearyl alcohol, oleic acid, lauric acid, linoleic acid, linolenic acid, myristic acid, stearic acid, octanoic acid, decanoic acid, undecanoic acid, methyl myristate, methyl oleate, 1-nonanol, 1-decanol, 2-undecanol, 1-nonanal, 1-undecanol, undecanal, lauric aldehyde, 2-methylundecanal, oleamide, linoleamide, palmitamide, stearylamide, 2-ethyl-1-hexanol, 2-hexyl-1-decanol, 2-octyl-1-dodecanol, and mixtures thereof. For the processes and systems described herein and as illustrated in the figures, extractant may be added to the fermentor or an external extractor.

In some embodiments, the extractant may be selected based upon certain properties. For example, the extractant may have a high K_{d}. K_{d} refers to the partition coefficient of the fermentation product (e.g., product alcohol) between the extractant phase (e.g., organic phase) and aqueous phase. In some embodiments, the extractant may have a high selectivity. For example, selectivity refers to the relative amounts of product alcohol to water taken up by the extractant.

In some embodiments, the extractant may be biocompatible. In some embodiments, biocompatible refers to a measure of the ability of a microorganism to utilize fermentable carbon sources in the presence of an extractant. In some embodiments, the extractant may be a mixture of biocompatible and non-biocompatible extractants. In some embodiments, a non-biocompatible extractant refers to an extractant that interferes with the ability of a microorganism to utilize fermentable carbon sources. For example, in the presence of a non-biocompatible extractant, the microorganism does not utilize fermentable carbon sources at a rate greater than about 50% of the rate when the extractant is not present. In some embodiments, in the presence of a non-biocompatible extractant, the microorganism does not utilize fermentable carbon sources at a rate greater than about 25% of the rate when the extractant is not present. Examples of mixtures of biocompatible and non-biocompatible extractants include, but are not limited to, oleyl alcohol and nonanol, oleyl alcohol and 1-undecanol, oleyl alcohol and 2-undecanol, oleyl alcohol and 1-nonanal, oleyl alcohol and decanol, and oleyl alcohol and dodecanol. Additional examples of biocompatible and non-biocompatible extractants are described in U.S. Patent Application Publication No. 2011/0097773; the entire contents of which are herein incorporated by reference.

Extractant 34 contacts the fermentation broth forming stream 36 comprising a biphasic mixture (i.e., aqueous phase and organic phase). In the case that the fermentation product is a product alcohol, product alcohol present in the fermentation broth is transferred to extractant 34 forming extractant rich with product alcohol (e.g., organic phase). In some embodiments, stream 36 may be discharged through an outlet in fermentation 30. Product alcohol may be separated from the extractant in stream 36 using conventional techniques. Feed stream may be added to fermentation 30. Fermentation 30 can be any suitable fermentor known in the art.

In some embodiments, where extractant 34 is not added to the fermentation broth, stream 36 comprises fermentation broth and product alcohol. Stream 36 or a portion thereof comprising product alcohol and fermentation broth may be discharged from fermentation 30 and further processed for recovery of product alcohol. In some embodiments, the processed fermentation broth may be recycled to fermentation 30.

In some embodiments, simultaneous saccharification and fermentation (SSF) may occur in fermentation 30. Any known saccharification process utilized by the industry may be used including, but not limited to, an acid process, an enzyme process, or an acid-enzyme process. In some embodiments, enzyme 38 such as glucoamylase, may be introduced to hydrolyze sugars (e.g., oligosaccharides) in feedstock slurry 16 or aqueous solution 22 to form monosaccharides. Examples of glucoamylases that may be used in the processes and systems of the present invention are described in U.S. Pat. No. 7,413,887; U.S. Pat. No. 7,723,079; U.S. Patent Application Publication No. 2009/0275080; U.S. Patent Application Publication No. 2010/0267114; U.S. Patent Application Publication No. 2011/0014681; U.S. Patent Application Publication No. 2011/0020899, the entire contents of each are herein incorporated by reference. In some embodiments, the glucoamylase may be expressed by a recombinant microorganism that also produces the fermentation product (e.g., product alcohol).

In some embodiments, enzymes such as glucoamylases may be added to liquefaction. The addition of enzymes such as glucoamylases to liquefaction may reduce the viscosity of the feedstock slurry or liquefied mash, and may improve separation efficiency. In some embodiments, any enzyme capable of reducing the viscosity of the feedstock slurry may be used (e.g., Viscozyme®, Sigma-Aldrich, St. Louis, Mo.). Viscosity of the feedstock may be measured by any method known in the art, including the method described in Example 22.

In some embodiments, stream 35 may be discharged from an outlet in fermentation 30. The discharged stream 35 may include microorganism 32 such as yeast. Microorganism 32 may be separated from the stream 35, for example, by centrifugation (not shown). Microorganism 32 may then be recycled to fermentation 30 which over time can increase the production rate of product alcohol, thereby resulting in an increase in the efficiency of product alcohol production.

When a portion of stream 35 exits fermentation 30, stream 35 may include no more than about 50% by weight of the undissolved solids present in the feedstock slurry, no more than about 45% by weight of the undissolved solids present in the feedstock slurry, no more than about 40% by weight of the undissolved solids present in the feedstock slurry, no more than about 35% by weight of the undissolved solids present in the feedstock slurry, no more than about 30% by weight of the undissolved solids present in the feedstock slurry, no more than about 25% by weight of the undissolved solids present in the feedstock slurry, no more than about 20% by weight of the undissolved solids present in the feedstock slurry, no more than about 15% by weight of the undissolved solids present in the feedstock slurry, no more than about 10% by weight of the undissolved solids present in the feedstock slurry, no more than about 5% by weight of the undissolved solids present in the feedstock slurry, or no more than about 1% by weight of the undissolved solids present in the feedstock slurry.

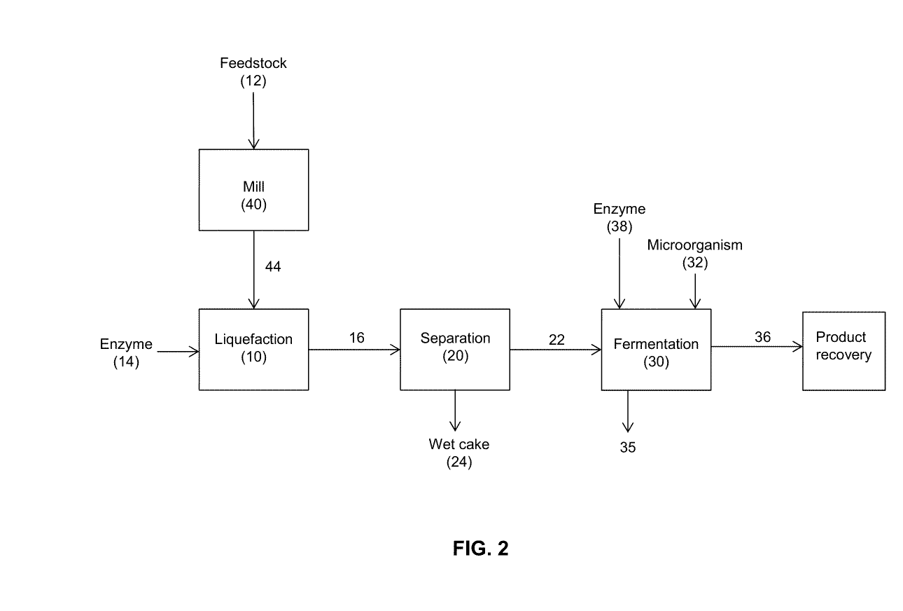

In some embodiments, as shown, for example, in FIG. 2, the processes and systems of the present invention may include mill 40 configured to dry mill feedstock 12. Feedstock 12 may enter mill 40 through an inlet, and mill 40 may mill or grind feedstock 12. In some embodiments, feedstock 12 may be unfractionated. In some embodiments, feedstock 12 may be unfractionated corn kernels. Mill 40 may be any suitable known mill, for example, a hammer mill. Dry milled feedstock 44 is discharged from mill 40 through an outlet and enters liquefaction 10. The remainder of FIG. 2 is similar to FIG. 1, and therefore will not be described in detail again. In other embodiments, the feedstock may be fractionated and/or wet milled as is known in the industry as an alternative to being unfractionated and/or dry milled.

Wet milling is a multi-step process that separates biomass into several components such as germ, pericarp fiber, starch, and gluten in order to capture value from each co-product separately. Using corn as a feedstock, this process produces several co-products: starch, gluten feed, gluten meal, and corn oil streams. These streams may be recombined and processed to produce customized products for the feed industry. As an example of a wet milling process, feedstock (e.g., corn) may be conducted to steeping tanks where it is soaked, for example, in a sodium dioxide solution for about 30-50 hours at about 120-130° F. (about 50-55° C.). Nutrients released into the water may be collected and evaporated to produce condensed fermented extractives (or steep liquor). Germ may be removed from the soaked feedstock and further processed to recover oil and germ meal. After removal of the germ, the remaining portion of feedstock may be processed to remove bran and to produce a starch and gluten slurry. The slurry may be further processed to separate the starch and gluten protein which may be dried to form gluten meal. The starch stream may be further processed via fermentation to produce a fermentation product (e.g., product alcohol) or may be utilized by the food, paper, or textile industries. For example, the starch stream may be used to produce sweeteners. The gluten meal and gluten feed stream which both contain protein, fat, and fiber, may be used in feeds for dairy and beef cattle, poultry, swine, livestock, equine, aquaculture, and domestic pets. Gluten feed may also be used as a carrier for added micronutrients. Gluten meal also contains methionine and xanthophylls which may be used a pigment ingredient in, for example, poultry feeds (e.g., xanthophylls provide yellow pigmentation for egg yolks). Condensed fermented extractives which contain protein, growth factors, B vitamins, and minerals may be used as a high energy liquid feed ingredient. Condensed extractives may also be used as a pellet binder. This process provides a purified starch stream; however, it is costly and includes the separation of the biomass into its non-starch components which may not be necessary for fermentation production.

Fractionation removes fiber and germ, which contains a majority of the lipids (e.g., oil) present in ground whole corn resulting in a fractionated corn that has a higher starch (endosperm) content. In some embodiments, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of the oil may be removed from the germ by fractionation. In some embodiments, fractionation may reduce the undissolved solids content of the feedstock to at least about 0.5%, at least about 1%, at least about 2%, at least about 3%, at least about 4%, or at least about 5% of the feedstock.

Dry fractionation does not separate the germ from fiber and therefore, it is less expensive than wet milling. The benefits of fractionation may include, for example, improved yield of product, increased volume (i.e., space) in the fermentor, smaller column diameters, lower enzyme loadings and increased efficiency for saccharification, improved oil removal, decreased equipment clogging due to the removal of oil, fewer cleaning shutdowns, increased protein levels in DDGS, reduced drying time for DDGS, and reduced energy consumption. However, fractionation does not remove the entirety of the fiber or germ, and does not result in total elimination of solids. Furthermore, there is some loss of starch in fractionation.

Dry milling may also be utilized for feedstock processing. Feedstock may be milled, for example, using a hammermill to generate a meal that may then be mixed with water to form a slurry. The slurry may be subjected to liquefaction by the addition of enzymes such as amylases to hydrolyze starch to sugars, forming a mash. The mash may be heated (“cooked”) to inactivate the enzyme and then cooled for addition to fermentation. Cooled mash, microorganism, and enzyme such as glucoamylase may be added to fermentation for the production of fermentation product (e.g., product alcohol). Following fermentation, the fermentation broth may be conducted to distillation for recovery of the fermentation product. If the undissolved solids have not been removed, the bottoms stream of the distillation column is whole stillage containing unfermented solids (e.g., distillers grain solids), dissolved materials, and water which may be collected for further processing. For example, the whole stillage may be separated into solids (e.g., wet cake) and thin stillage. Separation may be accomplished by a number of means including, but not limited to, centrifugation, filtration, screen separation, hydrocyclone, or any other means or separation device for separating liquids from solids. Thin stillage may be conducted to evaporation forming condensed distillers solubles (CDS) or syrup. Thin stillage may comprise soluble nutrients, small grain solids (or fine particles), and microorganisms. The solids (e.g., wet cake) may be combined with syrup and then dried to form DDGS. Syrup contains protein, fat, and fiber as well as vitamins and minerals such as phosphorus and potassium; and may be added to animal feeds for its nutritional value and palatability. DDGS contains protein, fat, and fiber; and provides a source of bypass proteins. DDGS may be used in animal feeds for dairy and beef cattle, poultry, swine, livestock, equine, aquaculture, and domestic pets.

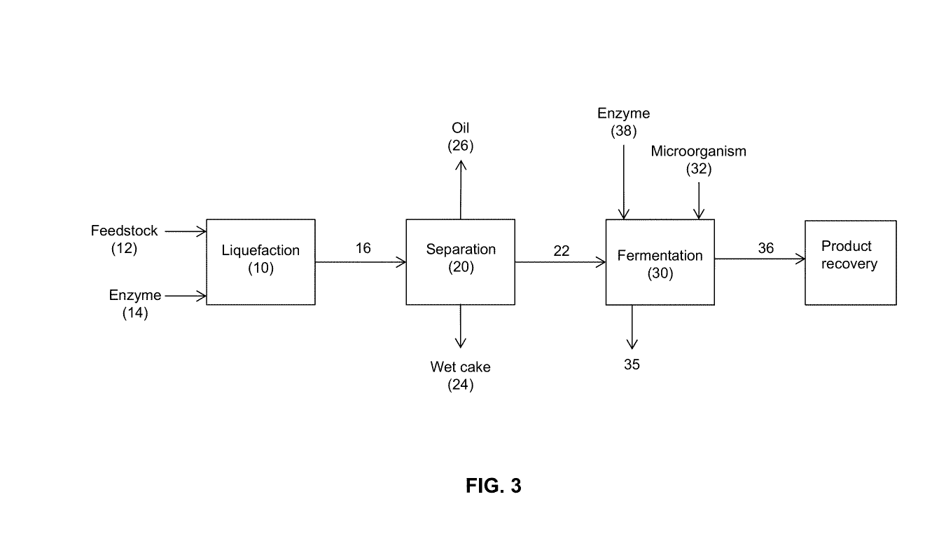

In some embodiments, as shown, for example, in FIG. 3, the processes and systems of the present invention may include discharging oil 26 from an outlet of separation 20. FIG. 3 is similar to FIG. 1, except for oil stream 26 exiting separation 20, and therefore will not be described in detail again.

Feedstock slurry 16, conducted to separation 20, may be separated into a first liquid phase or aqueous solution 22 containing a fermentable sugar, a second liquid phase containing oil 26, and a solid phase or wet cake 24 containing undissolved solids. Any suitable separation device can be used to discharge aqueous solution 22 (or aqueous stream), wet cake 24 (or solid stream), and oil 26 (or oil stream), for example, a three-phase centrifuge. In some embodiments, feedstock 12 is corn and oil 26 is corn oil (e.g., free corn oil). The term free corn oil as used herein means corn oil that is freed from the corn germ. In some embodiments, oil 26 may be conducted to a storage tank or any vessel that is suitable for oil storage. In some embodiments, a portion of oil from feedstock 12 such as corn oil when the feedstock is corn, remains in wet cake 24.

In some embodiments, when oil 26 is removed via separation 20, the fermentation broth in fermentation 30 includes a reduced amount of oil. In some embodiments after oil removal, the feedstock slurry may comprise about 1 wt %, about 2 wt %, about 3 wt %, or about 4 wt % oil.

In some embodiments, the process and system of FIG. 2 may be modified to include discharge of an oil stream from separation 20 as described herein in connection to the process and system of FIG. 3.

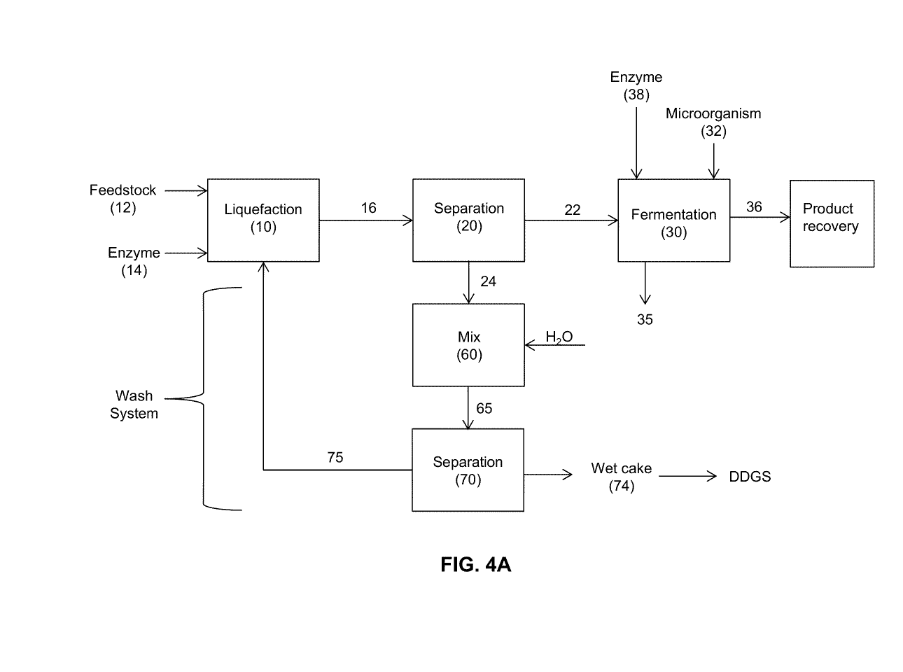

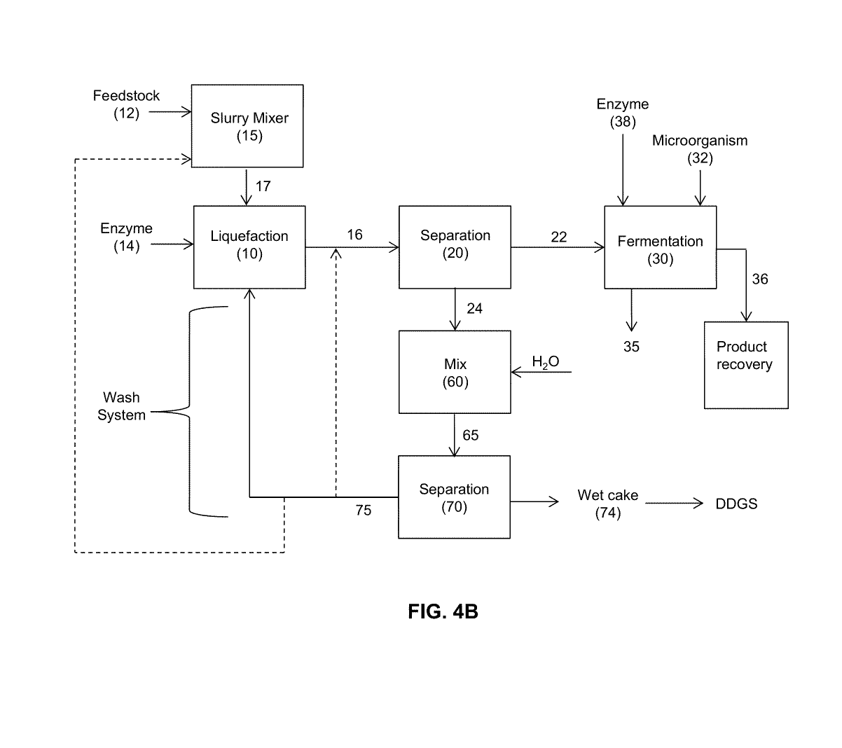

As illustrated in FIG. 4A, if oil is not discharged separately, it may be removed with wet cake 24. When wet cake 24 is separated via separation 20, in some embodiments, a portion of the oil from feedstock 12, such as corn oil when the feedstock is corn, remains in wet cake 24. Wet cake 24 may be conducted to mix 60 and combined with water or other solvents (i.e., re-slurried) forming wet cake mixture 65. In some embodiments, the water may be fresh water, backset, cook water, process water, lutter water, evaporation water, or any water source available in the fermentation processing facility, or any combination thereof. Wet cake mixture 65 may be conducted to separation 70 producing wash centrate 75 comprising fermentable sugars recovered from wet cake 24, and wet cake 74. Wash centrate 75 may be recycled to liquefaction 10. The remainder of FIG. 4A is similar to FIG. 1, and therefore will not be described in detail again.

In some embodiments, separation 70 may be any separation device capable of separating solids and liquids including, for example, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof. In some embodiments, separation may be a single step process.

In some embodiments, wet cake may be subjected to one or more wash cycles or wash systems. For example, wet cake 74 may be further processed by conducting wet cake 74 to a second wash system. In some embodiments, wet cake 74 may be conducted to a second mix 60′ forming wet cake mixture 65′. Wet cake mixture 65′ may be conducted to a second separation 70′ producing wash centrate 75′ and wet cake 74′. Wash centrate 75′ may be recycled to liquefaction 10 and/or wash centrate 75′ may be combined with wash centrate 75 and the combined wash centrates may be recycled to liquefaction 10. Wet cake 74′ may be combined with wet cake 74 for further processing as described herein. In some embodiments, separation 70′ may be any separation device capable of separating solids and liquids including, for example, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof. In some embodiments, separation may be a single step process. In some embodiments, the wet cake may be subjected to one, two, three, four, five, or more wash cycles or wash systems.

Wet cake 74 may be combined with solubles and then dried to form DDGS through any suitable known process. The formation of the DDGS from wet cake 74 has several benefits. For example, since the undissolved solids are not added to the fermentor, the undissolved solids are not subjected to the conditions of the fermentor and therefore, the undissolved solids do not contact the microorganisms present in the fermentor, and fermentation product such as product alcohol or other components such as extractant are not trapped in the undissolved solids. These effects provide benefits to subsequent processing and use of DDGS, for example, as animal feed because the DDGS would not contain microorganism or other components (e.g., product alcohol, extractant) of the fermentation broth.

As shown in FIG. 4A, oil is not discharged separately from the wet cake, but rather oil is included as part of the wet cake and is ultimately present in the DDGS. If corn is utilized as feedstock, corn oil contains triglycerides, diglycerides, monoglycerides, fatty acids, and phospholipids, which provide a source of metabolizable energy for animals. The presence of oil in the wet cake and ultimately DDGS may provide a desirable animal feed, for example, a high fat content animal feed.

In some embodiments, oil may be separated from the DDGS and converted to an ISPR extractant for subsequent use in the same or different fermentation processes. Methods for deriving extractants from biomass are described in U.S. Patent Application Publication No. 2011/0312043, U.S. Patent Application Publication No. 2011/0312044, U.S. Patent Application Publication No. 2012/0156738, and PCT International Publication No. WO 2011/159998; the entire contents of each are herein incorporated by reference. Oil may be separated from DDGS using any suitable known process including, for example, a solvent extraction process. In one embodiment of the invention, DDGS may be added to an extraction vessel and washed with a solvent such as hexane to remove oil. Other solvents that may be utilized include, for example, isobutanol, isohexane, ethanol, butanol, isododecane, hexadecane, isohexadecane, heptane, triisobutylene, diisobutylene, tetraisobutylene, isooctane, petroleum distillates such as petroleum ether, or mixtures thereof. After oil extraction, DDGS may be treated to remove any residual solvent. For example, DDGS may be heated to vaporize any residual solvent using any method known in the art. Following solvent removal, DDGS may be subjected to a drying process to remove any residual water. The processed DDGS may be used as a feed supplement for animals such as dairy and beef cattle, poultry, swine, livestock, equine, aquaculture, and domestic pets.

After extraction from DDGS, the resulting oil and solvent mixture may be collected for separation of oil from the solvent. In one embodiment, the oil/solvent mixture may be processed by evaporation whereby the solvent is evaporated and may be collected and recycled. The recovered oil may be converted to an ISPR extractant for subsequent use in the same or different fermentation processes.

In some embodiments, oil may also be separated from the feedstock or feedstock slurry using a solvent extraction process. For example, the feedstock or feedstock slurry may be added to an extraction vessel and washed with a solvent to remove oil. Solvents that may be utilized include, for example, isobutanol, isohexane, ethanol, butanol, isododecane, hexadecane, isohexadecane, heptane, triisobutylene, diisobutylene, tetraisobutylene, isooctane, petroleum distillates such as petroleum ether, or mixtures thereof. After oil extraction, the feedstock or feedstock slurry may be separated generating a wet cake and an aqueous solution. By removing oil from the feedstock or feedstock slurry, separation of solids and aqueous solution may be improved. For example, the amount of solids in the aqueous solution may be decreased.

In an embodiment of the process shown in FIG. 4A, wash centrate 75 or a portion thereof (shown as dotted lines) may be recycled or combined with other streams as illustrated in FIG. 4B. For example, wash centrate 75 may be combined with other streams as a means to reduce the amount of total suspended solids (TSS) in mash. As shown in FIG. 4B, wash centrate 75 or a portion thereof may be combined with feedstock 12 in slurry mixer 15 producing feedstock stream 17 comprising a fermentable carbon source and undissolved solids including TSS. Feedstock stream 17 may be conducted to liquefaction 10 configured to liquefy a feedstock stream, producing feedstock slurry 16 which may be further processed as described herein. In another embodiment, wash centrate 75 or a portion thereof may be combined with feedstock slurry 16. By diluting feedstock slurry 16 with wash centrate 75, the amount of TSS in feedstock slurry 16 may be further reduced. In some embodiments, the amount of TSS in feedstock slurry 16 may be reduced by about 1 wt %, about 2 wt %, about 3 wt %, about 4 wt %, about 5 wt %, about 6 wt %, about 7 wt %, about 8 wt %, about 9 wt %, about 10 wt %, or more as compared to the amount of TSS (wt %) in mash (e.g., feedstock stream 17).

As described herein, wet cake may be subjected to one or more wash cycles or wash systems. For example, wet cake 74 may be further processed by conducting wet cake 74 to a second wash system. In some embodiments, wet cake 74 may be conducted to a second mix 60′ forming wet cake mixture 65′. Wet cake mixture 65′ may be conducted to a second separation 70′ producing wash centrate 75′ and wet cake 74′. Wash centrate 75′ or a portion thereof may be recycled to slurry mixer 15 and/or wash centrate 75′ or a portion thereof may be combined with wash centrate 75 and the combined wash centrates may be recycled to slurry mixer 15. In some embodiments, wash centrate 75′ or a portion thereof may be combined with feedstock slurry 16 and/or wash centrate 75′ or a portion thereof may be combined with wash centrate 75 and the combined wash centrates may in turn, be combined with feedstock slurry 16.

Removal of the oil component of the feedstock is advantageous to production because oil present in the fermentor may be hydrolyzed to fatty acids and glycerin. Glycerin can accumulate in water and reduce the amount of water that is available for recycling throughout the fermentation system. Thus, removal of the oil component of feedstock increases the efficiency of production by increasing the amount of water that can be recycled through the system. In addition, removing oil can result in energy savings for the production plant due to more efficient fermentation, less fouling due to the removal of the oil, increased fermentor volume efficiency, and decreased energy requirements, for example, the energy needed to dry distillers grains.

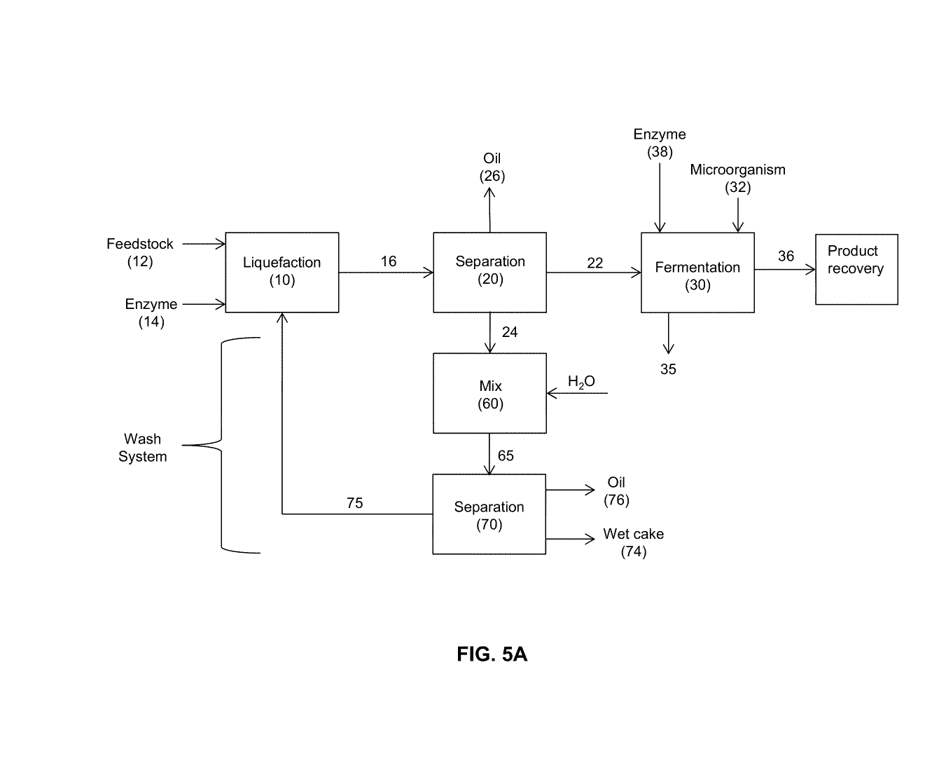

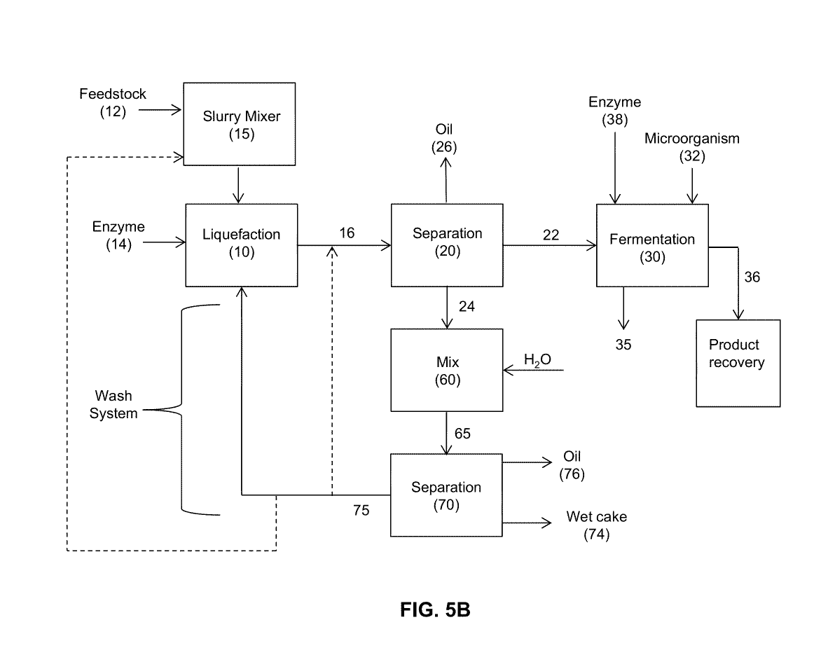

As illustrated in FIG. 5A, oil may be removed at various points during the processes described herein. Feedstock slurry 16 may be separated, for example, using a three-phase centrifuge, into a first liquid phase or aqueous solution 22 (or aqueous stream), a second liquid phase comprising oil 26 (or oil stream), and a solid phase or wet cake 24 (or solid stream). Wet cake 24 may be further processed to recover fermentable sugars and oil. Wet cake 24 may be conducted to mix 60 and combined with water or other solvents forming wet cake mixture 65. In some embodiments, water may be backset, cook water, process water, lutter water, water collected from evaporation, or any water source available in the fermentation processing facility, or any combination thereof. Wet cake mixture 65 may be conducted to separation 70 (e.g., three-phase centrifuge) producing wash centrate 75 comprising fermentable sugars, oil 76, and wet cake 74. In some embodiments, separation may be a single step process. Wash centrate 75 may be recycled to liquefaction 10. Oil 76 and oil 26 may be combined and further processed for the manufacture of various consumer products. In some embodiments, the oil (e.g., oil 26, oil 76) may be further processed to generate extractant. For example, the oil may be treated chemically or enzymatically to generate extractant. In some embodiments where the oil is corn oil, the corn oil may be treated chemically or enzymatically to generated fatty acids (e.g., corn oil fatty acids) that may be used as extractant. In some embodiments where the oil is treated enzymatically, the enzymatic reaction may be subjected to a treatment (e.g., heat) post conversion to deactivate the enzyme. In some embodiments, the oil may be enzymatically treated utilizing enzymes such as esterases, lipases, phospholipases, lysophospholipases, or combinations thereof. In some embodiments, the oil may be chemically treated with ammonium hydroxide, anhydrous ammonia, ammonium acetate, hydrogen peroxide, toluene, glacial acetic acid, or combinations thereof. Methods for deriving extractants from biomass are described in U.S. Patent Application Publication No. 2011/0312043 and U.S. Patent Application Publication No. 2011/0312044, the entire contents of each are herein incorporated by reference. In some embodiments where the oil is corn oil, the feedstock slurry may comprise at least about 1 wt %, at least about 2 wt %, at least about 3 wt %, at least about 4 wt % corn oil, or at least about 5 wt % corn oil. The remainder of FIG. 5A is similar to FIG. 1, and therefore will not be described in detail again.

As described herein, wet cake may be subjected to one or more wash cycles or wash systems. In some embodiments, wet cake 74 may be conducted to a second mix 60′ forming wet cake mixture 65′. Wet cake mixture 65′ may be conducted to a second separation 70′ producing wash centrate 75′, oil 76′, and wet cake 74′. Wash centrate 75′ may be recycled to liquefaction 10 and/or wash centrate 75′ may be combined with wash centrate 75 and the combined wash centrates may be recycled to liquefaction 10. Wet cake 74′ may be combined with wet cake 74 for further processing as described herein. Oil 76, oil 76′, and oil 26 may be combined and further processed for the manufacture of various consumer products. In some embodiments, oil (e.g., oil 26, oil 76, oil 76′) may be further processed to generate extractant as described herein. Methods for deriving extractants from biomass are described in U.S. Patent Application Publication No. 2011/0312043 and U.S. Patent Application Publication No. 2011/0312044, the entire contents of each are herein incorporated by reference.

In an embodiment of the process shown in FIG. 5A, wash centrate 75 or a portion thereof (shown as dotted lines) may be recycled or combined with other streams as illustrated in FIG. 5B. For example, wash centrate 75 may be combined with other streams as a means to reduce the amount of total suspended solids (TSS) in mash. As shown in FIG. 5B, wash centrate 75 or a portion thereof may be combined with feedstock 12 in slurry mixer 15 producing feedstock stream 17 comprising a fermentable carbon source and undissolved solids including TSS. Feedstock stream 17 may be conducted to liquefaction 10 configured to liquefy a feedstock stream, producing feedstock slurry 16 which may be further processed as described herein. In another embodiment, wash centrate 75 or a portion thereof may be combined with feedstock slurry 16. By diluting feedstock slurry 16 with wash centrate 75, the amount of TSS in feedstock slurry 16 may be further reduced. In some embodiments, the amount of TSS in feedstock slurry 16 may be reduced by about 1 wt %, about 2 wt %, about 3 wt %, about 4 wt %, about 5 wt %, about 6 wt %, about 7 wt %, about 8 wt %, about 9 wt %, about 10 wt %, or more as compared to the amount of TSS (wt %) in mash (e.g., feedstock stream 17).

As described herein, wet cake may be subjected to one or more wash cycles or wash systems. For example, wet cake 74 may be further processed by conducting wet cake 74 to a second wash system. In some embodiments, wet cake 74 may be conducted to a second mix 60′ forming wet cake mixture 65′. Wet cake mixture 65′ may be conducted to a second separation 70′ producing wash centrate 75′ and wet cake 74′. Wash centrate 75′ or a portion thereof may be recycled to slurry mixer 15 and/or wash centrate 75′ or a portion thereof may be combined with wash centrate 75 and the combined wash centrates may be recycled to slurry mixer 15. In some embodiments, wash centrate 75′ or a portion thereof may be combined with feedstock slurry 16 and/or wash centrate 75′ or a portion thereof may be combined with wash centrate 75 and the combined wash centrates may in turn, be combined with feedstock slurry 16.

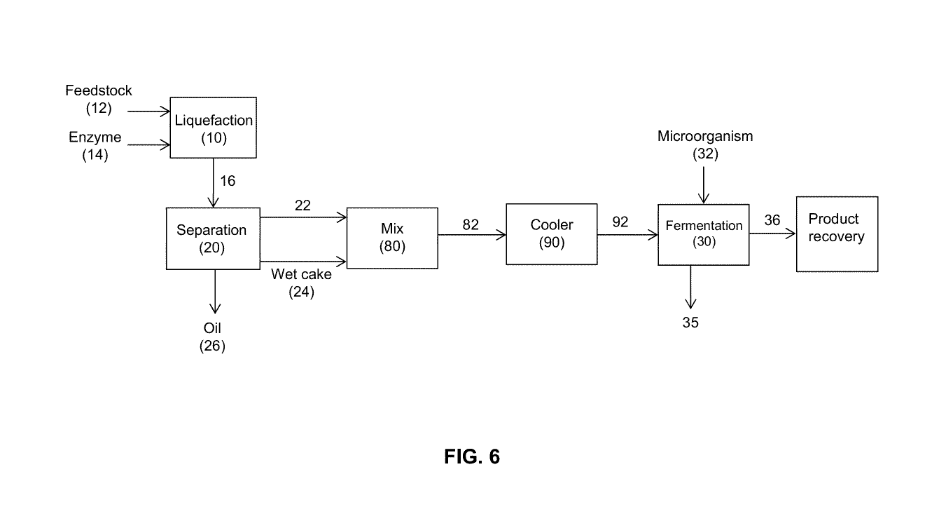

As illustrated in FIG. 6, aqueous solution 22 and wet cake 24 may be combined, cooled, and conducted to fermentation 30. Feedstock slurry 16 may be separated, for example, using a three-phase centrifuge, into a first liquid phase or aqueous solution 22, a second liquid phase comprising oil 26, and a solid phase or wet cake 24. In some embodiments, oil 26 (or oil stream) may be conducted to a storage tank or any vessel that is suitable for oil storage. Aqueous solution 22 (or aqueous stream) and wet cake 24 (solid stream) may be conducted to mix 80 and re-slurried forming aqueous solution/wet cake mixture 82. Mixture 82 may be conducted to cooler 90 producing cooled mixture 92 which may be conducted to fermentation 30. In some embodiments, when oil 26 is removed via separation 20 from feedstock 12, mixtures 82 and 92 include a reduced amount of corn oil. The remainder of FIG. 6 is similar to FIG. 1, and therefore will not be described in detail again.

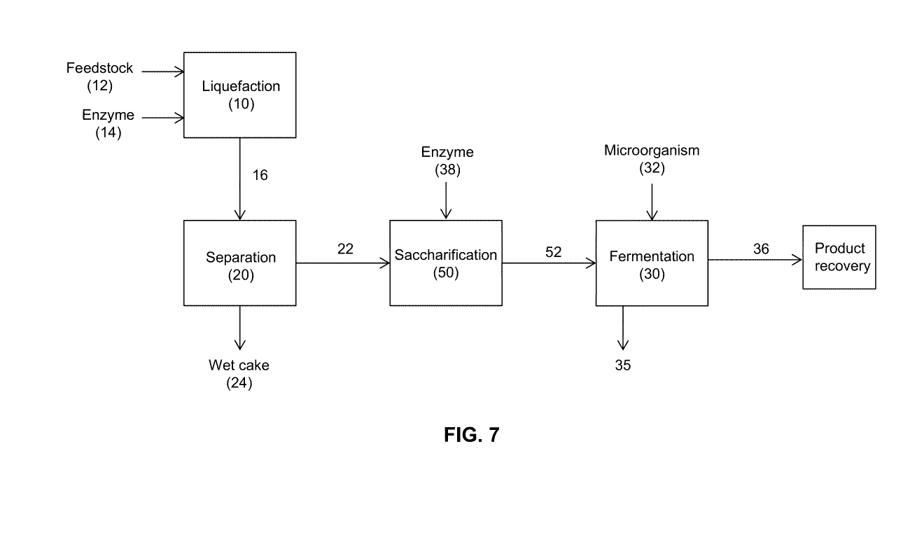

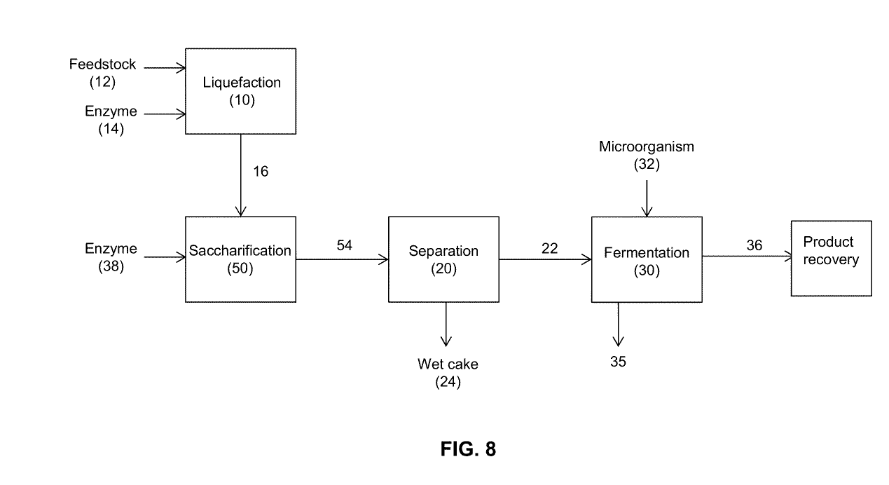

In some embodiments, as shown, for example, in FIGS. 7 and 8, saccharification may occur in a separate saccharification system 50 which is located between separation 20 and fermentation 30 (FIG. 7) or between liquefaction 10 and separation 20 (FIG. 8). FIGS. 7 and 8 are similar to FIG. 1 except for the inclusion of a separate saccharification 50 and fermentation 30 does not receive enzyme 38. In some embodiments, enzyme 38 may also be added to fermentation 30.

Any known saccharification processes utilized by the industry may be used including, but not limited to, an acid process, an enzyme process, or an acid-enzyme process. Saccharification 50 may be conducted in any suitable saccharification vessel. In some embodiments, enzyme 38 such as glucoamylase, may be introduced to hydrolyze sugars (e.g., oligosaccharides) in feedstock slurry 16 or aqueous solution 22 to form monosaccharides. For example, in FIG. 7, oligosaccharides present in aqueous solution 22 discharged from separation 20 and conducted to saccharification 50 through an inlet are hydrolyzed to monosaccharides. Aqueous solution 52 containing monosaccharides is discharged from saccharification 50 through an outlet and conducted to fermentation 30. Alternatively, as shown in FIG. 8, oligosaccharides present in feedstock slurry 16 discharged from liquefaction 10 and conducted to saccharification 50 through an inlet are hydrolyzed to monosaccharides. Feedstock slurry 54 containing monosaccharides is discharged from saccharification 50 through an outlet and conducted to separation 20. In some embodiments of the processes illustrated in FIGS. 7 and 8, feedstock slurry 16 and feedstock slurry 54 may be separated via separation 20 to form a first liquid phase or aqueous solution 22, a second liquid phase comprising oil, and a solid phase or wet cake 24. Aqueous solution 22, wet cake 24, and the oil stream may be processed as described herein.

In some embodiments, the system and processes of FIGS. 1-6 may be modified to include a separate saccharification system as described herein in connection to the systems and processes of FIGS. 7 and 8.

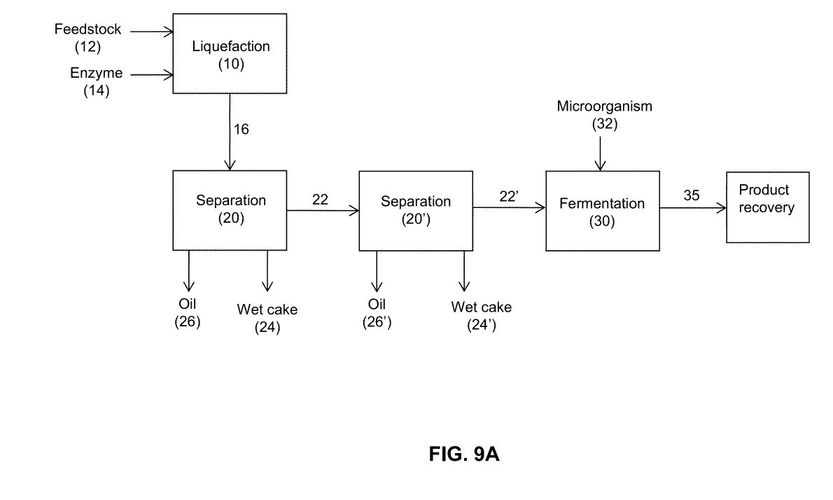

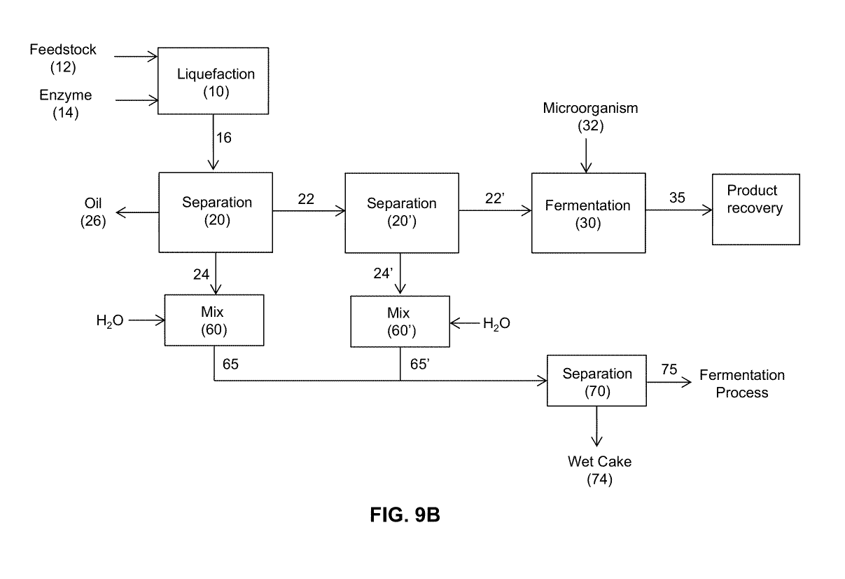

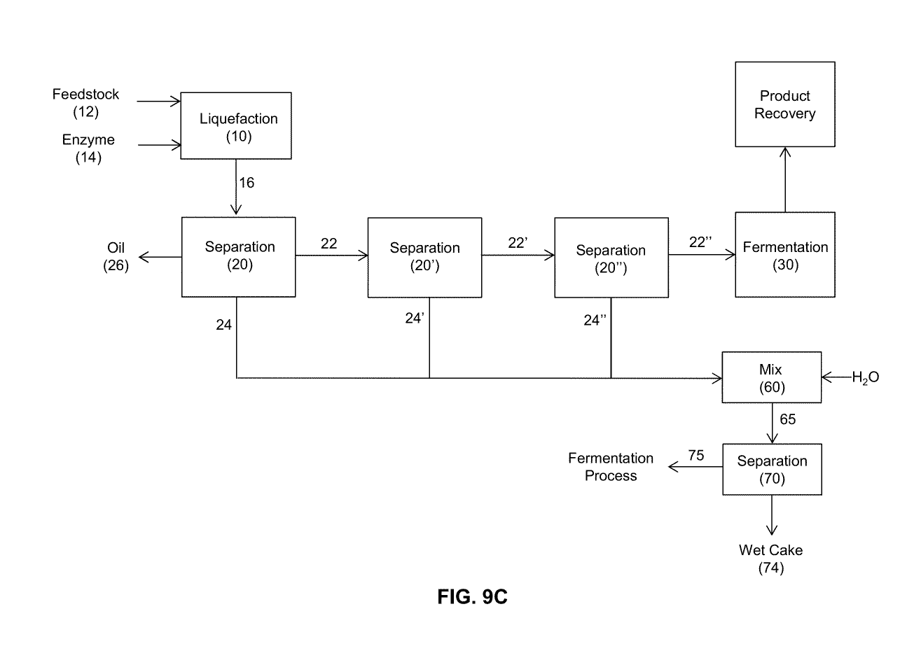

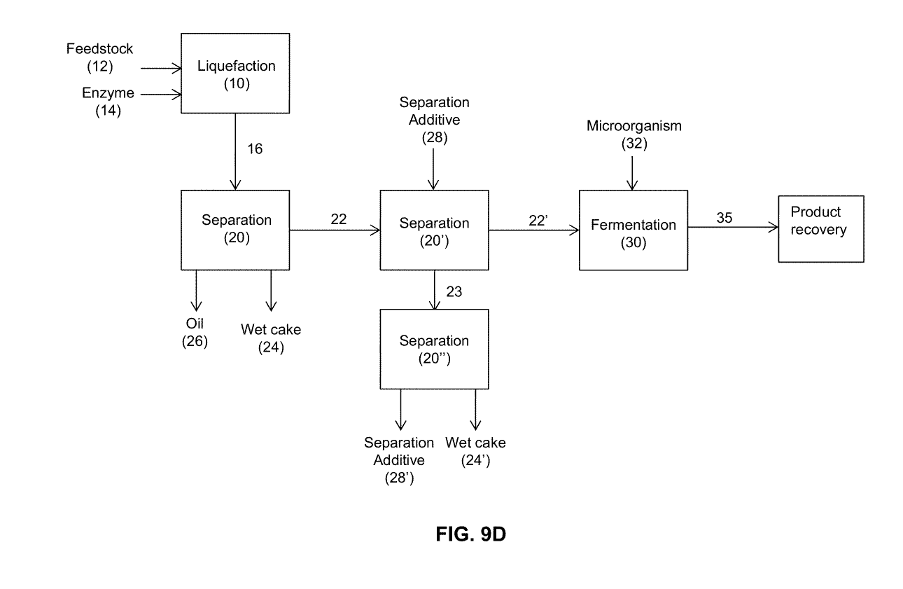

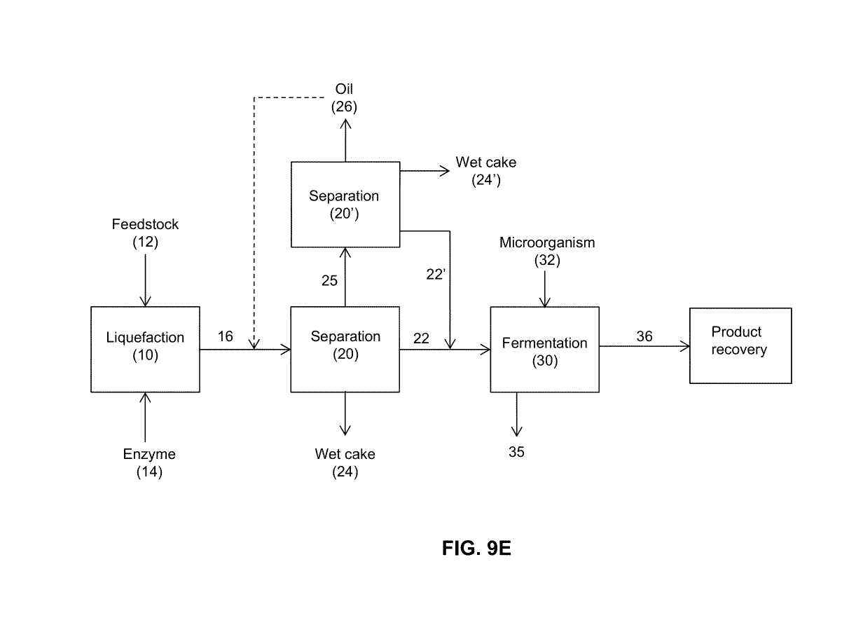

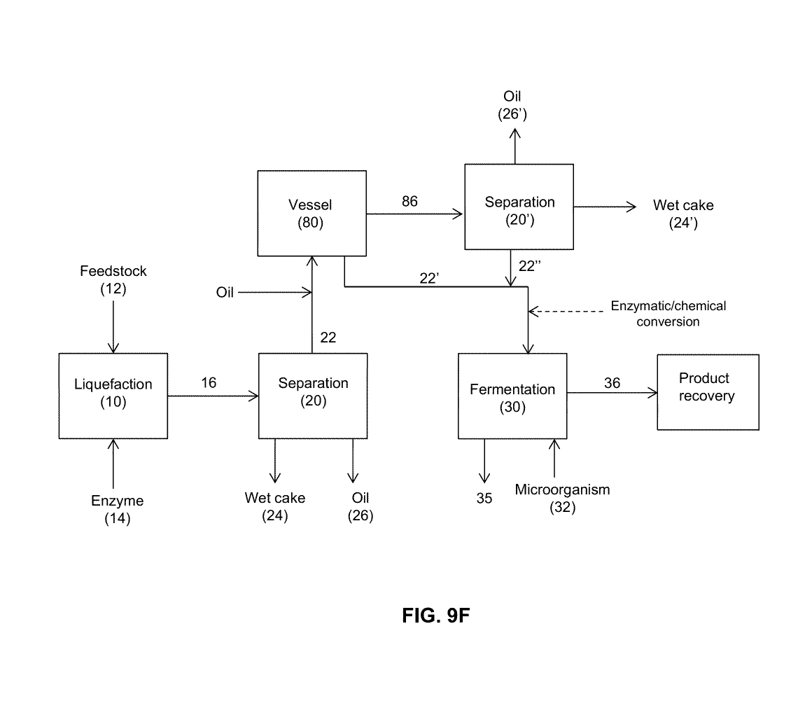

In some embodiments, as shown, for example, in FIGS. 9A-9F, the systems and processes of the present invention may include a series of two or more separation devices and/or wash systems. FIGS. 9A-9F are similar to FIG. 1, except for the addition of separation systems and/or wash systems, and therefore will not be described in detail again.

Referring to FIG. 9A, aqueous solution 22 discharged from separation 20 may be conducted to separation 20′. Separation 20′ may be identical or different to separation 20, and may operate in the same manner or different manner Separation 20′ may remove undissolved solids and oil not separated from aqueous solution 22 to generate (i) aqueous solution 22′ similar to aqueous solution 22, but containing reduced amounts of undissolved solids and oil in comparison to aqueous solution 22, (ii) wet cake 24′ similar to wet cake 24, and (iii) oil 26′ similar to oil 26. Aqueous solution 22′ may then be introduced to fermentation 30. In some embodiments, there may be one or more additional separation devices following separation 20′. In some embodiments, separation may be a single step process.

In some embodiments, separation 20 and separation 20′ may be any separation device capable of separating solids and liquids including, for example, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof.

In some embodiments, stream 35 may be discharged from an outlet in fermentation 30. The absence or minimization of the undissolved solids exiting fermentation 30 via stream 35 has several additional benefits. For example, the need for units and operations in downstream processing may be decreased or eliminated, for example, beer columns or distillation columns, thereby resulting in an increased efficiency for production. Also, some or all of the centrifuges used to process whole stillage may be eliminated as a result of less undissolved solids in the stream exiting the fermentor.

In some embodiments, the systems and processes of the present invention may include a series of two, three, four, or more separation devices and/or wash systems.

Referring to FIG. 9B, feedstock slurry 16 may be conducted to separation 20 and may be separated into a first liquid phase or aqueous solution 22 containing a fermentable sugar, a solid phase or wet cake 24 containing undissolved solids, and optionally a second liquid phase containing oil 26. Wet cake 24 may be conducted to mix 60 and combined with water or other solvents forming wet cake mixture 65. Aqueous solution 22 may be conducted to separation 20′ and may be separated into aqueous solution 22′ containing a fermentable sugar and wet cake 24′ containing undissolved solids. In some embodiments, a second liquid phase containing oil 26′ may be formed via separation 20′. Aqueous solution 22′ may be conducted to fermentation 30. Wet cake 24′ may be conducted to mix 60′ and combined with water or other solvents forming wet cake mixture 65′. In some embodiments, the water combined with wet cake 24 and wet cake 24′ may be fresh water, backset, cook water, process water, lutter water, evaporation water, or any water source available in the fermentation processing facility, or any combination thereof. Wet cake mixture 65 and wet cake mixture 65′ may be combined, and may be conducted to separation 70 producing wash centrate 75 comprising fermentable sugars, and wet cake 74. Wash centrate 75 may be recycled to the fermentation process, for example, liquefaction 10 or combined with feedstock slurry 16. In embodiments where oil 26 and/or 26′ are separated, oil may be returned to the fermentation process or may be conducted to a storage tank or any vessel that is suitable for oil storage. In some embodiments, oil may be separated via separation 20 and/or separation 20′.

Separation 20 and separation 20′ may be identical or may be different, and may operate in the same manner or different manner. In some embodiments, separation may be a single step process. In some embodiments, separation 20 and separation 20′ may be any separation device capable of separating solids and liquids including, for example, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof. In some embodiments, separation 20 and separation 20′ may be centrifugation. In some embodiments, separation 20 and separation 20′ may be three-phase centrifuges. In some embodiments, separation 20 may be a three-phase centrifuge and separation 20′ may be a disk stack centrifuge. In some embodiments, separation 20 may be a three-phase centrifuge and separation 20′ may be a decanter centrifuge. In some embodiments, separation 20 may be centrifugation and separation 20′ may be filtration. In some embodiments, separation 20 may be a three-phase centrifuge and separation 20′ may be crossflow filtration. In some embodiments, separation 20 may be a three-phase centrifuge and separation 20′ may be a drum filter. In some embodiments, separation 20 may be a three-phase centrifuge and separation 20′ may be a rotary screen. In some embodiments, separation 20 may be filtration and separation 20′ may be centrifugation. In some embodiments, separation 20 may be a rotary screen and separation 20′ may be a three-phase centrifuge.

In some embodiments, there may be three or more separation devices and/or wash systems. For example, aqueous stream 22′ may be conducted to separation 20″ and may be separated into aqueous solution 22″ containing a fermentable sugar and wet cake 24″ containing undissolved solids. In some embodiments, a second liquid phase containing oil 26″ may be formed via separation 20″. Aqueous solution 22″ may be conducted to fermentation 30. Wet cake 24″ may be conducted to mix 60″ and combined with water or other solvents forming wet cake mixture 65″. In some embodiments, the water combined with wet cake 24″ may be fresh water, backset, cook water, process water, lutter water, evaporation water, or any water source available in the fermentation processing facility, or any combination thereof. Wet cake mixture 65″ may be combined with wet cake mixture 65 and wet cake mixture 65′, and the combined wet cake mixture may be conducted to separation 70 producing wash centrate 75 comprising fermentable sugars, and wet cake 74. Wash centrate 75 may be recycled to the fermentation process, for example, liquefaction 10 or combined with feedstock slurry 16. In some embodiments, oil may be separated via separation 20, separation 20′, and/or separation 20″. In embodiments where oil 26, oil 26′ and/or 26″ are separated, oil may be returned to the fermentation process or may be conducted to a storage tank or any vessel that is suitable for oil storage.

Separation 20″ may be identical or may be different to separation 20 and separation 20′, and may operate in the same manner or different manner. In some embodiments, separation may be a single step process. In some embodiments, separation 20″ may be any separation device capable of separating solids and liquids including, for example, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof. In some embodiments, separation 20, separation 20′, and separation 20″ may be centrifugation. In some embodiments, separation 20, separation 20′, and separation 20″ may be three-phase centrifuges. In some embodiments, separation 20 may be a three-phase centrifuge, and separation 20′ and separation 20″ may be disk stack centrifuges. In some embodiments, separation 20 may be a three-phase centrifuge, and separation 20′ and separation 20″ may be decanter centrifuges. In some embodiments, separation 20, separation 20′, and separation 20″ may be a combination of centrifugation and filtration. In some embodiments, separation 20 may be a three-phase centrifuge, separation 20′ may be a rotary screen, and separation 20″ may be a disk stack centrifuge. In some embodiments, separation 20 may be a three-phase centrifuge, separation 20′ may be a disk stack centrifuge, and separation 20″ may be a rotary screen. In some embodiments, separation 20 may be a rotary screen, separation 20′ may be a three-phase centrifuge, and separation 20″ may be a disk stack centrifuge. In some embodiments, separation 20, separation 20′, and separation 20″ may be a three-phase centrifuge, crossflow filtration, and a decanter centrifuge. In some embodiments, separation 20, separation 20′, and separation 20″ may be a three-phase centrifuge, a rotary screen, and a disk stack centrifuge. In some embodiments, separation 20, separation 20′, and separation 20″ may be a three-phase centrifuge, crossflow filtration, and a decanter centrifuge.

Separation 70 may be any separation device capable of separating solids and liquids including, for example, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof.

In some embodiments, mix 60, mix 60′, and mix 60″ may comprise one or more agitator devices. In some embodiments, the agitator devices may be blade turbines. In some embodiments, the blade turbines may be pitch blade turbines. In some embodiments, the blade turbines may be flat blade turbines. In some embodiments, mix 60, mix 60′, and mix 60″ may comprise one or more pitch blade turbines and/or one or more flat blade turbines. In some embodiments, mix 60, mix 60′, and mix 60″ may comprise clean-in-place capability.

FIG. 9C illustrates another embodiment of the processes and systems described herein. Feedstock slurry 16 may be conducted to separation 20 and may be separated into a first liquid phase or aqueous solution 22 containing a fermentable sugar, a solid phase or wet cake 24 containing undissolved solids, and optionally a second liquid phase containing oil 26. Aqueous solution 22 may be conducted to separation 20′ and may be separated into aqueous solution 22′ containing a fermentable sugar and wet cake 24′ containing undissolved solids. In some embodiments, a second liquid phase containing oil 26′ may be formed via separation 20′. Aqueous solution 22′ may then be conducted to separation 20″ and may be separated into aqueous solution 22″ containing a fermentable sugar and wet cake 24″ containing undissolved solids. In some embodiments, a second liquid phase containing oil 26″ may be formed via separation 20″. Aqueous solution 22″ may be conducted to fermentation 30.

Wet cake 24, wet cake 24′, and wet cake 24″ may be combined, and the combined wet cakes may be conducted to mix 60 and combined with water or other solvents forming wet cake mixture 65. In some embodiments, the water may be fresh water, backset, cook water, process water, lutter water, evaporation water, or any water source available in the fermentation processing facility, or any combination thereof. Wet cake mixture 65 may be conducted to separation 70 producing wash centrate 75 comprising fermentable sugars, and wet cake 74. Wash centrate 75 may be recycled to the fermentation process, for example, liquefaction 10 or combined with feedstock slurry 16. In some embodiments, oil may be separated via separation 20, separation 20′, and/or separation 20″. In embodiments where oil 26, oil 26′ and/or oil 26″ are separated, oil may be returned to the fermentation process or may be conducted to a storage tank or any vessel that is suitable for oil storage.

Separation 20, separation 20′, and separation 20″ may be identical or may be different, and may operate in the same manner or different manner. In some embodiments, separation may be a single step process. In some embodiments, separation 20, separation 20′, and separation 20″ may be any separation device capable of separating solids and liquids including, for example, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof. In some embodiments, separation 20, separation 20′, and separation 20″ may be centrifugation. In some embodiments, separation 20, separation 20′, and separation 20″ may be three-phase centrifuges. In some embodiments, separation 20 may be a three-phase centrifuge, and separation 20′ and separation 20″ may be disk stack centrifuges. In some embodiments, separation 20 may be a three-phase centrifuge, and separation 20′ and separation 20″ may be decanter centrifuges. In some embodiments, separation 20, separation 20′, and separation 20″ may be a combination of centrifugation and filtration. In some embodiments, separation 20 may be a three-phase centrifuge, separation 20′ may be a rotary screen, and separation 20″ may be a disk stack centrifuge. In some embodiments, separation 20 may be a three-phase centrifuge, separation 20′ may be a disk stack centrifuge, and separation 20″ may be a rotary screen. In some embodiments, separation 20 may be a rotary screen, separation 20′ may be a three-phase centrifuge, and separation 20″ may be a disk stack centrifuge. In some embodiments, separation 20, separation 20′, and separation 20″ may be a three-phase centrifuge, crossflow filtration, and a decanter centrifuge. In some embodiments, separation 20, separation 20′, and separation 20″ may be a three-phase centrifuge, a rotary screen, and a disk stack centrifuge. In some embodiments, separation 20, separation 20′, and separation 20″ may be a three-phase centrifuge, crossflow filtration, and a decanter centrifuge.

In some embodiments, mix 60 may comprise one or more agitator devices. In some embodiments, the agitator devices may be blade turbines. In some embodiments, the blade turbines may be pitch blade turbines. In some embodiments, the blade turbines may be flat blade turbines. In some embodiments, mix 60 may comprise one or more pitch blade turbines and/or one or more flat blade turbines. In some embodiments, mix 60 may comprise clean-in-place capability.

Separation 70 may be any separation device capable of separating solids and liquids including, for example, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof. In some embodiments, separation may be a single step process.

In another embodiment of FIG. 9C, there may be two separation devices and/or wash systems. Feedstock slurry 16 may be conducted to separation 20 and may be separated into a first liquid phase or aqueous solution 22 containing a fermentable sugar, a solid phase or wet cake 24 containing undissolved solids, and optionally a second liquid phase containing oil 26. Aqueous solution 22 may be conducted to separation 20′ and may be separated into aqueous solution 22′ containing a fermentable sugar and wet cake 24′ containing undissolved solids. In some embodiments, a second liquid phase containing oil 26′ may be formed via separation 20′. Aqueous solution 22′ may then be conducted to fermentation 30. Wet cake 24 and wet cake 24′ may be combined, and the combined wet cakes may be conducted to mix 60 and combined with water or other solvents forming wet cake mixture 65. In some embodiments, the water may be fresh water, backset, cook water, process water, lutter water, evaporation water, or any water source available in the fermentation processing facility, or any combination thereof. Wet cake mixture 65 may be conducted to separation 70 producing wash centrate 75 comprising fermentable sugars, and wet cake 74. Wash centrate 75 may be recycled to the fermentation process, for example, liquefaction 10 or combined with feedstock slurry 16. In some embodiments, oil may be separated via separation 20 and separation 20′. In embodiments where oil 26 and oil 26′ are separated, oil may be returned to the fermentation process or may be conducted to a storage tank or any vessel that is suitable for oil storage. Separation 20 and separation 20′ may be identical or may be different, and may operate in the same manner or different manner. Separation 20 and separation 20′ may be any separation device capable of separating solids and liquids as described herein.

In another embodiment, referring to FIG. 9D, aqueous solution 22 discharged from separation 20 may be conducted to separation 20′. Separation 20′ may be identical or different to separation 20. Separation 20′ may operate in a manner which could include separation additive 28. Separation additive 28 may aid in the removal of oil or solids. In some embodiments, separation additive 28 may be an extractant or flocculant. Separation 20′ may remove undissolved solids and oil not separated from aqueous solution 22 to generate (i) aqueous solution 22′ similar to aqueous solution 22, but containing reduced amounts of undissolved solids and oil in comparison to aqueous solution 22, and (ii) stream 23. In some embodiments, stream 23 may be similar to a combined stream of oil 26 and wet cake 24, and may contain separation additive 28. Stream 23 may be conducted to separation 20″ and may generate a stream that contains separation additive 28′ and wet cake 24′. Separation 20″ may be identical or different to separation 20 and separation 20′. Aqueous solution 22′ may be introduced to fermentation 30.

In some embodiments, separation 20, separation 20′, and separation 20″ may be any separation device capable of separating solids and liquids including, for example, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof. In some embodiments, separation may be a single step process.

Referring to FIG. 9E, feedstock slurry 16 may be discharged from liquefaction 10 and conducted to separation 20. Feedstock slurry 16 may be separated to generate streams: (i) aqueous solution 22, (ii) wet cake 24, and (iii) stream 25 comprising oil, solids, and an aqueous stream comprising a fermentable carbon source. In some embodiments, the solids of stream 25 may be light solids. In some embodiments, light solids may be solids that are less dense than water but more dense than oil. In some embodiments, light solids may be coated in oil, resulting in solids that are less dense than water. In some embodiments, solids may have lipophilic and/or hydrophilic properties. In some embodiments, the solids of stream 25 may comprise one or more of the following: germ, fiber, starch, and gluten. In some embodiments, the solids of stream 25 may comprise fine particles. In some embodiments, the solids of stream 25 may comprise germ, gluten, and fiber. In some embodiments, aqueous solution 22 comprising a fermentable carbon source may be conducted to fermentation 30 for production of a fermentation product as described herein. In some embodiments, wet cake 24 may be further processed as described herein, for example, processed to form DDGS.

Stream 25 discharged from separation 20 may be conducted to separation 20′. Separation 20′ may be identical or different to separation 20. In some embodiments, separation 20 and separation 20′ may be any separation device capable of separating solids and liquids including, for example, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof. In some embodiments, separation may be a single step process.

Stream 25 may be separated by separation 20′ to generate streams: (i) aqueous solution 22′, (ii) wet cake 24′, and (iii) oil 26. In some embodiments, aqueous solution 22′ may be combined with aqueous solution 22, and the combined aqueous solution may be conducted to fermentation 30. In some embodiments, the amount of solids in aqueous solution 22′ is reduced compared to the amount of solids in aqueous solution 22. In some embodiments, aqueous solution 22′ may comprise oil and the oil in aqueous solution 22′ may be further processed to generate an extractant. For example, aqueous solution 22′ may be treated chemically or enzymatically to generate an extractant. In some embodiments, aqueous solution 22′ may be treated chemically or enzymatically to generate fatty acids (e.g., corn oil fatty acids) that may be used as an extractant. In some embodiments where aqueous solution 22′ is treated enzymatically, the enzymatic reaction may be subjected to a treatment (e.g., heat) post conversion to deactivate the enzyme. Converting the oil in aqueous solution 22′ which has a relatively small stream volume to fatty acids may reduce the capital cost of generating extractant via enzymatic or chemical conversion. Methods for deriving extractants from biomass are described in U.S. Patent Application Publication No. 2011/0312043 and U.S. Patent Application Publication No. 2011/0312044, the entire contents of each are herein incorporated by reference.

In some embodiments, wet cake 24′ may be combined with wet cake 24, and the combined wet cake may be further processed as described herein. In some embodiments, wet cake 24′ may comprise oil and this oil-rich wet cake may be combined with wet cake 24 to produce a wet cake with increased fat content (e.g., increased triglyceride content) which would provide a metabolizable energy source for animal feed. In some embodiments, this wet cake with increased fat content may be combined with syrup to generate a high triglyceride, high protein, low carbohydrate DDGS. In some embodiments, wet cake 24′ comprising oil may be further processed as described herein, for example, to produce DDGS.

In some embodiments, oil 26 may be conducted to a storage tank or any vessel that is suitable for oil storage. In some embodiments, oil 26 or a portion thereof may be combined with feedstock slurry 16 (dotted line, FIG. 9E). The addition of oil to the feedstock slurry may improve solids removal via stream 25 by increasing the amount of solids captured.

In some embodiments, oil 26 may be further processed to generate extractant as described herein. Converting oil 26, which has a relatively small stream volume and would have a reduced flow rate compared to feedstock slurry 16, may reduce the capital cost as well as energy requirements of generating extractant via enzymatic or chemical conversion. In some embodiments, the flow rate of oil 26 may be about 1% to about 10% of the flow rate of feedstock slurry 16. Methods for deriving extractants from biomass are described in U.S. Patent Application Publication No. 2011/0312043 and U.S. Patent Application Publication No. 2011/0312044, the entire contents of each are herein incorporated by reference.

In some embodiments, stream 25 may be generated by adjusting one or more parameters of the separation device. For example, stream 25 may be generated by adjusting the weir (or dip weir) of a centrifuge such as a decanter centrifuge or three-phase centrifuge.

As described herein, solids may interfere with liquid-liquid extraction and therefore, utilizing an extraction method may not be technically or economically viable. During the extraction process, a rag layer may form at the interface of the aqueous and organic phases, and the rag layer, composed of solids (e.g., light solids), can accumulate and possibly interfere with phase separation. To mitigate the formation of the rag layer, removal of solids via stream 25 prior to fermentation may reduce or eliminate the formation of the rag layer and thereby improve downstream processing of the fermentation broth and recovery of fermentation products.

In another embodiment to mitigate the formation of the rag layer, oil may be added to the aqueous solution as a means to selectively capture solids that form the rag layer. Referring to FIG. 9F, feedstock slurry 16 may be discharged from liquefaction 10 and conducted to separation 20. Feedstock slurry 16 may be separated to generate streams: (i) aqueous solution 22, (ii) wet cake 24, and (iii) oil 26. In some embodiments, wet cake 24 may be further processed as described herein, for example, processed to form DDGS. In some embodiments, oil may be added to aqueous solution 22 and the resulting mixture may be conducted to vessel 80 where the mixture settles or separates forming (i) oil layer comprising solids 86 and (ii) aqueous solution 22′. In some embodiments, aqueous solution 22′ may be conducted to fermentation 30 for production of a fermentation product as described herein. In some embodiments, the amount of solids in aqueous solution 22′ is reduced compared to the amount of solids in aqueous solution 22. In some embodiments, solids-rich oil layer 86 may be conducted to separation 20′ and may be separated to generate streams: (i) aqueous solution 22″, (ii) wet cake 24′, and (iii) oil 26′ (a solids-lean oil). In some embodiments, solids-rich oil layer 86 may be removed (e.g., skimmed from the mixture) and filtered to remove the solids from the oil layer. In some embodiments, the oil added to aqueous solution 22 may be the oil 26 separated from feedstock slurry 16, oil 26′, an external oil source, or combinations thereof.

In some embodiments, wet cake 24′ may be combined with wet cake 24, and the combined wet cake may be further processed as described herein. In some embodiments, wet cake 24′ may comprise oil and this oil-rich wet cake may be combined with wet cake 24 to produce a wet cake with increased fat content and may be further processed as described herein. In some embodiments, wet cake 24′ comprising oil may be further processed as described herein.

In some embodiments, aqueous solution 22″ may be combined with aqueous solution 22′, and the combined aqueous solution may be conducted to fermentation 30. The amount of solids in this combined aqueous solution would be reduced compared to aqueous solution 22, and with reduced solids, the formation of the rag layer would be mitigated. In some embodiments, aqueous solution 22′ and aqueous solution 22″ may comprise oil and the oil may be further processed to generate an extractant. For example, aqueous solution 22′ and aqueous solution 22″ may be combined and may be treated chemically or enzymatically (dotted line in FIG. 9F) to generate an extractant as described herein. Methods for deriving extractants from biomass are described in U.S. Patent Application Publication No. 2011/0312043 and U.S. Patent Application Publication No. 2011/0312044, the entire contents of each are herein incorporated by reference.

In some embodiments, separation 20 and separation 20′ may be any separation device capable of separating solids and liquids including, for example, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof. In some embodiments, separation may be a single step process. In some embodiments, vessel 80 may be a static mixer, mixer-settler, decanter, gravity settler, or combinations thereof. In some embodiments, the process may be maintained at temperatures to minimize contamination (e.g., 70-110° C.).

In some embodiments, the systems and processes described herein may further comprise a liquefaction conditioning tank. In some embodiments, following liquefaction, the feedstock slurry (or liquefied mash) or a portion thereof may be conducted to a liquefaction conditioning tank for further conditioning. For example, the pH of the feedstock slurry may be adjusted. In some embodiments, the pH of the feedstock slurry may be adjusted by the addition of an acid such as sulfuric acid. In some embodiments, enzymes may be added to the feedstock slurry. In some embodiments, the enzymes may be amylases and/or glucoamylases. Examples of amylases that may be used in the processes and systems of the present invention are described in U.S. Pat. No. 7,541,026; U.S. Patent Application Publication No. 2009/0209026; U.S. Patent Application Publication No. 2009/0238923; U.S. Patent Application Publication No. 2009/0252828; U.S. Patent Application Publication No. 2009/0314286; U.S. Patent Application Publication No. 2010/02278970; U.S. Patent Application Publication No. 2010/0048446; and U.S. Patent Application Publication No. 2010/0021587, the entire contents of each are herein incorporated by reference. Examples of glucoamylases that may be used in the processes and systems of the present invention are described in U.S. Pat. No. 7,413,887; U.S. Pat. No. 7,723,079; U.S. Patent Application Publication No. 2009/0275080; U.S. Patent Application Publication No. 2010/0267114; U.S. Patent Application Publication No. 2011/0014681; U.S. Patent Application Publication No. 2011/0020899, the entire contents of each are herein incorporated by reference. In some embodiments, the feedstock slurry may be heated or cooled.

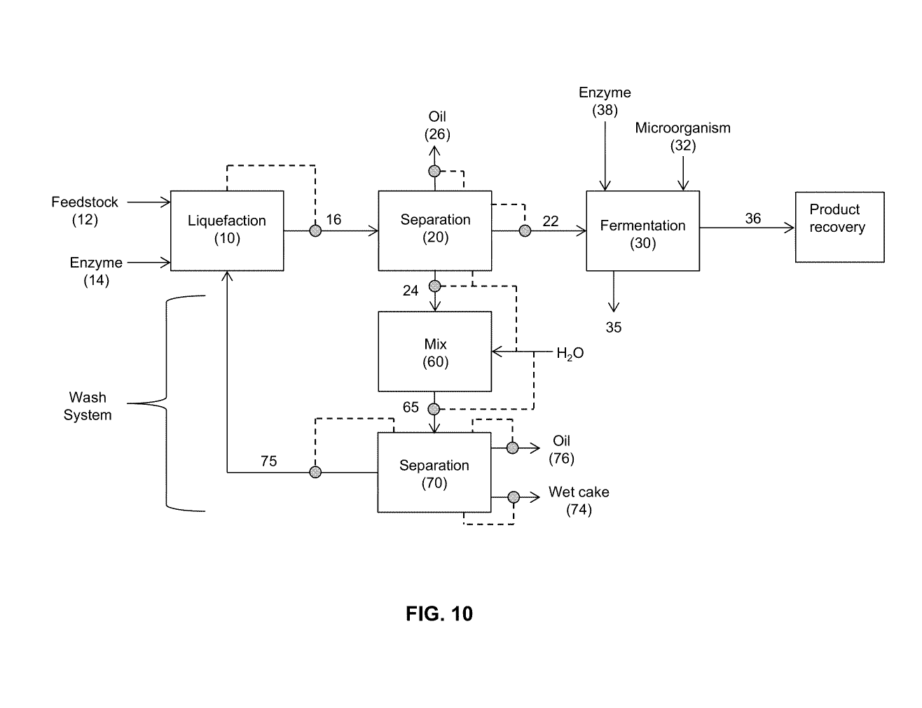

In some embodiments, as shown, for example, in FIG. 10, the systems and processes of the present invention may include means for on-line, in-line, at-line, and/or real-time measurements (circles represent measurement devices and dotted lines represent feedback loops). FIG. 10 is similar to FIG. 5A, except for the addition of measurement devices for on-line, in-line, at-line, and/or real-time measurements, and therefore will not be described in detail again.

The processes described herein may be integrated fermentation processes using on-line, in-line, at-line, and/or real-time measurements, for example, of concentrations and other physical properties of the various streams generated during fermentation (e.g., feedstock slurry, aqueous solution, oil stream, wet cake, wet cake mixtures, wash centrate, etc.). These measurements may be used, for example, in feed-back loops to adjust and control the conditions of the fermentation and/or the conditions of the fermentors, liquefaction units, saccharification units, separation units, and mixing units. In some embodiments, the concentration of fermentation products and/or other metabolites and substrates in the fermentation broth may be measured using any suitable measurement device for on-line, in-line, at-line, and/or real-time measurements. In some embodiments, the measurement device may be one or more of the following: Fourier transform infrared spectroscope (FTIR), near-infrared spectroscope (NIR), Raman spectroscope, high pressure liquid chromatography (HPLC), viscometer, densitometer, tensiometer, droplet size analyzer, particle analyzers, pH meter, dissolved oxygen (DO) probe, and the like. In some embodiments, off-gas venting from the fermentor may be analyzed, for example, by an in-line mass spectrometer. Measuring off-gas venting from the fermentor may be used as a means to identify species or reaction products present in the fermentation reaction. The concentration of fermentation products and other metabolites and substrates may also be measured using the techniques and devices described herein.

In some embodiments, measured inputs may be sent to a controller and/or control system, and conditions within the fermentor (temperature, pH, nutrients, enzyme and/or substrate concentration), liquefaction units, saccharification units, separation units, and mixing units may be varied to maintain a concentration or concentration profile of the various streams. By utilizing such a control system, process parameters may be maintained in such a way to improve overall plant productivity and economic goals. In some embodiments, real-time control of fermentation may be achieved by variation of concentrations of components (e.g., biomass, sugars, enzymes, nutrients, microorganisms, and the like) in the fermentors, liquefaction units, saccharification units, separation units, and mixing units. In some embodiments, automated systems may be used to adjust separation and mixing conditions, flow rates to and from the separation and mixing operations, solids and oil removal, and sugar and starch recovery.

During the fermentation processes, it is possible for units of operation to perform at sub-optimal levels over time. It may be necessary to adjust flow rates, mixing rates, equipment settings, and the like for operations such as liquefaction, saccharification, and separation in order to maintain overall plant productivity. The processes and systems described herein may be integrated using on-line, in-line, at-line, and/or real-time measurements for monitoring the concentrations and other physical properties of fermentation streams such as feedstock slurry 16, aqueous solution 22, oil 26, and wet cake 24. These measurements may be used, for example, in feed-back loops to adjust and control the conditions of fermentation, separation of feedstock slurry, and wash cycle performance. By utilizing on-line, in-line, at-line, and/or real-time measurements, immediate feedback and adjustments of process conditions may be made, resulting in an overall improved fermentation process. For example, the amount of fermentable carbon source (e.g., starch, sugars), oil, and solids may be monitored in feedstock slurry 16, aqueous solution 22, and streams 65 and 75 using, for example, FTIR or NIR. By monitoring these parameters, enzyme concentrations and residence time for liquefaction and saccharification may be adjusted to improve preparation of feedstock slurry 16 and separation and mixing conditions may be adjusted to, for example, increase the amount of fermentable carbon source, oil, and/or solids in aqueous solution 22 and streams 65 and 75.

As another example, washing wet cake 24 allows for recovery of sugars, starch, and oil in the wet cake, minimizing the yield loss of these fermentable carbon sources and oil. By monitoring moisture, sugar, starch, and oil content of streams 24, 65, 74, and 75, the washing performance may be adjusted to improve sugar, starch, and oil recovery. For example, real-time measurement of sugar, starch, and oil content of these streams may be performed by FTIR and NIR, and these measurements allow for immediate feedback and adjustment of mixing (60) and separation (20, 70) conditions. Differential speed, feed rate, bowl speed, scroll differential speed, impeller position, weir position, scroll pitch, residence time, and discharge volume of a separation device may be adjusted to modify the moisture, sugar, starch, and oil content of streams 24, 65, 74, and 75. Mixing (60) conditions such as pump rate or agitator speed may also be adjusted to modify the moisture, sugar, starch, and oil content of streams 24, 65, 74, and 75. In addition, the wash ratio (e.g., ratio between water and wet cake) may also be adjusted to modify the moisture, sugar, starch, and oil content of streams 24, 65, and 74. FTIR measurements and droplet imaging may be used to monitor the water content in oil streams (26, 76). In addition, color and turbidity of oil streams (26, 76) may be monitored to assess the quality of the oil. This real-time measurement would allow for adjustment to separation (20, 70) conditions resulting in a cleaner oil stream (e.g., less water).

In another embodiment of the processes and systems described herein, moisture content of wet cake 24 may be monitored using real-time measurements. Real-time measurement of moisture content of the wet cake may be performed by NIR, and these measurements allow for immediate feedback and adjustment of separation (20, 70) conditions. By decreasing the water content of the wet cake, less energy is needed to dry the wet cake and therefore, overall energy usage may be improved for the production process. In addition, a lower water content of the wet cake may result in improved starch recovery.

As another example of process control strategy using real-time measurements, solids in aqueous solution 22 and oil 26, 76 may be measured using particle size analysis such as a process particle analyzer (JM Canty, Inc., Buffalo, N.Y.), focused beam reflectance measurement (FBRM®), or particle vision and measurement (PVM®) technologies (Mettler-Toledo, LLC, Columbus Ohio). By monitoring solids in real time, process steps may be adjusted to improve solids removal and thereby, minimize the amount of solids in the aqueous solution and oil streams, maximize the recovery of solids, and improve the overall fermentation process including downstream processing.

The processes and systems disclosed in FIGS. 1-10 include removing undissolved solids and/or oil from feedstock slurry 16 and as a result, improving the processing productivity and cost effectiveness. The improved productivity can include increased efficiency of fermentation product production and/or increased extraction activity relative to processes and systems that do not remove undissolved solids and/or oil prior to fermentation.

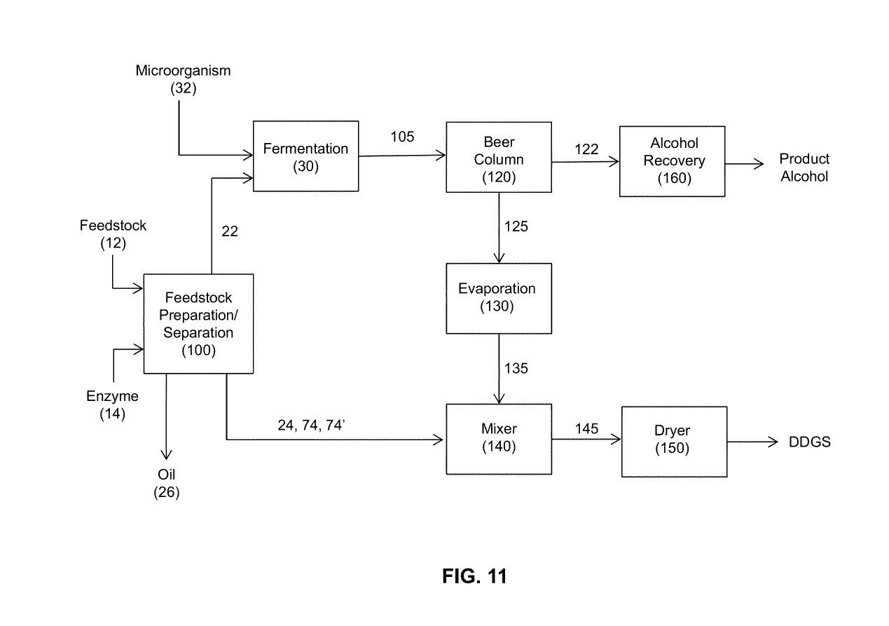

An exemplary fermentation process of the present invention including downstream processing is described in FIG. 11. Some processes and streams in FIG. 11 have been identified using the same name and numbering as used in FIGS. 1-10 and represent the same or similar processes and streams as described in FIGS. 1-10.

Feedstock 12 may be processed and undissolved solids and/or oil separated (100) as described herein with reference to FIGS. 1-10. Briefly, feedstock 12 may be liquefied to generate feedstock slurry comprising undissolved solids, fermentable carbon sources, and oil. For example, milled grain and one or more enzymes may be combined to generate a feedstock slurry. This feedstock slurry may be heated (or cooked), liquefied, and/or flashed with flash vapor producing a “cooked” feedstock slurry or mash. In some embodiments, the feedstock slurry may be heated to at least about 100° C. In some embodiments, the feedstock slurry may be heated for about thirty minutes. In some embodiments, the feedstock slurry may be subjected to raw starch hydrolysis (also known as cold cooking or cold hydrolysis). In the raw starch hydrolysis process, the heating (or cooking) step is eliminated, and the elimination of this step reduces energy consumption and steam load (e.g., water consumption). In some embodiments, liquefaction and/or saccharification may be conducted at fermentation temperatures (e.g., about 30° C. to about 55° C.). In some embodiments, liquefaction and/or saccharification may be conducted utilizing raw starch enzymes or low temperature hydrolysis enzymes such as Stargen™ (Genencor International, Palo Alto, Calif.) and BPX™ (Novozymes, Franklinton, N.C.). In some embodiments, liquefaction and/or saccharification may be conducted at temperatures less than about 50° C.

The feedstock slurry may then be subjected to separation generating wet cake 24, oil 26, and aqueous solution 22 comprising fermentable carbon source, for example, dissolved fermentable sugars. In some embodiments, separation may be a single step process. Separation may be accomplished by a number of means including, but not limited to, decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof. This separation step may remove at least about 80%, at least about 85%, at least about 90%, at least about 95%, at least about 96%, at least about 97%, at least about 98%, or at least about 99% of the undissolved solids from the feedstock slurry. In some embodiments, aqueous solution 22 may comprise at least about 0.5%, at least about 1%, or at least about 2% undissolved solids.

Wet cake 24 may be re-slurried or washed with water and subjected to separation to remove additional fermentable sugars, generating washed wet cake (e.g., 74, 74′ as described in FIGS. 1-10). In some embodiments, about 1%, about 2%, about 3%, about 4%, about 5%, about 10%, or about 15% fermentable sugars may be recovered from the washed wet cake. The wash process may be repeated a number of times, for example, one, two, three, four, five, or more times. The water used to re-slurry or wash the wet cake may be recycled water generated during the fermentation process (e.g., backset, cook water, process water, lutter water, evaporation water). In some embodiments, the wet cake may be re-slurried or washed with beer. The wash centrates (e.g., 75, 75′ as described in FIGS. 4A, 4B, 5A, 9B, and 9C) produced by the wash/separation process may be returned to the mix step to form a slurry with the milled grain or used in the liquefaction process. In some embodiments, the wash centrates may be heated or cooled prior to the mix step.

Aqueous solution 22 may be further processed as described herein. For example, aqueous solution 22 may be heated with steam or process-to-process heat exchange. A saccharification enzyme may be added to aqueous solution 22 and the dissolved fermentable sugars of aqueous solution 22 may be partially or completely saccharified. The saccharified aqueous solution 22 may be cooled by a number of means such as process-to-process exchange, exchange with cooling water, or exchange with chilled water.

Aqueous solution 22 and microorganism 32 may be added to fermentation 30 where the fermentable sugars are metabolized by microorganism 32 to produce stream 105 comprising fermentation products (e.g., product alcohol). In some embodiments, microorganism 32 may be a recombinant microorganism capable of producing product alcohol such as 1-butanol, 2-butanol, or isobutanol. In some embodiments, ammonia and recycle streams may also be added to fermentation 30. In some embodiments, the process may include at least one fermentor, at least two fermentors, at least three fermentors, at least four fermentors, at least five fermentors, or more fermentors. In some embodiments, carbon dioxide generated during fermentation may be vented to a scrubber in order to reduce air emissions (e.g., alcohol air emissions) and to increase product yield.

Stream 105 comprising product alcohol may be conducted to beer column 120 to produce alcohol-rich stream 122 and bottoms stream 125. Alcohol-rich stream 122 may be sent to alcohol recovery 160 for recovery of product alcohol. Product alcohol may be recovered from alcohol-rich stream 122 using methods known in the art including, but not limited to, distillation, adsorption (e.g., by resins), separation by molecular sieves, pervaporation, gas stripping, extraction, and the like. Bottoms stream 125 comprising thin stillage, with most of the solids removed prior to fermentation may be concentrated by evaporation via evaporation 130 to form syrup 135. Methods for evaporation are described in U.S. Patent Application Publication No. 2011/0315541, the entire contents of which are herein incorporated by reference. Syrup 135 may be combined with wet cake (24, 74, 74′as described herein) in mixer 140, and the combined stream 145 of wet cake and syrup may then be dried in a dryer 150 to produce DDGS.

In some embodiments, stream 105 may be degassed. In some embodiments, stream 105 may be heated before degassing, for example, by process-to-process exchange with hot mash. In some embodiments, vapors may be vented to a condenser and then, to a scrubber. Degassed stream 105 may be heated further, for example, by process-to-process heat exchange with other streams in the distillation and/or alcohol recovery area.

In another embodiment of FIG. 11, aqueous solution 22, microorganism 32, and extractant may be added to fermentation 30 to produce a biphasic stream. In some embodiments, extractant may be added to fermentation 30 via a recycled loop. In some embodiments, extractant may be added downstream of fermentation 30 or external to fermentation 30. A stream comprising fermentation broth and fermentation product (e.g., product alcohol) may be conducted to an external extractor to produce a stream comprising product alcohol and a bottoms stream. In some embodiments, the stream comprising product alcohol may be conducted to alcohol recovery 160 for recovery of the product alcohol. In some embodiments, the bottoms stream may be conducted to a separation device to separate the bottoms stream into thin stillage and extractant. In some embodiments, the recovered extractant may be recycled for extraction of product alcohol. The thin stillage, with most of the solids removed prior to fermentation, may be concentrated by evaporation 130 to form a syrup. The syrup may be combined with wet cake (24, 74, 74′ as described herein) in mixer 140, and the combined stream 145 of wet cake and syrup may then be dried in a dryer 150 to produce DDGS.

In some embodiments, aqueous solution 22, microorganism 32, and extractant may be added to fermentation 30 to form a single liquid phase stream. In some embodiments, the biphasic stream or single liquid phase stream may be withdrawn batchwise from fermentation 30 or may be withdraw continually from fermentation 30. In some embodiments, extractant may be added downstream of fermentation 30 or external to fermentation 30 to form a single liquid phase stream. In some embodiments, extractant may be added to an external extractor to form a single liquid phase stream.

An exemplary process for alcohol recovery is described herein, and additional methods for recovering product alcohols from fermentation broth are described in U.S. Patent Application Publication No. 2009/0305370; U.S. Patent Application Publication No. 2010/0221802; U.S. Patent Application Publication No. 2011/0097773; U.S. Patent Application Publication No. 2011/0312044; U.S. Patent Application Publication No. 2011/0312043; U.S. Patent Application Publication No. 2012/0035398; U.S. Patent Application Publication No. 2012/0156738; PCT International Publication No. WO 2011/159998; and PCT International Publication No. WO 2012/030374; the entire contents of each are herein incorporated by reference. For example, vacuum vaporization may be used to recover product alcohol from the fermentation broth. Preheated beer (e.g., aqueous stream 22) and solvent (e.g., extractant) may enter a preflash column. In some embodiments, the preflash column may be a retrofit of a beer column in a conventional dry grind fuel ethanol plant. This column may be operated at sub-atmospheric pressure, driven by water vapor taken from an evaporator train or from the mash cook step. The overheads of the preflash column may be condensed by heat exchange with some combination of cooling water and process-to-process heat exchange including heat exchange with the preflash column feed. The liquid condensate may be directed to an alcohol/water decanter.

The preflash column bottoms may be advanced to a solvent decanter. The preflash column bottoms may be substantially stripped of product alcohol. The decanter may be a still well, a centrifuge, or a hydrocyclone. Water may be separated from the solvent phase in this decanter, generating a water phase. The water phase including suspended and dissolved solids may be centrifuged to produce a wet cake and thin stillage. The wet cake may be combined with other streams and dried to produce DDGS, it may be dried and sold separate from other streams which produce DDGS, or it may be sold as a wet cake. The water phase may be split to provide a backset which is used in part to re-slurry the wet cake described herein. The split also provides thin stillage which may be conducted to evaporators for further processing.

The organic phase produced in the solvent decanter may be an ester of an alcohol. The solvent may be hydrolyzed to regenerate reactive solvent and to recover additional alcohol. Alternatively, the organic phase may be filtered and sold as a product. Hydrolysis may be thermal driven, homogeneously catalyzed, or heterogeneously catalyzed. The heat input to this process may be a fired heater, hot oil, electrical heat input, or high pressure steam. Water added to drive the hydrolysis may be from a recycled water stream, fresh water, or steam.

Cooled hydrolyzed solvent may be pumped into a sub-atmospheric solvent column where it may be substantially stripped of product alcohol with steam. This steam may be water vapor from evaporators, it may be steam from the flash step of the mash process, or it may be steam from a boiler (see, e.g., U.S. Patent Application Publication No. 2009/0171129, the entire contents of which are herein incorporated by reference). A rectifier column from a conventional dry grind ethanol plant may be suitable as a solvent column. The rectifier column may be modified to serve as a solvent column. The bottoms of the solvent column may be cooled, for example, by cooling water or process-to-process heat exchange. The cooled bottoms may be decanted to remove residual water and this water may be recycled to other steps of the process or recycled to liquefaction.

The solvent column overheads may be cooled by exchange with cooling water or by process-to-process heat exchange, and the condensate may be directed to a vented alcohol/water decanter which may be shared with the preflash column overheads. Other mixed water and product alcohol streams may be added to this decanter including the scrubber bottoms and condensate from the degas step. The vent which comprises carbon dioxide, may be directed to a water scrubber. The aqueous layer of this decanter may also be fed to the solvent column or may be stripped of product alcohol in a small dedicated distillation column. The aqueous layer may be preheated by process-to-process exchange with the preflash column overheads, solvent column overheads, or solvent column bottoms. This dedicated column may be modified from the side stripper of a conventional dry grind fuel ethanol process.

The organic layer of the alcohol/water decanter may be pumped to an alcohol column. This column may be a super-atmospheric column and may be driven by steam condensation within a reboiler. The feed to the column may be heated by process-to-process heat exchange in order to reduce the energy demand to operate the column. This process-to-process heat exchanger may include a partial condenser of the preflash column, a partial condenser of a solvent column, the product of the hydrolyzer, water vapor from the evaporators, or the alcohol column bottoms. The condensate of the alcohol column vapor may be cooled and may be returned to the alcohol/water decanter. The alcohol column bottoms may be cooled by process-to-process heat exchange including exchange with the alcohol column feed and may be further cooled with cooling water, filtered, and sold as product alcohol.

Thin stillage generated from the preflash column bottoms as described herein may be directed to a multiple effect evaporator (see e.g., U.S. Patent Application Publication No. 2011/0315541, the entire contents of which are herein incorporated by reference). This evaporator may have two, three, or more stages. The evaporator may have a configuration of four bodies by two effects similar to the conventional design of a fuel ethanol plant, it may have three bodies by three effects, or it may have other configurations. Thin stillage may enter at any of the effects. At least one of the first effect bodies may be heated with vapor from the super-atmospheric alcohol column. The vapor may be taken from the lowest pressure effect to provide heat in the form of water vapor to the sub-atmospheric preflash column and solvent column. Syrup from the evaporators may be added to the distillers grain dryer.

Carbon dioxide emissions from the fermentor, degasser, alcohol/water decanter, and other sources may be directed to a water scrubber. The water supplied to the top of this scrubber may be fresh water or may be recycled water. The recycled water may be treated (e.g., biologically digested) to remove volatile organic compounds and may be chilled. Scrubber bottoms may be sent to the alcohol/water decanter, to the solvent column, or may be used with other recycled water to re-slurry the wet cake described herein. Condensate from the evaporators may be treated with anaerobic biological digestion or other processes to purify the water before recycling to re-slurry the wet cakes.

Oil may be separated from the process streams at any of several points. For example, a centrifuge may be operated to produce an oil stream following filtration of cooked mash or the preflash column water phase centrifuge may be operated to produce an oil stream. Intermediate concentration syrup or final syrup may be centrifuged to produce an oil stream.

In another embodiment, the multi-phase material may leave the bottom of the preflash column and may be processed in a separation system as described herein. The concentrated solids may be redispersed in the aqueous stream and this combined stream may be used to re-pulp and pump the low starch solids that were separated and washed from liquefied mash.

In another exemplary process for product alcohol recovery, an extractant may be utilized to remove the product alcohol from the fermentation broth during fermentation to maintain the product alcohol in the fermentation broth below a certain concentration. In some embodiments, product alcohol removal may be achieved by esterification with carboxylic acid in the presence of a catalyst to produce alcohol esters. A description of processes and systems for extracting alcohol by formation of alcohol esters may be found in U.S. Patent Application Publication No. 2012/0156738, the entire contents of which are herein incorporated by reference. For example, oil separated from the feedstock slurry may be hydrolyzed by a catalyst such as an esterase (e.g., lipase) converting the triglycerides in the oil to fatty acids such as carboxylic acids. These fatty acids may be used an extractant for the recovery of the product alcohol. In some embodiments, the hydrolysis of the oil may occur in the fermentor by the addition of a catalyst to the fermentor. In some embodiments, the hydrolysis of the oil may occur in a separate vessel, and the fatty acids may be added to the fermentor. For example, the feedstock slurry may be conducted to a vessel or tank, and an esterase such as lipase may be added to the vessel, converting the oil present in the feedstock slurry to fatty acids. The feedstock slurry comprising fatty acids may be conducted to the fermentor.

The product alcohol produced by fermentation may react with the fatty acids to produce alcohol esters. In some embodiments, these alcohol esters may be extracted from the fermentation broth. For example, the fermentation broth comprising the alcohol esters may be transferred to a separation device such as a three-phase centrifuge to separate the fermentation broth into three streams: undissolved solids (including microorganism), aqueous stream, and organic stream comprising alcohol esters. In some embodiments, the fermentation broth may be separated into two streams: undissolved solids (including microorganism) and a biphasic mixture comprising an aqueous phase and an organic phase. This separation of the fermentation broth may occur continuously during fermentation, for example, by removing a portion of the fermentation broth for separation, or in batch mode, for example, the entire contents of the fermentor may be removed for separation.

In some embodiments, the biphasic mixture may be separated into an alcohol ester-containing organic phase and aqueous phase and this separation may be achieved using any methods known in the art including, but not limited to, siphoning, aspiration, decantation, centrifugation, gravity settler, membrane-assisted phase splitting, hydrocyclone, and the like. The alcohol ester-containing organic phase may be further processed to recover product alcohol. For example, the alcohol ester-containing organic phase may be transferred to a vessel, where the alcohol esters may be hydrolyzed in the presence of a catalyst to form product alcohol and fatty acids, and this mixture of product alcohol and fatty acids may be processed by distillation to separate the product alcohol and fatty acids.

In some embodiments, the fatty acids may be recycled to the fermentor or an extractor column. In some embodiments, the aqueous stream and undissolved solids may be recycled to the fermentor.

In some embodiments, extraction of the product alcohol may occur downstream of the fermentor or external to the fermentor. In some embodiments, the fermentation system may include an external extraction system that includes, for example, a mixing device and a separation system. Fermentation broth may be conducted to the mixing device, and extractant may be added to the mixing device and combined with fermentation broth to produce a biphasic mixture. The biphasic mixture may be introduced to a separation system, in which separation of biphasic mixture produces an alcohol-containing organic phase and an aqueous phase. In some embodiments, the aqueous phase or a portion thereof may be returned to the fermentor. In some embodiments, the alcohol-containing organic phase may be conducted to an extractant column. The biomass processing productivity in these embodiments is substantially improved by the separation of biomass feed stream components after liquefaction but prior to fermentation. In particular, decreasing the amount of undissolved solids and/or oil provides increased efficiency of external alcohol extraction systems.

In some embodiments, oil may be separated from the feedstock or feedstock slurry and may be stored in an oil storage vessel. For example, oil may be separated from the feedstock or feedstock slurry using any suitable means for separation including three-phase centrifugation or mechanical extraction. To improve the removal of oil from the feedstock or feedstock slurry, oil extraction aids such surfactants, anti-emulsifiers, or flocculants as well as enzymes may be utilized. Examples of oil extraction aids include, but are not limited to, non-polymeric, liquid surfactants; talcum powder; microtalcum powder; enzymes such as Pectinex® Ultra SP-L, Celluclast®, and Viscozyme® L (Sigma-Aldrich, St. Louis, Mo.), and NZ 33095 (Novozymes, Franklinton, N.C.); salt (NaOH); and calcium carbonate. Another means to improve oil removal may be pH adjustments such as raising or lowering the pH. Additional benefits for oil removal include increased oil yield, improved oil quality, reduced system deposition, and reduced downtime. In addition, oil removal may also result in cleaner, higher-quality oil.

The remaining feedstock or feedstock slurry may then be further treated to remove any residual oil. For example, the feedstock or feedstock slurry after oil separation may be conducted to a vessel or tank and a catalyst such as an esterase (e.g., lipase) may be added to the vessel, converting the oil present in the feedstock or feedstock slurry to fatty acids. Removing oil from the feedstock or feedstock slurry may improve enzyme efficiencies as well as reduce the amount of enzyme needed for the processes described herein. The feedstock or feedstock slurry may then be conducted to a fermentor and microorganisms may also be added to the fermentor for the production of product alcohol. In some embodiments, the catalyst may be deactivated, for example, by heating. In some embodiments, deactivation may be conducted in a separate vessel, for example, a deactivation vessel. The deactivated feedstock or feedstock slurry may be conducted to a fermentor and microorganisms may also be added to the fermentor for production of product alcohol. Removing oil from the feedstock or feedstock slurry by converting the oil to fatty acids can result in energy savings for the production plant due to more efficient fermentation, less fouling due to the removal of the oil, and decreased energy requirements, for example, the energy needed to dry distillers grains. Following fermentation, the fermentation broth comprising product alcohol may be conducted to an external vessel, for example, an external extractor or external extraction loop for the recovery of product alcohol. Removal of oil as presented here differs from known techniques in that embodiments of the present invention separate oil from the feedstock slurry such that stable emulsions are less likely to occur by virtue of the feed stream separation process, and the need for the addition of protic solvents to break emulsions formed with recovery entrapped bio-oil after fermentation is less likely (see e.g., U.S. Pat. No. 7,601,858; U.S. Pat. No. 8,192,627). In some embodiments of the present invention, an emulsion may form, but is readily broken by mechanical processing or by other conventional means.

If extractants are used to recover product alcohol, removing oil prior to the fermentation process can reduce the amount of oil taken up by the extractant and thus extend the effectiveness of the extractant for recovering product alcohol. Oil taken up by the extractant can reduce the K_{d} as well as the selectivity of the extractant, and in turn can increase the operating costs of the production process. As the extractant may be recycled in the production process, each fermentation cycle exposes the extractant to more oil which is taken up by the extractant and over time can result in a significant decrease in the K_{d} and selectivity of the extractant. The processes and systems described herein provide a means to maintain the K_{d} and selectivity of the extractant by removing oil from the feedstock or feedstock slurry and/or converting the oil to fatty acids by the addition of a catalyst.

The processes and systems described herein can lead to increased extraction activity and/or efficiency in fermentation product (e.g., product alcohol) production as a result of the removal of the undissolved solids. For example, extractive fermentation without the presence of the undissolved solids can lead to increased mass transfer rates of the product alcohol from the fermentation broth to the extractant, better phase separation, and lower hold-up of extractant as a result of increased extractant droplet rise velocities. Also, for example, extractant droplets held up in the fermentation broth during fermentation will disengage from the fermentation broth faster and more completely, thereby resulting in less free extractant in the fermentation broth. In addition, lower hold-up of extractant can decrease the amount of extractant lost in the process. Additional benefits of solids removal include, for example, elimination of agitators in the fermentor and downstream processing equipment such as beer columns and centrifuges resulting in a reduction of capital costs and energy use; increased fermentor volume resulting in increased fermentor productivity; decreased extractant hold-up resulting in increased production efficiency, increased recovery and recycling of extractant, reduced flow rate of extractant which will lower operating costs, and the potential to use continuous fermentation or smaller fermentors. In some embodiments, the volume of the fermentor available for the fermentation may be increased by at least about 5%, at least about 10%, or more.

Examples of increased extraction efficiency include, for example, stabilization of the partition coefficient, enhanced phase separation, enhanced mass transfer coefficient, operation at a lower titer, increased process stream recyclability, increased fermentation volume efficiency, increased feedstock load feeding, increased product alcohol titer tolerance of the microorganism, water recycling, reduction in energy, increased recycling of extractant, and recycling of the microorganism. For example, because oil in the fermentation broth can be reduced by removing solids from feedstock slurry prior to fermentation, the extractant is exposed to less oil which can combine with the extractant and lower the partition coefficient of the extractant. Therefore, a reduction of oil in the fermentation broth results in a more stable partition coefficient over multiple fermentation cycles. In some embodiments, the partition coefficient may be decreased by less than about 10%, less than about 5%, or less than about 1% over ten or more fermentation cycles. As another example of increased extraction efficiency, a higher mass transfer rate (e.g., in the form of a higher mass transfer coefficient) can result in an increased efficiency of product alcohol production. In some embodiments, the mass transfer coefficient may be increased at least 2-fold, at least 3-fold, at least 4-fold, or at least 5-fold.

An increase in phase separation between fermentation broth and extractant reduces the likelihood of emulsion formation resulting in an increased efficiency of product alcohol production. For example, in the absence of an emulsion, phase separation can occur more quickly and can be more complete. In some embodiments, phase separation may occur where previously no appreciable phase separation was observed. In some embodiments, phase separation may occur in 24 hours. In some embodiments, phase separation may occur at least about two times (2×) as quickly, at least about five times (5×) as quickly, or at least about ten times (10×) as quickly as compared to phase separation where solids have not been removed or emulsions have formed.

As described herein, nutrients such as nitrogen, minerals, trace elements, and/or vitamins may be added to the feedstock slurry or the fermentor. These added nutrients as well as nutrients naturally occurring in the feedstock may be soluble in oil, and thus the presence of oil in the feedstock or feedstock slurry may reduce the concentrations of these nutrients in the fermentation broth. Removing oil from the feedstock or feedstock slurry can minimize the loss of nutrients. In addition, the presence of solids in the fermentation broth may also lead to a reduction in the concentrations of nutrients. Removing the solids and/or oil from the feedstock or feedstock slurry can minimize the loss of nutrients.

For the processes and systems described herein, the presence of oil in the fermentation process may have an effect on the partition coefficient of the extractant over the course of multiple fermentations. Removing oil from the feedstock or feedstock slurry can reduce the variability of the partition coefficient of the extractant over the course of multiple fermentations, and therefore improve the scalability of the processes and systems described herein. Scalability refers to the ability to modify (e.g., expand or condense) a process or system to accommodate, for example, manufacturing demands (e.g., operating volume) without a penalty in functionality.

In addition, removing solids from feedstock or feedstock slurry can also have an effect on scalability. For example, if an external extractor is utilized, reduced solids (e.g., reduced total suspended solids, TSS) can results in improved performance and better scalability. Reduced solids enhance the rate of mass transfer of product alcohol between the aqueous phase and organic phase (e.g., fermentation broth and extractant). Solid particles coat the surface of the extractant droplets effectively reducing the area for mass transfer. Solid particles also inhibit phase separation by increasing viscosity and the tendency for emulsification. Since the presence of solids can impede the operation of the external extractor, removing solids from the feedstock or feedstock slurry provides for improved scalability and reliability of an external extractor.

Therefore, removing solids and/or oil may improve the scalability of the unit operations of processes and systems described herein. For example, removing solids and/or oil may improve unit operations such as, but not limited to, extractor performance, distillation column performance, heat exchanger performance, and/or evaporator performance.

As an example of improved unit operations, referring to FIG. 11, stream 105 may be conducted to beer column 120 to produce alcohol-rich stream 122 and bottoms stream 125. Bottoms stream 125 comprising thin stillage, with most of the solids removed prior to fermentation may be concentrated via evaporation 130. By removing solids prior to fermentation, less solids may be sent to the evaporators which can result in a lower feed rate to the evaporators. A lower feed rate requires less energy and therefore, lower costs due to lower energy requirements.

In some embodiments, there may be a need to remove water from the oil recovered from feedstock or feedstock slurry. In some embodiments, water may be removed by a number of methods including gravity separation, coalescing separator, centrifugation (e.g. decanter), adsorption or absorption, distillation, heating, vacuum dehydration, and/or air stripping (e.g., air, nitrogen). Examples of adsorption media include, but are not limited to, activated alumina, bentonite clay, calcium chloride, calcium sulfate, cellulose, magnesium sulfate, molecular sieve, polymers, and/or silica gel. In some embodiments, the adsorption media may be continuously stirred with the oil, or the oil may flow through a packed bed with adsorption media.

From time to time, it may be necessary to clean and/or sterilize the equipment used in the production of the fermentation products such as product alcohols. Examples of equipment include, but are not limited to, fermentors, liquefaction vessels, saccharification vessels, holding tanks, storage tanks, heat exchangers, pipelines, equipment connections, nozzles, fittings, and valves. Cleaning and sterilization can reduce or eliminate microbial contamination as well as minimize the accumulation of residues (e.g., carbohydrates, sugars) on equipment. Residue build-up on equipment can provide a nutritional source for unwanted microorganisms, leading to the proliferation of these microorganisms. There are a number of methods utilized to clean and/or sterilize fermentation equipment including clean-in-place (CIP) and sterilization-in-place (SIP). CIP and SIP may be performed manually or automated systems are also available. A suitable as well as efficient CIP or SIP can maximize the profitability of the production plant by minimizing the need to shut down operations due to, for example, a microbial contamination.

In the processes and systems described herein, removal of solids and oil from feedstock or feedstock slurry, for example, prior to fermentation can improve the efficiency of CIP and SIP. The lack of solids in the fermentation equipment would allow for less rinse water, less cleaning solution, and less time to perform CIP and SIP. Thus, removal of solids may improve the efficiency and reduce the costs of CIP and SIP processes. Also, the caustic agents used for CIP (e.g., sodium hydroxide) may react with the triglycerides in oil forming soap (i.e., saponification) which can have an effect on the efficiency of CIP. Therefore, the removal of oil can reduce the formation of soap during CIP and improve the efficiency of CIP.

During the fermentative process, fouling can have an impact on the productivity and efficiency of the production process. In general, fouling refers to the deposit of extraneous materials or particles, for example, the deposit of materials on the surface of heat exchangers and distillation column reboilers. This deposit of materials on the surface of the heat exchanger can interfere with the transfer of heat and reduce the operational capability of the heat exchanger. For example, the material deposit may interfere with the flow of fluid through the exchanger resulting in an increase in flow resistance. Also, the deposit of material on the surface of heat exchangers or other equipment may require additional cleaning. These additional cleaning requirements may necessitate plant shutdowns which can result in a reduction in plant productivity. In the processes and systems described herein, removal of oil and/or solids from the feedstock or feedstock slurry, for example, prior to fermentation can minimize the deposition of materials and lower the rate of deposition of materials on the surfaces of equipment such as heat exchangers. Thus, solids and oil removal can lower the rate of fouling of the heat exchangers and minimize the effect of fouling on heat transfer and operational capability.

In another example of an embodiment of the processes and systems of the invention, the material discharged from the fermentor may be processed in a separation system that involves devices such as a centrifuge, settler, hydrocyclone, etc., and combinations thereof to effect the recovery of microorganisms in a concentrated form that may be recycled for reuse in a subsequent fermentations either directly or following re-conditioning. The ability to recycle microorganisms can increase the overall rate of fermentation product production such as product alcohol production, lower the overall titer requirement, and/or lower the aqueous titer requirement, thereby leading to healthier microorganisms and a higher production rate. This separation system may also produce an organic stream that comprises fermentation product (e.g., product alcohol) and other by-products produced from the fermentation, and an aqueous stream containing only trace levels of immiscible organics. This aqueous stream may be used either before or after it is stripped of product alcohol content to wash the solids that were separated from feedstock slurry. This has the advantage of avoiding what might otherwise be a long belt-driven conveying system to transfer these solids from the liquefaction area to the grain drying and syrup blend area. Furthermore, whole stillage produced after product alcohol has been stripped will need to be separated into thin stillage and wet cake fractions either using existing or new separation devices. The thin stillage may form in part the backset that may be combined with cook water for preparing a new batch of fermentable mash. Another advantage of this embodiment is that any residual fermentable sugars that were retained in the solids separated from feedstock slurry would in part be captured and recovered through this backset. Alternatively, microorganisms contained in the solids stream may be redispersed in the aqueous stream and this combined stream distilled of any product alcohol content remaining from fermentation. If the microorganisms are nonviable, the non-viable microorganisms may further be separated for use as a nutrient, for example, in a propagation process.

In some embodiments of the processes and systems described herein, by-products (or co-products) of the fermentation process may be further processed, for example, undissolved solids may be processed to generate DDGS. Other by-products such as fatty acid esters which may have an inhibitory effect on the microorganisms may be recovered from the fermentation broth and/or by-product streams resulting in an increase in the yield of product alcohol. Recovery of fatty acid esters or other lipids may be accomplished by using a solvent to extract fatty acid esters from the by-product streams. In some embodiments, several by-product streams may be combined and fatty acid esters may be recovered from the combined streams.

In an embodiment of solvent extraction of lipids (e.g., fatty acid esters), solids may be separated from whole stillage (“separated solids”) since this stream would contain a large portion of fatty acid esters. These separated solids may then be fed to an extractor and washed with solvent. In some embodiments, the separated solids may be washed at least two or more times. After washing, the resulting mixture of lipid and solvent, known as miscella, may be collected for separation of the extracted lipid from the solvent. For example, the resulting mixture of lipid and solvent may be conducted to a separator or extractor for further processing. During the extraction process, the solvent not only extracts lipid into solution, but it also collects fine particles (“fines”). These fines are generally undesirable impurities in the miscella and in one embodiment, the miscella may be discharged from the separator or extractor and conducted to a separation device that separates or scrubs the fines from the miscella.

In order to separate lipid and solvent contained in the miscella, the miscella may be subjected to a distillation step. In this step, the miscella can, for example, be processed through an evaporator which heats the miscella to a temperature that is high enough to cause vaporization of the solvent, but is not sufficiently high to adversely affect or vaporize the extracted lipid. As the solvent evaporates, it may be collected, for example, in a condenser, and recycled for future use. Separation of the solvent from the miscella results in a stock of crude lipid which may be further processed to separate water, fatty acid esters (e.g., fatty acid isobutyl esters), fatty acids, and triglycerides. A solvent-based extraction system for recovering triglycerides is described in U.S. Patent Application Publication No. 2010/0092603, the entire contents of which are herein incorporated by reference.

After extraction of the lipids, the solids may be conveyed from the extractor and may be conducted to a stripping device (e.g., desolventizer) to remove residual solvent. Recovery of residual solvent can be important to process economics. In some embodiments, the solids may be conveyed to a desolventizer in a vapor tight environment to preserve and collect solvent that may transiently evaporate from the solids. As the solids enter the desolventizer, the solids may be heated to vaporize and remove the residual solvent. In order to heat the solids, the desolventizer may include a mechanism for distributing the solids over one or more trays, and the solids may be heated directly such as through direct contact with heated air or steam, or indirectly such as by heating the tray carrying the solids. In order to facilitate transfer of the solids from one tray to another, the trays carrying the solids may include openings that allow the solids to pass from one tray to the next tray. From the desolventizer, the solids may optionally be conveyed to a mixer where the solids are mixed with other by-products before being conveyed to a dryer. In some embodiments, the solids are conducted to a desolventizer and the solids are contacted by steam. In some embodiments, the flows of steam and solids in the desolventizer may be countercurrent. In some embodiments, vapor exiting the desolventizer may be condensed, optionally mixed with miscella, and then fed to a decanter forming a water-rich phase. This water-rich phase exiting the decanter may be fed to a distillation column where solvent is removed from the water-rich stream. In some embodiments, a solvent-depleted water-rich stream may exit the bottom of the distillation column and may be recycled to the fermentation process, for example, it may be used to process the feedstock. In some embodiments, overhead and bottom products of the distillation column may be recycled to the fermentation process. For example, the lipid-rich bottoms may be added to the feed of a hydrolyzer. The overheads may be, for example, condensed and fed to a decanter forming a solvent-rich stream and a water-rich phase. The solvent-rich stream exiting this decanter may optionally be used as the solvent feed to an extractor, and the water-rich phase exiting this decanter may be fed to a stripping column to strip solvent from water.

In some embodiments, by-products or co-products may be derived from feedstock slurry used in the fermentation process. As described herein, if corn is used as feedstock, corn oil may be separated from the feedstock slurry prior to fermentation. The benefits of removing corn oil prior to the fermentation process are: recovering more corn oil as compared to corn oil removal at the end of the fermentation process (e.g., from the syrup), recovering higher quality and therefore higher value oil as compared to corn oil removal at end of the fermentation process, generating corn oil as a co-product, and conversion of corn oil to other products.

For example, as corn oil contains triglycerides, diglycerides, monoglycerides, fatty acids, phytosterols, vitamin E, carotenoids (e.g., β-carotene, β-cryptoxanthin, lutein zeaxanthin), phospholipids, and antioxidants such as tocopherols, it may be added to other co-products at different concentrations or rates, creating the ability to vary the amount of these components in the resulting co-product. In this manner, the fat content of the resulting co-product may be controlled, for example, to yield a lower fat, high protein animal feed that would better suit the needs of dairy cows compared to a high fat product. In another embodiment where a high fat animal feed may be desired, corn oil may be used as a component of animal feed because its high triglyceride content would provide a source of metabolizable energy. In addition, the natural antioxidants in corn oil provide a source of vitamin E as well as reduce the development of rancidity.

Corn oil separated from feedstock may be further processed to produce refined corn oil or edible oil for consumer use. For example, crude corn oil may be further processed to produce refined corn oil by degumming to remove phosphatides, alkali refining to neutralize free fatty acids, decolorizing for removal of color bodies and trace elements, winterizing to remove waxes, and deodorization (see, e.g., Corn Oil, 5^{th} Edition, Corn Refiners Association, Washington, D.C., 2006). The refined corn oil may be used, for example, by food manufacturers for the production of food products. The free fatty acids removed by alkali refining may be used as soapstock and waxes recovered from the winterizing step may be utilized in animal feeds.

Corn oil may be used in the manufacture of resins, plastics, polymers, lubricants, paints, varnishes printing inks, soap, and textiles; and may also be utilized by the pharmaceutical industry as a component of drug formulations. Corn oil may also be used as feedstock for biodiesel or renewable diesel.

In some embodiments, oils such as corn oil may be used as a feedstock for the generation of extractant for extractive fermentation. For example, oil derived from biomass may be converted into an extractant available for removal of a product alcohol such as butanol from a fermentation broth. The glycerides in the oil may be chemically or enzymatically converted into a reaction product, such as fatty acids, fatty alcohols, fatty amides, fatty acid alkyl esters, fatty acid glycol esters, and hydroxylated triglycerides, or mixtures thereof, which may be used a fermentation product extractant. Using corn oil as an example, corn oil triglycerides may be reacted with a base such as ammonia hydroxide or sodium hydroxide to obtain fatty amides, fatty acids, and glycerol. These fatty amides, fatty acids, or mixtures thereof may be used an extractant. In some embodiments, plant oil such as corn oil may be hydrolyzed by an enzyme such as lipase to form fatty acids (e.g., corn oil fatty acids). Methods for deriving extractants from biomass are described in U.S. Patent Application Publication No. 2011/0312043, U.S. Patent Application Publication No. 2011/0312044, and PCT International Publication No. WO 2011/159998. In some embodiments, extractant may be used, all or in part, as a component of an animal feed or it can be used as feedstock for biodiesel or renewable diesel.

In some embodiments, corn oil can also be used as feedstock for biodiesel or renewable diesel. In some embodiments, oils or a combination of oils can also be used as feedstock for biodiesel or renewable diesel. Examples of oils include canola, castor, corn, jojoba, karanja, mahua, linseed, soybean, palm, peanut, rapeseed, rice, safflower, and sunflower oils. Biodiesel may be derived from either the transesterification or esterification of plant oils with alcohols such as methanol, ethanol, and butanol. For example, biodiesel may be produced by acid-catalyzed, alkali-catalyzed, or enzyme-catalyzed transesterification or esterification (e.g., transesterification of plant oil-derived triglycerides or esterification of plant oil-derived free fatty acids). Inorganic acids such as sulfuric acid, hydrochloric acid, and phosphoric acid; organic acids such as toluenesulfonic acid and naphthalenesulfonic acid; solid acids such as Amberlyst® sulfonated polystyrene resins; or zeolites may be used as a catalyst for acid-catalyzed transesterification or esterification. Bases such as potassium hydroxide, potassium methoxide, sodium hydroxide, sodium methoxide, or calcium hydroxide may be used as a catalyst for alkali-catalyzed transesterification or esterification. In some embodiments, biodiesel may be produced by an integrated process, for example, acid-catalyzed esterification of free fatty acids followed by base-catalyzed transesterification of triglycerides.

Enzymes such as lipases or esterases may be used to catalyze transesterification or esterification reactions. Lipases may be derived from bacteria or fungi, for example, Pseudomonas, Thermomyces, Burkholderia, Candida, and Rhizomucor. In some embodiments, lipases may be derived Pseudomonas fluorescens, Pseudomonas cepacia, Rhizomucor miehei, Burkholderia cepacia, Thermomyces lanuginosa, or Candida antarctica. In some embodiments, the enzyme may be immobilized on a soluble or insoluble support. The immobilization of enzymes may be performed using a variety of techniques including 1) binding of the enzyme to a porous or non-porous carrier support, via covalent support, physical adsorption, electrostatic binding, or affinity binding; 2) crosslinking with bifunctional or multifunctional reagents; 3) entrapment in gel matrices, polymers, emulsions, or some form of membrane; and 4) a combination of any of these methods. In some embodiments, lipase may be immobilized, for example, on acrylic resin, silica, or beads (e.g., polymethacrylate beads). In some embodiments, the lipases may be soluble.

In some embodiments, biodiesel described herein may comprise one or more of the following fatty acid alkyl esters (FAAE): fatty acid methyl esters (FAME), fatty acid ethyl esters (FAEE), and fatty acid butyl esters (FABE). In some embodiments, biodiesel described herein may comprise one or more of the following: myristate, palmitate, stearate, oleate, linoleate, linolenate, arachidate, and behenate.

In some embodiments, oil from the fermentation process may be recovered by evaporation forming a non-aqueous stream. This non-aqueous stream may comprise fatty acid esters and fatty acids and this stream may be conducted to a hydrolyzer to recover product alcohol and fatty acids. In some embodiments, this stream may be used as feedstock for biodiesel production.

In some embodiments, the biodiesel described herein meets the specifications of the American Society for Testing and Materials (ASTM) D6751. In some embodiments, the biodiesel described herein meets the specifications of the European standard EN 14214.

In some embodiments, reactor configurations for the production of biodiesel include, for example, batch-stirred tank reactors, continuous-stirred tank reactors, packed bed reactors, fluid bed reactors, expanding bed reactors, and recirculation membrane reactors.

In some embodiments, a composition may comprise at least 2% biodiesel, at least 5% biodiesel, at least 10% biodiesel, at least 20% biodiesel, at least 30% biodiesel, at least 40% biodiesel, at least 50% biodiesel, at least 60% biodiesel, at least 70% biodiesel, at least 80% biodiesel, at least 90% biodiesel, or 100% biodiesel.

In some embodiments, the biodiesel described herein may be blended with a petroleum-based diesel fuel to form a biodiesel blend. In some embodiments, a biodiesel blend may comprise at least 2% by volume biodiesel, at least 3% by volume biodiesel, at least 4% by volume biodiesel, at least 5% by volume biodiesel, at least 6% by volume biodiesel, at least 7% by volume biodiesel, at least 8% by volume biodiesel, at least 9% by volume biodiesel, at least 10% by volume biodiesel, at least 11% by volume biodiesel, at least 12% by volume biodiesel, at least 13% by volume biodiesel, at least 14% by volume biodiesel, at least 15% by volume biodiesel, at least 16% by volume biodiesel, at least 17% by volume biodiesel, at least 18% by volume biodiesel, at least 19% by volume biodiesel, or at least 20% by volume biodiesel. In some embodiments, a biodiesel blend may comprise up to about 20% by volume biodiesel.

A by-product of biodiesel production is glycerol. In addition, glycerol may also be a by-product of the generation of extractant from oils and a by-product of the fermentation process. A feedstock for biodiesel may be produced by reacting a fatty acid such as COFA with glycerol. The reaction may be catalyzed by strong inorganic acids such as sulfuric acid or by solid acid catalysts such as Amberlyst™ polymeric catalysts and ion exchange resins. High conversions may be obtained by withdrawing water from the reaction mass. The reaction product may contain monoglycerides, diglycerides, and triglycerides in a proportion determined by the ratio of reactants and the extent of reaction. The glyceride mix may be used in lieu of the triglyceride feed normally used to make biodiesel. In some embodiments, the glycerides may be used as a surfactant or as a feedstock for biodiesel.

In some embodiments, solids may be separated from feedstock slurry and may comprise triglycerides and fatty acids. These solids may be used as an animal feed, either recovered as discharge from centrifugation or after drying. The solids may be particularly suited as feed for ruminants (e.g., dairy cows) because of its high content of available lysine and by-pass or rumen undegradable protein. For example, these solids may be of particular value in a high protein, low fat feed. In some embodiments, these solids may be used as a base, that is, other by-products such as syrup may be added to the solids to form a product that may be used as an animal feed. In some embodiments, different amounts of other by-products may be added to the solids to tailor the properties of the resulting product to meet the needs of a certain animal species (e.g., dairy and beef cattle, poultry, swine, livestock, equine, aquaculture, and domestic pets).

In some embodiments where a low fat animal feed is desired, oil may be removed from the feedstock prior to fermentation. By removing the corn oil, the DDGS produced would have a low fat, high protein content. If the corn oil is not removed, the oil present in the wet cake can be oxidized by the drying process. This oxidation causes a darkening effect and produces DDGS with a darker color. If the oil is removed from the feedstock prior to fermentation, the DDGS produced would be lighter in color and this lighter color DDGS may be desirable for some animal feed products.

The composition of solids separated from whole stillage may include, for example, crude protein, fatty acids, and fatty acid esters. In some embodiments, this composition may be used, wet or dry, as an animal feed where, for example, a high protein (e.g., high lysine), low fat, and high fiber content is desired. In some embodiments, fat may be added to this composition, for example, from another by-product stream if a higher fat, low fiber animal feed is desired. In some embodiments, this higher fat, low fiber animal feed may be used for swine or poultry. In some embodiments, a non-aqueous composition of CDS may include, for example, protein, fatty acids, and fatty acid esters as well as other dissolved and suspended solids such as salts and carbohydrates. This CDS composition may be used, for example, as animal feed, either wet or dry, where a high protein, low fat, high mineral salt feed component is desired. In some embodiments, this composition may be used as a component of a dairy cow ration.

In some embodiments, one or more streams generated by the production of a product alcohol via a fermentation process may be combined to generate a composition comprising at least about 90% fatty acids which may be used as fuel source such as biodiesel.

The various streams generated by the production of a product alcohol via a fermentation process may be combined in many ways to generate a number of co-products. For example, if crude corn from mash is used to generate fatty acids to be utilized as extractant and lipid is extracted by evaporators, then the remaining streams may be combined and processed to create a co-product composition comprising crude protein, crude fat, triglycerides, fatty acids, and fatty acid esters. In another example, if oil such as corn oil is removed from feedstock slurry, the oil may be added to distillers grains to produce, for example, an animal feed product.

In some embodiments, compositions of the processes and systems described herein may comprise at least about 20-35 wt % crude protein, at least about 1-20 wt % crude fat, at least about 0-5 wt % triglycerides, at least about 4-10 wt % fatty acids, and at least about 2-6 wt % fatty acid esters. In some embodiments, compositions may comprise about 25 wt % crude protein, about 10 wt % crude fat, about 0.5 wt % triglycerides, about 6 wt % fatty acids, and about 4 wt % fatty acid esters. In some embodiments, compositions may comprise at least about 25-31 wt % crude protein, at least about 6-10 wt % crude fat, at least about 4-8 wt % triglycerides, at least about 0-2 wt % fatty acids, and at least about 1-3 wt % fatty acid esters. In some embodiments, compositions may comprise about 28 wt % crude protein, about 8 wt % crude fat, about 6 wt % triglycerides, about 0.7 wt % fatty acids, and about 1 wt % fatty acid esters. In some embodiments, the fatty acid esters may be fatty acid methyl esters, fatty acid ethyl esters, fatty acid butyl esters, or fatty acid isobutyl ester.

In some embodiments, solids separated from whole stillage and oil extracted from feedstock slurry may be combined and the resulting composition may comprise crude protein, crude fat, triglycerides, fatty acids, fatty acid esters, lysine, neutral detergent fiber (NDF), and acid detergent fiber (ADF). In some embodiments, compositions may comprise at least about 26-34 wt % crude protein, at least about 15-25 wt % crude fat, at least about 12-20 wt % triglycerides, at least about 1-2 wt % fatty acids, at least about 2-4 wt % fatty acid esters, at least about 1-2 wt % lysine, at least about 11-23 wt % NDF, and at least about 5-11 wt % ADF. In some embodiments, compositions may comprise about 29 wt % crude protein, about 21 wt % crude fat, about 16 wt % triglycerides, about 1 wt % fatty acids, about 3 wt % fatty acid esters, about 1 wt % lysine, about 17 wt % NDF, and about 8 wt % ADF. In some embodiments, the fatty acid esters may be fatty acid methyl esters, fatty acid ethyl esters, fatty acid butyl esters, or fatty acid isobutyl ester. The high fat, triglyceride, and lysine content and the lower fiber content of this composition may be desirable as feed for swine and poultry.

As described herein, the various streams generated by the production of a product alcohol via a fermentation process may be combined in many ways to generate a composition comprising crude protein, crude fat, triglycerides, fatty acids, and fatty acid esters. For example, a composition comprising at least about 6% crude fat and at least about 28% crude protein may be utilized as an animal feed product for dairy animals. A composition comprising at least about 6% crude fat and at least about 26% crude protein may be utilized as an animal feed product for feedlot cattle whereas a composition comprising at least about 1% crude fat and at least about 27% crude protein may be utilized as an animal feed product for wintering cattle. A composition comprising at least about 13% crude fat and at least about 27% crude protein may be utilized as an animal feed product for poultry. A composition comprising at least about 18% crude fat and at least about 22% crude protein may be utilized as an animal feed product for monogastric animals. The various streams may be combined in such a way as to customize a feed product for a specific animal species (e.g., livestock, ruminant, cattle, dairy animal, swine, goat, sheep, poultry, equine, aquaculture, or domestic pet such as dogs, cats, and rabbits).

The DDGS generated by the processes of the present invention may be modified to produce a customized high value feed product by the addition of one or more of the following: protein, fat, fiber, ash, lipid, amino acids, vitamins, and minerals. Amino acids include, for example, essential amino acids such as histidine, isoleucine, leucine, lysine, methionine, phenylalanine, threonine, tryptophan, and valine as well as other amine acids such as alanine, arginine, aspartic acid, asparagine, cysteine, glutamic acid, glutamine, glycine, hydroxylysine, hydroxyproline, ornithine, proline, serine, and tyrosine. Minerals include, for example, calcium, chloride, cobalt, copper, fluoride, iodine, iron, magnesium, manganese, phosphorus, potassium, selenium, sodium, sulfur, and zinc. Vitamins include, for example, vitamins A, C, D, E, K, and B (thiamine, riboflavin, niacin, pantothenic acid, biotin, vitamin B6, vitamin B12 and folate).

As described herein, the processes and systems of the present invention provide a number of benefits that can result in improved production of a product alcohol such as butanol. For example, an improvement in mass transfer enables operation at a lower aqueous titer resulting in a “healthier” microorganism. A better phase separation can lead to improved fermentor volume efficiency as well as the possibility of processing less reactor contents through beer columns, distillation columns, etc. In addition, there is less solvent loss via solids and there is the possibility of cell recycling. The processes and systems of the present invention may also provide a higher quality of DDGS.

The processes and systems described herein also provide for the removal of oil prior to fermentation which would then allow the controlled addition of oil to the fermentation. Furthermore, the removal of oil prior to fermentation would allow flexibility in the amount of oil present in DDGS. That is, oil may be added in different amounts to DDGS resulting in the production of DDGS with different fat contents depending on the nutritional needs of a particular animal species.

The processes and systems described herein may be demonstrated using computational modeling such as Aspen modeling (see, e.g., U.S. Pat. No. 7,666,282). For example, the commercial modeling software Aspen Plus® (Aspen Technology, Inc., Burlington, Mass.) may be used in conjunction with physical property databases such as DIPPR (Design Institute for Physical Property Research), available from American Institute of Chemical Engineers, Inc. (New York, N.Y.) to develop an Aspen model for an integrated alcohol fermentation, purification, and water management process. This process modeling can perform many fundamental engineering calculations, for example, mass and energy balances, vapor/liquid equilibrium, and reaction rate computations. In order to generate an Aspen model, information input may include, for example, experimental data, water content and composition of feedstock, temperature for mash cooking and flashing, saccharification conditions (e.g., enzyme feed, starch conversion, temperature, pressure), fermentation conditions (e.g., microorganism feed, glucose conversion, temperature, pressure), degassing conditions, solvent columns, preflash columns, condensers, evaporators, centrifuges, etc.

### Recombinant Microorganisms

While not wishing to be bound by theory, it is believed that the processes described herein are useful in conjunction with any alcohol-producing microorganism, particularly recombinant microorganisms which produce alcohol at titers above their tolerance levels.

Alcohol-producing microorganisms are known in the art. For example, fermentative oxidation of methane by methanotropic bacteria (e.g., Methylosinus trichosporium) produces methanol, and the yeast strain CEN.PK113-7D (CBS 8340, the Centraal Buro voor Schimmelculture; van Dijken, et al., Enzyme Microb. Techno. 26:706-714, 2000) produces ethanol. Recombinant microorganisms which produce alcohol are also known in the art (e.g., Ohta, et al., Appl. Environ. Microbiol. 57:893-900, 1991; Underwood, et al., Appl. Environ. Microbiol. 68:1071-1081, 2002; Shen and Liao, Metab. Eng. 10:312-320, 2008; Hahnai, et al., Appl. Environ. Microbiol. 73:7814-7818, 2007; U.S. Pat. No. 5,514,583; U.S. Pat. No. 5,712,133; PCT Application Publication No. WO 1995/028476; Feldmann, et al., Appl. Microbiol. Biotechnol. 38: 354-361, 1992; Zhang, et al., Science 267:240-243, 1995; U.S. Patent Application Publication No. 2007/0031918 A1; U.S. Pat. No. 7,223,575; U.S. Pat. No. 7,741,119; U.S. Pat. No. 7,851,188; U.S. Patent Application Publication No. 2009/0203099 A1; U.S. Patent Application Publication No. 2009/0246846 A1; and PCT Application Publication No. WO 2010/075241, which are all herein incorporated by reference).

In addition, microorganisms may be modified using recombinant technologies to generate recombinant microorganisms capable of producing product alcohols such as ethanol and butanol. Microorganisms that may be recombinantly modified to produce a product alcohol via a biosynthetic pathway include members of the genera Clostridium, Zymomonas, Escherichia, Salmonella, Serratia, Erwinia, Klebsiella, Shigella, Rhodococcus, Pseudomonas, Bacillus, Lactobacillus, Enterococcus, Alcaligenes, Klebsiella, Paenibacillus, Arthrobacter, Corynebacterium, Brevibacterium, Schizosaccharomyces, Kluyveromyces, Yarrowia, Pichia, Candida, Hansenula, Issatchenkia, or Saccharomyces. In some embodiments, recombinant microorganisms may be selected from the group consisting of Escherichia coli, Lactobacillus plantarum, Kluyveromyces lactis, Kluyveromyces marxianus and Saccharomyces cerevisiae. In some embodiments, the recombinant microorganism is yeast. In some embodiments, the recombinant microorganism is crabtree-positive yeast selected from Saccharomyces, Zygosaccharomyces, Schizosaccharomyces, Dekkera, Torulopsis, Brettanomyces, and some species of Candida. Species of crabtree-positive yeast include, but are not limited to, Saccharomyces cerevisiae, Saccharomyces kluyveri, Schizosaccharomyces pombe, Saccharomyces bayanus, Saccharomyces mikitae, Saccharomyces paradoxus, Zygosaccharomyces rouxii, and Candida glabrata.

Saccharomyces cerevisiae are known in the art and are available from a variety of sources including, but not limited to, American Type Culture Collection (Rockville, Md.), Centraalbureau voor Schimmelcultures (CBS) Fungal Biodiversity Centre, LeSaffre, Gert Strand AB, Ferm Solutions, North American Bioproducts, Martrex, and Lallemand. Saccharomyces cerevisiae include, but are not limited to, BY4741, CEN.PK 113-7D, Ethanol Red® yeast, Ferm Pro™ yeast, Bio-Ferm® XR yeast, Gert Strand Prestige Batch Turbo alcohol yeast, Gert Strand Pot Distillers yeast, Gert Strand Distillers Turbo yeast, FerMax™ Green yeast, FerMax™ Gold yeast, Thermosacc® yeast, BG-1, PE-2, CAT-1, CBS7959, CBS7960, and CBS7961.

In some embodiments, the microorganism may be immobilized or encapsulated. For example, the microorganism may be immobilized or encapsulated using alginate, calcium alginate, or polyacrylamide gels, or through the induction of biofilm formation onto a variety of high surface area support matrices such as diatomite, celite, diatomaceous earth, silica gels, plastics, or resins. In some embodiments, ISPR may be used in combination with immobilized or encapsulated microorganisms. This combination may improve productivity such as specific volumetric productivity, metabolic rate, product alcohol yields, tolerance to product alcohol. In addition, immobilization and encapsulation may minimize the effects of the process conditions such as shearing on the microorganisms.

The production of butanol utilizing fermentation, as well as microorganisms which produce butanol, is disclosed, for example, in U.S. Pat. No. 7,851,188, and U.S. Patent Application Publication Nos. 2007/0092957; 2007/0259410; 2007/0292927; 2008/0182308; 2008/0274525; 2009/0155870; 2009/0305363; and 2009/0305370, the entire contents of each are herein incorporated by reference. In some embodiments, the microorganism is engineered to contain a biosynthetic pathway. In some embodiments, the biosynthetic pathway is an engineered butanol biosynthetic pathway. In some embodiments, the biosynthetic pathway converts pyruvate to a fermentation product. In some embodiments, the biosynthetic pathway converts pyruvate as well as amino acids to a fermentation product. In some embodiments, at least one, at least two, at least three, or at least four polypeptides catalyzing substrate to product conversions of a pathway are encoded by heterologous polynucleotides in the microorganism. In some embodiments, all polypeptides catalyzing substrate to product conversions of a pathway are encoded by heterologous polynucleotides in the microorganism. In some embodiments, the polypeptide catalyzing the substrate to product conversions of acetolactate to 2,3-dihydroxyisovalerate and/or the polypeptide catalyzing the substrate to product conversion of isobutyraldehyde to isobutanol are capable of utilizing reduced nicotinamide adenine dinucleotide (NADH) as a cofactor.

### Biosynthetic Pathways

Biosynthetic pathways for the production of isobutanol that may be used include those described in U.S. Pat. No. 7,851,188, which is incorporated herein by reference. In one embodiment, the isobutanol biosynthetic pathway comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by acetohydroxy acid reductoisomerase;
c) 2,3-dihydroxyisovalerate to α-ketoisovalerate, which may be catalyzed, for example, by acetohydroxy acid dehydratase;
d) α-ketoisovalerate to isobutyraldehyde, which may be catalyzed, for example, by a branched-chain α-keto acid decarboxylase; and
e) isobutyraldehyde to isobutanol, which may be catalyzed, for example, by a branched-chain alcohol dehydrogenase.

In another embodiment, the isobutanol biosynthetic pathway comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by ketol-acid reductoisomerase;
c) 2,3-dihydroxyisovalerate to α-ketoisovalerate, which may be catalyzed, for example, by dihydroxyacid dehydratase;
d) α-ketoisovalerate to valine, which may be catalyzed, for example, by transaminase or valine dehydrogenase;
e) valine to isobutylamine, which may be catalyzed, for example, by valine decarboxylase;
f) isobutylamine to isobutyraldehyde, which may be catalyzed by, for example, omega transaminase; and
g) isobutyraldehyde to isobutanol, which may be catalyzed, for example, by a branched-chain alcohol dehydrogenase.

In another embodiment, the isobutanol biosynthetic pathway comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by acetohydroxy acid reductoisomerase;
c) 2,3-dihydroxyisovalerate to α-ketoisovalerate, which may be catalyzed, for example, by acetohydroxy acid dehydratase;
d) α-ketoisovalerate to isobutyryl-CoA, which may be catalyzed, for example, by branched-chain keto acid dehydrogenase;
e) isobutyryl-CoA to isobutyraldehyde, which may be catalyzed, for example, by acylating aldehyde dehydrogenase; and
f) isobutyraldehyde to isobutanol, which may be catalyzed, for example, by a branched-chain alcohol dehydrogenase.

Biosynthetic pathways for the production of 1-butanol that may be used include those described in U.S. Patent Application Publication No. 2008/0182308, which is incorporated herein by reference. In one embodiment, the 1-butanol biosynthetic pathway comprises the following substrate to product conversions:
a) acetyl-CoA to acetoacetyl-CoA, which may be catalyzed, for example, by acetyl-CoA acetyltransferase;
b) acetoacetyl-CoA to 3-hydroxybutyryl-CoA, which may be catalyzed, for example, by 3-hydroxybutyryl-CoA dehydrogenase;
c) 3-hydroxybutyryl-CoA to crotonyl-CoA, which may be catalyzed, for example, by crotonase;
d) crotonyl-CoA to butyryl-CoA, which may be catalyzed, for example, by butyryl-CoA dehydrogenase;
e) butyryl-CoA to butyraldehyde, which may be catalyzed, for example, by butyraldehyde dehydrogenase; and
f) butyraldehyde to 1-butanol, which may be catalyzed, for example, by butanol dehydrogenase.

Biosynthetic pathways for the production of 2-butanol that may be used include those described in U.S. Patent Application Publication No. 2007/0259410 and U.S. Patent Application Publication No. 2009/0155870, which are incorporated herein by reference. In one embodiment, the 2-butanol biosynthetic pathway comprises the following substrate to product conversions:
a) pyruvate to alpha-acetolactate, which may be catalyzed, for example, by acetolactate synthase;
b) alpha-acetolactate to acetoin, which may be catalyzed, for example, by acetolactate decarboxylase;
c) acetoin to 3-amino-2-butanol, which may be catalyzed, for example, acetonin aminase;
d) 3-amino-2-butanol to 3-amino-2-butanol phosphate, which may be catalyzed, for example, by aminobutanol kinase;
e) 3-amino-2-butanol phosphate to 2-butanone, which may be catalyzed, for example, by aminobutanol phosphate phosphorylase; and
f) 2-butanone to 2-butanol, which may be catalyzed, for example, by butanol dehydrogenase.

In another embodiment, the 2-butanol biosynthetic pathway comprises the following substrate to product conversions:
a) pyruvate to alpha-acetolactate, which may be catalyzed, for example, by acetolactate synthase;
b) alpha-acetolactate to acetoin, which may be catalyzed, for example, by acetolactate decarboxylase;
c) acetoin to 2,3-butanediol, which may be catalyzed, for example, by butanediol dehydrogenase;
d) 2,3-butanediol to 2-butanone, which may be catalyzed, for example, by dial dehydratase; and
e) 2-butanone to 2-butanol, which may be catalyzed, for example, by butanol dehydrogenase.

Biosynthetic pathways for the production of 2-butanone that may be used include those described in U.S. Patent Application Publication No. 2007/0259410 and U.S. Patent Application Publication No. 2009/0155870, which are incorporated herein by reference. In one embodiment, the 2-butanone biosynthetic pathway comprises the following substrate to product conversions:
a) pyruvate to alpha-acetolactate, which may be catalyzed, for example, by acetolactate synthase;
b) alpha-acetolactate to acetoin, which may be catalyzed, for example, by acetolactate decarboxylase;
c) acetoin to 3-amino-2-butanol, which may be catalyzed, for example, acetonin aminase;
d) 3-amino-2-butanol to 3-amino-2-butanol phosphate, which may be catalyzed, for example, by aminobutanol kinase; and
e) 3-amino-2-butanol phosphate to 2-butanone, which may be catalyzed, for example, by aminobutanol phosphate phosphorylase.

In another embodiment, the 2-butanone biosynthetic pathway comprises the following substrate to product conversions:
a) pyruvate to alpha-acetolactate, which may be catalyzed, for example, by acetolactate synthase;
b) alpha-acetolactate to acetoin which may be catalyzed, for example, by acetolactate decarboxylase;
c) acetoin to 2,3-butanediol, which may be catalyzed, for example, by butanediol dehydrogenase; and
d) 2,3-butanediol to 2-butanone, which may be catalyzed, for example, by diol dehydratase.

The terms “acetohydroxyacid synthase,” “acetolactate synthase,” and “acetolactate synthetase” (abbreviated “ALS”) are used interchangeably herein to refer to a polypeptide (or polypeptides) having enzyme activity that catalyzes the conversion of pyruvate to acetolactate and CO₂. Example acetolactate synthases are known by the EC number 2.2.1.6 (Enzyme Nomenclature 1992, Academic Press, San Diego). These unmodified enzymes are available from a number of sources, including, but not limited to, Bacillus subtilis (GenBank Nos: CAB15618 (SEQ ID NO: 1), Z99122 (SEQ ID NO: 2), NCBI (National Center for Biotechnology Information) amino acid sequence, NCBI nucleotide sequence, respectively), Klebsiella pneumoniae (GenBank Nos: AAA25079 (SEQ ID NO: 3), M73842 (SEQ ID NO: 4)), and Lactococcus lactis (GenBank Nos: AAA25161 (SEQ ID NO: 5), L16975 (SEQ ID NO: 6)).

The term “ketol-acid reductoisomerase” (“KARI”), “acetohydroxy acid isomeroreductase,” and “acetohydroxy acid reductoisomerase” will be used interchangeably and refer to a polypeptide (or polypeptides) having enzyme activity that catalyzes the reaction of (S)-acetolactate to 2,3-dihydroxyisovalerate. Example KARI enzymes may be classified as EC number EC 1.1.1.86 (Enzyme Nomenclature 1992, Academic Press, San Diego), and are available from a vast array of microorganisms, including, but not limited to, Escherichia coli (GenBank Nos: NP_{—}418222 (SEQ ID NO: 7), NC_{—}000913 (SEQ ID NO: 8)), Saccharomyces cerevisiae (GenBank Nos: NP_{—}013459 (SEQ ID NO: 9), NC_{—}001144 (SEQ ID NO: 10)), Methanococcus maripaludis (GenBank Nos: CAF30210 (SEQ ID NO: 11), BX957220 (SEQ ID NO: 12)), and Bacillus subtilis (GenBank Nos: CAB14789 (SEQ ID NO: 13), Z99118 (SEQ ID NO: 14)). KARIs include Anaerostipes caccae KARI variants “K9G9” and “K9D3” (SEQ ID Nos: 15 and 16, respectively). Ketol-acid reductoisomerase (KARI) enzymes are described in U.S. Patent Application Publication Nos. 2008/0261230, 2009/0163376, and 2010/0197519, and PCT Application Publication No. WO/2011/041415, which are incorporated herein by reference. Examples of KARIs disclosed therein are those from Lactococcus lactis, Vibrio cholera, Pseudomonas aeruginosa PAO1, and Pseudomonas fluorescens PF5 mutants. In some embodiments, the KARI utilizes NADH. In some embodiments, the KARI utilizes reduced nicotinamide adenine dinucleotide phosphate (NADPH).

The term “acetohydroxy acid dehydratase” and “dihydroxyacid dehydratase” (“DHAD”) refers to a polypeptide (or polypeptides) having enzyme activity that catalyzes the conversion the conversion of 2,3-dihydroxyisovalerate to α-ketoisovalerate. Example acetohydroxy acid dehydratases are known by the EC number 4.2.1.9. Such enzymes are available from a vast array of microorganisms, including, but not limited to, Escherichia coli (GenBank Nos: YP_{—}026248 (SEQ ID NO: 17), NC000913 (SEQ ID NO: 18)), Saccharomyces cerevisiae (GenBank Nos: NP_{—}012550 (SEQ ID NO: 19), NC 001142 (SEQ ID NO: 20), M. maripaludis (GenBank Nos: CAF29874 (SEQ ID NO: 21), BX957219 (SEQ ID NO: 22)), B. subtilis (GenBank Nos: CAB14105 (SEQ ID NO: 23), Z99115 (SEQ ID NO: 24)), L. lactis, and N. crassa. U.S. Patent Application Publication No. 2010/0081154, and U.S. Pat. No. 7,851,188, which are incorporated herein by reference, describe dihydroxyacid dehydratases (DHADs), including a DHAD from Streptococcus mutans.

The term “branched-chain α-keto acid decarboxylase,” “α-ketoacid decarboxylase,” “α-ketoisovalerate decarboxylase,” or “2-ketoisovalerate decarboxylase” (“KIVD”) refers to a polypeptide (or polypeptides) having enzyme activity that catalyzes the conversion of α-ketoisovalerate to isobutyraldehyde and CO₂. Example branched-chain α-keto acid decarboxylases are known by the EC number 4.1.1.72 and are available from a number of sources, including, but not limited to, Lactococcus lactis (GenBank Nos: AAS49166 (SEQ ID NO: 25), AY548760 (SEQ ID NO: 26); CAG34226 (SEQ ID NO: 27), AJ746364 (SEQ ID NO: 28), Salmonella typhimurium (GenBank Nos: NP_{—}461346 (SEQ ID NO: 29), NC_{—}003197 (SEQ ID NO: 30)), Clostridium acetobutylicum (GenBank Nos: NP_{—}149189 (SEQ ID NO: 31), NC_{—}001988 (SEQ ID NO: 32)), M. caseolyticus (SEQ ID NO: 33), and L. grayi (SEQ ID NO: 34).

The term “branched-chain alcohol dehydrogenase” (“ADH”) refers to a polypeptide (or polypeptides) having enzyme activity that catalyzes the conversion of isobutyraldehyde to isobutanol. Example branched-chain alcohol dehydrogenases are known by the EC number 1.1.1.265, but may also be classified under other alcohol dehydrogenases (specifically, EC 1.1.1.1 or 1.1.1.2). Alcohol dehydrogenases may be NADPH-dependent or NADH-dependent. Such enzymes are available from a number of sources, including, but not limited to, Saccharomyces cerevisiae (GenBank Nos: NP_{—}010656 (SEQ ID NO: 35), NC_{—}001136 (SEQ ID NO: 36), NP_{—}014051 (SEQ ID NO: 37), NC_{—}001145 (SEQ ID NO: 38)), Escherichia coli (GenBank Nos: NP_{—}417484 (SEQ ID NO: 39), NC_{—}000913 (SEQ ID NO: 40)), C. acetobutylicum (GenBank Nos: NP_{—}349892 (SEQ ID NO: 41), NC_{—}003030 (SEQ ID NO: 42); NP_{—}349891 (SEQ ID NO: 43), NC_{—}003030 (SEQ ID NO: 44)). U.S. Patent Application Publication No. 2009/0269823 describes SadB, an alcohol dehydrogenase (ADH) from Achromobacter xylosoxidans. Alcohol dehydrogenases also include horse liver ADH and Beijerinkia indica ADH (as described by U.S. Patent Application Publication No. 2011/0269199, which is incorporated herein by reference).

The term “butanol dehydrogenase” refers to a polypeptide (or polypeptides) having enzyme activity that catalyzes the conversion of isobutyraldehyde to isobutanol or the conversion of 2-butanone and 2-butanol. Butanol dehydrogenases are a subset of a broad family of alcohol dehydrogenases. Butanol dehydrogenase may be NAD- or NADP-dependent. The NAD-dependent enzymes are known as EC 1.1.1.1 and are available, for example, from Rhodococcus ruber (GenBank Nos: CAD36475, AJ491307). The NADP dependent enzymes are known as EC 1.1.1.2 and are available, for example, from Pyrococcus furiosus (GenBank Nos: AAC25556, AF013169). Additionally, a butanol dehydrogenase is available from Escherichia coli (GenBank Nos: NP 417484, NC_{—}000913) and a cyclohexanol dehydrogenase is available from Acinetobacter sp. (GenBank Nos: AAG10026, AF282240). The term “butanol dehydrogenase” also refers to an enzyme that catalyzes the conversion of butyraldehyde to 1-butanol, using either NADH or NADPH as cofactor. Butanol dehydrogenases are available from, for example, C. acetobutylicum (GenBank Nos: NP_{—}149325, NC_{—}001988; this enzyme possesses both aldehyde and alcohol dehydrogenase activity); NP_{—}349891, NC_{—}003030; and NP_{—}349892, NC_{—}003030) and Escherichia coli (GenBank Nos: NP_{—}417-484, NC_{—}000913).

The term “branched-chain keto acid dehydrogenase” refers to a polypeptide (or polypeptides) having enzyme activity that catalyzes the conversion of α-ketoisovalerate to isobutyryl-CoA (isobutyryl-coenzyme A), typically using NAD⁺ (nicotinamide adenine dinucleotide) as an electron acceptor. Example branched-chain keto acid dehydrogenases are known by the EC number 1.2.4.4. Such branched-chain keto acid dehydrogenases are comprised of four subunits and sequences from all subunits are available from a vast array of microorganisms, including, but not limited to, Bacillus subtilis (GenBank Nos: CAB14336 (SEQ ID NO: 45), Z99116 (SEQ ID NO: 46); CAB14335 (SEQ ID NO: 47), Z99116 (SEQ ID NO: 48); CAB14334 (SEQ ID NO: 49), Z99116 (SEQ ID NO: 50); and CAB14337 (SEQ ID NO: 51), Z99116 (SEQ ID NO: 52)) and Pseudomonas putida (GenBank Nos: AAA65614 (SEQ ID NO: 53), M57613 (SEQ ID NO: 54); AAA65615 (SEQ ID NO: 55), M57613 (SEQ ID NO: 56); AAA65617 (SEQ ID NO: 57), M57613 (SEQ ID NO: 58); and AAA65618 (SEQ ID NO: 59), M57613 (SEQ ID NO: 60)).

The term “acylating aldehyde dehydrogenase” refers to a polypeptide (or polypeptides) having enzyme activity that catalyzes the conversion of isobutyryl-CoA to isobutyraldehyde, typically using either NADH or NADPH as an electron donor. Example acylating aldehyde dehydrogenases are known by the EC numbers 1.2.1.10 and 1.2.1.57. Such enzymes are available from multiple sources, including, but not limited to, Clostridium beijerinckii (GenBank Nos: AAD31841 (SEQ ID NO: 61), AF157306 (SEQ ID NO: 62)), Clostridium acetobutylicum (GenBank Nos: NP_{—}149325 (SEQ ID NO: 63), NC_{—}001988 (SEQ ID NO: 64); NP_{—}149199 (SEQ ID NO: 65), NC_{—}001988 (SEQ ID NO: 66)), Pseudomonas putida (GenBank Nos: AAA89106 (SEQ ID NO: 67), U13232 (SEQ ID NO: 68)), and Thermus thermophilus (GenBank Nos: YP_{—}145486 (SEQ ID NO: 69), NC_{—}006461 (SEQ ID NO: 70)).

The term “transaminase” refers to a polypeptide (or polypeptides) having enzyme activity that catalyzes the conversion of α-ketoisovalerate to L-valine, using either alanine or glutamate as an amine donor. Example transaminases are known by the EC numbers 2.6.1.42 and 2.6.1.66. Such enzymes are available from a number of sources. Examples of sources for alanine-dependent enzymes include, but are not limited to, Escherichia coli (GenBank Nos: YP_{—}026231 (SEQ ID NO: 71), NC_{—}000913 (SEQ ID NO: 72)) and Bacillus licheniformis (GenBank Nos: YP_{—}093743 (SEQ ID NO: 73), NC_{—}006322 (SEQ ID NO: 74)). Examples of sources for glutamate-dependent enzymes include, but are not limited to, Escherichia coli (GenBank Nos: YP_{—}026247 (SEQ ID NO: 75), NC_{—}000913 (SEQ ID NO: 76)), Saccharomyces cerevisiae (GenBank Nos: NP_{—}012682 (SEQ ID NO: 77), NC_{—}001142 (SEQ ID NO: 78)) and Methanobacterium thermoautotrophicum (GenBank Nos: NP_{—}276546 (SEQ ID NO: 79), NC_{—}000916 (SEQ ID NO: 80)).

The term “valine dehydrogenase” refers to a polypeptide (or polypeptides) having enzyme activity that catalyzes the conversion of α-ketoisovalerate to L-valine, typically using NAD(P)H as an electron donor and ammonia as an amine donor. Example valine dehydrogenases are known by the EC numbers 1.4.1.8 and 1.4.1.9 and such enzymes are available from a number of sources, including, but not limited to, Streptomyces coelicolor (GenBank Nos: NP_{—}628270 (SEQ ID NO: 81), NC_{—}003888 (SEQ ID NO: 82)) and Bacillus subtilis (GenBank Nos: CAB14339 (SEQ ID NO: 83), Z99116 (SEQ ID NO: 84)).

The term “valine decarboxylase” refers to a polypeptide (or polypeptides) having enzyme activity that catalyzes the conversion of L-valine to isobutylamine and CO₂. Example valine decarboxylases are known by the EC number 4.1.1.14. Such enzymes are found in Streptomyces, such as for example, Streptomyces viridifaciens (GenBank Nos: AAN10242 (SEQ ID NO: 85), AY116644 (SEQ ID NO: 86)).

The term “omega transaminase” refers to a polypeptide (or polypeptides) having enzyme activity that catalyzes the conversion of isobutylamine to isobutyraldehyde using a suitable amino acid as an amine donor. Example omega transaminases are known by the EC number 2.6.1.18 and are available from a number of sources, including, but not limited to, Alcaligenes denitrificans (AAP92672 (SEQ ID NO: 87), AY330220 (SEQ ID NO: 88)), Ralstonia eutropha (GenBank Nos: YP_{—}294474 (SEQ ID NO: 89), NC_{—}007347 (SEQ ID NO: 90)), Shewanella oneidensis (GenBank Nos: NP_{—}719046 (SEQ ID NO: 91), NC_{—}004347 (SEQ ID NO: 92)), and Pseudomonas putida (GenBank Nos: AAN66223 (SEQ ID NO: 93), AE016776 (SEQ ID NO: 94)).

The term “acetyl-CoA acetyltransferase” refers to a polypeptide (or polypeptides) having enzyme activity that catalyzes the conversion of two molecules of acetyl-CoA to acetoacetyl-CoA and coenzyme A (CoA). Example acetyl-CoA acetyltransferases are acetyl-CoA acetyltransferases with substrate preferences (reaction in the forward direction) for a short chain acyl-CoA and acetyl-CoA and are classified as E.C. 2.3.1.9 [Enzyme Nomenclature 1992, Academic Press, San Diego]; although, enzymes with a broader substrate range (E.C. 2.3.1.16) will be functional as well. Acetyl-CoA acetyltransferases are available from a number of sources, for example, Escherichia coli (GenBank Nos: NP_{—}416728, NC_{—}000913; NCBI (National Center for Biotechnology Information) amino acid sequence, NCBI nucleotide sequence), Clostridium acetobutylicum (GenBank Nos: NP_{—}349476.1, NC_{—}003030; NP_{—}149242, NC_{—}001988, Bacillus subtilis (GenBank Nos: NP_{—}390297, NC_{—}000964), and Saccharomyces cerevisiae (GenBank Nos: NP_{—}015297, NC_{—}001148).

The term “3-hydroxybutyryl-CoA dehydrogenase” refers to a polypeptide (or polypeptides) having enzyme activity that catalyzes the conversion of acetoacetyl-CoA to 3-hydroxybutyryl-CoA. Example 3-hydroxybutyryl-CoA dehydrogenases may be NADH-dependent, with a substrate preference for (S)-3-hydroxybutyryl-CoA or (R)-3-hydroxybutyryl-CoA. Examples may be classified as E.C. 1.1.1.35 and E.C. 1.1.1.30, respectively. Additionally, 3-hydroxybutyryl-CoA dehydrogenases may be NADPH-dependent, with a substrate preference for (S)-3-hydroxybutyryl-CoA or (R)-3-hydroxybutyryl-CoA and are classified as E.C. 1.1.1.157 and E.C. 1.1.1.36, respectively. 3-Hydroxybutyryl-CoA dehydrogenases are available from a number of sources, for example, Clostridium acetobutylicum (GenBank Nos: NP_{—}349314, NC_{—}003030), Bacillus subtilis (GenBank Nos: AAB09614, U29084), Ralstonia eutropha (GenBank Nos: YP_{—}294481, NC_{—}007347), and Alcaligenes eutrophus (GenBank Nos: AAA21973, J04987).

The term “crotonase” refers to a polypeptide (or polypeptides) having enzyme activity that catalyzes the conversion of 3-hydroxybutyryl-CoA to crotonyl-CoA and H₂O. Example crotonases may have a substrate preference for (S)-3-hydroxybutyryl-CoA or (R)-3-hydroxybutyryl-CoA and may be classified as E.C. 4.2.1.17 and E.C. 4.2.1.55, respectively. Crotonases are available from a number of sources, for example, Escherichia coli (GenBank Nos: NP_{—}415911, NC_{—}000913), Clostridium acetobutylicum (GenBank Nos: NP_{—}349318, NC_{—}003030), Bacillus subtilis (GenBank Nos: CAB13705, Z99113), and Aeromonas caviae (GenBank Nos: BAA21816, D88825).

The term “butyryl-CoA dehydrogenase” refers to a polypeptide (or polypeptides) having enzyme activity that catalyzes the conversion of crotonyl-CoA to butyryl-CoA. Example butyryl-CoA dehydrogenases may be NADH-dependent, NADPH-dependent, or flavin-dependent and may be classified as E.C. 1.3.1.44, E.C. 1.3.1.38, and E.C. 1.3.99.2, respectively. Butyryl-CoA dehydrogenases are available from a number of sources, for example, Clostridium acetobutylicum (GenBank Nos: NP_{—}347102, NC_{—}003030), Euglena gracilis (GenBank Nos: Q5EU90, AY741582), Streptomyces collinus (GenBank Nos: AAA92890, U37135), and Streptomyces coelicolor (GenBank Nos: CAA22721, AL939127).

The term “butyraldehyde dehydrogenase” refers to a polypeptide (or polypeptides) having enzyme activity that catalyzes the conversion of butyryl-CoA to butyraldehyde, using NADH or NADPH as cofactor. Butyraldehyde dehydrogenases with a preference for NADH are known as E.C. 1.2.1.57 and are available from, for example, Clostridium beijerinckii (GenBank Nos: AAD31841, AF157306) and Clostridium acetobutylicum (GenBank Nos: NP.sub.—149325, NC.sub.—001988).

The term “isobutyryl-CoA mutase” refers to a polypeptide (or polypeptides) having enzyme activity that catalyzes the conversion of butyryl-CoA to isobutyryl-CoA. This enzyme uses coenzyme B₁₂ as cofactor. Example isobutyryl-CoA mutases are known by the EC number 5.4.99.13. These enzymes are found in a number of Streptomyces, including, but not limited to, Streptomyces cinnamonensis (GenBank Nos: AAC08713 (SEQ ID NO: 95), U67612 (SEQ ID NO: 96); CAB59633 (SEQ ID NO: 97), AJ246005 (SEQ ID NO: 98)), Streptomyces coelicolor (GenBank Nos: CAB70645 (SEQ ID NO: 99), AL939123 (SEQ ID NO: 100); CAB92663 (SEQ ID NO: 101), AL939121 (SEQ ID NO: 102)), and Streptomyces avermitilis (GenBank Nos: NP_{—}824008 (SEQ ID NO: 103), NC_{—}003155 (SEQ ID NO: 104); NP_{—}824637 (SEQ ID NO: 105), NC_{—}003155 (SEQ ID NO: 106)).

The term “acetolactate decarboxylase” refers to a polypeptide (or polypeptides) having enzyme activity that catalyzes the conversion of alpha-acetolactate to acetoin. Example acetolactate decarboxylases are known as EC 4.1.1.5 and are available, for example, from Bacillus subtilis (GenBank Nos: AAA22223, L04470), Klebsiella terrigena (GenBank Nos: AAA25054, L04507) and Klebsiella pneumoniae (GenBank Nos: AAU43774, AY722056).

The term “acetoin aminase” or “acetoin transaminase” refers to a polypeptide (or polypeptides) having enzyme activity that catalyzes the conversion of acetoin to 3-amino-2-butanol. Acetoin aminase may utilize the cofactor pyridoxal 5′-phosphate or NADH or NADPH. The resulting product may have (R) or (S) stereochemistry at the 3-position. The pyridoxal phosphate-dependent enzyme may use an amino acid such as alanine or glutamate as the amino donor. The NADH- and NADPH-dependent enzymes may use ammonia as a second substrate. A suitable example of an NADH-dependent acetoin aminase, also known as amino alcohol dehydrogenase, is described by Ito, et al. (U.S. Pat. No. 6,432,688). An example of a pyridoxal-dependent acetoin aminase is the amine:pyruvate aminotransferase (also called amine:pyruvate transaminase) described by Shin and Kim (J. Org. Chem. 67:2848-2853, 2002).

The term “acetoin kinase” refers to a polypeptide (or polypeptides) having enzyme activity that catalyzes the conversion of acetoin to phosphoacetoin. Acetoin kinase may utilize ATP (adenosine triphosphate) or phosphoenolpyruvate as the phosphate donor in the reaction. Enzymes that catalyze the analogous reaction on the similar substrate dihydroxyacetone, for example, include enzymes known as EC 2.7.1.29 (Garcia-Alles, et al., Biochemistry 43:13037-13046, 2004).

The term “acetoin phosphate aminase” refers to a polypeptide (or polypeptides) having enzyme activity that catalyzes the conversion of phosphoacetoin to 3-amino-2-butanol O-phosphate. Acetoin phosphate aminase may use the cofactor pyridoxal 5′-phosphate, NADH, or NADPH. The resulting product may have (R) or (S) stereochemistry at the 3-position. The pyridoxal phosphate-dependent enzyme may use an amino acid such as alanine or glutamate. The NADH-dependent and NADPH-dependent enzymes may use ammonia as a second substrate. Although there are no reports of enzymes catalyzing this reaction on phosphoacetoin, there is a pyridoxal phosphate-dependent enzyme that is proposed to carry out the analogous reaction on the similar substrate serinol phosphate (Yasuta, et al., Appl. Environ. Microbial. 67:4999-5009, 2001).

The term “aminobutanol phosphate phospholyase,” also called “amino alcohol O-phosphate lyase,” refers to a polypeptide (or polypeptides) having enzyme activity that catalyzes the conversion of 3-amino-2-butanol O-phosphate to 2-butanone. Amino butanol phosphate phospho-lyase may utilize the cofactor pyridoxal 5′-phosphate. There are reports of enzymes that catalyze the analogous reaction on the similar substrate 1-amino-2-propanol phosphate (Jones, et al., Biochem J. 134:167-182, 1973). U.S. Patent Application Publication No. 2007/0259410 describes an aminobutanol phosphate phospho-lyase from the organism Erwinia carotovora.

The term “aminobutanol kinase” refers to a polypeptide (or polypeptides) having enzyme activity that catalyzes the conversion of 3-amino-2-butanol to 3-amino-2-butanol O-phosphate. Amino butanol kinase may utilize ATP as the phosphate donor. Although there are no reports of enzymes catalyzing this reaction on 3-amino-2-butanol, there are reports of enzymes that catalyze the analogous reaction on the similar substrates ethanolamine and 1-amino-2-propanol (Jones, et al., supra). U.S. Patent Application Publication No. 2009/0155870 describes, in Example 14, an amino alcohol kinase of Erwinia carotovora subsp. Atroseptica.

The term “butanediol dehydrogenase” also known as “acetoin reductase” refers to a polypeptide (or polypeptides) having enzyme activity that catalyzes the conversion of acetoin to 2,3-butanediol. Butanedial dehydrogenases are a subset of the broad family of alcohol dehydrogenases. Butanediol dehydrogenase enzymes may have specificity for production of (R)— or (S)-stereochemistry in the alcohol product. (S)-specific butanediol dehydrogenases are known as EC 1.1.1.76 and are available, for example, from Klebsiella pneumoniae (GenBank Nos: BBA13085, D86412). (R)-specific butanediol dehydrogenases are known as EC 1.1.1.4 and are available, for example, from Bacillus cereus (GenBank Nos. NP 830481, NC_{—}004722; AAP07682, AE017000), and Lactococcus lactis (GenBank Nos. AAK04995, AE006323).

The term “butanediol dehydratase,” also known as “dial dehydratase” or “propanediol dehydratase” refers to a polypeptide (or polypeptides) having enzyme activity that catalyzes the conversion of 2,3-butanediol to 2-butanone. Butanediol dehydratase may utilize the cofactor adenosyl cobalamin (also known as coenzyme Bw or vitamin B12; although vitamin B12 may refer also to other forms of cobalamin that are not coenzyme B12). Adenosyl cobalamin-dependent enzymes are known as EC 4.2.1.28 and are available, for example, from Klebsiella oxytoca (GenBank Nos: AA08099 (alpha subunit), D45071; BAA08100 (beta subunit), D45071; and BBA08101 (gamma subunit), D45071 (Note all three subunits are required for activity), and Klebsiella pneumonia (GenBank Nos: AAC98384 (alpha subunit), AF102064; GenBank Nos: AAC98385 (beta subunit), AF102064, GenBank Nos: AAC98386 (gamma subunit), AF102064). Other suitable dial dehydratases include, but are not limited to, B 12-dependent dial dehydratases available from Salmonella typhimurium (GenBank Nos: AAB84102 (large subunit), AF026270; GenBank Nos: AAB84103 (medium subunit), AF026270; GenBank Nos: AAB84104 (small subunit), AF026270); and Lactobacillus collinoides (GenBank Nos: CAC82541 (large subunit), AJ297723; GenBank Nos: CAC82542 (medium subunit); AJ297723; GenBank Nos: CAD01091 (small subunit), AJ297723); and enzymes from Lactobacillus brevis (particularly strains CNRZ 734 and CNRZ 735, Speranza, et al., J. Agric. Food Chem. 45:3476-3480, 1997), and nucleotide sequences that encode the corresponding enzymes. Methods of dial dehydratase gene isolation are well known in the art (e.g., U.S. Pat. No. 5,686,276).

The term “pyruvate decarboxylase” refers to a polypeptide (or polypeptides) having enzyme activity that catalyzes the decarboxylation of pyruvic acid to acetaldehyde and carbon dioxide. Pyruvate dehydrogenases are known by the EC number 4.1.1.1. These enzymes are found in a number of yeast, including Saccharomyces cerevisiae (GenBank Nos: CAA97575 (SEQ ID NO: 107), CAA97705 (SEQ ID NO: 109), CAA97091 (SEQ ID NO: 111)).

It will be appreciated that microorganisms comprising an isobutanol biosynthetic pathway as provided herein may further comprise one or more additional modifications. U.S. Patent Application Publication No. 2009/0305363 (incorporated by reference) discloses increased conversion of pyruvate to acetolactate by engineering yeast for expression of a cytosol-localized acetolactate synthase and substantial elimination of pyruvate decarboxylase activity. In some embodiments, the microorganisms may comprise modifications to reduce glycerol-3-phosphate dehydrogenase activity and/or disruption in at least one gene encoding a polypeptide having pyruvate decarboxylase activity or a disruption in at least one gene encoding a regulatory element controlling pyruvate decarboxylase gene expression as described in U.S. Patent Application Publication No. 2009/0305363 (incorporated herein by reference), and/or modifications that provide for increased carbon flux through an Entner-Doudoroff Pathway or reducing equivalents balance as described in U.S. Patent Application Publication No. 2010/0120105 (incorporated herein by reference). Other modifications include integration of at least one polynucleotide encoding a polypeptide that catalyzes a step in a pyruvate-utilizing biosynthetic pathway. Other modifications include at least one deletion, mutation, and/or substitution in an endogenous polynucleotide encoding a polypeptide having acetolactate reductase activity. In some embodiments, the polypeptide having acetolactate reductase activity is YMR226c (SEQ ID NOs: 127, 128) of Saccharomyces cerevisiae or a homolog thereof. Additional modifications include a deletion, mutation, and/or substitution in an endogenous polynucleotide encoding a polypeptide having aldehyde dehydrogenase and/or aldehyde oxidase activity. In some embodiments, the polypeptide having aldehyde dehydrogenase activity is ALD6 from Saccharomyces cerevisiae or a homolog thereof A genetic modification which has the effect of reducing glucose repression wherein the yeast production host cell is pdc− is described in U.S. Patent Application Publication No. 2011/0124060, incorporated herein by reference. In some embodiments, the pyruvate decarboxylase that is deleted or down-regulated is selected from the group consisting of: PDC1, PDC5, PDC6, and combinations thereof. In some embodiments, the pyruvate decarboxylase is selected from those enzymes in Table 1. In some embodiments, microorganisms may contain a deletion or down-regulation of a polynucleotide encoding a polypeptide that catalyzes the conversion of glyceraldehyde-3-phosphate to glycerate 1,3, bisphosphate. In some embodiments, the enzyme that catalyzes this reaction is glyceraldehyde-3-phosphate dehydrogenase.

TABLE 1
SEQ ID Numers of PDC Target Gene coding regions and Proteins

| | SEQ ID NO: | SEQ ID NO: |
|---|---|---|
| Description | (Amino Acid) | (Nucleic Acid) |
| PDC1 pyruvate | 107 | 108 |
| decarboxylase from | | |
| Saccharomyces cerevisiae | | |
| PDC5 pyruvate | 109 | 110 |
| decarboxylase from | | |
| Saccharomyces cerevisiae | | |
| PDC6 pyruvate | 111 | 112 |
| decarboxylase | | |
| Saccharomyces cerevisiae | | |
| pyruvate decarboxylase | 113 | 114 |
| from Candida glabrata | | |
| PDC1 pyruvate | 115 | 116 |
| decarboxylase from Pichia | | |
| stipitis | | |
| PDC2 pyruvate | 117 | 118 |
| decarboxylase from Pichia | | |
| stipitis | | |
| pyruvate decarboxylase | 119 | 120 |
| from Kluyveromyces lactis | | |
| pyruvate decarboxylase | 121 | 122 |
| from Yarrowia lipolytica | | |
| pyruvate decarboxylase | 123 | 124 |
| from Schizosaccharomyces | | |
| pombe | | |
| pyruvate decarboxylase | 125 | 126 |
| from Zygosaccharomyces | | |
| rouxii | | |

In some embodiments, any particular nucleic acid molecule or polypeptide may be at least 80%, 85%, 90%, 95%, 96%, 97%, 98%, or 99% identical to a nucleotide sequence or polypeptide sequence described herein. The term “percent identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. Identity and similarity can be readily calculated by known methods, including but not limited to those disclosed in: Computational Molecular Biology (Lesk, A. M., Ed.) Oxford University: NY (1988); Biocomputing: Informatics and Genome Projects (Smith, D. W., Ed.) Academic: NY (1993); Computer Analysis of Sequence Data, Part I (Griffin, A. M., and Griffin, H. G., Eds.) Humania: NJ (1994); Sequence Analysis in Molecular Biology (von Heinje, G., Ed.) Academic (1987); and Sequence Analysis Primer (Gribskov, M. and Devereux, J., Eds.) Stockton: NY (1991).

Methods to determine identity are designed to give the best match between the sequences tested. Methods to determine identity and similarity are codified in publicly available computer programs. Sequence alignments and percent identity calculations may be performed using the MegAlign™ program of the LASERGENE bioinformatics computing suite (DNASTAR Inc., Madison, Wis.). Multiple alignment of the sequences may be performed using the Clustal method of alignment which encompasses several varieties of the algorithm including the Clustal V method of alignment corresponding to the alignment method labeled Clustal V (disclosed by Higgins and Sharp, CABIOS. 5:151-153, 1989; Higgins, et al., Comput. Appl. Biosci. 8:189-191, 1992) and found in the MegAlign™ program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a percent identity by viewing the sequence distances table in the same program. Additionally the Clustal W method of alignment is available and corresponds to the alignment method labeled Clustal W (described by Higgins and Sharp, CABIOS. 5:151-153, 1989; Higgins, et al., Comput. Appl. Biosci. 8:189-191, 1992) and found in the MegAlign™ v6.1 program of the LASERGENE bioinformatics computing suite (DNASTAR Inc.). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a percent identity by viewing the sequence distances table in the same program.

Standard recombinant DNA and molecular cloning techniques are well known in the art and are described by Sambrook, et al. (Sambrook, J., Fritsch, E. F. and Maniatis, T. (Molecular Cloning: A Laboratory Manual; Cold Spring Harbor Laboratory Press, Cold Spring Harbor, 1989) and by Ausubel, et al. (Ausubel, et al., Current Protocols in Molecular Biology, pub. by Greene Publishing Assoc. and Wiley-Interscience, 1987). Examples of methods to construct microorganisms that comprise a butanol biosynthetic pathway are disclosed, for example, in U.S. Pat. No. 7,851,188, and U.S. Patent Application Publication Nos. 2007/0092957; 2007/0259410; 2007/0292927; 2008/0182308; 2008/0274525; 2009/0155870; 2009/0305363; and 2009/0305370, the entire contents of each are herein incorporated by reference.

Further, while various embodiments of the present invention have been described herein, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the claims and their equivalents.

All publications, patents, and patent applications mentioned in this specification are indicative of the level of skill of those skilled in the art to which this invention pertains, and are herein incorporated by reference to the same extent as if each individual publication, patent, or patent application was specifically and individually indicated to be incorporated by reference.

### EXAMPLES

The following nonlimiting examples will further illustrate the invention. It should be understood that, while the following examples involve corn as feedstock, other biomass sources can be used for feedstock without departing from the present invention.

As used herein, the meaning of abbreviations used was as follows: “g” means gram(s), “kg” means kilogram(s), “lbm” means pound mass, “gpm” means gallons per minute, “gal” means gallon(s), “MMGPY” means million gallon per year, “L” means liter(s), “mL” means milliliter(s), “μL” means microliter(s), “mL/L” means milliliter(s) per liter, “mL/min” means milliliter(s) per min, “min” means minute(s), “hr” means hour(s), “DI” means deionized, “uM” means micrometer(s), “nm” means nanometer(s), “w/v” means weight/volume, “wt %” means weight percent, “OD” means optical density, “OD₆₀₀” means optical density at a wavelength of 600 nM, “dcw” means dry cell weight, “rpm” means revolutions per minute, “° C.” means degree(s) Celsius, “° C./min” means degrees Celsius per minute, “slpm” means standard liter(s) per minute, “ppm” means part per million, “cP” means centipoise, “ID” means inner diameter, and “GC” means gas chromatograph.

### Example 1

### Effect of Undissolved Solids on the Rate of Mass Transfer

The following experiment was performed to measure the effect of undissolved solids on the rate of mass transfer of i-BuOH from an aqueous phase that simulates the composition of a fermentation broth derived from corn mash, which is approximately half way through a simultaneous saccharification and fermentation (SSF) fermentation (i.e., about 50% conversion of the oligosaccharides) in order to mimic the average composition of the liquid phase for an SSF batch.

Approximately 100 kg of liquefied corn mash was prepared in three equivalent batches using a 30 L glass, jacketed resin kettle. The kettle was set up with mechanical agitation, temperature control, and pH control. The protocol used for the three batches was as follows: (a) mixing ground corn with tap water (30 wt % corn on a dry basis), (b) heating the slurry to 55° C. while agitating, (c) adjusting pH of the slurry to 5.8 with either NaOH or H₂SO₄, (d) adding alpha-amylase (0.02 wt % on a dry corn basis), (e) heating the slurry to 85° C., (f) adjusting pH to 5.8, (g) holding the slurry at 85° C. for 2 hr while maintaining pH at 5.8, and (h) cooling the slurry to 25° C.

Pioneer 3335 hybrid corn, whole kernel yellow corn, was used (Pioneer Hi-Bred International, Johnston, Iowa), and it was ground in a hammer-mill using a 1 mm screen. The moisture content of the ground corn was 12 wt %, and the starch content of the ground corn was 71.4 wt % on a dry corn basis. The alpha-amylase enzyme was Liquozyme® SC DS (Novozymes, Franklinton, N.C.). The total amounts of the ingredients used for the three batches combined were: 33.9 kg ground corn (12% moisture), 65.4 kg tap water, and 0.006 kg Liquozyme® SC DS. A total of 0.297 kg of NaOH (17 wt %) was added to control pH. No H₂SO₄ was required. The total amount of liquefied corn mash recovered from the three 30 L batches was 99.4 kg.

Solids were removed from the mash by centrifugation in a large floor centrifuge which contained six 1 L bottles. Mash (73.4 kg) was centrifuged at 8000 rpm for 20 min at 25° C. yielding 44.4 kg of centrate and 26.9 kg of wet cake. It was determined that the centrate contained <1 wt % suspended solids, and that the wet cake contained approximately 18 wt % suspended solids. This implies that the original liquefied mash contained approximately 7 wt % suspended solids. This is consistent with the corn loading and starch content of the corn used assuming most of the starch was liquefied. If all of the starch was liquefied, the 44.4 kg of centrate recovered directly from the centrifuge would have contained approximately 23 wt % dissolved oligosaccharides (liquefied starch). About 0.6 kg of i-BuOH was added to 35.4 kg of centrate to preserve it. The resulting 36.0 kg of centrate, which contained 1.6 wt % i-BuOH, was used as a stock solution. The centrate mimics the liquid phase composition at the beginning of SSF. Therefore, a portion of it was diluted with an equal amount of H₂O on a mass basis to generate centrate that mimics SSF at about 50% conversion. More i-BuOH was added to bring the final concentration of i-BuOH in the diluted centrate to 3.0 wt % (about 30 g/L).

The diluted centrate was prepared as follows: 18 kg of the centrate stock solution which contained 1.6 wt % i-BuOH, was mixed with 18 kg tap water and 0.82 kg i-BuOH was added. The resulting 36.8 kg solution of diluted centrate consisted of approximately 11 wt % oligosaccharides and approximately 30 g/L i-BuOH. This solution mimics the liquid phase of a corn mash fermentation (SSF) at approximately 50% conversion of the oligosaccharides and an aqueous titer of 30 g/L i-BuOH.

Mass transfer tests were conducted using this solution as the aqueous phase to mimic mass transfer performance in a broth derived from liquefied corn mash after most of the undissolved solids are removed. The objective of the mass transfer tests was to measure the effect of undissolved solids on the overall volumetric mass transfer coefficient (k_{L}a) for the transfer of i-BuOH from a simulated broth, derived from liquefied corn mash, to a dispersion of solvent (extractant) droplets rising through the simulated broth. Correlations of k_{L}a with key design of operating parameters can be used to scale up mass transfer operations. Examples of parameters that should be held constant as much as possible in order to generate correlations of k_{L}a from smaller scale data which are useful for scale up are the physical properties of both phases and design parameters that determine droplet size (e.g., nozzle diameter, velocity of the phase to be dispersed through the nozzle).

A 6-inch diameter, 7-foot tall glass, jacketed column was used to measure the k_{L}a for the transfer of i-BuOH from an aqueous solution of oligosaccharides (derived from liquefied corn mash), both with and without suspended mash solids, to a dispersion of oleyl alcohol (OA) droplets rising through the simulated broth. i-BuOH was added to the aqueous phase to give an initial concentration of i-BuOH of approximately 30 g/L. A certain amount of the aqueous phase (typically about 35 kg) which contained approximately 11 wt % oligosaccharides and approximately 30 g/L i-BuOH, was charged to the column, and the column was heated to 30° C. by flowing warm H₂O through the jacket. There was no flow of aqueous phase in or out of the column during the test.

Fresh oleyl alcohol (80/85% grade, Cognis Corporation, Cincinnati, Ohio) was sparged into the bottom of the column through a single nozzle to create a dispersion of extractant droplets which flowed up through the aqueous phase. After reaching the top of the aqueous phase, the extractant drops formed a separate organic phase which then overflowed from the top of the column and was collected into a receiver. Typically, 3 to 5 gallons of oleyl alcohol flowed through the column for a single test.

Samples of the aqueous phase were collected from the column at several times throughout the test, and a composite sample of the total “rich” oleyl alcohol collected from the overflow was collected at the end of the test. All samples were analyzed for i-BuOH using a HP-6890 GC (Agilent Technologies, Inc., Santa Clara, Calif.). The concentration profile for i-BuOH in the aqueous phase (i.e., i-BuOH concentration versus time) was used to calculate the k_{L}a at the given set of operating conditions. The final composite sample of the total “rich” oleyl alcohol collected during the test was used to check the mass balance for i-BuOH.

The nozzle size and nozzle velocity (average velocity of oleyl alcohol through the feed nozzle) were varied to observe the effects on the k_{L}a. A series of tests were done using an aqueous solution of oligosaccharides (diluted centrate obtained from liquefied corn mash) with the mash solids removed. A similar series of tests were done using the same aqueous solution of oligosaccharides after adding the mash solids back to simulate liquefied corn mash (including the undissolved solids) at the middle of SSF. It is noted that under some operating conditions (e.g., higher oleyl alcohol flow rates), poor phase separation was obtained at the top of the column which made it difficult to obtain a representative composite sample of the total “rich” oleyl alcohol collected during the test. It is also noted that under some operating conditions, samples of the aqueous phase contained a significant amount of organic phase. Special sample handling and preparation techniques were employed to obtain a sample of the aqueous phase that was as representative as possible of the aqueous phase in the column at the time the sample was collected.

It was determined that the aqueous phase in the column was “well mixed” for all practical purposes because the concentration of i-BuOH did not vary much along the length of the column at a given point in time. Assuming the solvent droplet phase is also well mixed, the overall mass transfer of i-BuOH from the aqueous phase to the solvent phase in the column can be approximated by the following equation:

$${r}_{B} = {k}_{L} a ( {C}_{B , broth} - \frac{{C}_{B , solvent}}{{K}_{B}} ) \qquad ( 1 )$$

where,
r_{B}=total mass of i-BuOH transferred from the aqueous phase to the solvent phase per unit time per unit volume of the aqueous phase, grams i-BuOH/liter aqueous phase/hr or g/L/hr.
k_{L}a=overall volumetric mass transfer coefficient describing the mass transfer of i-BuOH from the aqueous phase to the solvent phase, hr⁻¹.
C_{B,broth}=average concentration of i-BuOH in the simulated broth (aqueous) phase over the entire test, grams i-BuOH/Liter aqueous phase or g/L.
C_{B,solvent}=average concentration of i-BuOH in the solvent phase over the entire test, grams i-BuOH/Liter solvent phase or g/L.
K_{B}=average equilibrium distribution coefficient for i-BuOH between the solvent and aqueous phase, (grams i-BuOH/Liter solvent phase)/(grams i-BuOH/Liter aqueous phase).

The parameters r_{B}, C_{B,broth}, and C_{B,solvent} were calculated for each test from the concentration data obtained from the samples of the aqueous and solvent phases. The parameter K_{B} was independently measured by mixing aqueous centrate from liquefied corn mash, oleyl alcohol, and i-BuOH and vigorously mixing the system until the two liquid phases were at equilibrium. The concentration of i-BuOH was measured in both phases to determine K_{B}. After r_{B}, C_{B,broth}, C_{B,solvent}, and K_{B} were determined for a given test, the k_{L}a could be calculated by rearranging Equation (1):

$${k}_{L} a = \frac{{r}_{B}}{( {C}_{B , broth} - \frac{{C}_{B , solvent}}{{K}_{B}} )} \qquad ( 2 )$$

Mass transfer tests were conducted with two different size nozzles at nozzle velocities ranging from 5 ft/s to 21 ft/s using the diluted centrate (solids removed) as the aqueous phase. Three tests were done using a nozzle that has an ID of 0.76 mm, and three tests were done using a nozzle that has an ID of 2.03 mm. All tests were conducted at 30° C. in the 6-inch diameter column described above using oleyl alcohol as the solvent. The equilibrium distribution coefficient for i-BuOH between oleyl alcohol and the diluted centrate which was obtained from liquefied corn mash by removing the solids, was measured to be approximately 5. The results of the mass transfer tests using diluted centrate (with the solids removed) are shown in Table 2.

TABLE 2

| | 41 | 42 | 43 | 44 | 45 | 46 |
|---|---|---|---|---|---|---|
| | Diluted | Diluted | Diluted | Diluted | Diluted | Diluted |
| | Centrate | Centrate | Centrate | Centrate | Centrate | Centrate |
| | from Liq'd | from Liq'd | from Liq'd | from Liq'd | from Liq'd | from Liq'd |
| | Mash, | Mash, | Mash, | Mash, | Mash, | Mash, |
| | Solids | Solids | Solids | Solids | Solids | Solids |
| | Removed | Removed | Removed | Removed | Removed | Removed |
| MASS TRANSFER TEST CONDITIONS: | | | | | | |
| Aqueous Phase | 36.0 | 35.0 | 34.3 | 32.0 | 28.0 | 28.6 |
| Volume of Aqueous Phase, L: | | | | | | |
| Solvent Feed Rate, g/min: | 33.2 | 79.5 | 145.3 | 237.7 | 507.7 | 875 |
| Superficial Liq. Velocity (Us), ft/hr: | 0.42 | 1.01 | 1.84 | 3.02 | 6.45 | 11.11 |
| Nozzle I.D., mm: | 0.76 | 0.76 | 0.76 | 2.03 | 2.03 | 2.03 |
| Nozzle Velocity, ft/s | 4.7 | 11.3 | 20.6 | 4.7 | 10.1 | 17.4 |
| MASS TRANSFER RESULTS: | | | | | | |
| Initial [i-B] in Aq. Phase, g/L: | 28.2 | 27.0 | 29.1 | 31.3 | 38.7 | 30.1 |
| Final [i-B] in Aq. Phase, g/L: | 25.7 | 14.8 | 14.7 | 24.8 | 11.5 | 5.4 |
| Rich OA collected, kg: | 4.05 | 7.47 | 6.03 | 7.37 | 12.82 | 14.0 |
| [i-B] in OA collected, wt %: | 2.22 | 5.72 | 8.17 | 2.83 | 5.93 | 5.04 |
| Test time, min: | 122 | 94 | 41.5 | 31.0 | 25.3 | 16.0 |
| Overall i-BuOH M.T. Rate, g/L/hr | 1.23 | 7.81 | 20.76 | 12.62 | 64.52 | 92.52 |
| kLa, hr\hat{ }(−1) | 0.05 | 0.70 | 2.58 | 0.54 | 4.29 | 10.06 |
| (kLa/Us) | 0.12 | 0.69 | 1.40 | 0.18 | 0.67 | 0.91 |

An aqueous phase that simulates a fermentation broth from liquefied corn mash (containing undissolved solids) half way through SSF was synthesized by adding some of the wet cake (which was initially obtained by removing the solids from liquefied corn mash) to diluted centrate. Some water was also added to dilute the liquid phase held up in the wet cake because this liquid has the same composition as the concentrated centrate. Diluted supernate (17.8 kg), 13.0 kg wet cake (contains ˜18 wt % undissolved mash solids), 5.0 kg H₂O, and 0.83 kg i-BuOH were mixed together yielding 36.6 kg of a slurry containing approximately 6.3 wt % undissolved solids and a liquid phase consisting of approximately 13 wt % liquefied starch and approximately 2.4 wt % i-BuOH (balance H₂O). This slurry mimics the composition of a fermentation broth half way through SSF of corn to i-BuOH at approximately 30% corn loadings because the level of undissolved solids and oligosaccharides found in these types of broths is approximately 6-8 wt % and 10-12 wt %, respectively.

Mass transfer tests were conducted with two different size nozzles at nozzle velocities ranging from 5 ft/s to 22 ft/s using the slurry of diluted centrate and undissolved mash solids as the aqueous phase. Three tests were done using a nozzle that has an ID of 0.76 mm, and three tests were done using a nozzle that has an ID of 2.03 mm. All tests were conducted at 30° C. in the 6-inch diameter column described above using oleyl alcohol as the solvent. The results of the mass transfer tests using the slurry of diluted centrate and undissolved mash solids are shown in Table 3.

TABLE 3

| | 52 | 53 | 54 | 49 | 50 | 51 |
|---|---|---|---|---|---|---|
| | Diluted | Diluted | Diluted | Diluted | Diluted | Diluted |
| | Centrate | Centrate | Centrate | Centrate | Centrate | Centrate |
| | from Liq'd | from Liq'd | from Liq'd | from Liq'd | from Liq'd | from Liq'd |
| | Mash, | Mash, | Mash, | Mash, | Mash, | Mash, |
| | +6.3 wt % | +6.3 wt % | +6.3 wt % | +6.3 wt % | +6.3 wt % | +6.3 wt % |
| | Solids | Solids | Solids | Solids | Solids | Solids |
| MASS TRANSFER TEST CONDITIONS: | | | | | | |
| Aqueous Phase | 35.5 | 35.5 | 32.5 | 31.5 | 30 | 31.6 |
| Volume of Aqueous Phase, L: | | | | | | |
| Solvent Feed Rate, g/min: | 40 | 64 | 157 | 249 | 549 | 853 |
| Superficial Liq. Velocity (Us), ft/hr: | 0.51 | 0.81 | 1.99 | 3.16 | 6.97 | 10.83 |
| Nozzle I.D., mm: | 0.76 | 0.76 | 0.76 | 2.03 | 2.03 | 2.03 |
| Nozzle Velocity, ft/s | 5.7 | 9.1 | 22.3 | 4.9 | 10.9 | 17.0 |
| MASS TRANSFER RESULTS: | | | | | | |
| Initial [i-B] in Aq. Phase, g/L: | 28.1 | 26.0 | 26.2 | 27.6 | 26.3 | 36.8 |
| Final [i-B] in Aq. Phase, g/L: | 26.3 | 23.8 | 14.0 | 24.6 | 13.8 | 16.1 |
| Rich OA collected, kg: | 6.02 | 5.75 | 10.23 | 15.05 | 16.58 | 13.22 |
| [i-B] in OA collected, wt %: | 1.05 | 1.35 | 3.86 | 0.68 | 2.30 | 5.00 |
| Test time, min: | 150 | 90 | 65 | 60 | 30 | 15.5 |
| Overall i-BuOH M.T. Rate, g/L/hr | 0.71 | 1.46 | 11.2 | 3.0 | 25.0 | 80.0 |
| kLa, hr\hat{ }(−1) | 0.03 | 0.06 | 0.83 | 0.12 | 1.55 | 4.45 |
| (kLa/Us) | 0.06 | 0.07 | 0.42 | 0.04 | 0.22 | 0.41 |

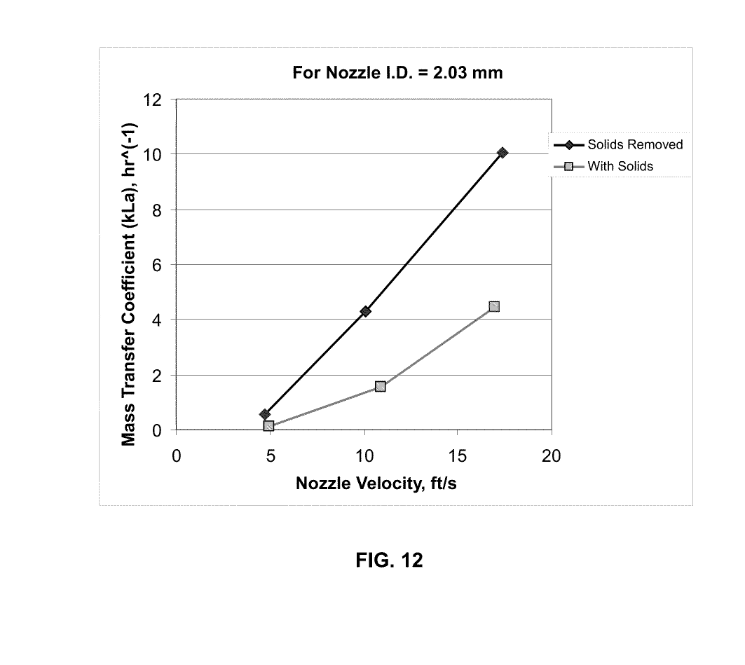

FIG. 12 illustrates the effect of the presence of undissolved corn mash solids on the overall volumetric mass transfer coefficient, k_{L}a, for the transfer of i-BuOH from an aqueous solution of liquefied corn starch (i.e., oligosaccharides) to a dispersion of oleyl alcohol droplets flowing up through a bubble column. The oleyl alcohol was fed to the column through a 2.03 mm ID nozzle. It was discovered that the ratio of the k_{L}a for a system where the solids have been removed to the k_{L}a for a system where the solids have not been removed is 2 to 5 depending on the nozzle velocity for a 2.03 mm nozzle.

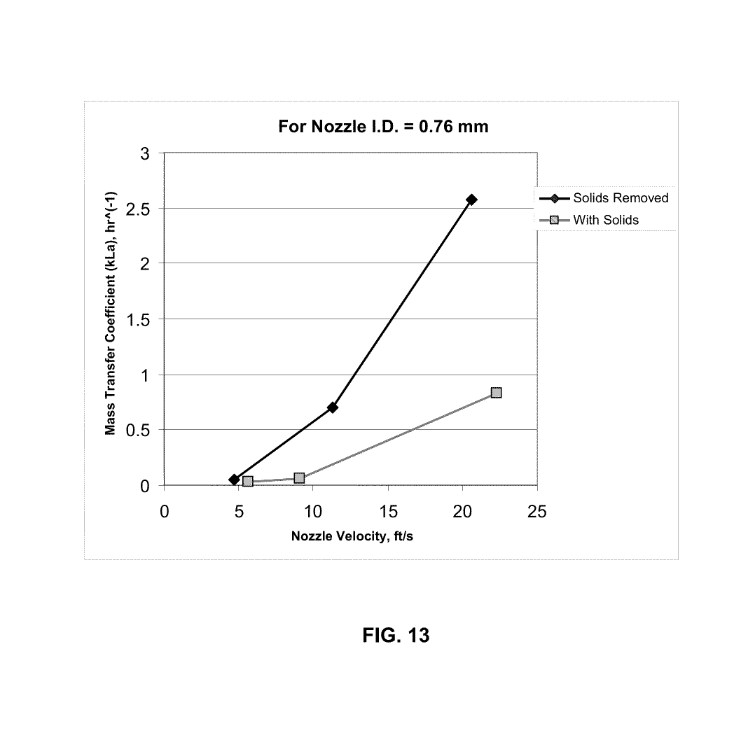

FIG. 13 illustrates the effect of the presence of undissolved corn mash solids on the overall volumetric mass transfer coefficient, k_{L}a, for the transfer of i-BuOH from an aqueous solution of liquefied corn starch (i.e., oligosaccharides) to a dispersion of oleyl alcohol droplets flowing up through a bubble column. The oleyl alcohol was fed to the column through a 0.76 mm ID nozzle. It was discovered that the ratio of the k_{L}a for a system where the solids have been removed to the k_{L}a for a system where the solids have not been removed is 2 to 4 depending on the nozzle velocity for a 0.76 mm nozzle.

### Example 2

### Effect of Removing Undissolved Solids on Phase Separation Between an Aqueous Phase and a Solvent Phase

This example illustrates improved phase separation between an aqueous solution of oligosaccharides derived from liquefied corn mash from which undissolved solids have been removed and a solvent phase as compared to an aqueous solution of oligosaccharides derived from liquefied corn mash from which no undissolved solids have been removed and the same solvent. Both systems contained i-BuOH. Adequate separation of the solvent phase from the aqueous phase is important for liquid-liquid extraction to be a viable separation method for practicing ISPR.

Approximately 900 g of liquefied corn mash was prepared in a 1 L glass, jacketed resin kettle. The kettle was set up with mechanical agitation, temperature control, and pH control. The following protocol was used: mixed ground corn with tap water (26 wt % corn on a dry basis), heated the slurry to 55° C. while agitating, adjusted pH to 5.8 with either NaOH or H₂SO₄, added alpha-amylase (0.02 wt % on a dry corn basis), continued heating to 85° C., adjusted pH to 5.8, held at 85° C. for 2 hr while maintaining pH at 5.8, cool to 25° C.

Pioneer 3335 hybrid corn, whole kernel yellow corn, was used (Pioneer Hi-Bred International, Johnston, Iowa), and it was ground in a hammer-mill using a 1 mm screen. The moisture content of the ground corn was 12 wt %, and the starch content of the ground corn was 71.4 wt % on a dry corn basis. The alpha-amylase enzyme was Liquozyme® SC DS from Novozymes (Franklinton, N.C.). The total amounts of the ingredients used were: 265.9 g ground corn (12% moisture), 634.3 g tap water, and 0.056 g Liquozyme® SC DS. The total amount of liquefied corn mash recovered was 883.5 g.

Part of the liquefied corn mash was used directly, without removing undissolved solids, to prepare the aqueous phase for phase separation tests involving solids. Part of the liquefied corn mash was centrifuged to remove most of the undissolved solids and used to prepare the aqueous phase for phase separation tests involving the absence of solids.

The solids were removed from the mash by centrifugation in a large floor centrifuge. Mash (583.5 g) was centrifuged at 5000 rpm for 20 min at 35° C. yielding 394.4 g centrate and 189.0 g wet cake. It was determined that the centrate contained approximately 0.5 wt % suspended solids, and that the wet cake contained approximately 20 wt % suspended solids. This implies that the original liquefied mash contained approximately 7 wt % suspended solids. This is consistent with the corn loading and starch content of the corn used assuming most of the starch was liquefied. If all of the starch was liquefied, the centrate recovered directly from the centrifuge would have contained approximately 20 wt % dissolved oligosaccharides (liquefied starch) on a solids-free basis.

The objective of the phase separation test was to measure the effect of undissolved solids on the degree of phase separation between a solvent phase and an aqueous phase that simulates a broth that is derived from liquefied corn mash. The aqueous liquid phase contained about 20 wt % oligosaccharides, and the organic phase contained oleyl alcohol (OA) in all tests. Furthermore, i-BuOH was added to all tests to give approximately 25 g/L in the aqueous phase when the phases were at equilibrium. Two shake tests were performed. The aqueous phase for the first test (with solids) was prepared by mixing 60.0 g liquefied corn mash with 3.5 g i-BuOH. The aqueous phase for the second test (solids removed) was prepared by mixing 60.0 g centrate which was obtained from the liquefied corn mash by removing the solids, with 3.5 g i-BuOH. Oleyl alcohol (15.0 g, 80/85% grade, Cognis Corporation, Cincinnati, Ohio) was added to each of the shake test bottles. The oleyl alcohol formed a separate liquid phase on top of the aqueous phase in both bottles resulting in a mass ratio of phases: Aq Phase/Solvent Phase to be about ¼. Both bottles were shaken vigorously for 2 min to intimately contact the aqueous and organic phases and enable the i-BuOH to approach equilibrium between the two phases. The bottles were allowed to set for 1 hr. Photographs were taken at various times (0, 15, 30, and 60 min) to observe the effect of undissolved solids on phase separation in systems that contain an aqueous phase derived from liquefied corn mash, a solvent phase containing oleyl alcohol, and i-BuOH. Time zero (0) corresponds to the time immediately after the two minute shake period was complete.

The degree of separation between the organic (solvent) phase and the aqueous phase as a function of time for the system with solids (from liquefied corn mash) and the system where solids were removed (liquid centrate from liquefied corn mash) appeared about the same in both systems at any point in time. The organic phase was a slightly darker and cloudier, and the interface was a little less distinct (thicker “rag” layer around the interface) for the case with solids. However, for an extractive fermentation where the solvent is operated continuously, the composition of the top of the organic phase is of interest for the process downstream of the extractive fermentation wherein the next step is a distillation.

It may be advantageous to minimize the amount of microorganisms in the top of the organic phase because the microorganisms will be thermally deactivated in the distillation column. It may be advantageous to minimize the amount of undissolved solvents in the top of the organic phase because they could plug the distillation column, foul the reboiler, cause poor phase separation in the solvent/water decanter located at the base of the column, or any combination of the previously mentioned concerns. It may be advantageous to minimize the amount of phase water in the top of the organic phase. Phase water is water that exists as a separate aqueous phase. Additional amounts of aqueous phase will increase the loading and energy requirement in the distillation column. Ten milliliter (10 mL) samples were removed from the top of the organic layers from the “With Solids” and “Solids Removed” bottles, and both samples were centrifuged to reveal and compare the composition of the organic phases in the “With Solids” and “Solids Removed” bottles after 60 min of settling time. The results show that the organic phases at the end of both shake tests contained some undesired phase(s) (both organic phases are cloudy). However, the results also show that the top layer from the phase separation test involving centrate, from which solids were removed, contained essentially no undissolved solids. On the other hand, undissolved solids are clearly seen at the bottom of the 10 mL sample collected from the top of the organic phase of the test involving mash. It was estimated that 3% of the sample collected from the top of the organic layer wash mash solids. If the rich solvent phase exiting the fermentor of an extractive fermentation process contained 3% undissolved solids, one or more of the following problems could occur: loss of significant amount of microorganisms, fouling of solvent column reboiler, plugging of solvent column. The results also show that the top layer from the phase separation test involving centrate contained less phase water. Table 4 shows an estimate of the relative amount of phases that were dispersed throughout the upper “organic” layers in both shake test bottles after 60 min of settling time.

TABLE 4
Approximate composition of organic (top) layer
from shake tests after 60 min

| | | Top Layer |
|---|---|---|
| | Top Layer from “With | from “Solids |
| | Solids” Shake Test | Removed” Shake Test |
| Organic (solvent) Phase: | 82% | 87% |
| Aqueous (water) Phase: | 15% | 13% |
| Undissolved Solids: | 3% | 0% |

This example shows that removing most of the undissolved solids from liquefied corn mash results in improved phase separation after the liquid, aqueous phase obtained from the mash is contacted with a solvent, such as oleyl alcohol. This example shows that the upper phase obtained after phase separation will contain significantly less undissolved solids if the solids are removed first before contacting the liquid part of mash with an organic solvent. This demonstrates advantages of minimizing the undissolved solids content of mash in the upper (“organic”) layer of the phase separation for an extractive fermentation.

Samples were also allowed to sit for several days after completion of sample preparation before repeating the phase separation shake test described in this example. The sample with solids consisted of liquefied corn mash, i-BuOH, and oleyl alcohol, and the sample with solids removed consisted of centrate which was produced by removing most of the undissolved solids from liquefied corn mash, i-BuOH, and oleyl alcohol. The liquefied mash contained approximately 7 wt % suspended solids, and the centrate produced from the mash contained approximately 0.5 wt % suspended solids. If all of the starch in the ground corn was liquefied, the liquid phase in the liquefied mash and the centrate produced from the mash would have contained approximately 20 wt % dissolved oligosaccharides (liquefied starch) on a solids-free basis. Both samples contained oleyl alcohol in an amount to give a mass ratio of phases: Solvent Phase/Aq Phase to be about ¼. Furthermore, i-BuOH was added to all tests to give approximately 25 g/L in the aqueous phase when the phases were at equilibrium.

The objective of the phase separation test was to measure the effect of undissolved solids on the degree of phase separation after the multi-phase mixtures sat at room temperature for several days to mimic the potential change in properties of the system throughout an extractive fermentation. Two shake tests were performed. Both bottles were shaken vigorously for 2 min to intimately contact the aqueous and organic phases. The bottles were allowed to sit for 1 hr. Photographs were taken at various times (0, 2, 5, 10, 20, and 60 min) to observe the effect of undissolved solids on phase separation in these systems which had aged for several days. Time zero (0) corresponds to the time immediately after the bottles were placed on the bench.

Phase separation started to occur in the sample where solids were removed after 2 min. It appeared that almost complete phase separation had occurred in the sample where solids had been removed after only 5-10 min based on the fact that the organic phase occupied approximately 25% of the total volume of the two phase mixture. It would be expected that complete separation would be indicated if the organic phase occupied approximately 20% of the total volume, since that corresponds to the initial ratio of phases. No apparent phase separation occurred in the sample where solids were not removed even after 1 hr.

The composition of the upper phase for both samples was also compared. The composition of the upper phase has implications for the process downstream of the extractive fermentation wherein the next step is a distillation. It is advantageous to minimize the amount of microorganisms in the top of the organic phase because the microorganisms will be thermally deactivated in the distillation column. Another component to minimize in the top of the organic phase is the amount of undissolved solids because the solids could plug the distillation column, foul the reboiler, cause poor phase separation in the solvent/water decanter located at the base of the column, or any combination of the previously mentioned concerns. In addition, another component to minimize in the top of the organic phase is the amount of phase water which is water that exists as a separate aqueous phase, because this additional amount of aqueous phase will increase the loading and energy requirement in the subsequent distillation column.

Ten milliliter (10 mL) samples were removed from the top of the organic layers from the “With Solids” and “Solids Removed” bottles, and both samples were centrifuged to reveal and compare the composition of the organic phases in the “With Solids” and “Solids Removed” bottles after 60 min of settling time. The composition of the sample collected from the top of the “With Solids” sample confirms that essentially no phase separation occurred in that sample within 60 min. Specifically, the ratio of the solvent phase to total aqueous phase (aqueous liquid+suspended solids) in the sample collected from the top of the “With Solids” shake test bottle is approximately ¼ w/w, which is the same ratio used to prepare the sample prior to the test. Also, the amount of undissolved solids in the sample collected from the top of the “With Solids” shake test bottle is approximately the same as what is found in liquefied corn mash, which shows that essentially no solids settled in this shake test bottle within 60 min. On the other hand, the top layer from the phase separation test involving centrate (“Solids Removed”) from which solids were removed, contained essentially no undissolved solids. The results also show that the top layer from the phase separation test involving centrate contained less phase water. This is indicated by the fact that the ratio of the solvent phase to aqueous phase in that sample bottle is approximately 1/1 w/w, which shows that the organic phase was enriched with solvent (oleyl alcohol) in the test where solids were removed. Table 5 shows an estimate of the relative amount of phases that were dispersed throughout the upper “organic” layers in both shake test bottles after 60 min of settling time.

TABLE 5
Approximate composition of organic (top) layer
from shake tests after 60 min

| | | Top Layer |
|---|---|---|
| | Top Layer from “With | from “Solids |
| | Solids” Shake Test | Removed” Shake Test |
| Organic (solvent) Phase: | 19% | 50% |
| Aqueous (water) Phase: | 47% | 50% |
| Undissolved Solids: | 34% | 0% |

This example shows that removing undissolved solids from liquefied corn mash that contains i-BuOH, contacting it with a solvent phase, letting it set for several days, and mixing the phases again results in improved phase separation when compared to a sample where undissolved solids were not removed from the liquefied mash. In fact, this example shows that essentially no phase separation occurs in the sample where undissolved solids were not removed even after 60 min. This example shows that the upper phase obtained after phase separation contains significantly less undissolved solids if the solids are removed first before contacting the liquid part of mash with an organic solvent. This is important because two of the most important species that should be minimized in the upper (“organic”) layer of the phase separation for an extractive fermentation are the level of microorganisms and the level of undissolved solids from mash. The previous example showed that removing solids from liquefied corn mash results in improved phase separation shortly after the aqueous phase is contacted with a solvent phase. This would allow extractive fermentation to be viable at earlier times in the fermentation. This example also shows that removing solids from liquefied corn mash results in improved phase separation in aged samples that contain an aqueous phase (oligosaccharide solution with solids removed) that has been contacted with a solvent phase. This would also allow extractive fermentation to be viable at later times in the fermentation.

### Example 3

### Effect of Removing Undissolved Solids on the Loss of ISPR Extraction Solvent—Disk Stack Centrifuge

This example demonstrates the potential for reducing solvent losses via DDGS generated by the extractive fermentation process by removing undissolved solids from the corn mash prior to fermentation using a semi-continuous disk-stack centrifuge.

Approximately 216 kg liquefied corn mash was prepared in a jacketed stainless steel reactor. The reactor was set up with mechanical agitation, temperature control, and pH control. The protocol used was as follows: mixed ground corn with tap water (25 wt % corn on a dry basis), heated the slurry to 55° C. while agitating at 400 rpm, adjusted pH to 5.8 with either NaOH or H₂SO₄, added alpha-amylase (0.02 wt % on a dry corn basis), continued heating to 85° C., adjusted pH to 5.8, held at 85° C. for 30 min while maintaining pH at 5.8, heated to 121° C. using live steam injection, held at 121° C. for 30 min to simulate a jet cooker, cooled to 85° C., adjusted pH to 5.8, added second charge of alpha-amylase (0.02 wt % on a dry corn basis), held at 85° C. for 60 min while maintaining pH at 5.8 to complete liquefaction. The mash was then cooled to 60° C. and transferred to the centrifuge feed tank.

Pioneer 3335 hybrid corn, whole kernel yellow corn, was used (Pioneer Hi-Bred International, Johnston, Iowa), and it was ground in a hammer-mill using a 1 mm screen. The moisture content of the ground corn was 12 wt %, and the starch content of the ground corn was 71.4 wt % on a dry corn basis. The alpha-amylase enzyme was Liquozyme® SC DS from Novozymes (Franklinton, N.C.). The amounts of the ingredients used were: 61.8 kg ground corn (12% moisture), 147.3 kg tap water, a solution of 0.0109 kg Liquozyme® SC DS in 1 kg water for first alpha-amylase charge, another solution of 0.0109 kg Liquozyme® SC DS in 1 kg water for second alpha-amylase charge (after the cook stage). About 5 kg H₂O was added to the batch via steam condensate during the cook stage. A total of 0.25 kg NaOH (12.5 wt %) and 0.12 kg H₂SO₄ (12.5 wt %) were added throughout the run to control pH. The total amount of liquefied corn mash recovered was 216 kg.

The composition of the final liquefied corn mash slurry was estimated to be approximately 7 wt % undissolved solids and 93 wt % liquid. The liquid phase contained about 19 wt % (190 g/L) liquefied starch (soluble oligosaccharides). The rheology of the mash is important regarding the ability to separate the slurry into its components. The liquid phase in the mash was determined to be a Newtonian fluid with a viscosity of about 5.5 cP at 30° C. The mash slurry was determined to be a shear-thinning fluid with a bulk viscosity of about 10 to 70 cP at 85° C. depending on shear rate.

The liquefied mash (209 kg, 190 L) was centrifuged using a disk-stack split-bowl centrifuge (Alfa Laval Inc., Richmond, Va.). The centrifuge operated in semi-batch mode with continuous feed, continuous centrate outlet, and batch discharge of the wet cake. Liquefied corn mash was continuously fed at a rate of 1 L/min, clarified centrate was removed continuously, and wet cake was periodically discharged every 4 min. To determine an appropriate discharge interval for the solids from the disk stack, a sample of the mash to be fed to the disk stack was centrifuged in a high-speed lab centrifuge. Mash (48.5 g) was spun at 11,000 rpm (about 21,000 g for about 10 min at room temperature. Clarified centrate (36.1 g) and 12.4 g pellet (wet cake) were recovered. It was determined that the clarified centrate contained about 0.3 wt % undissolved solids and that the pellet (wet cake) contained about 27 wt % undissolved solids. Based on this data, a discharge interval of 4 min was chosen for operation of the disk stack centrifuge.

The disk stack centrifuge was operated at 9000 rpm (6100 g) with a liquefied corn mash feed rate of 1 L/min and at about 60° C. Mash (209 kg) was separated into 155 kg clarified centrate and 55 kg wet cake. The split, defined as (amount of centrate)/(amount of mash fed), achieved by the semi-continuous disk stack was similar to the split achieved in the batch centrifuge. The split for the disk stack semi-batch centrifuge operating at 6100 g, 1 L/min feed rate, and 4 min discharge interval was (155 kg/209 kg)=74%, and the split for the lab batch centrifuge operating at 21,000 g for 10 min was (36.1 g/48.5 g)=74%.

A 45 mL sample of the clarified centrate recovered from the disk stack centrifuge was spun down in a lab centrifuge at 21,000 g for 10 min to estimate the level of suspended solids in the centrate. About 0.15-0.3 g undissolved solids were recovered from the 45 mL of centrate. This corresponds to 0.3-0.7 wt % undissolved solids in the centrate which is about a ten-fold reduction in undissolved solids from mash fed to the centrifuge. It is reasonable to assume that the ISPR extraction solvent losses via DDGS could be reduced by about an order of magnitude if the level of undissolved solids present in extractive fermentation is reduced by an order of magnitude using some solid/liquid separation device or combination of devices to remove suspended solids from the corn mash before fermentation. Minimizing solvent losses via DDGS is an important factor in the economics and DDGS quality for an extractive fermentation process.

### Example 4

### Effect of Removing Undissolved Solids on the Loss of ISPR Extraction Solvent—Bottle Spin Test

This example demonstrates the potential for reducing solvent losses via DDGS generated by the extractive fermentation process by removing undissolved solids from the corn mash prior to fermentation using a centrifuge.

A lab-scale bottle spin test was performed using liquefied corn mash. The test simulates the operating conditions of a typical decanter centrifuge used to remove undissolved solids from whole stillage in a commercial ethanol plant. Decanter centrifuges in commercial ethanol plants typically operate at a relative centrifugal force (RCF) of about 3000 g and a whole stillage residence time of about 30 seconds. These centrifuges typically remove about 90% of the suspended solids in whole stillage which contains about 5% to 6% suspended solids (after the beer column), resulting in thin stillage which contains about 0.5% suspended solids.

Liquefied corn mash was made according to the protocol described in Example 2. About 10 mL mash was placed in a centrifuge tube. The sample was centrifuged at an RCF of about 3000 g (4400 rpm rotor speed) for a total of 1 min. The sample spent about 30-40 seconds at 3000 g and a total of 20-30 seconds at speeds less than 3000 g due to acceleration and deceleration of the centrifuge. The sample temperature was about 60° C.

The mash (10 mL) which contained about 7 wt % suspended solids was separated into about 6.25 mL clarified centrate and 3.75 mL wet cake (pellet at the bottom of the centrifuge tube). The split, defined as (amount of centrate)/(amount of original mash charged), achieved by the bottle spin test was about 62%. It was determined that the clarified centrate contained about 0.5 wt % suspended solids which is more than a ten-fold decrease in suspend solids compared to the level of suspended solids in the original mash. It was also determined that the clarified pellet contained about 18 wt % suspended solids.

Table 6 summarizes the suspended (undissolved) solids mass balance for the bottle spin test at conditions representative of the operation of a decanter centrifuge to convert whole stillage to thin stillage in a commercial ethanol process. All values given in Table 6 are approximate.

TABLE 6

| | Volume, mL | Suspend Solids, wt % |
|---|---|---|
| Liquefied Corn Mash charge: | 10 | 7% |
| Clarified Centrate: | 6.25 | 0.5% |
| Wet Cake (pellet): | 3.75 | 18% |

Performance Summary

| | |
|---|---|
| Split: | 62% |
| Centrate Clarity: | 0.5 wt % suspended solids |
| Cake (pellet) Dryness: | 18 wt % suspended solids |
| % Removal of Suspended | 95% removal from liquefied mash |
| Solids: | |

It was also determined that the centrate contained about 190 g/L dissolved oligosaccharides (liquefied starch). This is consistent with the assumption that most of the starch in the ground corn was liquefied (i.e., hydrolyzed to soluble oligosaccharides) in the liquefaction process based on the corn loading used (about 26 wt % on a dry corn basis) and the starch content of the corn used to produce the liquefied mash (about 71.4 wt % starch on a dry corn basis). Hydrolyzing most of the starch in the ground corn at a 26% dry corn loading will result in about 7 wt % suspended (undissolved) solids in the liquefied corn mash charged to the centrifuge used for the bottle spin test.

The fact that the clarified centrate contained only about 0.5 wt % undissolved solids indicates that the conditions used in the bottle spin test resulted in more than a ten-fold reduction in undissolved solids from mash charged. If this same solids removal performance could be achieved by a continuous decanter centrifuge before fermentation, it is reasonable to assume that the ISPR extraction solvent losses in the DDGS could be reduced by about an order of magnitude. Minimizing solvent losses via DDGS is an important factor in the economics and DDGS quality for an extractive fermentation process.

### Example 5

### Removal of Corn Oil by Removing Undissolved Solids

This example demonstrates the potential to remove and recover corn oil from corn mash by removing the undissolved solids prior to fermentation. The effectiveness of the extraction solvent may be improved if corn oil is removed via removal of the undissolved solids. In addition, removal of corn oil via removal of the undissolved solids may also minimize any reduction in solvent partition coefficient and potentially resulting an improved extractive fermentation process.

Approximately 1000 g liquefied corn mash was prepared in a 1 L glass, jacketed resin kettle. The kettle was set up with mechanical agitation, temperature control, and pH control. The following protocol was used: mixed ground corn with tap water (26 wt % corn on a dry basis), heated the slurry to 55° C. while agitating, adjusted pH to 5.8 with either NaOH or H₂SO₄, added alpha-amylase (0.02 wt % on a dry corn basis), continued heating to 85° C., adjusted pH to 5.8, held at 85° C. for 2 hr while maintaining pH at 5.8, cool to 25° C.

Pioneer 3335 hybrid corn, whole kernel yellow corn, was used (Pioneer Hi-Bred International, Johnston, Iowa), and it was ground in a hammer-mill using a 1 mm screen. The moisture content of the ground corn was about 11.7 wt %, and the starch content of the ground corn was about 71.4 wt % on a dry corn basis. The alpha-amylase enzyme was Liquozyme® SC DS from Novozymes (Franklinton, N.C.). The total amounts of the ingredients used were: 294.5 g ground corn (11.7% moisture), 705.5 g tap water, and 0.059 g Liquozyme® SC DS. Water (4.3 g) was added to dilute the enzyme, and a total of 2.3 g of 20% NaOH solution was added to control pH. About 952 g of mash was recovered.

The liquefied corn mash was centrifuged at 5000 rpm (7260 g) for 30 min at 40° C. to remove the undissolved solids from the aqueous solution of oligosaccharides. Removing the solids by centrifugation also resulted in the removal of free corn oil as a separate organic liquid layer on top of the aqueous phase. Approximately 1.5 g of corn oil was recovered from the organic layer floating on top of the aqueous phase. It was determined by hexane extraction that the ground corn used to produce the liquefied mash contained about 3.5 wt % corn oil on a dry corn basis. This corresponds to about 9 g corn oil fed to the liquefaction process with the ground corn.

After recovering the corn oil from the liquefied mash, the aqueous solution of oligosaccharides was decanted away from the wet cake. About 617 g liquefied starch solution was recovered leaving about 334 g wet cake. The wet cake contained most of the undissolved solids that were in the liquefied mash. The liquefied starch solution contained about 0.2 wt % undissolved solids. The wet cake contained about 21 wt % undissolved solids. The wet cake was washed with 1000 g tap water to remove the oligosaccharides still in the cake. This was done by mixing the cake with the water to form a slurry. The slurry was then centrifuged under the same conditions used to centrifuge the original mash in order to recover the washed solids. Removing the washed solids by centrifuging the wash slurry also resulted in the removal of some additional free corn oil that must have remained with the original wet cake produced from the liquefied mash. This additional corn oil was observed as a separate, thin, organic liquid layer on top of the aqueous phase of the centrifuged wash mixture. Approximately 1 g of additional corn oil was recovered from the wash process.

The wet solids were washed two more times using a 1000 g tap water each time to remove essentially all of the liquefied starch. No visible additional corn oil was removed from the 2^{nd} and 3^{rd} water washes of the mash solids. The final washed solids were dried in a vacuum oven overnight at 80° C. and about 20 inches Hg vacuum. The amount of corn oil remaining in the dry solids, presumably still in the germ, was determined by hexane extraction. A sample of relatively dry solids (3.60 g, about 2 wt % moisture) contained 0.22 g corn oil. This result corresponds to 0.0624 g corn oil/g dry solids. This was for washed solids which means there are no residual oligosaccharides in the wet solids. After centrifuging the liquefied corn mash to separate the layer of free corn oil and the aqueous solution of oligosaccharides from the wet cake, it was determined that about 334 g wet cake containing about 21 wt % undissolved solids remained. This corresponds to the wet cake comprising about 70.1 g undissolved solids. At 0.0624 g corn oil/g dry solids, the solids in the wet cake should contain about 4.4 g corn oil.

In summary, approximately 1.5 g free corn oil was recovered by centrifuging the liquefied mash. An additional 1 g free corn oil was recovered by centrifuging the first (water) wash slurry which was generated to wash the original wet cake produced from the mash. Finally, it was determined that the washed solids still contained about 4.4 g corn oil. It was also determined that the corn charged to the liquefaction contained about 9 g corn oil. Therefore, a total of 6.9 g corn oil was recovered from the following process steps: liquefaction, removal of solids from liquefied mash, washing of the solids from the mash, and the final washed solids. Consequently, approximately 76% of the total corn oil in the corn fed to liquefaction was recovered during the liquefaction and solids removal process described here.

### Example 6

### Extractive Fermentation Using Mash and Centrate as the Sugar Source

This example describes extractive fermentations performed using corn mash and corn mash centrate as the sugar source. Corn mash centrate was produced by removing undissolved solids from the corn mash prior to fermentation. Four extractive fermentations were conducted side-by side, two with liquefied corn mash as the sugar source (solids not removed) and two with liquefied mash centrate (aqueous solution of oligosaccharides) obtained by removing most of the undissolved solids from liquefied corn mash. Oleyl alcohol (OA) was added to two of the fermentations, one with solids and one with solids removed, to extract the product (i-BuOH) from the broth as it was formed. A mixture of corn oil fatty acids (COFA) was added to the other two of the fermentations, one with solids and one with solids removed, to extract the product from the broth as it was formed. The COFA was made by hydrolyzing corn oil. The purpose of these fermentations was to test the effect of removing solids on phase separation between the solvent and broth and to measure the amount of residual solvent trapped in the undissolved solids recovered from fermentation broths where solids were or were not removed.

### Preparation of Liquefied Corn Mash

Approximately 31 kg liquefied corn mash was prepared in a 30 L jacketed glass resin kettle. The reactor was outfitted with mechanical agitation, temperature control, and pH control. The protocol used was as follows: mix ground corn with tap water (40 wt % corn on a dry basis), heat the slurry to 55° C. while agitating at 250 rpm, adjust pH to 5.8 with either NaOH or H₂SO₄, add a dilute aqueous solution of alpha-amylase (0.16 wt % on a dry corn basis), hold at 55° C. for 60 min, heat to 95° C., adjust pH to 5.8, hold at 95° C. for 120 min while maintaining pH at 5.8 to complete liquefaction. The mash was transferred into sterile centrifuge bottles to prevent contamination.

The corn used was whole kernel yellow corn (Pioneer Hi-Bred International, Johnston, Iowa), and it was ground in a pilot-scale hammer-mill using a 1 mm screen. The moisture content of the ground corn was about 12 wt %, and the starch content of the ground corn was about 71.4 wt % on a dry corn basis. The alpha-amylase enzyme used was Spezyme® Fred-L (Genencor®, Palo Alto, Calif.). The amounts of the ingredients used were: 14.1 kg ground corn (12% moisture), 16.9 kg tap water, a solution of alpha-amylase consisting of 19.5 g Spezyme® Fred-L in 2.0 kg water. The alpha-amylase was sterile filtered. A total of 0.21 kg NaOH (17 wt %) was added throughout the run to control pH.

It was estimated that the liquefied corn mash contained approximately 28 wt % (about 280 g/L) of liquefied starch based on the corn loading used, starch content of the corn, and assuming all the starch was hydrolyzed during liquefaction. The mash was prepared with a higher concentration of oligosaccharides than was desired in the fermentations to allow for dilution when adding the nutrients, inoculum, glucoamylase, and base to the initial fermentation broth. After dilution by addition of nutrients, inoculum, glucoamylase, and base, the expected total initial soluble sugars in the mash (solids not removed) was about 250 g/L.

About 13.9 kg liquefied mash was centrifuged using a bottle centrifuge which contained six 1 L bottles. The centrifuge was operated at 5000 rpm (7260 RCF) for 20 min at room temperature. The mash was separated into about 5.5 kg clarified centrate and about 8.4 kg wet cake (the pellet at the bottom of the centrifuge bottles). The split, defined as (amount of centrate)/(amount of mash fed), was about (5.5 kg/13.9 kg)=40%.

Solids were not removed from the mash charged to the 2010Y034 and 2010Y036 fermentations described below. The centrate charged to fermentations 2010Y033 and 2010Y035 (also described below) was produced by removing by centrifugation most of the suspended solids from mash according to the protocols above.

### General Methods for Fermentation

Seed Flask Growth

A Saccharomyces cerevisiae strain (with deletions of pdc1, pdc5, and pdc6) that was engineered to produce isobutanol from a carbohydrate source was grown to 0.55-1.1 g/L dcw (OD₆₀₀ 1.3-2.6—Thermo Helios α Thermo Fisher Scientific Inc., Waltham, Mass.) in seed flasks from a frozen culture. The Saccharomyces cerevisiae strain is described in U.S. Patent Application Publication No. 2012/0164302, incorporated herein by reference. The culture was grown at 26° C. in an incubator rotating at 300 rpm. The frozen culture was previously stored at −80° C. The composition of the first seed flask medium was:
3.0 g/L dextrose
3.0 g/L ethanol, anhydrous
3.7 g/L ForMedium™ Synthetic Complete Amino Acid (Kaiser) Drop-Out: without HIS, without URA (Reference No. DSCK162CK)
6.7 g/L Difco Yeast Nitrogen Base without amino acids (No. 291920).

Twelve milliliters (12 mL) from the first seed flask culture was transferred to a 2 L flask and grown at 30° C. in an incubator rotating at 300 rpm. The second seed flask has

220 mL of the following medium:
30.0 g/L dextrose
5.0 g/L ethanol, anhydrous
3.7 g/L ForMedium™ Synthetic Complete Amino Acid (Kaiser) Drop-Out: without HIS, without URA (Reference No. DSCK162CK)
6.7 g/L Difco Yeast Nitrogen Base without amino acids (No. 291920)
0.2M MES Buffer titrated to pH 5.5-6.0.

The culture was grown to 0.55-1.1 g/L dcw (OD₆₀₀ 1.3-2.6). An addition of 30 mL of a solution containing 200 g/L peptone and 100 g/L yeast extract was added at this cell concentration. Then an addition of 300 mL of 0.2 uM filter sterilized, 90-95% oleyl alcohol (Cognis Corporation, Cincinnati, Ohio) was added to the flask. The culture continued to grow to >4 g/L dcw (OD₆₀₀>10) before being harvested and added to the fermentation.

### Fermentation Preparation

Initial Fermentor Preparation

A glass jacked, 2 L fermentor (Sartorius AG, Goettingen, Germany) was charged with liquefied mash either with or without solids (centrate). A pH probe (Hamilton Easyferm Plus K8, part number: 238627, Hamilton Bonaduz AG, Bonaduz, Switzerland) was calibrated through the Sartorius DCU-3 Control Tower Calibration menu. The zero was calibrated at pH=7. The span was calibrated at pH=4. The probe was then placed into the fermentor, through the stainless steel head plate. A dissolved oxygen probe (pO₂ probe) was also placed into the fermentor through the head plate. Tubing used for delivering nutrients, seed culture, extracting solvent, and base were attached to the head plate and the ends were foiled. The entire fermentor was placed into an autoclave (Steris Corporation, Mentor, Ohio) and sterilized in a liquid cycle for 30 min.

The fermentor was removed from the autoclave and placed on a load cell. The jacket water supply and return line was connected to the house water and clean drain, respectively. The condenser cooling water in and water out lines were connected to a 6-L recirculating temperature bath running at 7° C. The vent line that transfers the gas from the fermentor was connected to a transfer line that was connected to a Thermo mass spectrometer (Prima dB, Thermo Fisher Scientific Inc., Waltham, Mass.). The sparger line was connected to the gas supply line. The tubing for adding nutrients, extract solvent, seed culture, and base was plumbed through pumps or clamped closed. The autoclaved material, 0.9% w/v NaCl was drained prior to the addition of liquefied mash.

Lipase Treatment Post-Liquefaction

The fermentor temperature was set to 55° C. instead of 30° C. after the liquefaction cycle was complete. The pH was manually controlled at pH=5.8 by making bolus additions of acid or base when needed. A lipase enzyme stock solution was added to the fermentor to a final lipase concentration of 10 ppm. The fermentor was held at 55° C., 300 rpm, and 0.3 slpm N₂ overlay for >6 hr. After the lipase treatment was complete the fermentor temperature was set to 30° C.

Nutrient Addition Prior to Inoculation

Added 7.0 mL/L (post-inoculation volume) of ethanol (200 proof, anhydrous) just prior to inoculation. Added thiamine to 20 mg/L final concentration just prior to inoculation. Added 100 mg/L nicotinic acid just prior to inoculation.

Fermentor Inoculation

The fermentor pO₂ probe was calibrated to zero while N₂ was being added to the fermentor. The fermentor pO₂ probe was calibrated to its span with sterile air sparging at 300 rpm. The fermentor was inoculated after the second seed flask was >4 g/L dcw. The shake flask was removed from the incubator/shaker for 5 min allowing a phase separation of the oleyl alcohol phase and the aqueous phase. The aqueous phase (55 mL) was transferred to a sterile, inoculation bottle. The inoculum was pumped into the fermentor through a peristaltic pump.

Oleyl Alcohol or Corn Oil Fatty Acids Addition after Inoculation

Added 1 L/L (post-inoculation volume) of oleyl alcohol or corn oil fatty acids immediately after inoculation

### Fermentor Operating Conditions

The fermentor was operated at 30° C. for the entire growth and production stages. The pH was allowed to decrease from a pH between 5.7-5.9 to a control set-point of 5.2 without adding any acid. The pH was controlled for the remainder of the growth and production stage at a pH=5.2 with ammonium hydroxide. Sterile air was added to the fermentor, through the sparger, at 0.3 slpm for the remainder of the growth and production stages. The pO₂ was set to be controlled at 3.0% by the Sartorius DCU-3 Control Box PID control loop, using stir control only, with the stirrer minimum being set to 300 rpm and the maximum being set to 2000 rpm. The glucose was supplied through simultaneous saccharification and fermentation of the liquefied corn mash by adding a α-amylase (glucoamylase). The glucose was kept excess (1-50 g/L) for as long as starch was available for saccharification.

### Sample A

Experiment identifier 2010Y033 included: Seed Flask Growth method, Initial Fermentor Preparation method with corn mash that excludes solids, Lipase Treatment Post-Liquefaction, Nutrient Addition Prior to Inoculation method, Fermentor Inoculation method, Fermentor Operating Conditions method, and all of the Analytical methods. Corn oil fatty acid was added in a single batch between 0.1-1.0 hr after inoculation.

### Sample B

Experiment identifier 2010Y034 included: Seed Flask Growth method, Initial Fermentor Preparation method with corn mash that includes solids, Lipase Treatment Post-Liquefaction, Nutrient Addition Prior to Inoculation method, Fermentor Inoculation method, Fermentor Operating Conditions method, and all of the Analytical methods. Corn oil fatty acid was added in a single batch between 0.1-1.0 hr after inoculation.

### Sample C

Experiment identifier 2010Y035 included: Seed Flask Growth method, Initial Fermentor Preparation method with corn mash that excludes solids, Nutrient Addition Prior to Inoculation method, Fermentor Inoculation method, Fermentor Operating Conditions method, and all of the Analytical methods. Oleyl alcohol was added in a single batch between 0.1-1.0 hr after inoculation.

### Sample D

Experiment identifier 2010Y036 included: Seed Flask Growth method, Initial Fermentor Preparation method with corn mash that includes solids, Nutrient Addition Prior to Inoculation method, Fermentor Inoculation method, Fermentor Operating Conditions method, and all of the Analytical methods. Oleyl alcohol was added in a single batch between 0.1-1.0 hr after inoculation. Results for Samples A-D are shown in Table 7.

TABLE 7
Fermentation conditions and results for Samples A-D

| | | | Post - | | | | |
|---|---|---|---|---|---|---|---|
| | | | Liquefaction | | Glucose | g/kg | |
| | | | Undissolved | | Equivalents | glucose | Effective |
| | Experimental | Active | Solids | Extracting | Charged | consumed | isobutanol |
| Sample | ID | Lipase | Removed | Solvent | g/kg | at EOR | g/L |
| A | 2010Y033 | Yes | Yes | Corn oil | 257 | 257 | 30.9 |
| | | | | fatty acids | | | |
| B | 2010Y034 | Yes | No | Corn oil | 239 | 239 | 17.3 |
| | | | | fatty acids | | | |
| C | 2010Y035 | No | Yes | Oleyl | 263 | 72 | 15.7 |
| | | | | alcohol | | | |
| D | 2010Y036 | No | No | Oleyl | 241 | 101 | 20 |
| | | | | alcohol | | | |

### Example 7

### Effect of Removing Undissolved Solids from the Fermentor Feed on Improvement in Fermentor Volume Efficiency

This example demonstrates the effect of removing undissolved solids from the mash prior to fermentation on fermentor volume efficiency. Undissolved solids in corn mash occupy at least 5% of the mash volume depending on corn loading and content starch content. Removing solids before fermentation enables at least 5% more sugar to be charged to the fermentor thus increasing batch productivity.

It was estimated that the liquefied corn mash produced in Example 5 contained approximately 28 wt % (280 g/L) liquefied starch based on the corn loading used (40% dry corn basis), starch content of the corn (71.4% dry corn basis), and assuming all the starch was hydrolyzed to soluble oligosaccharides during liquefaction. The mash was prepared with a higher concentration of oligosaccharides than was desired in the fermentations to allow for dilution when adding the nutrients, inoculum, glucoamylase, and base to the initial fermentation broth. The mash was diluted by approximately 10% after adding these ingredients. Therefore, the expected concentration of liquefied starch in the mash (including solids) at the beginning of fermentations 2010Y034 and 2010Y036 was about 250 g/L. The actual glucose equivalents charged to the 2010Y034 and 2010Y036 fermentations was measured to be 239 g/kg and 241 g/kg, respectively. By comparison, the glucose equivalents charged to the 2010Y033 and 2010Y035 fermentations was measured to be 257 g/kg and 263 g/kg, respectively. Note that the feed to these fermentations was centrate (mash from which most of the solids had been removed). Approximately 1.2 L of the sugar source (mash or centrate) was charged to each fermentation. Therefore, based on this data, approximately 8.3% more sugar was charged to the fermentors which used centrate (2010Y033 and 2010Y035) compared to mash (2010Y034 and 2010Y036). These results demonstrate that removing undissolved solids from corn mash prior to fermentation can result in a significant increase in sugar charged per unit volume. This implies that when solids are present, they occupy valuable fermentor volume. If solids are removed from the feed, more sugar may be added (“fit”) to the fermentor due to the absence of undissolved solids. This example demonstrates that fermentor volume efficiency can be significantly improved by removing undissolved solids from the mash prior to fermentation.

### Example 8

### Effect of Removing Undissolved Solids on Phase Separation Between the Extraction Solvent and the Broth—Extractive Fermentation

This example demonstrates improved separation between the solvent phase and the broth phase during and after an extractive fermentation process by removing undissolved solids from the corn mash prior to fermentation. Two extractive fermentations were conducted side-by side, one with liquefied corn mash as the sugar source (solids not removed) and one with centrate (aqueous solution of oligosaccharides) which was generated by removing most of the undissolved solids from liquefied corn mash. Oleyl alcohol (OA) was added to both fermentations to extract the product (i-BuOH) from the broth as it was formed. The fermentation broth referred to in this example where solids were not removed from the feed (used corn mash) was 2010Y036 as described in Example 6. The fermentation broth referred to in this example where solids were removed from the feed (used centrate produced from corn mash) was 2010Y035 as described in Example 6. Oleyl alcohol was the extraction solvent used in both fermentations. The rate and degree of phase separation was measured and compared throughout the fermentations as well as for the final fermentation broths. Adequate phase separation in an extractive fermentation process can lead to minimal loss of the microorganism and minimal solvent losses as well lower capital and operating cost of downstream processing.

### Phase Separation Between Solvent and Broth Phases During Fermentation

Approximately 10 mL samples were pulled from each fermentor at 5.3, 29.3, 53.3, and 70.3 hr, and phase separation was compared for the samples from the fermentation where solids were removed (2010Y035) from the samples and where solids were not removed (2010Y036). Phase separation was observed and compared for all samples from all run times by allowing the samples to set for about 2 hr and tracking the position of the liquid-liquid interface. Essentially no phase separation was observed for any of the samples pulled from the fermentation where solids were not removed. Phase separation was observed for all samples from the fermentation where solids were removed from the liquefied corn mash prior to fermentation. Separation started to occur within about 10-15 min of pulling the samples from the run where solids were removed for all fermentation times and continued to improve over a 2 hr period of time. Phase separation started to occur in the sample pulled at 5.3 hr fermentation run time from the centrate fermentation (solids removed) after about 7 min of settling time. Phase separation started to occur in the sample pulled at 53.3 hr from the centrate fermentation (solids removed) after about 17 min of settling time.

FIG. 13 is a plot of the position of the liquid-liquid interface in the fermentation sample tubes as a function of (gravity) settling time. The data is for the samples pulled from the extractive fermentation where centrate was fed (solids removed from corn mash) as the sugar source and oleyl alcohol was the ISPR extraction solvent (run 2010Y035 in Example 6). The phase separation data in this plot is for samples pulled at 5.3, 29.3, 53.3, and 70.3 hr run time from fermentation 2010Y035. The interface position is reported as a percentage of the total broth height in the sample tube. For example, the interface position in the sample pulled at 5.3 hr run time from the 2010Y035 fermentation (centrate/oleyl alcohol) increased from the bottom of the sample tube (no separation) to 3.5 mL after 120 min of settling time. There was about 10 mL of total broth in that particular sample tube. Therefore, the interface position for that sample was reported as 35% in FIG. 13. Similarly, the interface position in the sample pulled at 53.3 hr run time from the 2010Y035 fermentation (centrate/oleyl alcohol) increased from the bottom of the sample tube (no separation) to about 3.9 mL after 125 min of settling time. There was about 10 mL of total broth in that particular sample tube. Therefore, the interface position for that sample was reported as 39% in FIG. 13.

Phase Separation Between Solvent and Broth Phases after Completing Fermentation

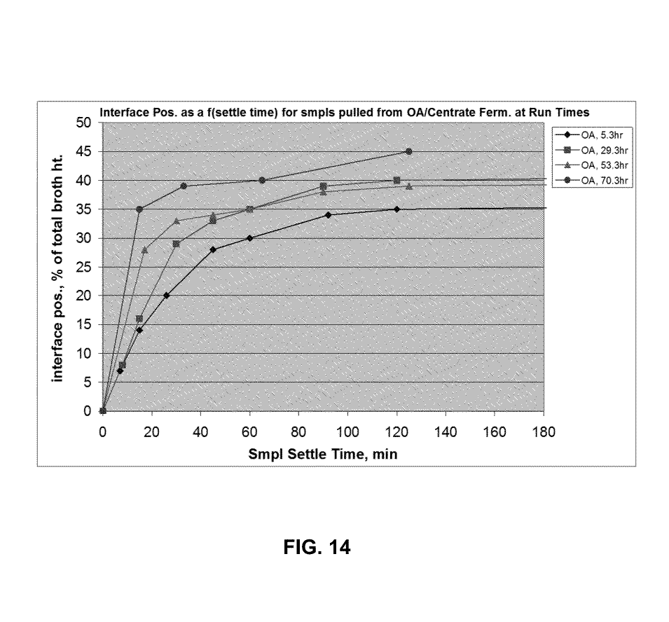

After 70 hr of run time, the fermentations were stopped, and the two broths from the oleyl alcohol extractive fermentations were transferred to separate 2 L glass graduated cylinders. The separation of the solvent and broth phases were observed and compared. Almost no phase separation was observed after about 3 hr for the broth where solids were not removed prior to fermentation (run 2010Y036). Phase separation was observed for the broth where solids were removed from the liquefied corn mash prior to fermentation (run 2010Y035). Separation started to occur after about 15 min of settling time and continued to improve over a 3 hr period of time. After 15 min, a liquid-liquid interface was established at a level that was about 10% of the total height of the two phase mixture. This indicates that the aqueous phase splits out from the dispersion first and starts to accumulate at the bottom of the mixture. As time proceeded, more aqueous phase accumulated at the bottom of the mixture causing the position of the interface to rise. After about 3 hr of settling time, the interface had increased to a level that was about 40% of the total height of the two phase mixture. This indicates that almost complete phase separation had occurred after about 3 hr of (gravity) settling time for the final two phase broth where solids were removed based on the amounts of centrate and oleyl alcohol initially charged to the fermentation. Approximately equal volumes of initial centrate and solvent were charged to both fermentations. Approximately 1.2 L of liquefied corn mash and approximately 1.1 L of oleyl alcohol were charged to fermentation 2010Y036. Approximately 1.2 L of centrate, which was produced from the same batch of mash, and approximately 1.1 L of oleyl alcohol were charged to fermentation 2010Y035. After accounting for the fact that approximately 100 g/kg of the initial sugar in the aqueous phase was consumed and the fact that about 75% of the i-BuOH produced was in the solvent phase, it would be expected that the relative volumes of the final aqueous and organic phases would be about 1:1 if complete separation occurred. FIG. 14 is a plot of the liquid-liquid interface position as a function of (gravity) settling time for the final two phase broth from the extractive fermentation where solids were removed (2010Y035). The interface position is reported as a percentage of the total broth height in the 2 L graduated cylinder used to observe phase separation of the final broth. The interface position of the final broth from the 2010Y035 fermentation increased from the bottom of the graduated cylinder (no separation) to a level that was about 40% of the total height of the two phase mixture after 175 min of settling time. Therefore, almost complete separation of the two phases in the final broth occurred after 3 hr of settling time. An interface position of approximately 50% would correspond to complete separation.

A 10 mL sample was pulled from the top of the organic phase of the final broth (which had settled for about 3 hr) from the fermentation where solids had been removed. The sample was spun in a high-speed lab centrifuge to determine the amount of aqueous phase that was present in the organic phase after allowing the broth to settle for 3 hr. The results showed that about 90% of the top layer of the final broth was solvent phase. About 10% of the top layer of the final broth was aqueous phase, including a relatively small amount of undissolved solids. Some solids were located at the bottom of the aqueous phase (more dense than the aqueous phase) and also a small amount of solids accumulated at the liquid-liquid interface.

A 10 mL sample was also pulled from the bottom phase of the final broth (which had settled for about 3 hr) from the fermentation where solids had been removed. The sample was spun in a high-speed lab centrifuge to determine the amount of organic phase that was present in the aqueous phase after allowing the broth to settle for 3 hr. It was determined that essentially no organic phase was present in the bottom (aqueous) phase of the final broth from the fermentation from which solids had been removed after the broth had settled for 3 hr. These results confirm that almost complete phase separation had occurred for the final broth from the fermentation where solids had been removed. Almost no phase separation was apparent for the final broth from the fermentation where solids had not been removed. This data implies that removing solids from liquefied corn mash before extractive fermentation may enable a significant improvement in phase separation during and after fermentation resulting in less loss of the microorganism, undissolved solids, and water to downstream processing.

A 10 mL sample was pulled from the top of the final broth from the fermentation from which solids had not been removed after the broth had set for about 3 hr. The sample was spun in a high-speed lab centrifuge to determine the relative amount of solvent and aqueous phases at the top of the final broth. This broth contained all solids from the liquefied corn mash solids. About half of the sample was aqueous phase, and about half was organic phase. The aqueous phase contained significantly more undissolved solids (from the liquefied mash) compared to the sample of the top layer from the broth where solids were removed. The amounts of aqueous and solvent phases in this sample are approximately the same indicating that essentially no phase separation occurred in the final broth where solids were not removed (even after 3 hr of settling time). This data implies that if solids are not removed from liquefied corn mash before an extractive fermentation, little to no phase separation is likely to occur during and after fermentation. This could result in a significant loss of the microorganism, undissolved solids, and water to downstream processing.

### Example 9

### Effect of Removing Undissolved Solids on the Loss of ISPR Extraction Solvent—Extractive Fermentation

This example demonstrates the potential for reducing solvent losses with the DDGS out the back end of an extractive fermentation process by removing undissolved solids from the corn mash prior to fermentation. Example 6 described two extractive fermentations conducted side-by side, one with liquefied corn mash as the sugar source (2010Y036—solids not removed) and one with liquefied mash centrate (2010Y035-aqueous solution of oligosaccharides) obtained by removing most of the undissolved solids from liquefied corn mash. Oleyl alcohol (OA) was added to both fermentations to extract the product isobutanol (i-BuOH) from the broth as it was formed. The amount of residual solvent trapped in the undissolved solids recovered from the final fermentation broths was measured and compared.

After completion of the fermentations 2010Y035 and 2010Y036 described in Example 6, the broths were harvested and used to conduct the phase separation tests. Then the undissolved solids (fines from the corn mash that did not get removed prior to fermentation) were recovered from each broth and analyzed for total extractable oils. The oil recovered from each lot of solids was analyzed for oleyl alcohol and corn oil. The following protocol was followed for both broths:
The broth was centrifuged to separate the organic, aqueous, and solid phases.
The organic and aqueous phases were decanted away from the solids leaving a wet cake at the bottom of the centrifuge bottle.
The wet cake was thoroughly washed with water to remove essentially all of the dissolved solids held up in the cake, such as residual oligosaccharides, glucose, salts, enzymes, etc.
The washed wet cake was dried in a vacuum oven overnight (house-vacuum at 80° C.) to remove essentially all of the water in the cake.
A portion of the dry solids was thoroughly contacted with hexane in a Soxhlet extractor to remove the oil from the solids.
The oil recovered from the solids was analyzed by GC to determine the relative amount of oleyl alcohol and corn oil present in the oil recovered from the solids.
A particle size distribution (PSD) was measured for the solids recovered from both fermentation broths.

The data for the recovery and hexane extraction of the undissolved solids from both fermentation broths is shown in Table 8. The data shows that approximately the same amount of oil was absorbed by the solids (per unit mass of solids) in both fermentations.

TABLE 8

| | Fermentation ID: | |
|---|---|---|
| | 2010Y036 | 2010Y035 |
| Solids removed from liquefied mash before | No (mash) | Yes (centrate) |
| fermentation | | |
| Washed wet cake recovered after removing organic | 290.6 g | 15.6 g |
| phase, aqueous phase, and washing the wet cake | | |
| with water, g: | | |
| Dry solids content in washed wet cake, wt %: | 23.6% | 25.8% |
| Dry solids recovered from washed wet cake, g: | 68.1 g | 4.02 g |
| Dry solids charged to Soxhlet, g: | 20.11 g | 3.91 g |
| Dry Content of solids charged to Soxhlet via | 97.9% | 98.1% |
| moisture analysis, wt %: | | |
| Total oil recovered from Soxhlet hexane extraction, | 2.30 g | 0.25 g |
| g: | | |
| Oil content of solids (dry solids basis), g oil per g | 0.12 g oil/g dry | 0.07 g oil/g dry |
| of dry solids: | solids | solids |
| Fraction of oil extracted from solids that is OA | 76% | 74% |
| (approximate value), wt %: | | |

### Example 10

### Recovery of Soluble Starch from a Wet Cake Generated from the Removal of Solids from Liquefied Corn Mash by Washing the Wet Cake with Water—Two Stage Process

This example demonstrated the recovery of soluble starch from a wet cake by washing the cake twice with water, where the cake was generated by centrifuging liquefied mash. Liquefied corn mash was fed to a continuous decanter centrifuge to produce a centrate stream (C-1) and a wet cake (WC-1). The centrate was a relatively solids-free, aqueous solution of soluble starch, and the wet cake was concentrated in solids compared to the feed mash. A portion of the wet cake was mixed with hot water to form a slurry (S-1). The slurry was pumped back through the decanter centrifuge to produce a wash water centrate (C-2) and a washed wet cake (WC-2). C-2 was a relatively solids-free, dilute aqueous solution of soluble starch. The concentration of soluble starch in C-2 was less than the concentration of soluble starch in the centrate produced from the separation of mash. The liquid phase held up in WC-2 was more dilute in starch than the liquid in the wet cake produced from the separation of mash. The washed wet cake (WC-2) was mixed with hot water to form a slurry (S-2). The ratio of water charged to the amount of soluble starch in the wet cake charged was the same in both wash steps. The second wash slurry was pumped back through the decanter centrifuge to produce a second wash water centrate (C-3) and a wet cake (WC-3) that had been washed twice. C-3 was a relatively solids-free, dilute aqueous solution of soluble starch. The concentration of soluble starch in C-3 was less than the concentration of soluble starch in the centrate produced in the first wash stage (C-2), and thus the liquid phase held up in WC-3 (second washed wet cake) was more dilute in starch than in WC-2 (first washed wet cake). The total soluble starch in the two wash centrates (C-2 and C-3) is the starch that was recovered and could be recycled back to liquefaction. The soluble starch in the liquid held up in the final washed wet cake is much less that in the wet cake produced in the original separation of the mash.

### Production of Liquefied Corn Mash

Approximately 1000 gallons of liquefied corn mash was produced in a continuous dry-grind liquefaction system consisting of a hammer mill, slurry mixer, slurry tank, and liquefaction tank. Ground corn, water, and alpha-amylase were fed continuously. The reactors were outfitted with mechanical agitation, temperature control, and pH control using either ammonia or sulfuric acid. The reaction conditions were as follows:
Hammer mill screen size: 7/64″
Feed Rates to Slurry Mixer
   Ground Corn: 560 lbm/hr (14.1 wt % moisture)
   Process Water: 16.6 lbm/min (200 F)
   Alpha-Amylase: 61 g/hr (Genecor: Spezyme® ALPHA)
Slurry Tank Conditions:
   Temperature: 185° F. (85° C.)
   pH: 5.8
   Residence Time: 0.5 hr
   Dry Corn Loading: 31 wt %
   Enzyme Loading: 0.028 wt % (dry corn basis)
Liquefaction Tank Conditions:
   Temperature: 185° F. (85° C.)
   pH: 5.8
   Residence Time: about 3 hr
   No additional enzyme added.

The production rate of liquefied corn mash was about 3 gpm. The starch content of the ground corn was measured to be about 70 wt % on a dry corn basis. The total solids (TS) of the liquefied mash was about 31 wt %, and the total suspended solids (TSS) was approximately 7 wt %. The liquid phase contained about 23-24 wt % liquefied starch as measured by HPLC (soluble oligosaccharides).

The liquefied mash was centrifuged in a continuous decanter centrifuge at the following conditions:
Bowl Speed: 5000 rpm (about 3600 g's)
Differential Speed: 15 rpm
Weir Diameter: 185 mm (weir plate removed)
Feed Rate: Varied from 5-20 gpm.

Approximately 850 gal of centrate and approximately 1400 lbm of wet cake were produced by centrifuging the mash. The total solids in the wet cake were measured to be about 46.3% (suspended+dissolved) by moisture balance. Knowing that the liquid phase contained about 23 wt % soluble starch, it was estimated that the total suspended solids in the wet cake was about 28 wt %. It was estimated that the wet cake contained approximately 12% of the soluble starch that was present in the liquefied mash prior to the centrifuge operation.

Recovery of Soluble Starch from Wet Cake by Washing the Solids with Water—1^{st} Wash

About 707 lbm of the wet cake recovered from separation of the liquefied mash was mixed with 165 gal of hot (91° C.) water in a 300 gallon stainless steel vessel. The resulting slurry was mixed for about 30 min. The slurry was continuously fed to a decanter centrifuge to remove the washed solids from the slurry. The centrifuge used to separate the wash slurry was the same one used to remove solids from the liquefied mash above, and it was rinsed with fresh water before feeding the slurry. The centrifuge was operated at the following conditions to remove solids from the wash slurry:
Bowl Speed: 5000 rpm (about 3600 g's)
Differential Speed: 5 rpm
Weir Diameter: 185 mm (weir plate removed)
Feed Rate: 5 gpm.

Approximately 600 lbm of washed wet cake was produced by the centrifuge, but only 400 lbm were recovered due to loss of material. The total solids in the wet cake were measured to be about 36.7% (suspended+dissolved) by moisture balance. The total soluble starch (sum of glucose, DP2, DP3, and DP4+) in the liquid phase of the slurry and in the wash water centrate (obtained from the slurry) was measured to be about 6.7 wt % and 6.9 wt %, respectively, by HPLC. DP2 refers to a dextrose polymer containing two glucose units (glucose dimer). DP3 refers to a dextrose polymer containing three glucose units (glucose trimer). DP4+ refers to a dextrose polymer containing four or more glucose units (glucose tetramer and higher). This confirmed that a well-mixed dilution wash stage was achieved. Therefore, the concentration of soluble starch in the liquid phase held up in the washed wet cake must have been about 6.8 wt % (by mass balance) for this dilution wash. Based on the total solids and dissolved oligosaccharide data, it was estimated that the total suspended solids in the washed wet cake was about 32 wt %. It was estimated that the washed wet cake contained approximately 2.6% of the soluble starch that was present in the original liquefied mash if all 600 lbm of the cake produced by the centrifuge could have been washed. This represents about a 78% reduction in soluble starch in the washed wet cake compared to the mash wet cake prior to washing. If the wet cake produced from the separation of liquefied mash was not washed, about 12% of the total starch in the mash would be lost as soluble (liquefied) starch. If the wet cake produced from the separation of mash is washed with water at the conditions demonstrated in this example, 2.6% of the total starch from the mash would be lost as soluble (liquefied) starch.

About 400 lbm of the washed wet cake recovered from the first re-slurry wash of the liquefied mash wet cake was mixed with 110 gal of hot (90° C.) water in a 300 gallon stainless steel vessel. The resulting slurry was mixed for about 30 min. The slurry was continuously fed to a decanter centrifuge to remove the washed solids from the slurry. The centrifuge used to separate the second wash slurry was the same one used in the first wash above, and it was rinsed with fresh water before feeding the second wash slurry. The centrifuge was operated at the following conditions to remove solids from the wash slurry:
Bowl Speed: 5000 rpm (about 3600 g's)
Differential Speed: 5 rpm
Weir Diameter: 185 mm (weir plate removed)
Feed Rate: 4 gpm.

Approximately 322 lbm of washed wet cake was produced by the centrifuge. The total solids in the wet cake from the second wash were measured to be about 37.4% (suspended+dissolved) by moisture balance. The total soluble starch (sum of glucose, DP2, DP3, and DP4+) in the liquid phase of the slurry and in the wash water centrate (obtained from the slurry) was measured to be about 1.6 wt % and 1.6 wt %, respectively, by HPLC. This confirmed that a well-mixed dilution wash stage was achieved in the second wash. Therefore, the concentration of soluble starch in the liquid phase held up in the washed wet cake must have been about 1.6 wt % (by mass balance) for this dilution wash. Based on the total solids and dissolved oligosaccharide data, it was estimated that the total suspended solids in the washed wet cake was about 36 wt %. It was estimated that the washed wet cake contained approximately 0.5% of the soluble starch that was present in the original liquefied mash if all 600 lbm of the cake produced in the first wash stage could have been washed. This represents an overall reduction in soluble starch in the doubly washed wet cake compared to the mash wet cake prior to washing of about 96%. If the wet cake produced from the separation of liquefied mash was not washed, about 12% of the total starch in the mash would be lost as soluble (liquefied) starch. If the wet cake produced from the separation of mash is washed twice with water at the conditions demonstrated in this example, 0.5% of the total starch from the mash would be lost as soluble (liquefied) starch.

### Example 11

### Effect of High Temperature Stage During Liquefaction on the Conversion of Starch in Corn Solids to Soluble (Liquefied) Starch

This example demonstrates that operating liquefaction with a high temperature (or “cook”) stage at some time in the middle of the reaction can result in higher conversion of the starch in corn solids to soluble (liquefied) starch. The “cook” stage demonstrated in this example involves raising the liquefaction temperature at some point after liquefaction starts, holding at the higher temperature for some period of time, and then lowering the temperature back to the original value to complete liquefaction.

A. Procedure to Measure Unhydrolyzed Starch Remaining in Solids after Liquefaction

Liquefied corn mash was prepared in one run according to the protocol in Example 1 (no intermediate high temperature stage). Liquefied corn mash was prepared in another run at the same conditions as in the first run except for the addition of an intermediate high temperature stage. The mash from both runs was worked up according to the following steps. It was centrifuged to separate the aqueous solution of liquefied starch from the undissolved solids. The aqueous solution of liquefied starch was decanted off to recover the wet cake. The wet cake contained most of the undissolved solids from the mash, but the solids were still wet with liquefied starch solution. The wet cake was thoroughly washed with water, and the subsequent slurry was centrifuged to separate the aqueous layer from the undissolved solids. The cake was washed a total of five times with enough water to remove approximately all of the soluble starch that was held up in the original wet cake recovered from liquefaction. Consequently, the liquid phase held up in the final washed wet cake consisted of water containing essentially no soluble starch.

The final washed wet cake was re-slurried in water, and large excesses of both alpha-amylase and glucoamylase were added. The slurry was mixed for at least 24 hr while controlling temperature and pH to enable the alpha-amylase to convert essentially all the unhydrolyzed starch remaining in the undissolved solids to soluble oligosaccharides. The soluble oligosaccharides generated from the residual starch (which was not hydrolyzed during liquefaction at the conditions of interest) were subsequently converted to glucose by the glucoamylase present. Glucose concentration was tracked with time by HPLC to make sure all the oligosaccharides generated from the residual starch were converted to glucose and that the glucose concentration was no longer increasing with time.

### B. Production of Liquefied Corn Mash

Two batches of liquefied corn mash were prepared (approximately 1 L each) at 85° C. using Liquozyme® SC DS (alpha-amylase from Novozymes, Franklinton, N.C.). Both batches operated at 85° C. for a little more than 2 hr. However, a “cook” period was added in the middle of the second batch (“Batch 2”). The temperature profile for Batch 2 was about 30 min at 85° C., raising the temperature from 85° C. to 101° C., holding at 101° C. for about 30 min, cooling down to 85° C., and continuing liquefaction for another 120 min. The ground corn used in both batches was the same as in Example 1. A corn loading of 26 wt % (dry corn basis) was used in both batches. The total amount of enzyme used in both runs corresponded to 0.08 wt % (dry corn basis). The pH was controlled at 5.8 during both liquefaction runs. The liquefactions were carried out in a glass, jacketed resin kettle. The kettle was set up with mechanical agitation, temperature control, and pH control.

The following protocol was followed to prepare liquefied corn mash for Batch 1:
The alpha-amylase was diluted in tap water (0.418 g enzyme in 20.802 g water)
Charged 704.5 g tap water to the kettle
Turned on agitator
Made first charge of ground corn, 198 g
Heated to 55° C. while agitating
Adjusted pH to 5.8 using H₂SO₄ or NaOH
Made first charge of alpha-amylase solution, 7.111 g
Heated to 85° C.
Held at 85° C. for 30 min
Made second charge of alpha-amylase solution, 3.501 g
Made second charge of ground corn, 97.5 g
Continued to run at 85° C. for another 100 min
After the liquefaction was complete, cooled to 60° C.
Dumped reactor and recovered 998.5 g of liquefied mash.

The following protocol was followed to prepare liquefied corn mash for Batch 2:
The alpha-amylase was diluted in tap water (0.3366 g enzyme in 16.642 g water)
Charged 562.6 g tap water to the kettle
Turned on agitator
Charged ground corn, 237.5 g
Heated to 55° C. while agitating
Adjusted pH to 5.8 using dilute H₂SO₄ or NaOH
Made first charge of alpha-amylase solution, 4.25 g
Heated to 85° C.
Held at 85° C. for 30 min
Heated to 101° C.
Held at 101° C. for 30 min
Lowered temperature of mash back to 85° C.
Adjusted pH to 5.8 using dilute H₂SO₄ or NaOH
Made second charge of alpha-amylase solution, 4.2439 g
Continued to run at 85° C. for another 120 min.
After the liquefaction was complete, cooled to 60° C.
   C. Removal of Undissolved Solids from the Liquefied Mash and Washing of the Wet Cake with Water to Remove Soluble Starch

Most of the solids were removed from both batches of liquefied mash by centrifuging them in a large floor centrifuge at 5000 rpm for 20 min at room temperature. Centrifugation of 500 g of mash from Batch 1 yielded 334.1 g of centrate and 165.9 g of wet cake. Centrifugation of 872 g of mash from Batch 2 yielded 654.7 g of centrate and 217 g of wet cake. The wet cakes recovered from each batch of liquefied mash were washed five times with tap water to remove essentially all of the soluble starch held up in the cakes. The washes were performed in the same bottle used to centrifuge the original mash to avoid transferring the cake between containers. For each wash stage, the cake was mixed with water, and the resulting wash slurry was centrifuged (5000 rpm for 20 min) at room temperature. This was done for all five wash stages performed on the wet cakes recovered from both batches of mash. Approximately 165 g of water was used in each of the five washes of the wet cake from Batch 1 resulting in a total of 828.7 g of water used to wash the wet cake from Batch 1. Approximately 500 g of water was used in each of the five washes of the wet cake from Batch 2 resulting in a total of 2500 g of water used to wash the wet cake from Batch 2. The total wash centrate recovered from all five water washes of the wet cake from Batch 1 was 893.1 g. The total wash centrate recovered from all five water washes of the wet cake from Batch 2 was 2566.3 g. The final washed wet cake recovered from Batch 1 was 101.5 g, and the final washed wet cake recovered from Batch 2 was 151.0 g. The final washed wet cakes obtained from each batch contained essentially no soluble starch; therefore, the liquid held up in each cake was primarily water. The total solids (TS) of the wet cakes was measured using a moisture balance. The total solids of the wet cake from Batch 1 was 21.63 wt %, and the TS for the wet cake from Batch 2 was 23.66 wt %.

D. Liquefaction/Saccharification of Washed Wet Cake to Determine the Level of Unhydrolyzed Starch Remaining in the Undissolved Solids after Liquefaction

The level of unhydrolyzed starch remaining in the solids present in both washed wet cakes was measured by re-slurrying the cakes in water and adding excess alpha-amylase and excess glucoamylase. The alpha-amylase converts residual unhydrolyzed starch in the solids to soluble oligosaccharides which dissolve in the aqueous phase of the slurry. The glucoamylase subsequently converts the soluble oligosaccharides generated by the alpha-amylase to glucose. The reactions were run at 55° C. (maximum recommended temperature for the glucoamylase) for at least 24 hr to ensure all of the residual starch in the solids was converted to soluble oligosaccharides and that all the soluble oligosaccharides were converted to glucose. The residual unhydrolyzed starch that was in the solids, which is the starch that did not get hydrolyzed during liquefaction, can be calculated from the amount of glucose generated by this procedure.

The alpha-amylase and glucoamylase enzymes used in the following protocols were Liquozyme® SC DS and Spirizyme® Fuel, respectively (Novozymes, Franklinton, N.C.). The vessel used to treat the washed wet cakes was a 250 mL jacketed glass resin kettle equipped with mechanical agitation, temperature control, and pH control. The amount of Liquozyme® used corresponds to an enzyme loading of 0.08 wt % on a “dry corn basis.” The amount of Spirizyme® used corresponds to an enzyme loading of 0.2 wt % on a “dry corn basis.” This basis is defined as the amount of ground corn required to give the amount of undissolved solids held up in the washed cakes assuming all the starch is hydrolyzed to soluble oligosaccharides. The undissolved solids held up in the washed cakes are considered to be mostly the non-starch, non-fermentable part of the corn. These enzyme loadings are at least four times higher than is required to give complete liquefaction and saccharification at 26% corn loading. The enzymes were used in large excess to ensure complete hydrolysis of the residual starch in the solids and complete conversion of the oligosaccharides to glucose.

The following protocol was followed to determine the level of unhydrolyzed starch in the solids present in the washed wet cake from Batch 1 mash:
The alpha-amylase was diluted in tap water (0.1297 g enzyme in 10.3607 g water)
The glucoamylase was diluted in tap water (0.3212 g enzyme in 15.6054 g water)
Charged 132 g tap water to the kettle
Turned on agitator
Charged 68 g of the washed wet cake produced from liquefaction Batch 1 (TS=21.63 wt %)
Heated to 55° C. while agitating
Adjusted pH to 5.5 using dilute H₂SO₄ or NaOH
Charged alpha-amylase solution, 3.4992 g
Charged glucoamylase solution, 5.319 g
Run at 55° C. for 24 hr while controlling pH at 5.5 and periodically sample the slurry for glucose.

The following protocol was followed to determine the level of unhydrolyzed starch in the solids present in the washed wet cake from Batch 2.
The alpha-amylase was diluted in tap water (0.2384 g enzyme in 11.709 g water)
The glucoamylase was diluted in tap water (0.3509 g enzyme in 17.5538 g water)
Charged 154.3 g tap water to the kettle
Turned on agitator
Charged 70.7 g of the washed wet cake produced from liquefaction Batch 1 (TS=23.66 wt %)
Heated to 55° C. while agitating
Adjusted pH to 5.5 using dilute H₂SO₄ or NaOH
Charged alpha-amylase solution, 2.393 g
Charged glucoamylase solution, 5.9701 g
Run at 55° C. for 24 hr while controlling pH at 5.5 and periodically sample the slurry for glucose.

### Comparison of Results for the Liquefaction/Saccharification of the Washed Wet Cakes

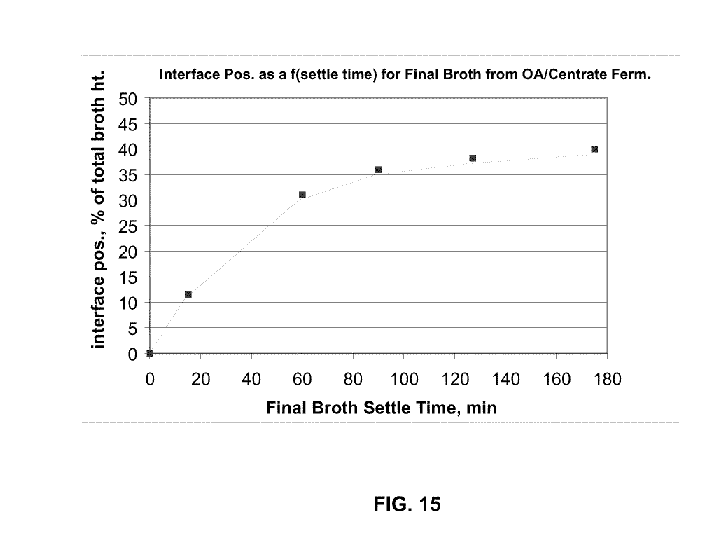

As described above, the washed wet cakes from Batches 1 and 2 were re-slurried in water, and large excesses of both alpha-amylase and glucoamylase were added to the slurries in order to hydrolyze any starch remaining in the solids and convert it to glucose. FIG. 15 shows the concentration of glucose in the aqueous phase of the slurries as a function of time.

The glucose concentration increased with time and leveled out at a maximum value at approximately 24 hr for both reactions. The slight decrease in glucose between 24 and 48 hr could have been from microbial contamination; therefore, the maximum level of glucose reached in each system was used to calculate the level of residual unhydrolyzed starch that was in the solids of the washed wet cake. The maximum level of glucose reached by reacting (in the presence of alpha-amylase and glucoamylase) the washed wet cake obtained from the Batch 1 liquefaction was 4.48 g/L. By comparison, the maximum level of glucose reached by reacting (in the presence of alpha-amylase and glucoamylase) the washed wet cake obtained from the Batch 2 liquefaction was 2.39 g/L.

The level of residual unhydrolyzed starch that was in the undissolved solids in the liquefied mash (that did not get hydrolyzed during liquefaction) was calculated based on the glucose data obtained from the washed wet cake obtained from the corresponding batch of mash.
Liquefaction Batch 1: The residual unhydrolyzed starch in the solids corresponds to 2.1% of the total starch in the corn fed to liquefaction. This implies that 2.1% of the starch in the corn was not hydrolyzed during Batch 1 liquefaction conditions. No intermediate high temperature (“cook”) stage occurred during liquefaction Batch 1.
Liquefaction Batch 2: The residual unhydrolyzed starch in the solids corresponds to 1.1% of the total starch in the corn fed to liquefaction. This implies that 1.1% of the starch in the corn was not hydrolyzed during Batch 2 liquefaction conditions. A high temperature (“cook”) stage did occur during liquefaction Batch 2.

This example demonstrates that the addition of a high temperature “cook” stage at some time during the liquefaction could result in higher starch conversion. This will result in less residual unhydrolyzed starch remaining in the undissolved solids in the liquefied corn mash and will lead to less starch loss in a process where undissolved solids are removed from the mash prior to liquefaction.

### Example 12

### Effect of High Temperature Stage During Liquefaction on the Conversion of Starch in Corn Solids to Soluble (Liquefied) Starch

Two batches of liquefied corn mash (Batch 3 and Batch 4) were prepared at 85° C. using Liquozyme® SC DS (alpha-amylase from Novozymes, Franklinton, N.C.). Both batches operated at 85° C. for a little more than 2 hr. However, a “cook” period was added in the middle of Batch 4. The temperature profile for Batch 4 was about 30 min at 85° C., raising the temperature from 85° C. to 121° C., holding at 121° C. for about 30 min, cooling down to 85° C., and continuing liquefaction for another 90 min. The ground corn used in both batches was the same as in Example 1. A corn loading of 26 wt % (dry corn basis) was used in both batches. The total amount of enzyme used in both runs corresponded to 0.04 wt % (dry corn basis). The pH was controlled at 5.8 during both liquefaction runs. The liquefaction for Batch 3 was carried out in a 1 L glass, jacketed resin kettle, and the liquefaction for Batch 4 was carried out in a 200 L stainless steel fermentor. Both reactors were outfitted with mechanical agitation, temperature control, and pH control.

The experimental conditions for this example were similar to those described for Example 9 with the following differences:

For the Production of Liquefied Corn Mash for Batch 3: 0.211 g of alpha-amylase was diluted in 10.403 g tap water. The first charge of alpha-amylase solution was 3.556 g. The second charge of alpha-amylase solution was 1.755 g and the reaction was allowed to continue to run at 85° C. for another 90 min.

For the Production of Liquefied Corn Mash for Batch 4: 22 g of alpha-amylase was diluted in 2 kg tap water, 147.9 kg of tap water was charged to the fermentor, and 61.8 kg of ground corn was charged. The first charge of alpha-amylase solution was 1.0 kg, the reaction was heated to 85° C. and held at 85° C. for 30 min, then the reaction was heated to 121° C. and held at 121° C. for 30 min. The second charge of alpha-amylase solution was 1 kg and the reaction was allowed to continue to run at 85° C. for another 90 min.

Removal of Undissolved Solids from the Liquefied Mash and Washing of the Wet Cake with Water to Remove Soluble Starch

Most of the solids were removed from both batches of liquefied mash by centrifuging them in a large floor centrifuge at 5000 rpm for 15 min at room temperature. Centrifugation of 500.1 g of mash from Batch 3 yielded 337.2 g of centrate and 162.9 g of wet cake. Centrifugation of 509.7 g of mash from Batch 4 yielded 346.3 g of centrate and 163.4 g of wet cake. The wet cakes recovered from each batch of liquefied mash were washed five times with tap water to remove essentially all of the soluble starch held up in the cakes. The washes were performed in the same bottle used to centrifuge the original mash to avoid transferring the cake between containers. For each wash stage, the cake was mixed with water, and the resulting wash slurry was centrifuged (5000 rpm for 15 min) at room temperature. This was done for all five wash stages performed on the wet cakes recovered from both batches of mash. Approximately 164 g of water was used in each of the five washes of the wet cake from Batch 3 resulting in a total of 819.8 g of water used to wash the wet cake from Batch 3. Approximately 400 g of water was used in each of the five washes of the wet cake from Batch 4 resulting in a total of 2000 g of water used to wash the wet cake from Batch 4. The total wash centrate recovered from all five water washes of the wet cake from Batch 3 was 879.5 g. The total wash centrate recovered from all five water washes of the wet cake from Batch 4 was 2048.8 g. The final washed wet cake recovered from Batch 3 was 103.2 g, and the final washed wet cake recovered from Batch 4 was 114.6 g. The final washed wet cakes obtained from each batch contained essentially no soluble starch; therefore, the liquid held up in each cake was primarily water. The total solids (TS) of the wet cakes were measured using a moisture balance. The total solids of the wet cake from Batch 3 was 21.88 wt %, and the TS for the wet cake from Batch 4 was 18.1 wt %.

The experimental conditions for this example were similar to those described for Example 9 with the following differences:

For the Liquefaction/Saccharification of Washed Wet Cake to Determine the Level of Unhydrolyzed Starch Remaining in the Undissolved Solids after Liquefaction for Batch 3: 68 g of the washed wet cake produced from liquefaction of Batch 3 was charged (TS=21.88 wt %). 3.4984 g of alpha-amylase solution and 5.3042 g of glucoamylase was charged. The reaction was ran at 55° C. for 47 hr while controlling pH at 5.5 and periodically sampling the slurry for glucose.

For the Liquefaction/Saccharification of Washed Wet Cake to Determine the Level of Unhydrolyzed Starch Remaining in the Undissolved Solids after Liquefaction for Batch 4: 0.1663 g of alpha-amylase was diluted in 13.8139 g tap water, and 0.213 g of glucoamylase was diluted in 20.8002 g tap water. 117.8 g of tap water was charged to the kettle. 82.24 g of the washed wet cake produced from liquefaction of Batch 4 was charged (TS=18.1 wt %). 3.4952 g of alpha-amylase solution and 10.510 g of glucoamylase was charged. The reaction was ran at 55° C. for 50 hr while controlling pH at 5.5 and periodically sampling the slurry for glucose.

### Comparison of Results for the Liquefaction/Saccharification of the Washed Wet Cakes

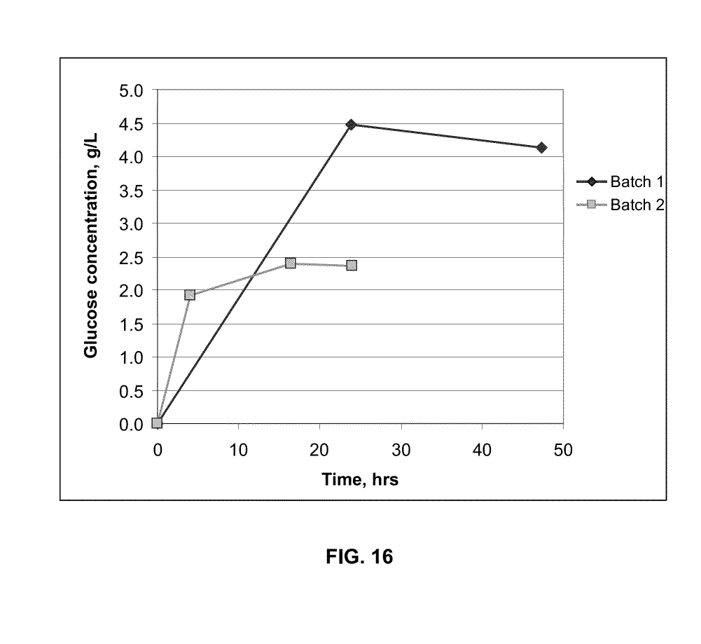

As described above, the washed wet cakes from Batches 3 and 4 were re-slurried in water, and large excesses of both alpha-amylase and glucoamylase were added to the slurries in order to hydrolyze any starch remaining in the solids and convert it to glucose. FIG. 16 shows the concentration of glucose in the aqueous phase of the slurries as a function of time.

The glucose concentration increased with time and leveled out at a maximum value at approximately 26 hr for the washed wet cake from Batch 3. For the Batch 4 washed wet cake, the glucose concentration continued to increase slightly between 24 hr and 47 hr. It is assumed that the glucose concentration measured at 47 hr for the Batch 4 wet cake is approximately equal to the maximum value. The maximum level of glucose reached by reacting (in the presence of alpha-amylase and glucoamylase) the washed wet cake obtained from the Batch 3 liquefaction was 8.33 g/L. By comparison, the maximum level of glucose reached by reacting (in the presence of alpha-amylase and glucoamylase) the washed wet cake obtained from the Batch 4 liquefaction was 4.92 g/L.

The level of residual unhydrolyzed starch that was in the undissolved solids in the liquefied mash (that did not get hydrolyzed during liquefaction) was calculated based on the glucose data obtained from “hydrolyzing” the washed wet cake (in the presence of excess alpha-amylase and glucoamylase) obtained from the corresponding batch of mash.
Liquefaction Batch 3: The residual unhydrolyzed starch in the solids corresponds to 3.8% of the total starch in the corn fed to liquefaction. This implies that 3.8% of the starch in the corn was not hydrolyzed during Batch 3 liquefaction conditions. No intermediate high temperature (“cook”) stage occurred during liquefaction Batch 3.
Liquefaction Batch 4: The residual unhydrolyzed starch in the solids corresponds to 2.2% of the total starch in the corn fed to liquefaction. This implies that 2.2% of the starch in the corn was not hydrolyzed during Batch 4 liquefaction conditions. A high temperature (“cook”) stage did occur during liquefaction Batch 4.

This example demonstrates that the addition of a high temperature “cook” stage at some time during the liquefaction could result in higher starch conversion. This will result in less residual unhydrolyzed starch remaining in the undissolved solids in the liquefied corn mash and will lead to less starch loss in a process where undissolved solids are removed from the mash prior to liquefaction.

### Summary and Comparison of Examples 11 and 12

Liquefaction conditions can influence the conversion of starch in the corn solids to soluble (liquefied) starch. Possible liquefaction conditions that could affect the conversion of starch in the ground corn to soluble starch are temperature, enzyme (alpha-amylase) loading, and +/−a high temperature (“cook”) stage occurs at some time during liquefaction. Examples 11 and 12 demonstrated that implementing a high temperature (“cook”) stage at some time during liquefaction can result in higher conversion of starch in the corn solids to soluble (liquefied) starch. The high temperature stage in the liquefactions described in Examples 11 and 12 involved raising the liquefaction temperature at some point after liquefaction starts, holding at the higher temperature for some period of time, and then lowering the temperature back to the original value to complete liquefaction.

The liquefaction reactions compared in Example 11 were run at a different enzyme loading than the reactions compared in Example 12. These examples demonstrate the effect of two key liquefaction conditions on starch conversion: (1) enzyme loading, and (2) +/− a high temperature stage is applied at some time during liquefaction.

The conditions used to prepare the four batches of liquefied corn mash described in Examples 11 and 12 are summarized below and in Table 9.

Conditions common for all batches:
Liquefaction temperature—85° C.
Total time at liquefaction temperature—approximately 2 hr
Screen size used to grind corn—1 mm
pH—5.8
Dry corn loading—26%
Alpha-amylase—Liquozyme® SC DS (Novozymes, Franklinton, N.C.).

TABLE 9

| | Batch 1 | Batch 2 | Batch 3 | Batch 4 |
|---|---|---|---|---|
| Described in Example: | 11 | 11 | 12 | 12 |
| High Temperature Stage | No | Yes | No | Yes |
| Implemented | | | | |
| Temperature of High | NA | 101° C. | NA | 121° C. |
| Temperature Stage, C: | | | | |
| Total Enzyme Loading, wt % | 0.08% | 0.08% | 0.04% | 0.04% |
| (dry corn basis): | | | | |
| Residual Unhydrolyzed | 2.1% | 1.1% | 3.8% | 2.3% |
| Starch in Undissolved Solids | | | | |
| after Liquefaction (as a | | | | |
| percentage of total starch in | | | | |
| corn feed): | | | | |

The temperature profile for Batches 2 and 4 was (all values are approximate): 85° C. for 30 min, High Temperature Stage for 30 min, 85° C. for 90 min. Half the enzyme was added before the initial 85° C. period, and half was added after the high temperature stage for the final 85° C. period.

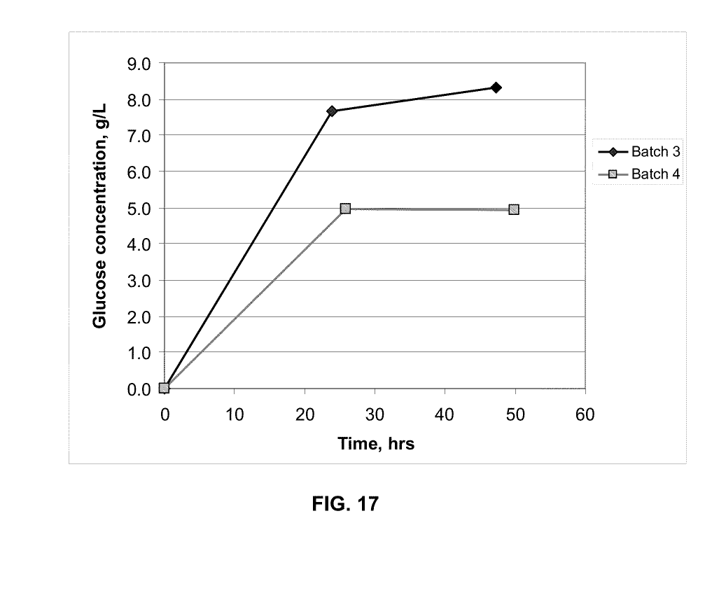

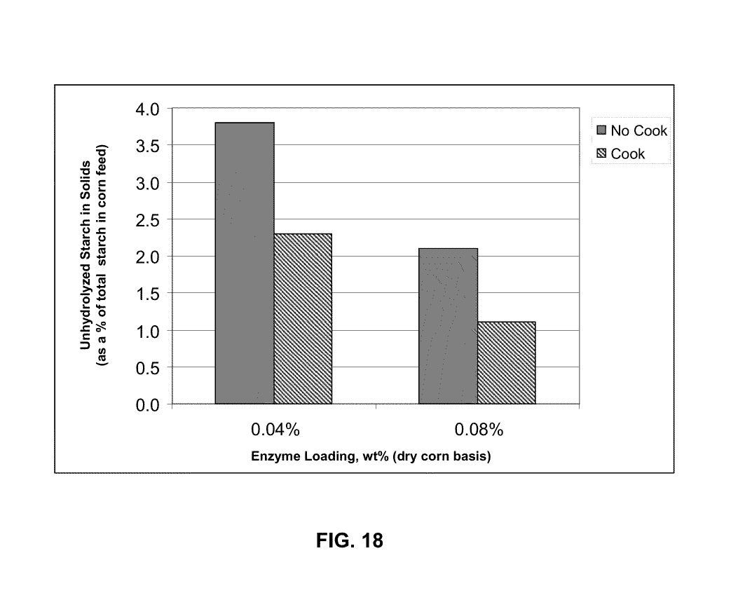

FIG. 17 illustrates the effect of enzyme loading and +/−a high temperature stage was applied at some time during the liquefaction on starch conversion. The level of residual unhydrolyzed starch in the solids is the starch that was not hydrolyzed during the liquefaction conditions of interest. FIG. 17 shows that the level of unhydrolyzed starch in the solids was reduced by almost half by applying a high temperature (“cook”) stage at some point during the liquefaction. This was demonstrated at two different enzyme loadings. The data in FIG. 17 also shows that doubling the enzyme loading resulted in almost half the level of unhydrolyzed starch remaining in the solids whether a high temperature stage was applied during liquefaction or not. These examples demonstrate that operating liquefaction with a higher enzyme (alpha-amylase) loading and/or the addition of a high temperature (“cook”) stage at some time during the reaction could result in a significant reduction in residual unhydrolyzed starch in the undissolved solids present in the liquefied corn mash and can reduce the loss of starch in a process where undissolved solids are removed from the mash prior to liquefaction. Any residual starch in the solids after liquefaction will not have the opportunity to hydrolyze during fermentation in a process where solids are removed prior to fermentation.

### Example 13

### Screen Separation of Starch and Nonsolubles Following 85° C. Enzyme Digestion

Mash (301 grams) prepared per the method described in Example 1 were maintained at pH 5.8 using drops of NaOH solution when adjustment was necessary, treated with a vendor-specified dose of approximately 0.064 grams of Liquozyme® alpha-amylase enzyme (Novozyme, Franklinton, N.C.) and held at 85° C. for five hours. The product was refrigerated.

Refrigerated product was warmed to approximately 50° C. and 48 g was poured onto a filter assembly containing a 100 mesh screen and connected to a house vacuum source at between −15 in Hg and −20 in Hg. The screen dish had an exposed screen surface area of 44 cm² and was sealed inside a plastic filter housing provided by Nalgene® (Thermo Fisher Scientific, Rochester, N.Y.). The slurry was filtered to form a wet cake on the screen and a yellow cloudy filtrate of 40.4 g in the receiver bottle. The wet cake was immediately washed in place with water and then discontinued while the vacuum source continued to pull any free moisture through the final washed cake. Filtration was ended when dripping ceased from the underside of the filter. An additional 28.5 g of wash filtrate were collected over 3 stages where the final stage of filtrate revealed the least color and turbidity. The final wet cake mass of 7.6 g was air dried to 2.1 g over 24 hours at room temperature. The 2.1 g were determined to contain 7.73% water after drying with a heat lamp. The vacuum filtration of this experiment produced a wet cake containing 25% total dry solids.

A sample of filtrate was combined with oleyl alcohol at room temperature, vigorously mixed and allowed to settle. The interface was restored in approximately 15 min but a hazy rag layer remained.

Lugol's solution (starch indicator) consisting of 1 g of >99.99% (trace metals basis) iodine, 2 g of ReagentPlus® grade (>99%) potassium iodide (both from Sigma-Aldrich, St. Louis, Mo.), and 17 g of house deionized water in the amount of one drop was added to samples of the filtrate, dried cake solids re-slurried in water and a control sample of water. The filtrate turned dark blue or purple, the solids slurry turned very dark blue and the water became light amber in color. Any color darker than amber indicates presence of oligosaccharides greater than 12 units long.

This experiment illustrated that most suspended solids could be separated from starch solution prepared as described above at a moderate rate on a 100 mesh screen and that starch remains with the filter cake solids. This is an indication of incomplete washing of the cake where a portion of hydrolyzed starch is left behind.

This experiment was repeated with 156 grams of mash on a 63 mm diameter 100 mesh screen. The maximum temperature was 102° C., the enzyme was Spezyme® and the slurry was held above 85° C. for three hours. The screening rate was measured and determined to be 0.004 or less gallons per minute per square foot of screen area.

### Example 14

### Screen Separation of Starch and Nonsolubles Following 115° C. Enzyme Digestion

House deionized water (200 g) were charged into an open Parr Model 4635 1 liter pressure vessel (Moline, Ill.) and heated to a temperature of 85° C. The water was agitated with a magnetic stir bar. Dry ground corn (90 g) prepared as described in Example 1 were added spoon-wise. The pH was raised from 5.2 to near 6.0 with stock aqueous ammonia solution. Approximately 0.064 grams of Liquozyme® solution were added with a small calibrated pipette. The lid of the pressure vessel was sealed and the vessel was pressurized to 50 psig with house nitrogen. The agitated mixture was heated to 110° C. within 6 min and held between 106 to 116° C. for a total of 20 min. The heating was reduced, the pressure was relieved, and the vessel was opened. An additional 0.064 g of Liquozyme® was added and the temperature was held at 63-75° C. for an additional 142 min.

A small amount of the slurry was taken from the Parr vessel and gravity screened through a stack of 100, 140, and 170 mesh screens. Solids were retained only on the 100 mesh screen.

A portion, about 40%, of the slurry was transferred while hot onto the top of a dual screen assembly of 100 and 200 mesh dishes of 75 millimeter diameter. Some gravity filtration took place. Vacuum, between −15 and −20 inches of mercury, was pulled on the filtrate receiver and steady filtration was established. The filtrate was yellow and cloudy but with a stable dispersion. The cake surface was exposed within 5 min. The cake was washed with a spray of deionized water for 2-3 min and repeated with a change of receiver until the turbidity of the filtrate was constant—a total of five sprayings. The screens were examined with the conclusion that all solids were on the 100 mesh screen and none were on the 200 mesh. The wet cake was 5 mm thick. The wet cake mass was determined to be 18.9 g and the combined filtrate masses were 192 g.

The remaining mass of slurry was transferred to the filter assembly with a 100 mesh screen in place at 65° C. and filtered over 5-10 min. The cake was washed with a spray of deionized water for 3-4 min and repeated with a change of receiver until the turbidity of the filtrate was constant—a total of eight sprayings. Vacuum was continued until no more drops were observed falling from the underside of the filter. The wet cake was 8 mm thick and 75 mm in diameter with a mass of 36.6 g. The combined filtrates weighed 261 g.

Three vials were tested for starch per the method described above. One vial contained water and the other two contained samples of wet cake slurried in deionized water. All vials turned yellow-amber in color. This was interpreted to mean that the filter cake was washed free of oligosaccharides of starch. These solids were later analyzed rigorously using prolonged liquefaction and subsequent saccharification to confirm that on a glucose basis, the wet cake contained no more than 0.2% of the total starch that was in the original corn.

A sample of filtrate was combined with oleyl alcohol in a vial, vigorously mixed and allowed to settle. A clear oil layer was quickly attained and the interface was well defined with little rag layer. This example illustrated that in a process in which corn mash is heated to hydrothermal conditions of ˜110° C. for 20 min of cooking and further liquefied for more than two hours at 85° C. before being filtered and washed, the total filtrate contains essentially all starch supplied in the grain. Furthermore, no significant interference is observed between the oleyl alcohol and the impurities contained in the filtrate.

This experiment was repeated with 247 grams of mash on a 75 mm diameter 80 mesh screen. The maximum cook temperature was 115° C., the enzyme was Liquozyme® and the slurry was held at or above 85° C. for three hours. The screening rate was measured and determined to be more than 0.1 gallons per minute per square foot of screen area.

### Example 15

This example illustrated the removal of solids from stillage and extraction by desolventizer to recover fatty acids, esters, and triglycerides from the solids. During fermentation, solids are separated from whole stillage and fed to a desolventizer where they are contacted with 1.1 tons/hr of steam. The flow rates for the whole stillage wet cake (extractor feed), solvent, the extractor miscella, and extractor discharge solids are as shown in Table 10. Table values are short tons/hr.

TABLE 10

| | Solids from | | | Extractor |
|---|---|---|---|---|
| | whole | | | discharge |
| | stillage | Solvent | Miscella | solids |
| Fatty acids | 0.099 | 0 | 0.0982 | 0.001 |
| Undissolved solids | 17.857 | 0 | 0.0009 | 17.856 |
| Fatty acid butyl esters | 2.866 | 0 | 2.837 | 0.0287 |
| Hexane | 0 | 11.02 | 10.467 | 0.555 |
| Triglyceride | 0.992 | 0 | 0.982 | 0.0099 |
| Water | 29.762 | 0 | 29.464 | 0.297 |

Solids exiting the desolventizer are fed to a dryer. The vapor exiting the desolventizer contains 0.55 tons/hr of hexane and 1.102 tons/hr of water. This stream is condensed and fed to a decanter. The water-rich phase exiting the decanter contains about 360 ppm of hexane. This stream is fed to a distillation column where the hexane is removed from the water-rich stream. The hexane enriched stream exiting the top of the distillation column is condensed and fed to the decanter. The organic-rich stream exiting the decanter is fed to a distillation column. Steam (11.02 tons/hr) is fed to the bottom of the distillation column. The composition of the overhead and bottom products for this column are shown in Table 11. Table values are tons/hr.

TABLE 11

| | Bottoms | Overheads |
|---|---|---|
| Fatty acids | 0.0981 | 0 |
| Fatty acid butyl esters | 2.8232 | 0 |
| Hexane | 0.0011 | 11.12 |
| Triglyceride | 0.9812 | 0 |
| Water | 0 | 11.02 |

### Example 16

### By-Product Recovery

This example illustrates the recovery of by-products from mash. Corn oil separated from mash under the conditions described in Example 6 with the exception that a three-phase centrifuge (Flottweg Tricanter® Z23-4 bowl diameter, 230 mm, length to diameter ratio 4:1) was used with these conditions:
Bowl Speed: 5000 rpm
Differential Speed: 10 rpm
Feed Rate: 3 gpm
Phase Separator Disk: 138 mm
Impeller Setting: 144 mm.

The corn oil separate had 81% triglycerides, 6% free fatty acids, 4% diglyceride, and 5% total of phospholipids and monoglycerides as determined by gas chromatography and thin layer chromatography (see, e.g., U.S. Patent Application Publication No. 2012/0164302).

The solids separated from mash under the conditions described above had a moisture content of 58% as determined by weight loss upon drying and had 1.2% triglycerides and 0.27% free fatty acids as determined by gas chromatography (see, e.g., U.S. Patent Application Publication No. 2012/0164302).

The composition of solids separated from whole stillage, oil extracted between evaporator stages, by-product extractant and Condensed Distillers Solubles (CDS) in Table 14 were calculated assuming the composition of whole stillage shown in Table 12 and the assumptions in Table 13 (separation at three-phase centrifuge). The values of Table 11 were obtained from an Aspen Plus® model (Aspen Technology, Inc., Burlington, Mass.). This model assumes that corn oil is not extracted from mash. It is estimated that the protein content on a dry basis of cells, dissolved solids, and suspended solids is approximately 50%, 22%, and 35.5%, respectively. The composition of by-product extractant is estimated to be 70.7% fatty acid and 29.3% fatty acid isobutyl ester on a dry basis.

TABLE 12

| Component | Mass % |
|---|---|
| Water | 57.386% |
| Cells | 0.502% |
| Fatty acids | 6.737% |
| Isobutyl esters of fatty acids | 30.817% |
| Triglyceride | 0.035% |
| Suspended solids | 0.416% |
| Dissolved solids | 4.107% |

TABLE 13

| | Hydrolyzer | Thin | |
|---|---|---|---|
| | feed | stillage | Solids |
| Organics | 99.175% | 0.75% | 0.08% |
| Water and dissolved solids | 1% | 96% | 3% |
| Suspended solids and cells | 1% | 2% | 97% |

TABLE 14

| Stream | C. protein | triglyceride | FFA | FABE |
|---|---|---|---|---|
| Whole stillage wet cake | 40% | trace | 0.5% | 2.2% |
| Oil at evaporator | 0% | 0.08% | 16.1% | 73.8% |
| CDS | 22% | trace % | 0.37% | 1.71% |

### Example 17

### Removal of Corn Oil from Liquefied Corn Mash

This example describes the use of a three-phase centrifuge to remove corn oil from liquefied corn mash. Whole corn kernels typically contain about 3-6 wt % corn oil, most of which resides in the germ. Corn oil is released from the germ during dry milling and liquefaction. Consequently, corn mash contains free corn oil.

Liquefied corn mash was generated using a standard continuous liquefaction process as used, for example, in a dry-grind corn-to-ethanol process. The ground corn contained 4.16 wt % corn oil (dry corn basis) and had a moisture content of 14.7 wt %. Ground corn and water were fed to a slurry tank at 10.2 lbm/min and 17.0 lbm/min, respectively, to give a dry corn loading of 32 wt %. Alpha-amylase was fed to the slurry tank at a rate that corresponded to an enzyme loading of about 0.025 wt % on a dry corn basis. The slurry and liquefaction tanks were both run at 85° C. and a pH of 5.8. The total residence time at 85° C. was about 2 hr. Mash was produced at a rate of about 3 gpm and contained about 1.3 wt % corn oil on a wet basis. A portion of this oil existed as free oil and a portion was in the undissolved solids. This corresponds to a total corn oil content of the mash to be roughly 2.0 lbm of corn oil/bushel of corn. The total solids (TS) in the mash was 32 wt % and the total suspended solids (TSS) was 7.7 wt %.

The liquefied corn mash was fed to a three-phase centrifuge (Model Z23-4/441, Flottweg Tricanter®, Flottweg AG, Vilsibiburg, Germany) at a rate of about 3 gpm. The feed temperature was about 80° C. The mash was separated into three streams: (1) corn oil, (2) aqueous solution of oligosaccharides (liquefied starch), and (3) wet cake. The operating conditions of the Tricanter® were as follows:
Bowl Speed: 5000 rpm
G-force: approximately 4000 g's
Differential speed: 10 rpm
Impeller setting: approximately 145
Phase separator disk: approximately 138 mm.

Table 15 summarizes data (flow rate, density, solids content, and corn oil content) measured for the feed stream and the three exit streams from the Tricanter®.

TABLE 15

| | Feed | Aqueous | | |
|---|---|---|---|---|
| | Mash | Centrate | Wet Cake | Corn Oil |
| Flow Rate, lbm/min: | 27.2 | 19.5 | 7.6 | 0.14 |
| Density, g/ml: | 1.1008 | ~1.09 | | 0.875 |
| Total Solids, wt %: | 32.0 | 28.7 | 39.1 | ~0 |
| Total Suspended | 7.7 | 4.3 | 16.6 | ~0 |
| Solids, wt %: | | | | |
| Corn Oil Content | 1.3 | 0.38 | 1.95 | 99.4* |
| (wet basis), wt %: | | | | |
| Corn Oil Content, | 2.0 | 0.4 | 0.8 | 0.8 |
| lbm/bushel: | | | | |
| % of Corn Oil in feed: | NA | 20 | 41 | 39 |

*Balance is water

The corn oil removed from the mash by the Tricanter® accounted for 39% of the total corn oil in the mash feed. The corn oil removal rate was equal to about 0.8 lbm/bushel of corn. The corn oil separated and recovered from the liquefied corn mash contained about 85 wt % glycerides.

In a process where about 0.8 lb corn oil/bushel of corn is removed, the mash flow rate would decrease by 3.9 gallons per minute:

$$3.7 \frac{bu corn}{min} * 0.8 \frac{lb oil}{bu corn} / 7.65 \frac{lb oil}{gal} = 3.9 gallons oil / minute$$

In a production plant where the total mash flow to fermentation is about 700 gpm, the oil that was removed would make about 0.55% of the total mash flow. Assuming that the production plant proportionally raises throughput to take advantage of the extra volume, the yearly production would increase by 0.55%, which means that a 56 MMGPY plant would produce an additional 310,000 gallons of ethanol.

### Example 18

### Removal of Corn Oil from Liquefied Corn Mash—Feed Rate Adjustment

In this example, liquefied corn mash was fed to a three-phase centrifuge at a feed rate of 1 gpm. Liquefied corn mash was generated using a standard continuous liquefaction process as used, for example, in a dry-grind corn-to-ethanol process. The ground corn contained 4.16 wt % corn oil (dry corn basis) and had a moisture content of 14.7 wt %. Ground corn and water were fed to a slurry tank at 8.2 lbm/min and 19.0 lbm/min, respectively, to give a dry corn loading of approximately 26 wt %. Alpha-amylase was fed to the slurry tank at a rate of 50 g/hr, which corresponded to an enzyme loading of about 0.026 wt % on a dry corn basis. The slurry and liquefaction tanks were both run at 85° C. and a pH of 5.8. No jet cooker was used. The total residence time at 85° C. was about 2 hr. Mash was produced at a rate of about 3 gpm and inventoried into a 1500 gal tank for centrifuge testing. The mash contained about 1.1 wt % corn oil on a wet basis. A portion of this oil existed as free oil and a portion was in the undissolved solids. This corresponds to a total corn oil content of the mash to be roughly 2.0 lbm of corn oil/bushel of corn. The TS in the mash was 25.6 wt % and the TSS was 5.3 wt %.

The liquefied corn mash was fed from a feed tank to a three-phase centrifuge (Model Z23-3, Flottweg Tricanter®, Flottweg AG, Vilsibiburg, Germany) at a rate of about 1 gpm. The feed temperature was about 80° C. The mash was separated into three streams: (1) corn oil, (2) aqueous solution of oligosaccharides (liquefied starch), and (3) wet cake. The operating conditions of the Tricanter® were as follows:
Bowl Speed: 5000 rpm
G-force: approximately 4000 g's
Differential speed: 12 rpm
Impeller setting: approximately 156
Phase separator disk: approximately 140 mm.

Table 16 summarizes data (flow rate, density, solids content, and corn oil content) measured for the feed stream and the three exit streams from the Tricanter®. The quality of the corn oil mass balance was 102% and the quality of the total solids mass balance was 105%.

TABLE 16

| | Feed | Aqueous | | |
|---|---|---|---|---|
| | Mash | Centrate | Wet Cake | Corn Oil |
| Flow Rate, lbm/min: | 9.2 | 6.2 | 3.0 | 0.016 |
| Density, g/ml: | ~1.10 | ~1.09 | | ~0.9 |
| Total Solids, wt %: | 25.6 | 21.6 | 37.4 | ~0 |
| Total Suspended | 5.3 | 1.1 | 13.8 | ~0 |
| Solids, wt %: | | | | |
| Corn Oil Content | 1.1 | 0.28 | 2.2 | >99* |
| (wet basis), wt %: | | | | |
| Corn Oil Content, | 2.0 | 0.36 | 1.34 | 0.34 |
| lbm/bushel: | | | | |
| % of the Corn Oil | NA | 18 | 67 | 17 |
| in the feed: | | | | |

*Balance is water

The corn oil removed from the mash by the Tricanter® accounted for 17% of the total corn oil in the mash feed. This corn oil removal rate was equal to about 0.34 lbm/bushel of corn. The corn oil separated and recovered from the liquefied corn mash contained about 81.4 wt % glycerides and 8.3 wt % free fatty acids.

### Example 19

### Removal of Corn Oil from Liquefied Corn Mash—Feed Rate Adjustment

In this example, liquefied corn mash was fed to a three-phase centrifuge at a feed rate of 10.1 gpm. Liquefied corn mash was generated using a standard continuous liquefaction process as used, for example, in a dry-grind corn-to-ethanol process. The ground corn contained 4.16 wt % corn oil (dry corn basis) and had a moisture content of 14.7 wt %. Ground corn and water were fed to a slurry tank at 8.2 lbm/min and 19.0 lbm/min, respectively, to give a dry corn loading of approximately 26 wt %. Alpha-amylase was fed to the slurry tank at a rate of 50 g/hr, which corresponded to an enzyme loading of about 0.026 wt % on a dry corn basis. The slurry and liquefaction tanks were both run at 85° C. and a pH of 5.8. No jet cooker was used. The total residence time at 85° C. was about 2 hr. Mash was produced at a rate of about 3 gpm and inventoried into a 1500 gal tank for centrifuge testing. The mash contained about 1.1 wt % corn oil on a wet basis. A portion of this oil existed as free oil and a portion was in the undissolved solids. This corresponds to a total corn oil content of the mash to be roughly 2.0 lbm of corn oil/bushel of corn. The TS in the mash was 26.2 wt % and the TSS was 6.7 wt %.

The liquefied corn mash was fed from the feed tank to a three-phase centrifuge (Model Z23-4/441, Flottweg Tricanter®, Flottweg AG, Vilsibiburg, Germany) at a rate of about 10.1 gpm. The feed temperature was about 80° C. The mash was separated into three streams: (1) corn oil, (2) aqueous solution of oligosaccharides (liquefied starch), and (3) wet cake. The operating conditions of the Tricanter® were as follows:
Bowl Speed: 5000 rpm
G-force: approximately 4000 g's
Differential speed: 20 rpm
Impeller setting: approximately 148
Phase separator disk: approximately 138 mm.

Table 17 summarizes data (flow rate, density, solids content, and corn oil content) measured for the feed stream and the three exit streams from the Tricanter®. The quality of the corn oil mass balance was 95%.

TABLE 17

| | Feed | Aqueous | | |
|---|---|---|---|---|
| | Mash | Centrate | Wet Cake | Corn Oil |
| Flow Rate, lbm/min: | 92.2 | 73.1 | 18.9 | 0.177 |
| Density, g/ml: | ~1.10 | ~1.09 | | ~0.9 |
| Total Solids, wt %: | 26.2 | 23.3 | 36.9 | ~0 |
| Total Suspended | 6.7 | 1.9 | 25.2 | ~0 |
| Solids, wt %: | | | | |
| Corn Oil Content | 1.1 | 0.71 | 1.4 | >99* |
| (wet basis), wt %: | | | | |
| Corn Oil Content, | 2.0 | 1.02 | 0.52 | 0.36 |
| lbm/bushel: | | | | |
| % of the Corn Oil | NA | 51 | 26 | 18 |
| in the feed: | | | | |

*Balance is water

The corn oil removed from the mash by the Tricanter® accounted for 18% of the total corn oil in the mash feed. This corn oil removal rate was equal to about 0.36 lbm/bushel of corn. The corn oil separated and recovered from the liquefied corn mash contained about 81.4 wt % glycerides and 8.3 wt % free fatty acids.

### Example 20

### Effect of Liquefied Corn Mash pH on the Recovery of Corn Oil from Mash

Liquefied corn mash was generated using a standard continuous liquefaction process as typically used in a dry-grind corn-to-ethanol process. The ground corn contained 4.6 wt % corn oil (dry corn basis) and had a moisture content of 12.5 wt %. Ground corn and water were fed to the slurry tank at rates produce corn mash at 3 gpm with a dry corn loading of 25.9 wt %. The slurry tank was operated at 85° C. with a 30 min residence time. The slurry was then heated to 105° C. using live steam in a jet cooker and held at that temperature for about 30 min. After exiting the hold tube, the slurry was fed into a liquefaction tank which was operated at 85° C. with a 90 min residence time. Alpha-amylase (Spezyme® ALPHA, Genencor®, Palo Alto, Calif.) was continuously fed to the process at a rate that corresponded to an overall enzyme loading of 0.04 wt % enzyme on a dry corn basis. Forty percent (40%) of the total enzyme was added to the slurry tank, and 60% was added to the liquefaction tank. The slurry and liquefaction tanks were both run at a pH of 5.8. Mash was produced at a rate of about 3 gpm and inventoried into a 1500 gal tank for centrifuge testing. The liquefied corn mash contained about 1.12 wt % corn oil on a wet basis. This corresponds to a total corn oil content of the mash to be roughly 2.2 lbm of corn oil/bushel of corn. Some of this oil existed as free oil; some still was in the undissolved solids. The ratio of glycerides to free fatty acids in the corn oil in the mash was about 7.6 to 1. The total solids (TS) in the mash were 25.9 wt %, and the total suspended solids (TSS) were 4.7 wt %. The DE (dextrose equivalent) and the pH of the final mash was 15.9 and 5.75, respectively. The density of the mash was 1.08 g/mL.

The liquefied mash was separated using a three-phase centrifuge (Model 223-4/441, Flottweg Tricanter®, Flottweg AG, Vilsibiburg, Germany) at three different feed flow rates: 1.24 gal/min, 5.1 gal/min and 10 gal/min. The feed temperature was about 80° C. The mash was separated into three streams: (1) corn oil, (2) aqueous solution of oligosaccharides (liquefied starch), and (3) wet cake. The bowl speed was held constant at about 5000 rpm (approximately 4000 g's). Table 18 compares the corn oil recovery as a function of mash feed rate to the Tricanter® for a mash pH of 5.8. The data shown in Table 18 shows that there is an effect of feed rate to the Tricanter® on the recovery rate of corn oil at pH=5.8.

TABLE 18

| | Mash | | | Corn | | |
|---|---|---|---|---|---|---|
| | Feed | | Impeller | Oil in | Corm Oil | Corn Oil |
| | Rate, | Differential | Setting, | Mash, | Recovered, | Recovery |
| Test | gpm | Speed, rpm | mm | g/min | g/min | % |
| A | 1.2 | 5.2 | 144 | 63.3 | 8.3 | 13 |
| B | 5.1 | 10.5 | 146 | 248.4 | 73.2 | 29 |
| C | 10 | 9.8 | 149 | 487.1 | 100.3 | 21 |

Corn oil recovery is based on the total oil contained in the mash (both free oil and oil in the solids). The mash fed to the Tricanter® contained 1.1-1.2 wt % corn oil (includes free oil and oil in the solids).

The data in Table 18 shows that there is an effect of mash feed rate on corn oil recovery rate (at the conditions tested). Table 19 summarizes the amount of oil phase in the aqueous centrate, aqueous phase in the oil centrate, and solids in the oil centrate for the three conditions tested.

TABLE 19

| | Mash | | Corn Oil in | Aqueous | | Density |
|---|---|---|---|---|---|---|
| | Feed | Corn Oil | Aqueous | Phase in | Solids in | of Corn |
| | Rate, | Recovery | Centrate, | Corn Oil, | Corn Oil, | Oil, |
| Test | gpm | % | vol %* | vol %* | vol %* | g/mL |
| A | 1.2 | 13 | 0 | 0 | 1.8 | 0.892 |
| B | 5.1 | 29 | 0 | 0 | 1.7 | 0.892 |
| C | 10 | 21 | 0 | 3 | 1.5 | 0.906 |

*Measured using a LuMiSizer ® (L.U.M GmbH, Berlin, Germany)

The data in Table 19 shows that the corn oil recovered was fairly clean since it contained very little aqueous phase and very little solids. The corn oil separated and recovered from the liquefied corn mash contained about 85.1 wt % glycerides and 8.0 wt % free fatty acids. The balance was solids, aqueous phase, and other extractables (e.g. phospholipids, sterols, etc.,).

The pH of the remaining liquefied corn mash in the Tricanter® feed tank was lowered to about 3. The acidic mash was separated using a three-phase centrifuge (Model Z23-4/441, Flottweg Tricanter®, Flottweg AG, Vilsibiburg, Germany) at two different feed flow rates: 1.3 gal/min and 5 gal/min. The feed temperature was about 80° C. The mash was separated into three streams: (1) corn oil, (2) aqueous solution of oligosaccharides (liquefied starch), and (3) wet cake. The bowl speed was held constant at about 5000 rpm (approximately 4000 g's). Table 20 compares the corn oil recovery as a function of mash feed rate to the Tricanter® for a mash pH of 3.0.

TABLE 20

| | Mash | | | Corn | | |
|---|---|---|---|---|---|---|
| | Feed | | Impeller | Oil in | Corn Oil | Corn Oil |
| | Rate, | Differential | Setting, | Mash, | Recovered, | Recovery |
| Test | gpm | Speed, rpm | mm | g/min | g/min | % |
| D | 1.3 | 5.2 | 144.5 | 47.3 | 20.8 | 44 |
| E | 5.0 | 10.3 | 146 | 181.9 | 72.8 | 40 |

Mash was produced at pH=5.8, and the pH of the final mash was then lowered to 3 before feeding the centrifuge. Corn oil recovery is based on the total oil contained in the mash (both free oil and oil in the solids). The mash fed to the Tricanter® contained about 0.9 wt % corn oil (includes free oil and oil in the solids). Total Solids of mash fed to Tricanter® were 27.1 wt %, and Total Suspended Solids of mash were 5.5 wt %.

Table 21 summarizes the amount of oil phase in the aqueous centrate, aqueous phase in the oil centrate, and solids in the oil centrate for the two conditions tested. The data in Table 21 shows that the corn oil recovered was fairly clean since it contained very little aqueous phase and very little solids.

TABLE 21

| | Mash | | Corn Oil in | Aqueous | | Density |
|---|---|---|---|---|---|---|
| | Feed | Corn Oil | Aqueous | Phase in | Solids in | of Corn |
| | Rate, | Recovery | Centrate, | Corn Oil, | Corn Oil, | Oil, |
| Test | gpm | % | vol %* | vol %* | vol %* | g/mL |
| D | 1.3 | 44 | 0 | 0.2 | 0 | 0.895 |
| E | 5.0 | 40 | 0 | 0 | 0 | 0.895 |

*Measured using a LuMiSizer ® (L.U.M GmbH, Berlin, Germany)

Comparing the results of Test A (Table 18) to Test D (Table 20) and comparing the results of Test B (Table 19) to Test E (Table 21) show an effect of mash pH on the corn oil recovery using a Tricanter®. The data suggests that reducing the pH of the mash before separating it with a Tricanter® results in higher corn oil recovery. These comparisons are shown in Table 22.

TABLE 22

| | |
|---|---|
| | pH of Mash fed to |
| Mash Feed Rate | Tricanter ® |

| gpm | pH = 5.8 | pH = 3.0 |
|---|---|---|
| 1.3 | 13% | 44% |
| 5.1 | 29% | 40% |

Percentages shown in Table 22 are corn oil recoveries based on the total oil contained in the mash (both free oil and oil in the solids). The composition of corn oil in the mash fed to the Tricanter® ranged from 0.9% to 1.2 wt % corn oil (includes free oil and oil in the solids) for all the tests described in this example. The Tricanter® was operated at 5000 rpm (˜4000 G's), and the differential speed and impeller setting were 5-10 rpm and 145 mm, respectively.

### Example 21

### Recovery of Corn Oil and Solids from Corn Mash

Liquefied corn mash was generated using a standard continuous liquefaction process as used in a dry-grind corn-to-ethanol process with 30-31 wt % on a dry corn basis. Recycle water consisting of cook water and backset was used, which elevated the total solids (TS) to approximately 33 wt %. Alpha-amylase (Spezyme® RSL, Genencor®, Palo Alto, Calif.) was added to the slurry tank (85° C., pH approximately 5.8, 30 min residence time) at a rate that corresponded to approximately 0.02 wt % dry corn base enzyme load. A jet cooker was used to elevate the temperature to 105-110° C. with 18 min cook time. The liquefaction tank was run at 85° C. with a pH of approximately 5.8. Spezyme® RSL (Genencor®, Palo Alto, Calif.) was also added to the liquefaction tank at a rate that corresponded to approximately 0.005 wt % dry corn base enzyme load, and the total residence time in the liquefaction tank was about 90 min. A side stream of mash was collected from the liquefaction tank and diverted to a small dilution tank, where process condensate was added to achieve the desired dilution. The original mash contained about 1.55 wt % corn oil on a wet basis. A portion of this oil existed as free oil and a portion was in the undissolved solids. This corresponds to a total corn oil content of the original mash to be roughly 3.0 lbm of corn oil/bushel of corn. The TS in the original mash was 33.2 wt % and the total suspended solids (TSS) was 6.5 wt %. The dilution with process condensate lowered the TS to approximately 27 wt %, the TSS to approximately 5.5 wt %, and the oil content to approximately 1.3 wt % (wet basis).

The liquefied corn mash was fed from the feed tank to a three-phase centrifuge (Model Z23-4/441, Flottweg Tricanter®, Flottweg AG, Vilsibiburg, Germany) at a rate between 9 and 11 gpm. The feed temperature was about 85° C. The mash was separated into three streams: (1) corn oil, (2) aqueous solution of oligosaccharides (liquefied starch), and (3) wet cake. The operating conditions of the three-phase centrifuge were as follows:
Bowl Speed: 5000 rpm
G-force: approximately 3200 g
Differential speed: 25 rpm
Impeller setting: see table
Phase separator disk: approximately 138 mm

Table 23 summarizes three-phase centrifuge conditions and properties following separation. Streams at both corn loads 33 wt % and 26 wt % were separated into a very clean corn oil stream and wet cakes at 38-41 wt % total solids. The suspended solids concentration in the heavy phase was strongly affected by the corn load. The 33 wt % sample generated a centrate TSS of approximately 3.5-4 wt % while the 26 wt % TS generate a lower TSS centrate at approximately 1.7-2 wt %.

TABLE 23
Feed Properties

| | | | | |
|---|---|---|---|---|
| TS (wt %) | 33 | 33 | 26 | 26 |
| Feed rate (gpm) | 9 | 11.2 | 9 | 11.3 |

Centrifuge Conditions

| Bowl speed (rpm) | 5000 | | 5000 | (4400-5400) | 5000 | 5000 | |
|---|---|---|---|---|---|---|---|
| Differential speed (rpm) | 25 | (25-50) | 25 | (25-50) | 25 | 15 | (15-25) |
| Impeller Speed (mm) | 155 | (145-158) | 155 | (155-160) | 155 | 153 | (153-155) |

Light Centrate Properties

| Water content (ppm) | Very low | | Very low | | Very low | | Very low | |
|---|---|---|---|---|---|---|---|---|
| TSS (wt %) | Very low | | Very low | | Very low | | Very low | |
| Flow rate (mL/min) | 230 | (150-330) | 300 | (195-360) | 280 | (170-280) | 364 | (364-459) |
| Recovery (on total basis) (%) | 43 | (30-60) | 43 | (30-54) | 53 | (30-53) | 54 | (54-68) |

Heavy Centrate Properties

| | | | | | | | | |
|---|---|---|---|---|---|---|---|---|
| TSS (wt %) | 3.5 | (3.5-4) | 4.3 | (3.6-4.7) | 1.7 | (1.7-3.8) | 2 | (2-3.2) |

Wet Cake Properties

| | | | | | | | | |
|---|---|---|---|---|---|---|---|---|
| TS (wt %) | 41 | (36-42) | 39 | (37-39) | 38.7 | (38.5-38.7) | 39 | (30-40) |

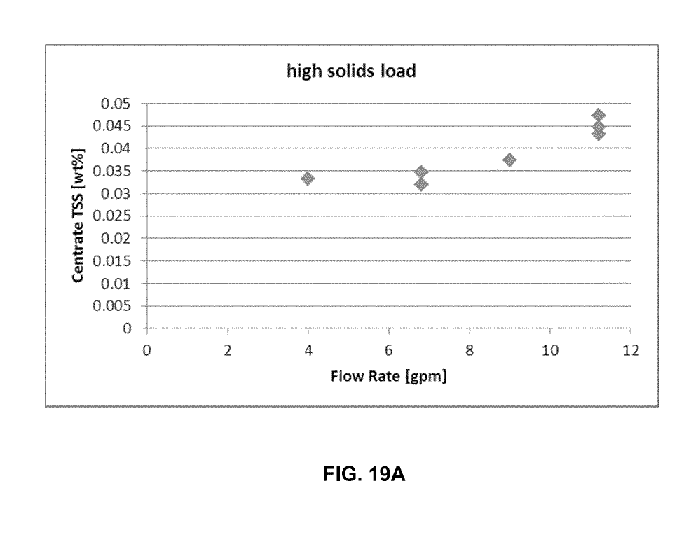

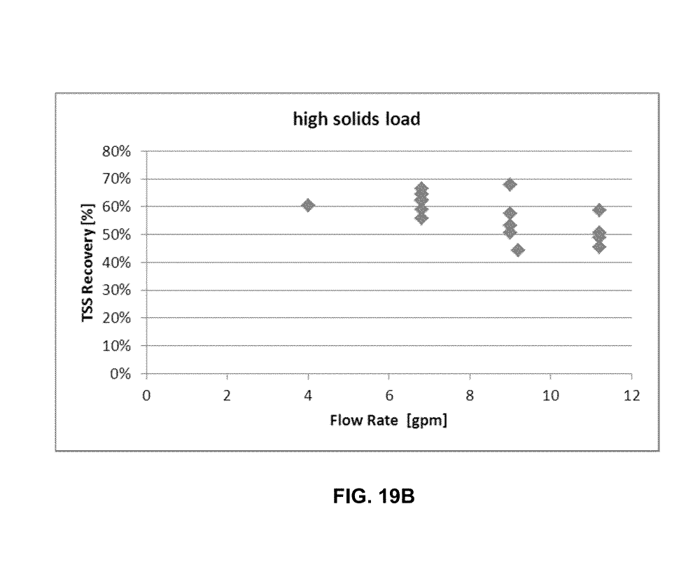

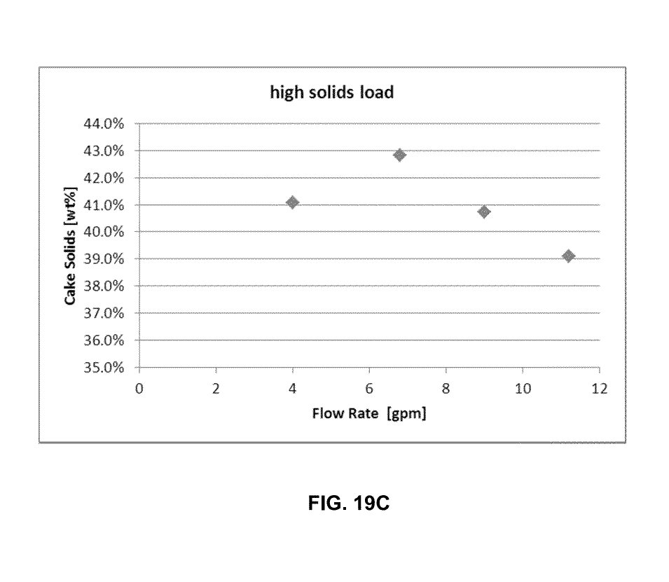

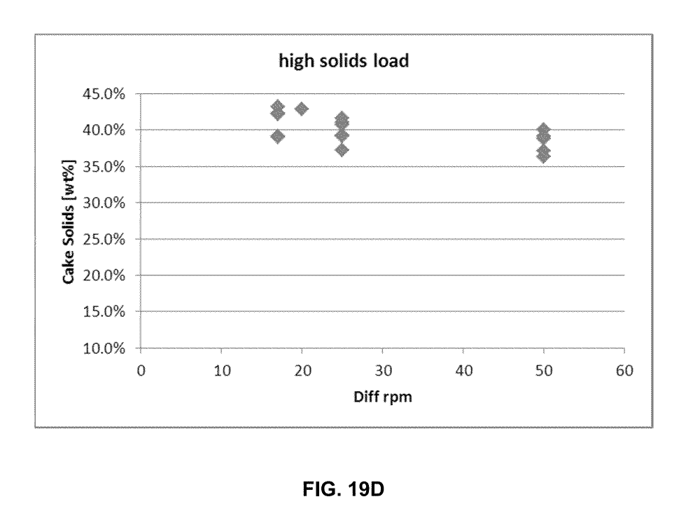

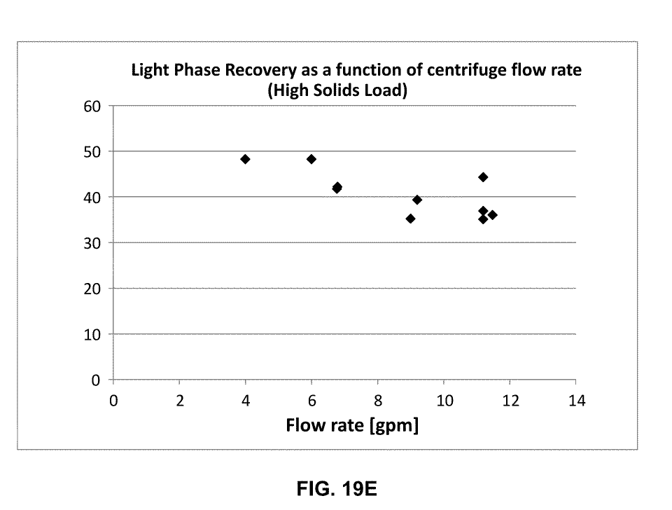

Results are also shown in FIGS. 19A to 19E. FIG. 19A shows that at low flow rates of approximately 4 gpm, the centrate TSS was about 3.3%, and the centrate TSS increased to about 4.2-4.7% with a flow rate of 11.5 gpm.

FIG. 19B shows the suspended solids recovery as a function of flow rate. At low flow rates of approximately 4 gpm, approximately 60% of the suspended solids were recovered in the wet cake. By increasing the flow rate to about 11.5 gpm, the recovery rate decreased to about 40-50%.

FIG. 19C shows the wet cake total solids as a function of flow rate. At low flow rates of approximately 4 gpm, wet cake total solids were about 41%. By increasing the flow rate to about 11.5 gpm, the wet cake total solids decreased to about 39%.

FIG. 19D shows the impact of differential rpm on the total wet cake solids. The wet cake solids decreased with increased differential rpm.

FIG. 19E shows the effect of feed rate on corn oil recovery. At low flow rates, oil recovery was about 48%. When the flow rate was increased to approx. 11.5 gpm, oil recovery decreases to about 35%. It appears that less oil is separated from the feed stream with higher flow rate.

### Example 22

### Rheological Characteristics of Corn Mash

The viscosity of corn mash is measured using an AR-G2 rotational rheometer (TA Instruments, New Castle, Del.) configured with vane geometry. A slurry of ground corn and water is prepared, mixed, and heated in a resin kettle to 55° C. The pH is adjusted to 5.8, and enzyme (e.g., alpha-amylase and/or glucoamylase) is added to the slurry. The slurry is heated to 65° C. at a rate of 2° C./min. A sample is removed and transferred to the rheometer equipped with a narrow gap concentric cylinder geometry that is preheated to 65° C. A temperature ramp is then performed raising the temperature from 65° C. to 85° C. at a rate of 2° C./min. The temperature ramp is conducted at a fixed shear rate (e.g., 75-200 s⁻¹). Viscosity is measured as a function of time.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A method for producing a fermentation product comprising:
providing a feedstock slurry comprising fermentable carbon source, undissolved solids, and oil;
separating a portion of the undissolved solids and oil from the feedstock slurry whereby an aqueous solution comprising fermentable carbon source, a wet cake comprising solids and an oil stream are formed; and
adding the aqueous solution to a fermentation broth comprising microorganisms whereby the fermentation product is produced.

2. The method of claim 1, further comprising the step of recovering the oil stream.

3. The method of claim 1, further comprising the step of washing the wet cake wherein an aqueous stream comprising carbohydrate is generated.

4. The method of claim 3, further comprising the step of adding the aqueous stream to the fermentation broth.

5. The method of claim 1, further comprising the step of combining the aqueous solution with a portion of the wet cake to produce a mixture of the aqueous solution and wet cake and adding the mixture to the fermentation broth.

6. The method of claim 1, wherein separating the feedstock slurry is a single step process.

7. The method of claim 1, wherein the undissolved solids and oil are separated from feedstock slurry by a separation device selected from decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, and combination thereof.

8. The method of claim 7, wherein one or more control parameters of the separation device is adjusted to improve separation of the feedstock slurry.

9. The method of claim 8, wherein the one or more control parameters are selected from differential speed, bowl speed, flow rate, impeller position, weir position, scroll pitch, residence time, and discharge volume.

10. The method of claim 1, wherein the fermentation product is a product alcohol selected from ethanol, propanol, butanol, pentanol, hexanol, and fusel alcohols.

11. The method of claim 1, wherein the microorganism comprises a 1-butanol biosynthetic pathway, a 2-butanol biosynthetic pathway, or an isobutanol biosynthetic pathway.

12. The method of claim 1, wherein real-time measurements are used to monitor separation of the feedstock slurry.

13. The method of claim 12, wherein separation is monitored by Fourier transform infrared spectroscopy, near-infrared spectroscopy, Raman spectroscopy, high pressure liquid chromatography, viscometers, densitometers, tensiometers, droplet size analyzers, particle analyzers, or combinations thereof.

14. A method comprising:
a) providing a feedstock slurry comprising fermentable carbon source and undissolved solids;
b) separating at least a portion of the undissolved solids from the feedstock slurry whereby an aqueous solution comprising fermentable carbon source and a wet cake comprising solids are generated;
c) contacting the wet cake with a liquid to form a wet cake mixture; and
d) separating at least a portion of undissolved solids from the wet cake mixture whereby a second aqueous solution comprising fermentable carbon source and a second wet cake comprising solids are generated.

15. The method of claim 14, the liquid is selected from fresh water, backset, cook water, process water, lutter water, evaporation water, or combinations thereof.

16. The method of claim 14, wherein steps c) and d) are repeated.

17. A method comprising:
providing a feedstock slurry comprising fermentable carbon source, undissolved solids, and oil;
separating at least a portion of the oil and undissolved solids from the feedstock slurry whereby an aqueous solution comprising fermentable carbon source, an oil stream; and
a wet cake comprising solids are generated; and
c) contacting the wet cake with a liquid to form a wet cake mixture; and
d) separating at least a portion of undissolved solids and oil from the wet cake mixture whereby a second aqueous solution comprising fermentable carbon source, a second oil stream; and a second wet cake comprising solids are generated.

18. The method of claim 17, wherein separating the feedstock slurry is a single step process.

19. The method of claim 17, wherein the undissolved solids and oil are separated from feedstock slurry by a separation device selected from decanter bowl centrifugation, three-phase centrifugation, disk stack centrifugation, filtering centrifugation, decanter centrifugation, filtration, microfiltration, vacuum filtration, beltfilter, membrane filtration, crossflow filtration, drum filter, pressure filtration, filtration using a screen, screen separation, rotary screen, grating, porous grating, flotation, hydrocyclone, filter press, screwpress, gravity settler, vortex separator, or combination thereof.

20. The method of claim 19, wherein one or more control parameters of the separation device is adjusted to improve separation of the feedstock slurry.

21. The method of claim 20, wherein the one or more control parameters are selected from differential speed, bowl speed, flow rate, impeller position, weir position, scroll pitch, residence time, and discharge volume.

22. The method of claim 17, wherein real-time measurements are used to monitor separation of the feedstock slurry.

23. The method of claim 22, wherein separation is monitored by Fourier transform infrared spectroscopy, near-infrared spectroscopy, Raman spectroscopy, high pressure liquid chromatography, viscometers, densitometers, tensiometers, droplet size analyzers, particle analyzers, or combinations thereof.

24. A method comprising
providing a feedstock slurry comprising fermentable carbon source, undissolved solids, and oil;
separating the feedstock slurry whereby (i) a first aqueous solution comprising a fermentable carbon source, (ii) a first wet cake comprising solids, and (iii) a stream comprising oil, solids, and an aqueous stream comprising a fermentable carbon source are formed; and
adding the first aqueous solution to a fermentation broth comprising microorganisms whereby a fermentation product is produced.

25. The method of claim 24, further comprising
separating the stream comprising oil, solids, and aqueous stream comprising a fermentable carbon source whereby (i) a second aqueous solution comprising a fermentable carbon source, (ii) a second wet cake comprising solids, and (iii) an oil stream are formed.

26. The method of claim 25, wherein the first and second aqueous solutions are combined prior to the addition to the fermentation broth.

27. The method of claim 25, wherein the second aqueous solution further comprises oil.

28. The method of claim 27, wherein the oil of the second aqueous solution or portion thereof is treated to generate an extractant.

29. The method of claim 28, wherein the oil is treated chemically or enzymatically.

30. A method comprising
providing a feedstock slurry comprising a fermentable carbon source, undissolved solids, and oil;
separating the feedstock slurry whereby (i) a first aqueous solution comprising a fermentable carbon source and solids, (ii) a first wet cake comprising solids, and (iii) a first oil stream are formed; and
adding oil to the first aqueous solution whereby an oil layer comprising solids and a second aqueous solution comprising a fermentable carbon source are formed.

31. The method of claim 30, wherein the oil layer comprising solids is separated forming (i) a second oil stream, (ii) a second wet cake comprising solids, and (iii) a third aqueous solution comprising a fermentable carbon source.

32. The method of claim 31, wherein the second aqueous solution and the third aqueous solution are added to a fermentation broth comprising microorganisms whereby a fermentation product is produced.

33. The method of claim 31, wherein the second aqueous solution and the third aqueous solution further comprise oil.

34. The method of claim 33, wherein the second aqueous solution and the third aqueous solution are combined and the oil of the second aqueous solution and the third aqueous solution or portions thereof is treated to generate an extractant.

35. The method of claim 34, wherein the oil is treated chemically or enzymatically.
